(19) United States
(12) Patent Application Publication
(10) Pub. No.: US 2024/0189275 A1
(43) Pub. Date: Jun. 13, 2024

(54) METHODS FOR TREATING NON-ALCOHOLIC STEATOHEPATITIS WITH A SCD-1 INHIBITOR

(71) Applicant: Lipidio Pharmaceuticals, Inc., San Diego, CA (US)

(72) Inventors: David A. BULLOUGH, Encinitas, CA (US); Jonathan David ROTH, Vail, AZ (US); John Gordon FOULKES, Rancho Santa Fe, CA (US)

(73) Assignee: Lipidio Pharmaceuticals, Inc., San Diego, CA (US)

(21) Appl. No.: 18/546,913

(22) PCT Filed: Feb. 18, 2022
(86) PCT No.: PCT/US2022/016995

§ 371 Date: Aug. 17, 2023

Related U.S. Application Data
(60) Provisional application No. 63/151,511, filed on Feb. 19, 2021.

(51) Int. Cl. A61K 31/40; A61K 9/00; A61K 31/4439; A61K 31/501; A61P 1/16

(52) U.S. Cl. CPC A61K 31/40; A61K 9/0014; A61K 31/4439; A61K 31/501; A61P 1/16

(57) ABSTRACT

The present invention concerns compositions and methods for treating a patient with non-alcoholic steatohepatitis (NASH) by administration, particularly topical administration, of a pharmaceutical composition comprising a therapeutically effective amount of a stearoyl-CoA desaturase (SCD-1) inhibitor. In certain preferred embodiments, the method prevents the development or progression of, or reduces the extent of, liver fibrosis in the patient.

### TECHNICAL FIELD

This application relates to compositions and methods for treating patients with non-alcoholic steatohepatitis (NASH) by administering a stearoyl-CoA desaturase (SCD-1) inhibitor.

### BACKGROUND OF THE INVENTION

Non-alcoholic fatty liver disease (NAFLD) occurs when an excess of fat lipids is stored in liver cells. Nonalcoholic Steatohepatitis (NASH) is an advanced form of NAFLD in which hepatic steatosis is accompanied by hepatocyte injury and inflammation which may occur with or without fibrosis and/or which may progress to fibrosis and cirrhosis. Both NASH and NAFLD are tightly associated with comorbidities of metabolic syndrome, such as insulin resistance and type 2 diabetes, and cardiovascular complications linked to hypertension and dyslipidemia. It has been estimated that within the United States up to 45% of adults have NAFLD, i.e., between 75 to 100 million US adults (Clinical Gastroenterology and Hepatology (2015), 13, 655-657). Although NAFLD can be relatively benign, a percentage of individuals with NAFLD will develop NASH. As in other chronic liver diseases, the progressive severity of underlying hepatic fibrosis is related to clinical outcome(s) and patients with bridging fibrosis or cirrhosis are at the greatest risk of future liver-related morbidities. Increased hepatic fibrogenesis is evidenced by increased hepatic stellate cell activation, which can be shown by increased alpha smooth muscle actin (α-SMA) staining, collagen deposition on biopsy, and an upregulation in circulating serum fibrosis markers, and is associated with a greater risk of progression to cirrhosis and liver cancer (HCC). A recent meta-analysis estimated a 9.57-, 16.69- and 42.3-fold increase in liver-related mortality, in subjects with stage 2, 3 and 4 (cirrhosis) fibrosis vs. subjects with no (stage 0) fibrosis, with only a 1.41-fold mortality increase in patients with stage 1 fibrosis (Hepatology (2017), 65, 1557-1565). Other recent estimates suggest that approximately 20% of patients with NASH with either later stage fibrosis or compensated cirrhosis may progress to cirrhosis or develop decompensation, respectively, over a 2-year period (Hepatology (2019), 70(6), 1885-1888). Thus, the natural history of NAFLD-related liver morbidity and mortality correlates with histologic severity, as determined by the presence of NASH and the stage of fibrosis. To date, there are no medications approved for liver fibrosis or cirrhosis, and the only therapeutic option is liver transplantation for patients with end-stage liver disease (Current Opinion in Pharmacology (2019), 49, 60-70). Given these findings, a therapeutic agent that could prevent or slow the rate of fibrosis progression from benign NAFLD to more advanced disease stages of NASH and of fibrosis would be highly desirable. Treatment of both NASH and NAFLD with the SCD-1 inhibitors of the present invention is envisioned to be advantageous.

Stearoyl-CoA desaturase-1 (SCD-1) is a microsomal enzyme that catalyzes the de novo biosynthesis of monounsaturated fatty acids from saturated fatty acyl-CoA substrates in mammals. Specifically, SCD-1 introduces a cis-double bond in the C9-C10 position of saturated fatty acids such as palmitoyl-CoA (16:0) and stearoyl-CoA (18:0). The resulting mono unsaturated fatty acids, palmitoyl-CoA (16:ln7) and oleoyl-CoA (18:1n9), are in turn substrates for incorporation into a variety of lipids such as phospholipids, cholesterol esters, and triglycerides. Monounsaturated fatty acids are not only key components of cellular structures but are also mediators of important biological processes such as signal transduction and cellular differentiation. Studies in mice suggest that SCD-1 activity is important to maintaining the normal functioning of the skin as a result of its major role in lipid synthesis within sebaceous glands. Indeed, global gene deletion of SCD-1 in mice (Journal of Nutrition (2001), 131(9), 2260-2268) not only results in resistance to high fat diet-induced obesity, but also markedly atrophic sebaceous glands with severely reduced sebum output, SCD-1 expression has been confirmed in the sebaceous glands of human skin by immunohistochemistry (Prostaglandins Leukot Essent Fatty Acids (2003), 68(2), 113-21) and in the immortalized sebaceous gland cell line SZ95 by RT-PCR.

Systemic inhibition of SCD-1 presents an obstacle to the clinical development of oral SCD-1 inhibitors, notably, driven by its key role in the production of Meibomian fluid in the corresponding glands in the eye, with inhibition of this enzyme leading to significant dry eye toxicity. This issue resulted in many pharmaceutical companies abandoning SCD-1 as a potential drug target to treat various metabolic diseases (Scientific Reports (2018), 8(1), 1-8; Journal of Medicinal Chemistry (2011), 54(14), 5082-5096). Interestingly, skin-specific deletion of SCD-1 in mice can also result in the same resistance to high fat diet-induced obesity (Journal of Biological Chemistry (2009), 284(30), 19961-19973). This suggests that 100% inhibition of SCD-1 in the skin alone, if also inhibited in the entire body surface area of the skin (100% Body Surface Area or BSA), can have a metabolic impact to reduce body fat. Unexpectedly however, we have discovered that the topical delivery of SCD-1 inhibitors to just a small area of the skin can not only effect total body fat throughout the body, but surprisingly can also effect the development and progression of fibrosis in NASH. We also discovered that SCD-1 was only inhibited at the site of application to the skin further demonstrating that only partial inhibition of SCD-1 in the skin was sufficient for a therapeutic effect. Also, unexpectedly, these beneficial effects occur without evidence of either skin or eye toxicity.

Described herein are compositions of SCD-1 inhibitor compounds that are suitable for topical delivery to the skin and methods of using such compositions.

### SUMMARY OF THE INVENTION

The present invention provides methods for treating a patient with non-alcoholic steatohepatitis (NASH), and preventing or slowing its progression, by topical delivery of a SCD-1 inhibitor compound.

Some embodiments disclosed herein provide a method of treating non-alcoholic steatohepatitis (NASH) in a patient topically administering a therapeutically effective amount of an SCD-1 inhibitor compound or pharmaceutically acceptable salt thereof to the patient.

Other embodiments provide a method of treating fibrosis associated with non-alcoholic steatohepatitis (NASH) in a patient comprising topically administering an SCD-1 inhibitor compound or pharmaceutically acceptable salt thereof to the patient in a therapeutically effective amount.

Still other embodiments provide a method of preventing the progression of fibrosis associated with non-alcoholic steatohepatitis (NASH) in a patient comprising administering an effective amount of an SCD-1 inhibitor compound or pharmaceutically acceptable salt thereof to the patient.

Another embodiment provides a method of treating a patient with non-alcoholic steatohepatitis (NASH) but without fibrosis so as to prevent the development of fibrosis which comprises topically applying to the patient's skin an amount of an SCD-1 inhibitor compound effective to prevent the development of fibrosis in the patient.

In another embodiment, the patient with NASH but without fibrosis is characterized by a NASH CRN fibrosis score of 0, and the prevention of the development of fibrosis is characterized by no increase in such histologic score in the patient.

Another embodiment provides a method of treating a patient with NASH and with early-stage fibrosis so as to prevent, or slow the rate of progression of, the patient's fibrosis, which comprises topically applying to the patient's skin an amount of an SCD-1 inhibitor compound effective to prevent, or slow the rate of progression of, the fibrosis to a later-stage fibrosis in the patient.

In another embodiment, the patient with NASH and with early-stage fibrosis is characterized by a NASH CRN fibrosis score of 1-2 and the prevention of the patient's early-stage fibrosis progressing to a later-stage fibrosis is characterized by no increase in the NASH CRN fibrosis score in said patient or the slowing of the rate of progression of the patient's early-stage fibrosis to a later-stage fibrosis is characterized by a reduced rate of increase in the NASH CRN fibrosis score in the patient.

Another embodiment provides a method of treating a patient with NASH and with later-stage fibrosis, so as to reduce the extent of the patient's fibrosis, which comprises topically applying to the patient's skin an amount of an SCD-1 inhibitor compound effective to reduce the extent of fibrosis in the patient.

In one case of this embodiment, the patient with NASH and with later-stage fibrosis is characterized by a NASH CRN fibrosis score of 3 or 4, and the reduction of the extent of fibrosis is characterized by a decrease in the NASH CRN fibrosis score in said patient.

In another embodiment, the patient with NASH and with later-stage fibrosis is characterized a NASH CRN fibrosis score of 3 and the reduction of the extent of fibrosis is characterized by a decrease in such score in said patient.

In another embodiment, the patient with NASH and with later-stage fibrosis is characterized by a NASH CRN fibrosis score of 4, and the reduction of the extent of fibrosis is characterized by a decrease in the NASH CRN fibrosis score in said patient.

Another embodiment provides a method of treating a patient with early-stage fibrosis and is characterized by a NAS score of 2.

Another embodiment provides a method of treating a patient with later-stage fibrosis and is characterized by a NAS score of 1.

Another embodiment provides a method of treating a patient with later-stage fibrosis and is characterized by a NASH CRN fibrosis score of 3 or 4.

Another embodiment disclosed provides a method of preventing the development of later-stage fibrosis comprises prevention of progression, or a reduced rate of progression relative to a patient not treated with an SCD-1 inhibitor compound.

Another embodiment provides a method as described above which further comprises inhibiting progression of NASH or optimally resolution of NASH in the patient.

Another embodiment provides a method of treating the patient with an amount of an anti-NASH compound effective to inhibit progression of, or to effect resolution of, NASH.

Yet another embodiment provides a method of preventing the development of later-stage fibrosis which is characterized by no significant change in histologic score at one or more of 2, 4, 8, 24, 40, 52, 65, 72, or 96 weeks; or 2, 3, or 4 years after commencement of administration of a SCD-1 inhibitor compound.

In another embodiment there is provided a method of decreasing a histologic score at 2, 4, 8, 24, 40, 52, 65, 72, or 96 weeks; or at 2, 3, or 4 years after commencement of administration of the SCD-1 inhibitor compound.

In some embodiments, the no increase in, or slowed rate of increase in, a histological score is observed at 2, 4, 8, 24, 40, 52, 65, 72, or 96 weeks; or at 2, 3, or 4 years after commencement of administration of the SCD-1 inhibitor compound.

In some embodiments, the no increase in, or slowed rate of increase in the NASH CRN fibrosis score is observed at 2, 4, 8, 24, 40, 52, 65, 72, or 96 weeks; or at 2, 3, or 4 years after commencement of administration of the SCD-1 inhibitor compound.

In some embodiments, the decrease in the NASH CRN fibrosis score is observed at 2, 4, 8, 24, 40, 52, 65, 72, or 96 weeks; or at 2, 3, or 4 years after commencement of administration of the SCD-1 inhibitor compound.

Another embodiment provides a method of treatment with a SCD-1 inhibitor compound that results in an improvement in liver histology.

Yet another embodiment provides a method of treatment with a SCD-1 inhibitor compound that results in no worsening of liver histology.

In still another embodiment thee is provided a method of preventing, reducing the incidence of, delaying onset of, reducing severity or normalizing liver triglycerides, in a subject after initiation of treatment.

Another embodiment provides a method of preventing, reducing the incidence of, delaying onset of, reducing severity of, conditions associated with increased levels of liver triglycerides in the patient.

In another embodiment there is provided a method of preventing, reducing the incidence of, delaying onset of, reducing severity or normalizing the ratio of lipid-containing to non-lipid containing hepatocytes, in a subject after the initiation of treatment.

Another embodiment provides a method of preventing, reducing the incidence of, delaying onset of, reducing severity of, conditions associated with changes in the ratio of lipid-containing to non-lipid containing hepatocytes in the patient.

In another embodiment a method of preventing, reducing the incidence of, delaying onset of, reducing severity or normalizing the level of hepatic inflammation, in a subject after the initiation of treatment is provided

Another embodiment provides a method of preventing, reducing the incidence of, delaying onset of, reducing severity of, conditions associated with changes in the level of hepatic inflammation in the patient.

Another embodiment provides a method of preventing, reducing the incidence of, delaying onset of, reducing severity or normalizing fibrillar and matrix forming collagens (e.g., Collagen 1a1 content), in a subject after initiation of treatment.

In still another embodiment there is provided a method of preventing, reducing the incidence of, delaying onset of, or reducing severity of conditions associated with fibrillar and matrix forming collagens in the patient.

Another embodiment provides a method of preventing, reducing the incidence of, delaying onset of, reducing severity or normalizing hepatic stellate cell activation (e.g., α-SMA content), in a subject after the initiation of treatment.

In another embodiment a method of preventing, reducing the incidence of, delaying onset of, reducing severity of, conditions associated with hepatic stellate cell activation (e.g., α-SMA content) in the patient is provided.

Another embodiment provides a method of preventing, reducing the incidence of, delaying onset of, reducing severity or normalizing liver lipids, in a subject after the initiation of treatment.

Yet another embodiment provides a method of preventing, reducing the incidence of, delaying onset of, reducing severity of hepatocellular ballooning in the patient.

Another embodiment provides a method of preventing, reducing the incidence of, delaying onset of, reducing severity of hepatic steatosis in the patient.

In another embodiment there is provided a method of treating a patient with early-stage fibrosis with a SCD-1 inhibitor compound, wherein the SCD-1 inhibitor compound is topically applied to less than 30% of the patient's body surface area (BSA). In some embodiments, the SCD-1 inhibitor compound is topically applied to less than 20% of the patient's BSA. In other embodiments, the SCD-1 inhibitor is topically applied to less than 10% of the patient's BSA. In some embodiments, the SCD-1 inhibitor is topically applied to less than 5% of the patient's BSA. In a preferred embodiment, the SCD-1 inhibitor is topically applied to less than 2% of the patient's BSA, and in a particularly preferred embodiment, the SCD-1 inhibitor is topically applied to less than 1% of the patient's BSA.

Another embodiment provides a method of treating a patient with later-stage fibrosis with an SCD-1 inhibitor compound, wherein the SCD-1 inhibitor compound is topically applied to less than 30% of the patient's body surface area (BSA), desirably to less than 20% of the patient's BSA. In some embodiments, the SCD-1 inhibitor is topically applied to less than 10% of the patient's BSA, more desirably to less than 5% of the patient's BSA. In some embodiments, the SCD-1 inhibitor is preferably topically applied to less than 2% of the patient's BSA, and in some embodiments, the SCD-1 inhibitor is more preferably topically applied to less than 1% of the patient's BSA.

In another embodiment there is provided a method of treating a patient with late-stage fibrosis with a SCD-1 inhibitor compound, wherein the SCD-1 inhibitor compound is topically applied to 30% of the patient's body surface area (BSA), in some embodiments, the SCD-1 inhibitor compound is topically applied to 20% of the patient's BSA, in some embodiments, the SCD-1 inhibitor is topically applied to 10% of the patient's BSA, in some embodiments, the SCD-1 inhibitor is topically applied to 5% of the patient's BSA, in some embodiments, the SCD-1 inhibitor is topically applied to 2% of the patient's BSA, and in some embodiments, the SCD-1 inhibitor is topically applied to 1% of the patient's BSA.

Another embodiment provides a method of treating a patient with early-stage fibrosis with a SCD-1 inhibitor compound, wherein the SCD-1 inhibitor compound is topically applied to about 1-2% of the patient's body surface area (BSA).

Yet another embodiment provides a method of treating a patient with later-stage fibrosis with a SCD-1 inhibitor compound, wherein the SCD-1 inhibitor compound is topically applied to about 1-2% of the patient's body surface area (BSA).

Still another embodiment provides a method of treating a patient with later-stage fibrosis with a SCD-1 inhibitor compound, wherein the SCD-1 inhibitor compound is topically applied to about 1-2% of the patient's body surface area (BSA).

In another embodiment there is provided a method of treating a patient with early-stage fibrosis with a SCD-1 inhibitor compound, wherein the SCD-1 inhibitor compound is topically applied to the patient's skin once a day, twice a day, once every two days, once every week, or once every two weeks.

In another embodiment there is provided a method of treating a patient with later-stage fibrosis with a SCD-1 inhibitor compound, wherein the SCD-1 inhibitor compound is topically applied to the patient's skin once a day, twice a day, once every two days, once every week, or once every two weeks.

Another embodiment provides a method of treating a patient with late-stage fibrosis with a SCD-1 inhibitor compound, wherein the SCD-1 inhibitor compound is topically applied to the patient's skin once a day, twice a day, once every two days, once every week, or once every two weeks.

Another embodiment provides a method of treating a patient with early-stage fibrosis and is characterized by a statistically significant decrease in liver fibrosis as determined by either imaging or one or more biomarkers of liver fibrosis.

Yet another embodiment provides a method of treating a patient with later-stage fibrosis characterized by a statistically significant decrease in liver fibrosis determined by either imaging or one or more biomarkers of liver fibrosis.

In still another embodiment there is provided a method of treating a patient with late-stage fibrosis characterized by a statistically significant decrease in liver fibrosis determined by either imaging or one or more biomarkers of liver fibrosis.

Another embodiment provides a method of treating a patient with fearly-stage fibrosis characterized by at least a 20% decrease in fibrosis as compared to pretreatment levels; desirably at least a 25% decrease. In some embodiments, the decrease in fibrosis is at least 30%, desirably at least 35%; and more desirably the decrease in fibrosis is at least 40%. In preferred embodiments, the decrease in fibrosis is at least 45%; more preferably at least 50%. In particularly preferred embodiments, the decrease in fibrosis is at least 55%, at least 60%, at least 65%, or at least 70%. In tmbodhe most preferred embodiments, the decrease in fibrosis is at least 75%, at least 80%, or at least 85%. In the most preferred embodiments, the decrease in fibrosis is at least 90%, at least 95%, or 100%.

Another embodiment disclosed herein is a method of treating a patient with later-stage fibrosis and is characterized by at least 20% decreased in fibrosis by compared to pretreatment levels; in some embodiments, the decrease in fibrosis is at least 25%; in some embodiments, the decrease in fibrosis is at least 30%; in some embodiments, the decrease in fibrosis is at least 35%; in some embodiments, the decrease in fibrosis is at least 40%; in some embodiments, the decrease in fibrosis is at least 25%; in some embodiments, the decrease in fibrosis is at least 45%; in some embodiments, the decrease in fibrosis is at least 50%; in some embodiments, the decrease in fibrosis is at least 55%; in some embodiments, the decrease in fibrosis is at least 60%; in some embodiments, the decrease in fibrosis is at least 65%; in some embodiments, the decrease in fibrosis is at least 70%; in some embodiments, the decrease in fibrosis is at least 75%; in some embodiments, the decrease in fibrosis is at least 80%; in some embodiments, the decrease in fibrosis is at least 85%; in some embodiments, the decrease in fibrosis is at least 90%; in some embodiments, the decrease in fibrosis is at least 95%; and in some embodiments, the decrease in fibrosis is 100%.

Another embodiment disclosed herein is a method of treating a patient with late-stage fibrosis and is characterized by at least 20% decreased in fibrosis by compared to pretreatment levels; in some embodiments, the decrease in fibrosis is at least 25%; in some embodiments, the decrease in fibrosis is at least 30%; in some embodiments, the decrease in fibrosis is at least 35%; in some embodiments, the decrease in fibrosis is at least 40%; in some embodiments, the decrease in fibrosis is at least 25%; in some embodiments, the decrease in fibrosis is at least 45%; in some embodiments, the decrease in fibrosis is at least 50%; in some embodiments, the decrease in fibrosis is at least 55%; in some embodiments, the decrease in fibrosis is at least 60%; in some embodiments, the decrease in fibrosis is at least 65%; in some embodiments, the decrease in fibrosis is at least 70%; in some embodiments, the decrease in fibrosis is at least 75%; in some embodiments, the decrease in fibrosis is at least 80%; in some embodiments, the decrease in fibrosis is at least 85%; in some embodiments, the decrease in fibrosis is at least 90%; in some embodiments, the decrease in fibrosis is at least 95%; and in some embodiments, the decrease in fibrosis is 100%.

One embodiment disclosed herein involves administering an SCD-1 inhibitor compound having the structure of Formula I:

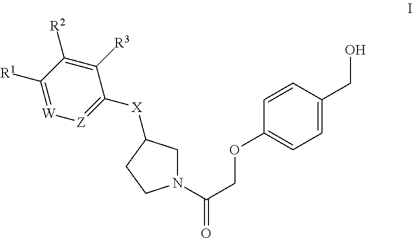

as well as prodrug forms and pharmaceutically acceptable salts thereof.

In some embodiments of Formula (I):
X is selected from the group consisting of O, NH, N-alkyl or N-acyl, S, SO and SO₂;
W is independently CR⁴ or N;
Z is independently CR⁵ or N;
each R¹, R², R³, R⁴ and R⁵ is independently selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, O-alkyl, O-cycloalkyl, O-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹⁰;
wherein adjacent substituents R¹, R², R³, R⁴ and R⁵ may form a saturated or unsaturated 5-membered or 6-membered carbocyclic or heterocyclic ring;
each R⁶, R⁷, R⁸, R⁹ and R¹⁰ is independently selected from the group consisting of H, OH, O-Rx, optionally substituted alkyl, cycloalkyl, heterocycloalkyl, alkylheterocycloalkyl, optionally substituted alkenyl, optionally substituted alkynyl, optionally substituted aryl, optionally substituted alkylaryl, optionally substituted heteroaryl, and optionally substituted alkylheteroaryl; and
Rx is selected from the group consisting of alkyl, cycloalkyl, alkylcycloalkyl, acyl, ester, or thioester.

In other embodiments of Formula (I):
X is O;
W is CR⁴;
Z is CR⁵; and
R¹, R², R⁴ and R⁵ are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments, the structure of Formula (I) is

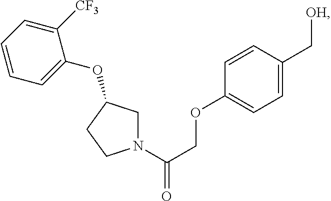 [SMILES: O=C(COc1ccc(CO)cc1)N1CC[C@H](Oc2ccccc2C(F)(F)F)C1]

or a pharmaceutically acceptable salt thereof.

One embodiment disclosed herein includes administering an SCD-1 inhibitor compound having the structure of Formula II:

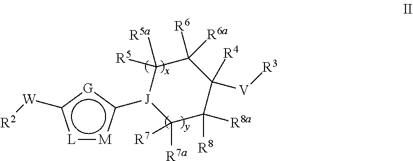

as well as prodrugs and pharmaceutically acceptable salts thereof.

One embodiment disclosed herein includes administering an SCD-1 inhibitor compound having the structure of Formula III:

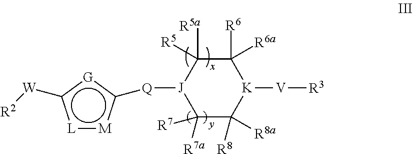

as well as prodrugs and pharmaceutically acceptable salts thereof.

One embodiment disclosed herein includes administering an SCD-1 inhibitor compound having the structure of Formula IV:

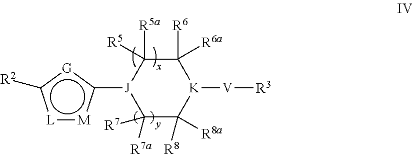

as well as prodrugs and pharmaceutically acceptable salts thereof.

One embodiment disclosed herein includes administering an SCD-1 inhibitor compound having the structure of Formula V:

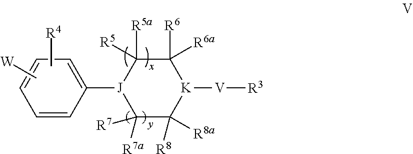

as well as prodrugs and pharmaceutically acceptable salts thereof.

One embodiment disclosed herein includes administering an SCD-1 inhibitor compound having the structure of Formula VI:

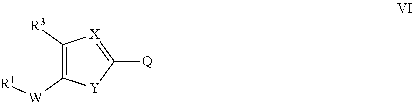

as well as prodrugs and pharmaceutically acceptable salts thereof.

One embodiment disclosed herein includes administering an SCD-1 inhibitor compound having the structure of Formula VII:

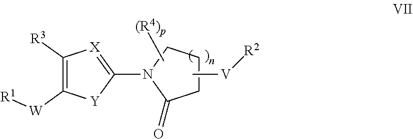

as well as prodrugs and pharmaceutically acceptable salts thereof.

One embodiment disclosed herein includes administering an SCD-1 inhibitor compound having the structure of Formula VIII:

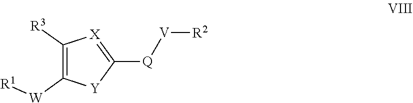

as well as prodrugs and pharmaceutically acceptable salts thereof.

One embodiment disclosed herein includes administering an SCD-1 inhibitor compound having the structure of Formula IX:

R¹—W—P-Q-R² IX

as well as prodrugs and pharmaceutically acceptable salts thereof.

One embodiment disclosed herein includes administering an SCD-1 inhibitor compound having the structure of Formula X:

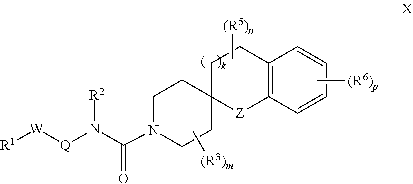

as well as prodrugs and pharmaceutically acceptable salts thereof.

One embodiment disclosed herein includes administering an SCD-1 inhibitor compound having the structure of Formula XI:

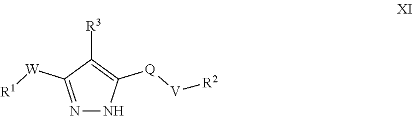

as well as prodrugs and pharmaceutically acceptable salts thereof.

One embodiment disclosed herein includes administering an SCD-1 inhibitor compound having the structure of Formula XII:

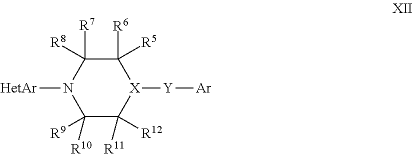

as well as prodrugs and pharmaceutically acceptable salts thereof.

One embodiment disclosed herein includes administering an SCD-1 inhibitor compound having the structure of Formula XIII:

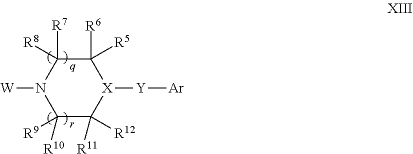

as well as prodrugs and pharmaceutically acceptable salts thereof.

One embodiment disclosed herein includes administering an SCD-1 inhibitor compound having the structure of Formula XIV:

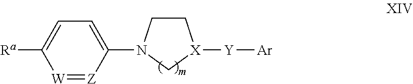

as well as prodrugs and pharmaceutically acceptable salts thereof.

One embodiment disclosed herein includes administering an SCD-1 inhibitor compound having the structure of Formula XV:

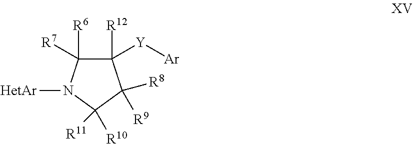

as well as prodrugs and pharmaceutically acceptable salts thereof.

One embodiment disclosed herein includes administering an SCD-1 inhibitor compound having the structure of Formula XVI:

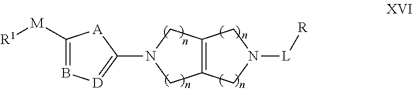

as well as prodrugs and pharmaceutically acceptable salts thereof.

One embodiment disclosed herein includes administering an SCD-1 inhibitor compound having the structure of Formula XVII:

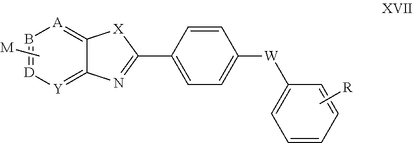

as well as prodrugs and pharmaceutically acceptable salts thereof.

One embodiment disclosed herein includes administering an SCD-1 inhibitor compound having the structure of Formula XVIII:

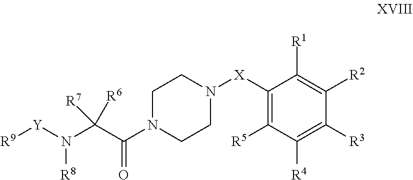

as well as prodrugs and pharmaceutically acceptable salts thereof.

One embodiment disclosed herein includes administering an SCD-1 inhibitor compound having the structure of Formula XIX:

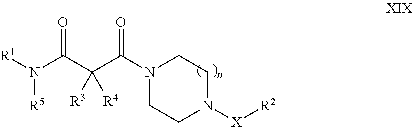

as well as prodrugs and pharmaceutically acceptable salts thereof.

One embodiment disclosed herein includes administering an SCD-1 inhibitor compound having the structure of Formula XX:

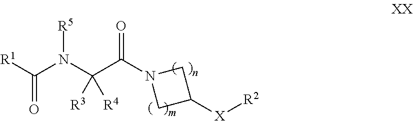

as well as prodrugs and pharmaceutically acceptable salts thereof.

One embodiment disclosed herein includes administering an SCD-1 inhibitor compound having the structure of Formula XXI:

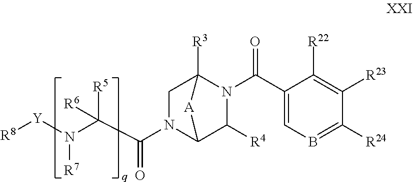

as well as prodrugs and pharmaceutically acceptable salts thereof.

One embodiment disclosed herein includes administering an SCD-1 inhibitor compound having the structure of Formula XXII:

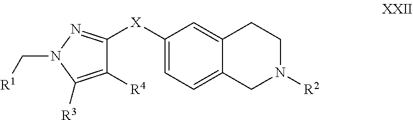

as well as prodrugs and pharmaceutically acceptable salts thereof.

One embodiment disclosed herein includes administering an SCD-1 inhibitor compound having the structure of Formula XXIII:

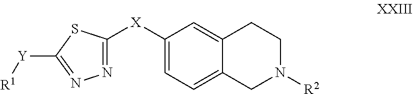

as well as prodrugs and pharmaceutically acceptable salts thereof.

One embodiment disclosed herein includes administering an SCD-1 inhibitor compound having the structure of Formula XXIV:

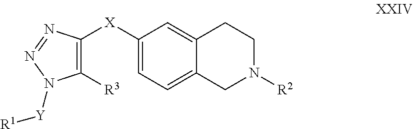

as well as prodrugs and pharmaceutically acceptable salts thereof.

One embodiment disclosed herein includes administering an SCD-1 inhibitor compound having the structure of Formula XXV:

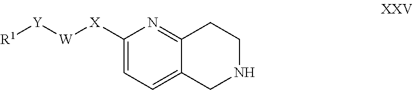

as well as prodrugs and pharmaceutically acceptable salts thereof.

One embodiment disclosed herein includes administering an SCD-1 inhibitor compound having the structure of Formula XXVI:

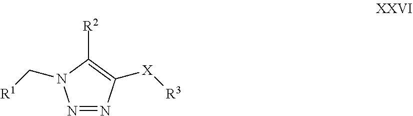

as well as prodrugs and pharmaceutically acceptable salts thereof.

One embodiment disclosed herein includes administering an SCD-1 inhibitor compound having the structure of Formula XXVII:

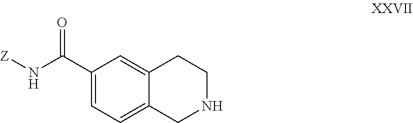

as well as prodrugs and pharmaceutically acceptable salts thereof.

One embodiment disclosed herein includes administering an SCD-1 inhibitor compound having the structure of Formula XXVIII:

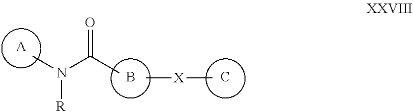

as well as prodrugs and pharmaceutically acceptable salts thereof.

One embodiment disclosed herein includes administering an SCD-1 inhibitor compound having the structure of Formula XXIX:

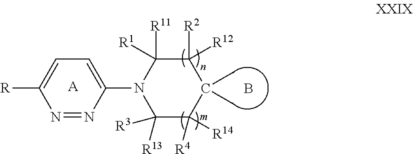

as well as prodrugs and pharmaceutically acceptable salts thereof.

One embodiment disclosed herein includes administering an SCD-1 inhibitor compound having the structure of Formula XXX:

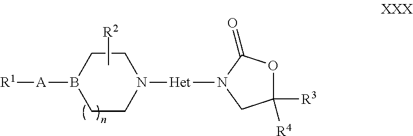

as well as prodrugs and pharmaceutically acceptable salts thereof.

One embodiment disclosed herein includes administering an SCD-1 inhibitor compound having the structure of Formula XXXI:

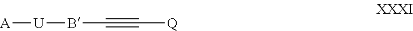

as well as prodrugs and pharmaceutically acceptable salts thereof.

One embodiment disclosed herein includes administering an SCD-1 inhibitor compound having the structure of Formula XXXII:

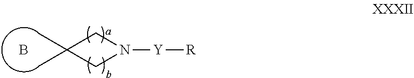

as well as prodrugs and pharmaceutically acceptable salts thereof.

One embodiment disclosed herein includes administering an SCD-1 inhibitor compound having the structure of Formula XXXIII:

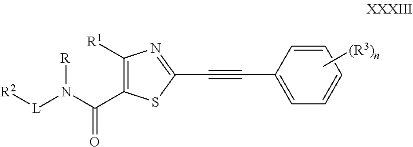

as well as prodrugs and pharmaceutically acceptable salts thereof.

One embodiment disclosed herein includes administering an SCD-1 inhibitor compound having the structure of Formula XXXIV:

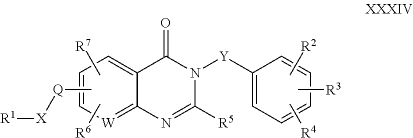

as well as prodrugs and pharmaceutically acceptable salts thereof.

One embodiment disclosed herein includes administering an SCD-1 inhibitor compound having the structure of Formula XXXV:

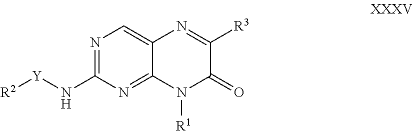

as well as prodrugs and pharmaceutically acceptable salts thereof.

One embodiment disclosed herein includes administering an SCD-1 inhibitor compound having the structure of Formula XXXVI:

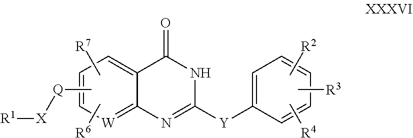

as well as prodrugs and pharmaceutically acceptable salts thereof.

One embodiment disclosed herein includes administering an SCD-1 inhibitor compound having the structure of Formula XXXVII:

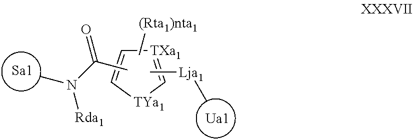

as well as prodrugs and pharmaceutically acceptable salts thereof.

One embodiment disclosed herein includes administering an SCD-1 inhibitor compound having the structure of Formula XXXVIII:

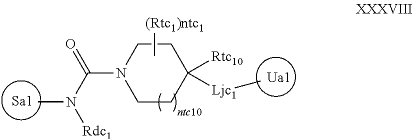

as well as prodrugs and pharmaceutically acceptable salts thereof.

One embodiment disclosed herein includes administering an SCD-1 inhibitor compound having the structure of Formula XXXIX:

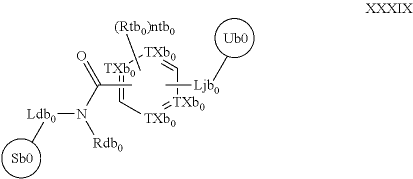

as well as prodrugs and pharmaceutically acceptable salts thereof.

One embodiment disclosed herein includes administering an SCD-1 inhibitor compound having the structure of Formula XL:

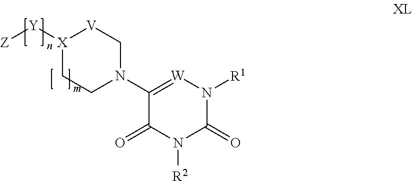

as well as prodrugs and pharmaceutically acceptable salts thereof.

One embodiment disclosed herein includes administering an SCD-1 inhibitor compound having the structure of Formula XLI:

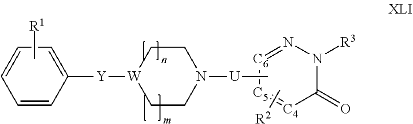

as well as prodrugs and pharmaceutically acceptable salts thereof.

One embodiment disclosed herein includes administering an SCD-1 inhibitor compound having the structure of Formula XLII:

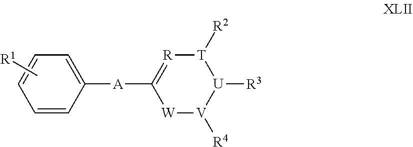

as well as prodrugs and pharmaceutically acceptable salts thereof.

One embodiment disclosed herein includes administering an SCD-1 inhibitor compound having the structure of Formula XLIII:

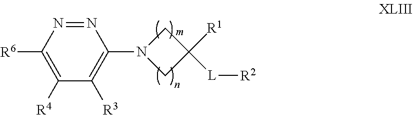

as well as prodrugs and pharmaceutically acceptable salts thereof.

One embodiment disclosed herein includes administering an SCD-1 inhibitor compound having the structure of Formula XLIV:

W—X-G XLIV

as well as prodrugs and pharmaceutically acceptable salts thereof.

Other embodiments disclosed herein include administering the compound of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV at least once a week, on a continuous daily dosing schedule, once a day, twice a day, or three times a day.

One embodiment includes a method of treating non-alcoholic steatohepatitis (NASH) with a compound of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV.

Some embodiments disclosed herein a method of treating fibrosis associated with non-alcoholic steatohepatitis (NASH) with a compound of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV.

Some embodiments disclosed herein a method of preventing the progression of fibrosis associated with non-alcoholic steatohepatitis (NASH) with a compound of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV.

It is to be understood that both the foregoing general description and the following detailed description are exemplary and explanatory only and are not restrictive of the disclosure, as claimed.

### BRIEF DESCRIPTION OF THE FIGURES

The effects of topical treatment of Compound 25 to 10% of the skin for 12 weeks were assessed in a diet-induced and biopsy-confirmed mouse model of NASH described in the literature (World Journal of Gastroenterology (2019), 25(33), 4904-4920). In this model, NASH is induced by maintaining mice on a high fat-high fructose diet (the so-called GAN [Novel Gubra Amylin] diet) for 33 weeks prior to drug administration. A biopsy is performed prior to drug treatment to eliminate any mice that do not develop the NASH phenotype. This biopsy is then compared to a post-drug treatment biopsy to assess changes in NAFLD activity and Fibrosis Stage. Qualitative scoring is completed with quantitative immunohistochemistry to assess effects on hepatic steatosis, inflammation (Galectin-3), fibrosis (Collagen 1a1 and Picro-Sirius Red Staining) and stellate cell activation (α-SMA). RNASeq was also performed on terminal liver samples from mice treated with placebo gel and compound 25 gel to examine key candidate genes involved in extracellular matrix formation (ECM) and fibrogenesis. Parametric (ANOVA) and non-parametric (Fisher's or Chi Square) statistics were used to analyze the data as appropriate.

FIG. 1 shows the concentration-dependent inhibition of both rat and human SCD-1 activity by compound 25 and the IC₅₀s determined for inhibition of rat and human SCD-1.

FIG. 2 is a bar graph showing the effects of compound 25 on body weight (expressed as percent baseline body weight) and body adiposity (expressed as percent body weight). Mice (16 animals per group) were dosed with either a placebo gel, or a gel containing 0.05% of compound 25, 0.25% of compound 25, 0.75% of compound 25 or 2.5% of compound 25 for 12 weeks. Body weight and adiposity were significantly reduced only in the 2.5% (p<0.001) compound 25 treated group only. Thus, in the lower dose groups, improvements in NASH histological parameters can be interpreted as being independent of a pharmacological effect to reduce body weight and body adiposity.

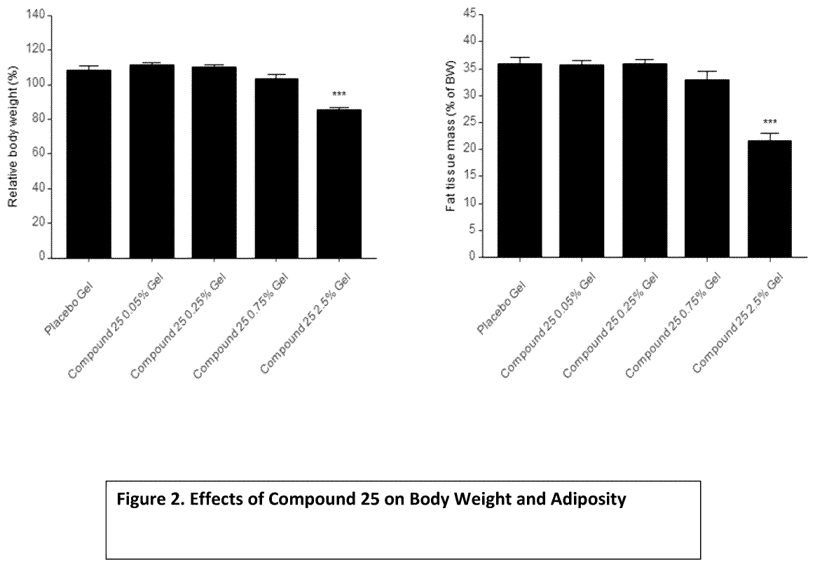

FIG. 3 is a stacked bar graph showing the number of mice in each treatment group that experienced either an improvement (lower score), no change (same score), or a worsening (higher score) in their NAFLD activity score (NAS), which is a scoring system used in clinical NASH trials. The NAS (scored 0-8) is a composite measure of steatosis (liver fat; scored 0-3), lobular inflammation (#inflammatory foci per field; scored 0-3) and hepatocyte ballooning (#cells; scored 0-2). Mice (16 animals per group) were dosed for 12 weeks with either a placebo gel, a gel containing 0.25% of compound 25, a gel containing 0.75% of compound 25 or a gel containing 2.5% of compound 25. The number of animals experiencing an improvement in NAS was significantly greater in both the 0.75% and 2.5% Compound 25 dose groups (p<0.05 compared to placebo), with none of the animals showing a higher NAS score and 15/16 animals a lower NAS score. This finding is consistent with a therapeutic effect both to prevent the progression of NASH and to decrease existing NAS.

FIG. 4 includes two bar graphs showing the liver lipid content quantified from hematoxylin and eosin-stained sections and the liver triglyceride content analyzed biochemically at week 12. Mice (16 animals per group) were dosed with either a placebo gel, a gel containing 0.25% of compound 25, a gel containing 0.75% of compound 25, or a gel containing 2.5% of compound 25. Liver lipid content was significantly reduced in the 0.75% (p<0.05) and 2.5% (p<0.001) compound 25 treated groups. Likewise, liver triglyceride content was significantly reduced in the compound 25 2.5% treated group (p<0.001). Hepatic steatosis and triglyceride accumulation in the cytoplasm of hepatocytes are hallmarks of NAFLD and NASH.

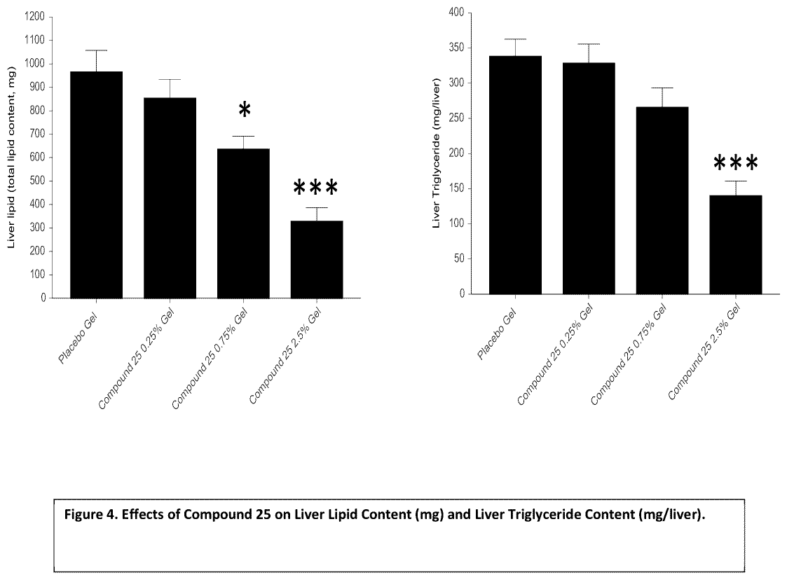

FIG. 5 is a bar graph showing the effects of compound 25 on the number of mice within each treatment group that experienced a worsening (higher score) in their Fibrosis Stage score during the 12 weeks of dosing. Mice (16 animals per group) were dosed with either a placebo gel, a gel containing 0.25% of compound 25, a gel containing 0.75% of compound 25 or a gel containing 2.5% of compound 25. Groups that were treated with 0.25%, 0.75% and 2.5% compound 25 had a dramatically lower number of mice exhibiting any worsening of the fibrosis stage (only 6-12% or 2/16, 2/16 and 1/16 animals, respectively) compared to almost 50% of the animals treated with the Placebo Gel control (7/16). These effects to reduce fibrosis stage scores suggest that compound 25 prevents the progression of fibrosis and fibrogenic processes in NASH.

FIG. 6 is a bar graph showing the liver content of Collagen 1a1 (in mg) in antibody-stained sections. Mice (16 animals per group) were dosed with either a placebo gel, or a gel containing 0.05% of compound 25, 0.25% of compound 25, 0.75% of compound 25 or 2.5% of compound 25 for 12 weeks. Collagen 1a1 is the major fibrillary collagen isoform involved in NASH fibrotic processes. Liver content levels were determined from Collagen 1a1 antibody-stained sections as described in a previous publication (WJG (2019) 25(33):4904-4920). A clear trend in reduction was seen at in quantitative levels of Collagen 1a1 at all doses of compound 25, with significant reduction in the 0.75% (p<0.05) and 2.5% (p<0.01) dose groups, consistent with both prevention of the Fibrosis stage worsening as well as reduced fibrosis. This was unexpected and again suggestive of a therapeutic fibrosis benefit in NASH.

FIG. 7 is a bar graph showing the liver content of Picro-Sirius Red (in mg). Mice (16 animals per group) were dosed with either a placebo gel, a gel containing 0.25% of compound 25, a gel containing 0.75% of compound 25 or a gel containing 2.5% of compound 25. Picro-Sirius Red is a commonly used rapid histological stain to visualize collagen in paraffin-embedded tissue sections. Compound 25 0.75% (p<0.05) and 2.5% (p<0.001) significantly reduced quantitative levels of Picro-Sirius Red. These findings are consistent with the aforementioned improvements in Fibrosis stage scores and quantitative reduction in liver Collagen 1a1 content.

FIG. 8 is a bar graph showing the liver content (in mg) of α-SMA in antibody-stained sections at week 12. Hepatic stellate cells once activated to myofibroblasts become the principal collagen and ECM-producing cells in fibrotic livers. The extent of activation and the presence of myofibroblasts in the liver can be determined by measuring α-SMA expression. Mice (16 animals per group) were dosed with either a placebo gel, a gel containing 0.05% of compound 25, a gel containing 0.25% of compound 25, a gel containing 0.75% of compound 25, or a gel containing 2.5% of compound 25. Compound 25 0.25% (p<0.05), 0.75% (p<0.01) and 2.5% (p<0.001) treated mice had significantly reduced activation of α-SMA consistent with decreased collagen markers and reduced fibrosis progression. These findings also suggest that upon longer term dosing with compound 25 beyond 12 weeks, an even greater degree of antifibrotic activity could be anticipated.

FIG. 9 is a bar graph showing the liver Galectin-3 content (in mg) in antibody-stained sections at week 12. Galectin-3 is a β-galactoside-binding molecule with an important role in hepatic inflammation and is a contributor to disease progression activation of stellate cells and fibrogenesis (Gastroenterology (2020): 158:1334-1345). Mice (16 animals per group) were dosed with either a placebo gel, a gel containing 0.25% of compound 25, a gel containing 0.75% of compound 25, or a gel containing 2.5% of compound 25. Compound 25 0.75% (p<0.05) and 2.5% (p<0.001) treated mice had significantly reduced levels of Galectin-3 content suggesting an anti-inflammatory therapeutic effect.

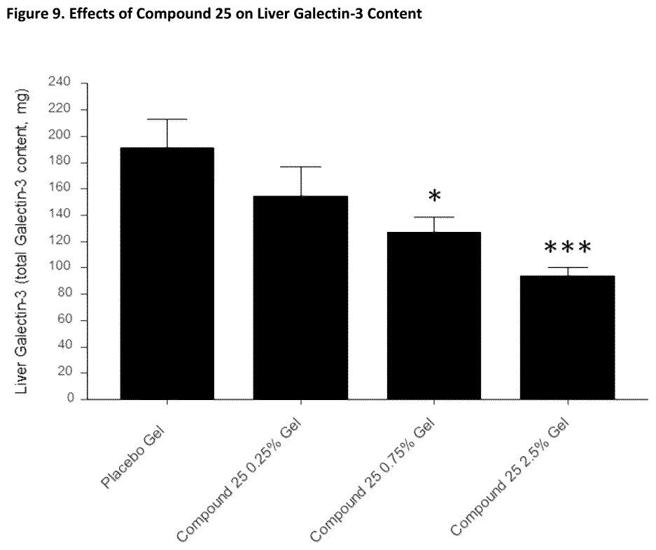

FIG. 10 includes a series of bar graphs showing mRNA expression levels for multiple genes involved in hepatic inflammation. Chemokine Ligand 2 and Chemokine Ligand 5 are pleiotropic cytokines that induce inflammatory recruitment and fibrosis. Epithelial Cell Adhesion Protein promotes adhesive interaction that mediate migration of cells to sites of inflammation. Cluster of Differentiation 68 is a routine marker of inflammation associated with the involvement monocytes and macrophages. Integrin X is a heterotrimeric receptor that is activated in inflammatory processes. Mice (16 animals per group) were dosed with either a placebo gel, or a gel containing 2.5% of compound 25. All of these genes were significantly downregulated in the liver by topical dosing of the animals on the skin with compound 25. Reduced Galectin-3 mRNA is highly consistent with the Galectin-3 histological finding (FIG. 9). Taken together, these other markers further suggest that compound 25 has beneficial effects upon multiple inflammatory processes in the liver.

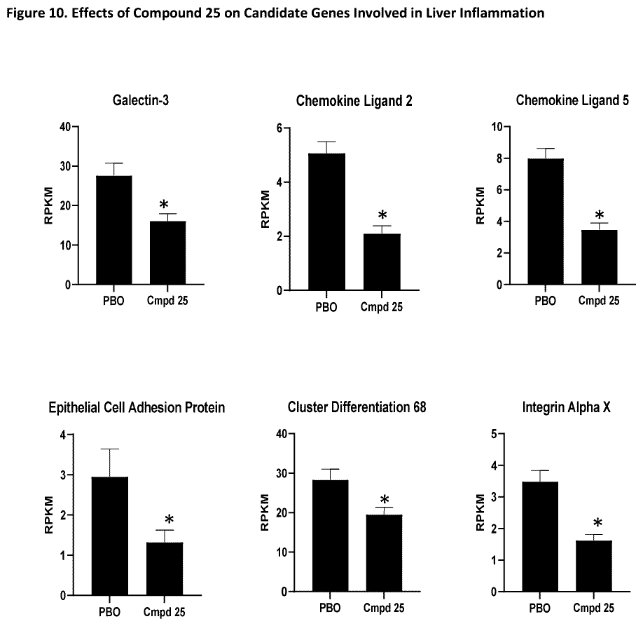

FIG. 11 includes a series of bar graphs showing mRNA expression levels for multiple liver genes involved in NASH fibrogenesis and extracellular matrix formation. Monocyte Chemoattractant Protein-1 attracts other molecules to the extracellular matrix. Osteonectin is a critical glycoprotein associated with the basement membrane and is increased in NASH. α-SMA is stimulated within activated stellate cells which produce fibrillar (Collagen 1a1, Collagen 3a1) and matrix-forming collagen (Collagen 4a1). Mice (16 animals per group) were dosed with either a placebo gel, or a gel containing 2.5% of compound 25. All of these genes were significantly downregulated in the liver by topical dosing of the animals on the skin with compound 25 and are highly consistent with the other measures demonstrating a clear beneficial effect of compound 25 on fibrosis.

FIG. 12 is a bar graph showing plasma alanine transaminase (ALT) (units/L) and plasma aspartate transaminase (AST) (units/L) at week 12. Mice (16 animals per group) were dosed with either a placebo gel, a gel containing 0.25% of compound 25, a gel containing 0.75% of compound 25, or a gel containing 2.5% of compound 25. ALT and AST are widely used clinical indicators of liver damage or injury from different types of diseases or conditions. Normally, circulating ALT and AST levels are low, but when the liver is damaged more ALT and AST are released into the blood. ALT and AST are widely regarded as clinically reliable and sensitive markers of liver disease progression across multiple manifestations including NAFLD/NASH, alcoholic liver disease, Hepatitis B and Hepatitis C Viral infection, drug induced hepatotoxicity, autoimmune and cholestatic liver diseases. The functional significance of the histological improvements with treatment is evident in the reduction in both of these enzymes in the compound 25 0.75% and 2.5% groups.

FIG. 13 includes a series of bar graphs showing mRNA expression levels for multiple liver genes that have been associated with NASH progression and poor prognosis in patients from bioinformatic, integrative and text-mining approaches in the literature (see for example Int. J. Mol. Sci. (2019), 20, 5594 and Gene (2020) 742, 144549). Many of these genes are involved in lipid metabolism, insulin resistance, inflammation and cancer and have been useful to predict whether patients will progress to liver cirrhosis or liver cancer which are often fatal sequelae of NASH. Hepatic aldehyde dehydrogenases (like Aldh8a1 and Aldh7a1) play important roles in catalyzing aldehydes to carboxylic acids critical in a wide variety of biological processes. Annexin A2 is an inducible, calcium-dependent phospholipid-binding protein known to promote liver fibrosis via von Willebrand factor secretion. Cluster of differentiation 36 (CD36) is a cell surface glycoprotein whose roles include uptake of triglycerides in the liver. Cellular nucleic acid binding protein (CNBP) is a single-stranded DNA-binding protein that preferentially binds to the sterol regulatory element sequences in multiple tissues including the liver. Cell death inducing DFFA like effector C (CIDEC) binds to lipid droplets regulating their enlargement to favor storage and restrict lipolysis and also plays a role in apoptosis. Cyclin D1 is a key regulator of progression through stages of the cell cycle in somatic cells. Fatty acid synthase (Fasn) catalyzes fatty acid synthesis and is known to be elevated in NASH. Heat shock factor binding protein 1 (Hsbp1) regulates cellular responses to thermal and chemical stressors. Kinesin family member 6 (KIF6) mediates the intracellular transport of organelles, complex proteins and mRNAs and has been used as a pharmacogenetic marker for lipid-lowering effects. Nuclear factor erythroid 2-related factor (Nfe212) regulates the expression of antioxidants triggered by injury and inflammation and is a pleiotropic regulator across metabolic, inflammation, autophagy and other responses. Perilipin 2, also known as adipose differentiation-related protein, plays roles in lipid and glucose homeostasis and is known to be elevated in liver diseases. Mice (16 animals per group) were dosed with either a placebo gel, or a gel containing 2.5% of compound 25. The significant regulation of these genes with compound 25, together with the histological improvements in steatosis, inflammation and fibrosis, that compound 25 may prevent poor prognosis and improve multiple long-term outcomes in NASH.

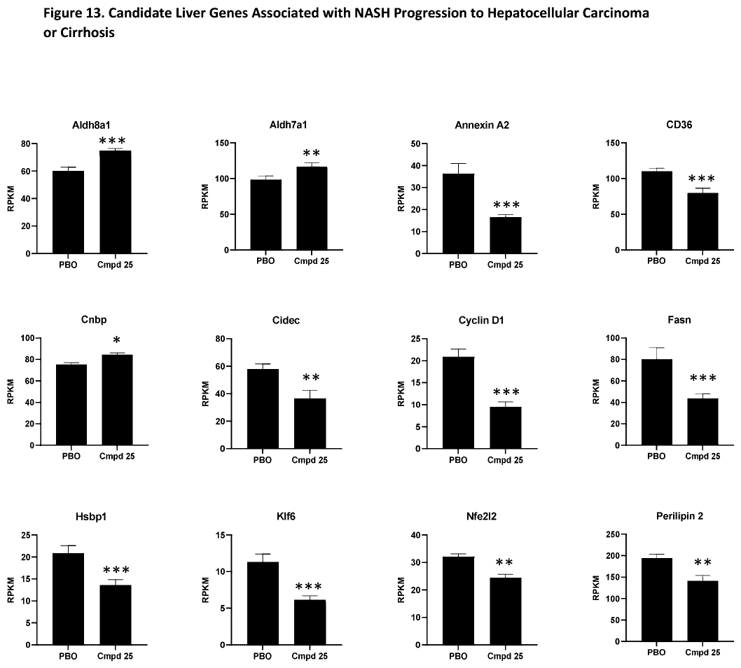

### DETAILED DESCRIPTION

Provided herein are compositions and methods for the prevention or treatment of fibrosis associated with non-alcoholic steatohepatitis (NASH) to reduce its progression by topical delivery to the skin of SCD-1 inhibitor compounds.

In addition to fibrosis associated with NASH, SCD-1 inhibitor compounds may be useful in the treatment of the progression of fibrosis associated with inflammation caused by excess body fat.

The toxic properties of fat may be directly related to its ability to synthesize, modulate, and secrete cytokines and adipokines. This association may be due to the large spectrum of cytokines and metabolites produced by adipose tissue which exhibit pro-inflammatory and cancer prone characteristics. In fact, cytokines produced by adipose tissue triggers insulin resistance and mediates proliferation, migration, angiogenesis and induction of oxidative stress. There are multiple molecular pathways to metabolize fat in the adipocytes. One of the mechanisms to process that fat is by peroxidation. However, when cell has to manage with medium or high rates of lipid peroxidation reaction turns into toxic conditions and oxidative stress abate DNA repair capability, and then cells induce apoptosis that leads to disease. (Journal of Translational Medicine (2016), 14(21), 1-12). A key pathogenic driver of NAFL and NASH, is that the liver's capacity to handle the primary metabolic energy substrates, carbohydrates and fatty acids, is overwhelmed, leading to accumulation of toxic lipid species (Nature Medicine (2018), 24(7), 908-922). These metabolites induce hepatocellular stress, injury and death, leading to fibrogenesis and genomic instability that predispose to cirrhosis and hepatocellular carcinoma.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “alkyl” means a branched, or straight chain chemical group containing only carbon and hydrogen, such as methyl, ethyl, n-propyl, iso-propyl, n-butyl, iso-butyl, sec-butyl, tert-butyl, n-pentyl, iso-pentyl, sec-pentyl and neo-pentyl. Alkyl groups can either be unsubstituted or substituted with one or more substituents. In some embodiments, alkyl groups include 1 to 9 carbon atoms (for example, 1 to 6 carbon atoms, 1 to 4 carbon atoms, or 1 to 2 carbon atoms).

As used herein, “alkylene” means a divalent alkyl, with alkyl as defined above.

As used herein, “alkenyl” means a straight or branched chain, saturated hydrocarbon radical which contains a carbon-carbon double bond. By way of example, the hydrocarbon chain may have from two to twenty carbons, two to sixteen carbons, two to fourteen carbons, two to twelve carbons, two to ten carbons, two to eight carbons, two to six carbons, two to four carbons, etc. “lower alkenyl” may refer to alkenyls having, e.g., two to six carbons, two to four carbons, etc. In certain examples, a straight chain alkenyl may have from two to six carbon atoms and a branched alkyl three to six carbon atoms, e.g., a vinyl group, an allyl group, butene (including all isomeric forms), pentene (including all isomeric forms), and the like. Alkenyl may be optionally substituted. In certain examples, alkenyl may be a C₂ to C₁₂ straight chain or branched chain hydrocarbon containing a carbon-carbon double bond, optionally substituted with oxygen, silicon or sulfur or optionally substituted with OH, 0-alkyl, SH, S-alkyl, NH₂ or NH-alkyl.

As used herein, “alkynyl” means a straight or branched chain, saturated hydrocarbon radical which contains a carbon-carbon triple bond. By way of example, the hydrocarbon chain may have from two to twenty carbons, two to sixteen carbons, two to fourteen carbons, two to twelve carbons, two to ten carbons, two to eight carbons, two to six carbons, two to four carbons, etc. “lower alkynyl” may refer to alkynyls having, e.g., two to six carbons, two to four carbons, etc. In certain examples, a straight chain alkynyl may have from two to six carbon atoms and a branched alkyl three to six carbon atoms, e.g., an acetylene group, a propargyl group, butyne (including all isomeric forms), pentyne (including all isomeric forms), and the like. Alkynyl may be optionally substituted. In certain examples, alkynyl may be a C₂ to C₁₂ straight chain or branched chain hydrocarbon containing a carbon-carbon triple bond, optionally substituted with oxygen, silicon or sulfur or optionally substituted with OH, 0-alkyl, SH, S-alkyl, NH₂ or NH-alkyl.

As used herein, “cycloalkyl” means a cyclic ring system containing only carbon atoms in the ring system backbone, such as cyclopropyl, cyclobutyl, cyclopentyl, cyclohexyl, and cyclohexenyl. Cycloalkyls may include multiple fused rings. Carbocyclyls may have any degree of saturation provided that none of the rings in the ring system are aromatic. Carbocyclyl groups can either be unsubstituted or substituted with one or more substituents. In some embodiments, carbocyclyl groups include 3 to 10 carbon atoms, for example, 3 to 6 carbon atoms.

As used herein, “alkylcycloalkyl” means a (alkylene)-R radical where R is cycloalkyl as defined above; e.g., cyclopropylmethyl, cyclobutylmethyl, cyclopentylethyl, or cyclohexylmethyl, and the like. In another example, alkylcycloalkyl has four to twelve carbon atoms, i.e., C₄-C₁₂ alkylcycloalkyl.

As used herein, “aryl” means a mono-, bi-, tri- or polycyclic group with only carbon atoms present in the ring backbone having 5 to 14 ring atoms, alternatively 5, 6, 9, or 10 ring atoms; and having 6, 10, or 14 pi electrons shared in a cyclic array; wherein at least one ring in the system is aromatic. Aryl groups can either be unsubstituted or substituted with one or more substituents. Examples of aryl include phenyl, naphthyl, tetrahydronaphthyl, 2,3-dihydro-1H-indenyl, and others. In some embodiments, the aryl is phenyl.

As used herein, “alkylaryl” means a (alkylene)-R radical where R is aryl as defined above. Alkylaryl may be optionally substituted. In certain examples, alkylaryl may be alkylphenyl, alkylsubstituted phenyl, alkylnaphthyl or alkylsubstituted naphthyl.

As used herein, the term “heteroaryl” means a mono-, bi-, tri-or polycyclic group having 5 to 14 ring atoms, alternatively 5, 6, 9, or 10 ring atoms; and having 6, 10, or 14 pi electrons shared in a cyclic array; wherein at least one ring in the system is aromatic, and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[3,2-c]pyridinyl, thieno[3,2-b]pyridinyl, thieno[2,3-c]pyridinyl, thieno[2,3-b]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

As used herein, “halo”, “halide” or “halogen” is a chloro, bromo, fluoro, or iodo atom radical. In some embodiments, a halo is a chloro, bromo or fluoro. For example, a halide can be fluoro.

As used herein, “haloalkyl” means a hydrocarbon substituent, which is a linear or branched, alkyl, alkenyl or alkynyl substituted with one or more chloro, bromo, fluoro, and/or iodo atom(s). In some embodiments, a haloalkyl is a fluoroalkyls, wherein one or more of the hydrogen atoms have been substituted by fluoro. In some embodiments, haloalkyls are of 1 to about 3 carbons in length (e.g., 1 to about 2 carbons in length or 1 carbon in length). The term “haloalkylene” means a diradical variant of haloalkyl, and such diradicals may act as spacers between radicals, other atoms, or between a ring and another functional group.

As used herein, “heterocyclyl” or “heterocycloalkyl” means a nonaromatic cyclic ring system comprising at least one heteroatom in the ring system backbone. Heterocyclyls may include multiple fused rings. Heterocyclyls may be substituted or unsubstituted with one or more substituents. In some embodiments, heterocycles have 3-11 members. The heterocyclyl ring is optionally fused to a (one) aryl or heteroaryl ring as defined herein. The heterocyclyl ring fused to monocyclic aryl or heteroaryl ring is also referred to in this application as “bicyclic heterocyclyl” ring. Additionally, one or two ring carbon atoms in the heterocyclyl ring can optionally be replaced by a —CO— group. In six membered monocyclic heterocycles, the heteroatom(s) are selected from one to three of O, N or S, and wherein when the heterocycle is five membered, it can have one or two heteroatoms selected from O, N, or S. Examples of heterocyclyl include azirinyl, aziridinyl, azetidinyl, oxetanyl, thietanyl, 1,4,2-dithiazolyl, dihydropyridinyl, 1,3-dioxanyl, 1,4-dioxanyl, 1,3-dioxolanyl, morpholinyl, thiomorpholinyl, piperazinyl, pyranyl, pyrrolidinyl, tetrahydrofuryl, tetrahydropyridinyl, oxazinyl, thiazinyl, thiinyl, thiazolidinyl, isothiazolidinyl, oxazolidinyl, isoxazolidinyl, piperidinyl, pyrazolidinyl imidazolidinyl, thiomorpholinyl, and others. In some embodiments, the heterocyclyl is selected from azetidinyl, morpholinyl, piperazinyl, pyrrolidinyl, and tetrahydropyridinyl.

As used herein, “alkylheterocycloalkyl” means an -(alkylene)-R radical where R is heterocyclyl ring as defined above e.g., tetraydrofuranylmethyl, piperazinylmethyl, morpholinylethyl, and the like. Alkylheterocycloalkyl also includes, e.g., where the heterocycle comprises one or two hetero atoms selected from O, S, or N and has three to eleven carbon atoms, i.e., C₃ to C₁₁ alkylheterocycloalkyl, and includes when N is present in the heterocyclic ring the nitrogen atom may be in the form of an amide, carbamate or urea.

As used herein, “alkylcycloalkyl” means a (alkylene)-R radical where R is cycloalkyl as defined above; e.g., cyclopropylmethyl, cyclobutylmethyl, cyclopentylethyl, or cyclohexylmethyl, and the like. In another example, alkylcycloalkyl has four to twelve carbon atoms, i.e., C₄-C₁₂ alkylcycloalkyl.

As used herein, “oxo” or “carbonyl” means a ═(O) group or C═O group, respectively.

As used herein, “0-alkyl” means an (oxygen)-R radical where R is alkyl as defined above. For example, 0-alkyl may be an oxygen atom bonded to a C₁ to C₆ straight chain or branched chain alkyl.

As used herein, “0-cycloalkyl” means an (oxygen)-R radical where R is cycloalkyl as defined above. For example, 0-cycloalkyl is an oxygen atom bonded to a C₃ to C₇ cycloalkyl.

As used herein, “0-alkylcycloalkyl” means an (oxygen)-R radical where R is alkylcycloalkyl as defined above. For example, 0-cycloalkyl is an oxygen atom bonded to a C₄ to C₈ alkylcycloalkyl.

The term “substituted” refers to moieties having substituents replacing a hydrogen on one or more non-hydrogen atoms of the molecule. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Substituents can include, for example, —(C₁₋₉ alkyl) optionally substituted with one or more of hydroxyl, —NH₂, —NH(C₁₋₃ alkyl), and —N(C₁₋₃ alkyl)₂; —(C₁₋₉ haloalkyl); a halide; a hydroxyl; a carbonyl [such as —C(═O)OR, and —C(═O)R]; a thiocarbonyl [such as —C(═S)OR, —C(═O)SR, and —C(═S)R]; —(C₁₋₉ alkoxy) optionally substituted with one or more of halide, hydroxyl, —NH₂, —NH(C₁₋₃ alkyl), and —N(C₁₋₃ alkyl)₂; —OPO(OH)₂; a phosphonate [such as —PO(OH)₂ and —PO(OR′)₂]; —OPO(OR′)R″; —NRR′; —C(O)NRR′; —C(NR)NR′R″; —C(NR′)R″; a cyano; a nitro; an azido; —SH; —S—R; —OSO₂(OR); a sulfonate [such as —SO₂(OH) and —SO₂(OR)]; —SO₂NR′R″; and —SO₂R; in which each occurrence of R, R′ and R″ are independently selected from H; —(C₁₋₉ alkyl); C₆₋₁₀ aryl optionally substituted with from 1-3R″′; 5-10 membered heteroaryl having from 1-4 heteroatoms independently selected from N, O, and S and optionally substituted with from 1-3 R″′; C₃₋₇ carbocyclyl optionally substituted with from 1-3 R″′; and 3-8 membered heterocyclyl having from 1-4 heteroatoms independently selected from N, O, and S and optionally substituted with from 1-3 R″′; wherein each R″′ is independently selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl). In some embodiments, the substituent is selected from —(C₁₋₆ alkyl), —(C₁₋₆ haloalkyl), a halide (e.g., F), a hydroxyl, —C(O)OR, —C(O)R, —(C₁₋₆ alkoxyl), —NRR′, —C(O)NRR′, and a cyano, in which each occurrence of R and R′ is independently selected from H and —(C₁₋₆ alkyl).

As used herein, when two groups are indicated to be “linked” or “bonded” to form a “ring”, it is to be understood that a bond is formed between the two groups and may involve replacement of a hydrogen atom on one or both groups with the bond, thereby forming a carbocyclyl, heterocyclyl, aryl, or heteroaryl ring. The skilled artisan will recognize that such rings can and are readily formed by routine chemical reactions. In some embodiments, such rings have from 3-7 members, for example, 5 or 6 members.

The skilled artisan will recognize that some chemical structures described herein may be represented on paper by one or more other resonance forms; or may exist in one or more other tautomeric forms, even when kinetically, the artisan recognizes that such tautomeric forms represent only a very small portion of a sample of such compound(s). Such compounds are clearly contemplated within the scope of this disclosure, though such resonance forms or tautomers are not explicitly represented herein.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

The present disclosure includes all pharmaceutically acceptable isotopically labeled compounds of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV wherein one or more atoms are replaced by atoms having the same atomic number, but an atomic mass or mass number different from the atomic mass or mass number which predominates in nature. Examples of isotopes suitable for inclusion in the compounds of the disclosure include, but are not limited to, isotopes of hydrogen, such as ²H (deuterium) and ³H (tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ¹⁵S.

The term “administration” or “administering” and “delivery” refers to a method of providing a dosage of a compound or pharmaceutical composition to a mammal, where the method is, e.g., topical.

The term “mammal” is used in its usual biological sense. Thus, it specifically includes humans, cattle, horses, monkeys, dogs, cats, mice, rats, cows, sheep, pigs, goats, and non-human primates, but also includes many other species.

The term “pharmaceutically acceptable carrier”, “pharmaceutically acceptable diluent” or “pharmaceutically acceptable excipient” includes any and all solvents, co-solvents, complexing agents, dispersion media, coatings, isotonic and absorption delaying agents and the like which are not biologically or otherwise undesirable. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, NJ. Considerations for the inclusion of various components in pharmaceutical compositions are described, e.g., in Brunton et al. (Eds.) (2017); Goodman and Gilman's: The Pharmacological Basis of Therapeutics, 13th Ed. The McGraw-Hill Companies.

The term “pharmaceutically acceptable salt” refers to salts that retain the biological effectiveness and properties of the compounds provided herein and, which are not biologically or otherwise undesirable. In many cases, the compounds provided herein are capable of forming acid and/or base salts by virtue of the presence of amino and/or carboxyl groups or groups similar thereto. Many such salts are known in the art, for example, as described in WO 87/05297. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and beryllium and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, histidine, arginine, lysine, benethamine, N-methyl-glucamine, and ethanolamine. Other acids include dodecylsufuric acid, naphthalene-1,5-disulfonic acid, naphthalene-2-sulfonic acid, and saccharin.

“Patient” as used herein, means a human or a non-human mammal, e.g., a dog, a cat, a cow, a sheep, a pig, a goat, a non-human primate. In some embodiments, the patient is a human.

A “therapeutically effective amount” of a compound as provided herein is one which is sufficient to achieve the desired physiological effect and may vary according to the nature and severity of the disease condition, and the potency of the compound. “Therapeutically effective amount” is also intended to include one or more of the compounds of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV in combination with one or more other agents that are effective to treat the diseases and/or conditions described herein. The combination of compounds can be a synergistic combination. Synergy, as described, for example, by Chou and Talalay, Advances in Enzyme Regulation (1984), 22, 27-55, occurs when the effect of the compounds when administered in combination is greater than the additive effect of the compounds when administered alone as a single agent. In general, a synergistic effect is most clearly demonstrated at sub-optimal concentrations of the compounds. It will be appreciated that different concentrations may be employed for prophylaxis than for treatment of an active disease. This amount can further depend upon the patient's height, weight, sex, age and medical history.

A therapeutic effect relieves, to some extent, one or more of the symptoms of the disease.

“Treat,” “treatment,” or “treating,” as used herein refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

The term “topical” refers to the application of a suitable compound (e.g., active agent) or composition comprising a compound (e.g., active agent) to the skin to treat diseases or conditions, for example NASH. In some embodiments of topical application, the compound or composition penetrates the epidermis or dermis without significant systemic exposure.

The term “dosing interval” refers to the time between administrations of the two sequential doses of a pharmaceutical's during multiple dosing regimens.

The term “about” when used to refer to a quantitative value means that a specified quantity may be greater than or less than the indicated amount by 1, 2, 3, 4, 5, 6, 7, 8, 9, 10, 11, 12, 13, 14, 15, 16, 17, 18, 19 or 20 percent of the stated numerical value.

### Compounds

The compounds and compositions described herein can be used as anti-fibrotic agents. In addition, the compounds can be used as inhibitors of stearoyl-CoA desaturase (SCD-1). SCD-1 is a microsomal enzyme that catalyzes the de novo biosynthesis of monounsaturated fatty acids from saturated fatty acyl-CoA substrates in mammals.

The compounds and compositions described herein may also be useful in the treatment of fibrosis associated with inflammation caused by fat throughout the body.

In some embodiments, compounds for use as SCD-1 inhibitors include the compounds set forth below as described in the following journal articles, U.S. patents and U.S. patent applications.

In some embodiments, an SCD-1 inhibitor compound is a compound described in U.S. Pat. Nos. 8,242,286; 8,524,761; and 8,980,936, and are hereby incorporated by reference in their entirety.

Some embodiments of the present disclosure include compounds of Formula I:

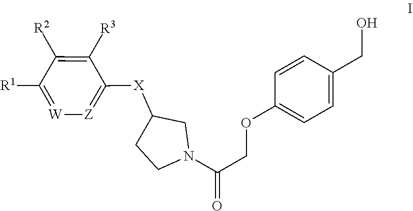

or salts, pharmaceutically acceptable salts, or prodrugs thereof.

In some embodiments of Formula (I), X is selected from the group consisting of O, NH, N-alkyl or N-acyl, S, SO, and SO₂.

In some embodiments of Formula (I), X is O; in some embodiments of Formula (I), X is NH; in some embodiments of Formula (I), X is N-alkyl; in some embodiments of Formula (I), X is N-acyl; in some embodiments of Formula (I), X is S; in some embodiments of Formula (I), X is SO; and in some embodiments of Formula (I), X is SO₂.

In some embodiments of Formula (I), X is selected from the group consisting of NH, N-alkyl and N-acyl.

In some embodiments of Formula (I), X is selected from the group consisting of S, SO, and SO₂.

In some embodiments of Formula (I), W is CR⁴ or N; in some embodiments of Formula (I), W is CR⁴; and in some embodiments of Formula (I), W is N.

In some embodiments of Formula (I), Z is CR⁵ or N; in some embodiments of Formula (I), Z is CR⁵; and in some embodiments of Formula (I), Z is N.

In some embodiments of Formula (I), W is CR⁴ and Z is CR⁵; in some embodiments of Formula (I), W is CR⁴ and Z is N; in some embodiments of Formula (I), W is N and Z is CR⁵; and in some embodiments of Formula (I), W is N and Z is N.

In some embodiments of Formula (I), each R¹, R², R³, R⁴ and R⁵ are independently selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, 0-alkyl, 0-cycloalkyl, 0-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹⁰.

In some embodiments of Formula (I), W is CH and Z is CH; one of R¹, R², or R³ is selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, 0-alkyl, 0-cycloalkyl, 0-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹°.

In some embodiments of Formula (I), W is N and Z is CH; one of R¹, R², or R³ is selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, 0-alkyl, 0-cycloalkyl, 0-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹°.

In some embodiments of Formula (I), W is CH and Z is N; one of R¹, R², or R³ is selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, 0-alkyl, 0-cycloalkyl, 0-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹⁰.

In some embodiments of Formula (I), W is N and Z is N; one of R¹, R², or R³ is selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, 0-alkyl, 0-cycloalkyl, 0-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹⁰.

In some embodiments of Formula (I), each R¹, R², R³, R⁴ and R⁵ are independently selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), W is CH and Z is CH; one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is CH and Z is CH; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is CH and Z is CH; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is CH and Z is CH; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), W is CH and Z is CH; R¹ and R² are H; and R³ is selected from the group consisting of CH₂F, CHF₂, and CF₃; in some embodiments of Formula (I), W is CH and Z is CH; R¹ and R³ are H; and R² is selected from the group consisting of CH₂F, CHF₂, and CF₃; in some embodiments of Formula (I), W is CH and Z is CH; R² and R³ are H; and R¹ is selected from the group consisting of CH₂F, CHF₂, and CF₃.

In some embodiments of Formula (I), W is N and Z is CH; one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is N and Z is CH; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is N and Z is CH; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is N and Z is CH; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), W is N and Z is CH; R¹ and R² are H; and R³ is selected from the group consisting of CH₂F, CHF₂, and CF₃; in some embodiments of Formula (I), W is N and Z is CH; R¹ and R³ are H; and R² is selected from the group consisting of CH₂F, CHF₂, and CF₃; in some embodiments of Formula (I), W is N and Z is CH; R² and R³ are H; and R¹ is selected from the group consisting of CH₂F, CHF₂, and CF₃.

In some embodiments of Formula (I), W is CH and Z is N; one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is CH and Z is N; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is CH and Z is N; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is CH and Z is N; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), W is CH and Z is N; R¹ and R² are H; and R³ is selected from the group consisting of CH₂F, CHF₂, and CF₃; in some embodiments of Formula (I), W is CH and Z is N; R¹ and R³ are H; and R² is selected from the group consisting of CH₂F, CHF₂, and CF₃; in some embodiments of Formula (I), W is CH and Z is N; R² and R³ are H; and R¹ is selected from the group consisting of CH₂F, CHF₂, and CF₃.

In some embodiments of Formula (I), W is N and Z is N; one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is N and Z is N; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is N and Z is N; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), W is N and Z is N; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), W is N and Z is N; R¹ and R² are H; and R³ is selected from the group consisting of CH₂F, CHF₂, and CF₃; in some embodiments of Formula (I), W is N and Z is N; R¹ and R³ are H; and R² is selected from the group consisting of CH₂F, CHF₂, and CF₃; in some embodiments of Formula (I), W is N and Z is N; R² and R³ are H; and R¹ is selected from the group consisting of CH₂F, CHF₂, and CF₃.

In some embodiments of Formula (I), W is N and Z is CR⁵; R¹, R², and R³ are H; and R^{s} is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), W is N and Z is CR⁵; R¹, R², and R³ are H; and R^{s} is selected from the group consisting of CH₂F, CHF₂, and CF₃.

In some embodiments of Formula (I), X is O; W is CH and Z is CH; and one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is CH and Z is CH; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is CH and Z is CH; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is CH and Z is CH; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), X is O; W is CH and Z is CH; R¹ and R² are H; and R³ is selected from the group consisting of CH₂F, CHF₂, and CF₃; in some embodiments of Formula (I), X is O; W is CH and Z is CH; R¹ and R³ are H; and R² is selected from the group consisting of CH₂F, CHF₂, and CF₃; in some embodiments of Formula (I), X is O; W is CH and Z is CH; R² and R³ are H; and R¹ is selected from the group consisting of CH₂F, CHF₂, and CF₃.

In some embodiments of Formula (I), X is O; W is N and Z is CH; and one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is N and Z is CH; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is N and Z is CH; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is N and Z is CH; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), X is O; W is N and Z is CH; R¹ and R² are H; and R³ is selected from the group consisting of CH₂F, CHF₂, and CF₃; in some embodiments of Formula (I), X is O; W is N and Z is CH; R¹ and R³ are H; and R² is selected from the group consisting of CH₂F, CHF₂, and CF₃; in some embodiments of Formula (I), X is O; W is N and Z is CH; R² and R³ are H; and R¹ is selected from the group consisting of CH₂F, CHF₂, and CF₃.

In some embodiments of Formula (I), X is O; W is CH and Z is N; and one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is CH and Z is N; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is CH and Z is N; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is CH and Z is N; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), X is O; W is CH and Z is N; R¹ and R² are H; and R³ is selected from the group consisting of CH₂F, CHF₂, and CF₃; in some embodiments of Formula (I), X is O; W is CH and Z is N; R¹ and R³ are H; and R² is selected from the group consisting of CH₂F, CHF₂, and CF₃; in some embodiments of Formula (I), X is O; W is CH and Z is N; R² and R³ are H; and R¹ is selected from the group consisting of CH₂F, CHF₂, and CF₃.

In some embodiments of Formula (I), X is O; W is N and Z is N; and one of R¹, R², or R³ is selected from the group consisting of H, F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is N and Z is N; R¹ and R² are H; and R³ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is N and Z is N; R¹ and R³ are H; and R² is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃; in some embodiments of Formula (I), X is O; W is N and Z is N; R² and R³ are H; and R¹ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), X is O; W is N and Z is N; R¹ and R² are H; and R³ is selected from the group consisting of CH₂F, CHF₂, and CF₃; in some embodiments of Formula (I), X is O; W is N and Z is N; R¹ and R³ are H; and R² is selected from the group consisting of CH₂F, CHF₂, and CF₃; in some embodiments of Formula (I), X is O; W is N and Z is N; R² and R³ are H; and R¹ is selected from the group consisting of CH₂F, CHF₂, and CF₃.

In some embodiments of Formula (I), X is O; W is N and Z is CR⁵; R¹, R², and R³ are H; and R⁵ is selected from the group consisting of F, Cl, C₁ to C₃ straight chain or branched chain alkyl, CH₂F, CHF₂, CF₃, 0(C₁₋₃ alkyl), OCH₂F, OCHF₂, and OCF₃.

In some embodiments of Formula (I), X is O; W is N and Z is CR⁵; R¹, R², and R³ are H; and R⁵ is selected from the group consisting of CH₂F, CHF₂, and CF₃.

In some embodiments of Formula (I), adjacent substituents R¹, R², R³, R⁴ and R⁵ may form a saturated or unsaturated 5-membered or 6-membered carbocyclic or heterocyclic ring.

In some embodiments of Formula (I), each R⁶, R⁷, R⁸, R⁹ and R¹⁰ are independently selected from the group consisting of H, OH, O-Rx, optionally substituted alkyl, cycloalkyl, heterocycloalkyl, alkylheterocycloalkyl, optionally substituted alkenyl, optionally substituted alkynyl, optionally substituted aryl, optionally substituted alkylaryl, optionally substituted heteroaryl, and optionally substituted alkylheteroaryl.

In some embodiments of Formula (I), Rx is selected from the group consisting of alkyl, cycloalkyl, alkylcycloalkyl, acyl, ester, or thioester.

In some embodiments of Formula (I), X is O; W is CR⁴ or N; Z is CR⁵ or N; each R¹, R², R³, R⁴ and R⁵ are independently selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, O-alkyl, O-cycloalkyl, O-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹⁰; adjacent substituents R¹, R², R³, R⁴ and R⁵ may form a saturated or unsaturated 5-membered or 6-membered carbocyclic or heterocyclic ring; and each R⁶, R⁷, R⁸, R⁹ and R¹⁰ are independently selected from the group consisting of H, optionally substituted alkyl, cycloalkyl, heterocycloalkyl, alkylheterocycloalkyl, optionally substituted alkenyl, optionally substituted alkynyl, optionally substituted aryl, optionally substituted alkylaryl, optionally substituted heteroaryl, and optionally substituted alkylheteroaryl.

Illustrative compounds of Formula (I) are shown in Table 1.

TABLE 1

| | |
|---|---|
| 1 | 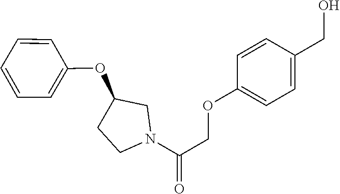 [SMILES: O=C(COc1ccc(CO)cc1)N1CC[C@@H](Oc2ccccc2)C1] |
| 2 | 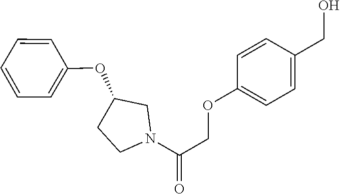 [SMILES: O=C(COc1ccc(CO)cc1)N1CC[C@H](Oc2ccccc2)C1] |
| 3 | 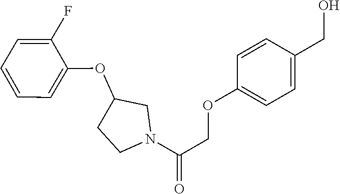 [SMILES: O=C(COc1ccc(CO)cc1)N1CCC(Oc2ccccc2F)C1] |
| 4 | 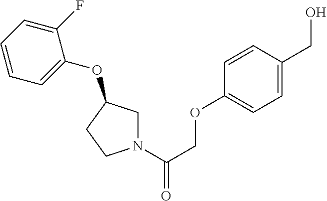 [SMILES: O=C(COc1ccc(CO)cc1)N1CC[C@@H](Oc2ccccc2F)C1] |
| 5 | 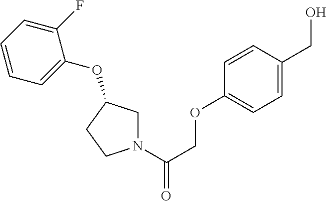 [SMILES: O=C(COc1ccc(CO)cc1)N1CC[C@H](Oc2ccccc2F)C1] |
| 6 | 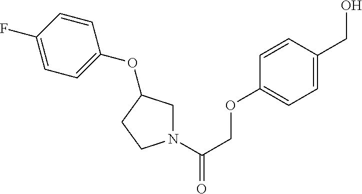 [SMILES: O=C(COc1ccc(CO)cc1)N1CCC(Oc2ccc(F)cc2)C1] |
| 7 | 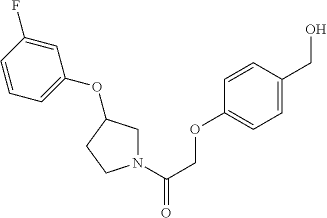 [SMILES: O=C(COc1ccc(CO)cc1)N1CCC(Oc2cccc(F)c2)C1] |
| 8 | 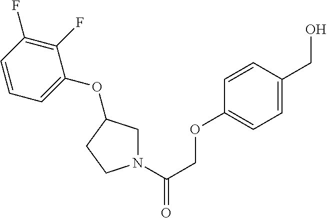 [SMILES: O=C(COc1ccc(CO)cc1)N1CCC(Oc2cccc(F)c2F)C1] |
| 9 | 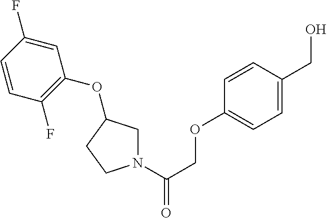 [SMILES: O=C(COc1ccc(CO)cc1)N1CCC(Oc2cc(F)ccc2F)C1] |
| 10 | 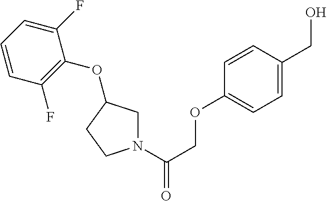 [SMILES: O=C(COc1ccc(CO)cc1)N1CCC(Oc2c(F)cccc2F)C1] |
| 11 | 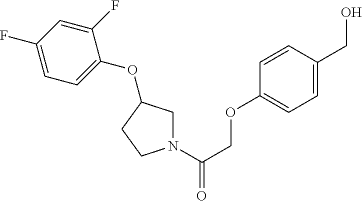 [SMILES: O=C(COc1ccc(CO)cc1)N1CCC(Oc2ccc(F)cc2F)C1] |
| 12 | 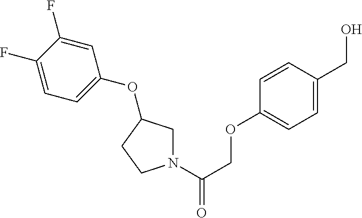 [SMILES: O=C(COc1ccc(CO)cc1)N1CCC(Oc2ccc(F)c(F)c2)C1] |
| 13 | 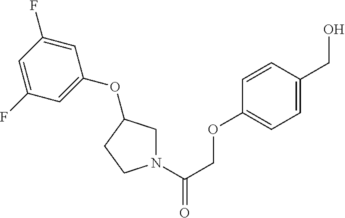 [SMILES: O=C(COc1ccc(CO)cc1)N1CCC(Oc2cc(F)cc(F)c2)C1] |
| 14 | 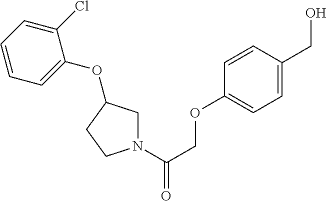 [SMILES: O=C(COc1ccc(CO)cc1)N1CCC(Oc2ccccc2Cl)C1] |
| 15 | 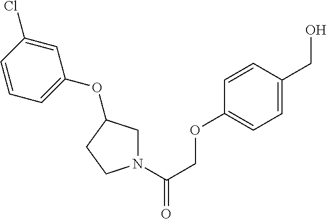 [SMILES: O=C(COc1ccc(CO)cc1)N1CCC(Oc2cccc(Cl)c2)C1] |
| 16 | 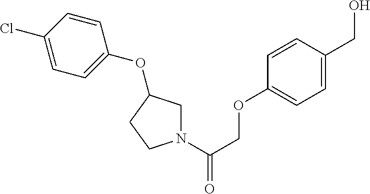 [SMILES: O=C(COc1ccc(CO)cc1)N1CCC(Oc2ccc(Cl)cc2)C1] |
| 17 | 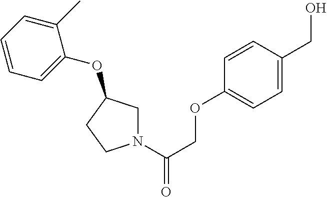 [SMILES: Cc1ccccc1O[C@@H]1CCN(C(=O)COc2ccc(CO)cc2)C1] |
| 18 | 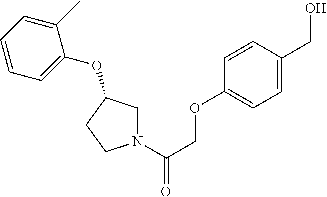 [SMILES: Cc1ccccc1O[C@H]1CCN(C(=O)COc2ccc(CO)cc2)C1] |
| 19 | 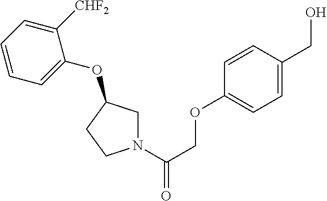 [SMILES: O=C(COc1ccc(CO)cc1)N1CC[C@@H](Oc2ccccc2C(F)F)C1] |
| 20 | 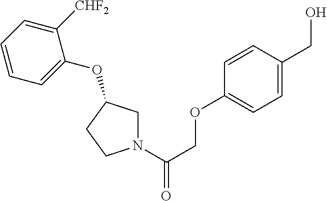 [SMILES: O=C(COc1ccc(CO)cc1)N1CC[C@H](Oc2ccccc2C(F)F)C1] |
| 21 | 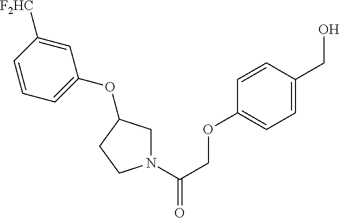 [SMILES: O=C(COc1ccc(CO)cc1)N1CCC(Oc2cccc(C(F)F)c2)C1] |
| 22 | 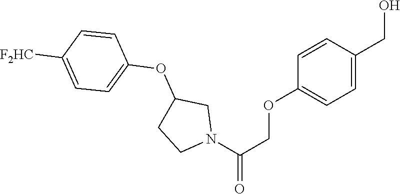 [SMILES: O=C(COc1ccc(CO)cc1)N1CCC(Oc2ccc(C(F)F)cc2)C1] |
| 23 | 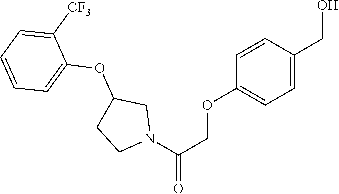 [SMILES: O=C(COc1ccc(CO)cc1)N1CCC(Oc2ccccc2C(F)(F)F)C1] |
| 24 | 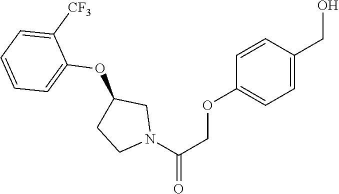 [SMILES: O=C(COc1ccc(CO)cc1)N1CC[C@@H](Oc2ccccc2C(F)(F)F)C1] |
| 25 | 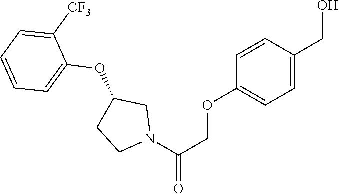 [SMILES: O=C(COc1ccc(CO)cc1)N1CC[C@H](Oc2ccccc2C(F)(F)F)C1] |
| 26 | 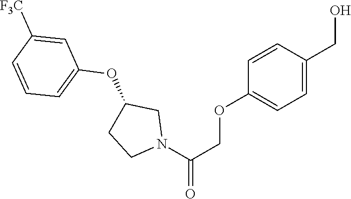 [SMILES: O=C(COc1ccc(CO)cc1)N1CC[C@H](Oc2cccc(C(F)(F)F)c2)C1] |
| 27 | 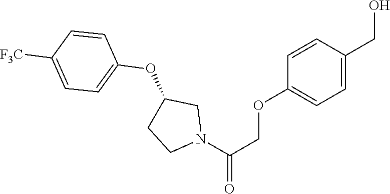 [SMILES: O=C(COc1ccc(CO)cc1)N1CC[C@H](Oc2ccc(C(F)(F)F)cc2)C1] |
| 28 | 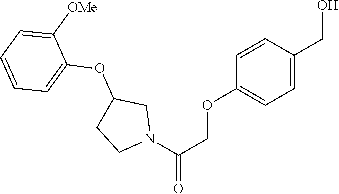 [SMILES: COc1ccccc1OC1CCN(C(=O)COc2ccc(CO)cc2)C1] |
| 29 | 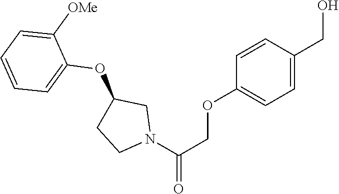 [SMILES: COc1ccccc1O[C@@H]1CCN(C(=O)COc2ccc(CO)cc2)C1] |
| 30 | 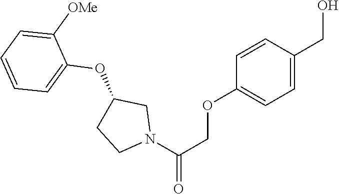 [SMILES: COc1ccccc1O[C@H]1CCN(C(=O)COc2ccc(CO)cc2)C1] |
| 31 | 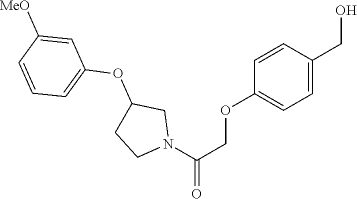 [SMILES: COc1cccc(OC2CCN(C(=O)COc3ccc(CO)cc3)C2)c1] |
| 32 | 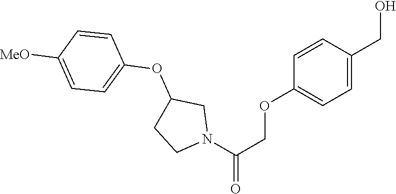 [SMILES: COc1ccc(OC2CCN(C(=O)COc3ccc(CO)cc3)C2)cc1] |
| 33 | 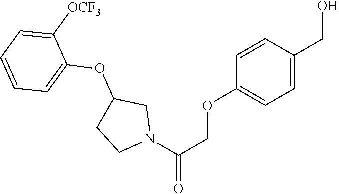 [SMILES: O=C(COc1ccc(CO)cc1)N1CCC(Oc2ccccc2OC(F)(F)F)C1] |
| 34 | 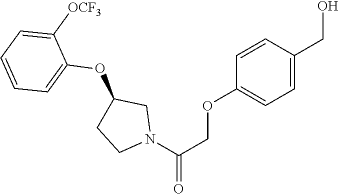 [SMILES: O=C(COc1ccc(CO)cc1)N1CC[C@@H](Oc2ccccc2OC(F)(F)F)C1] |
| 35 | 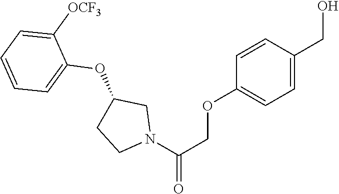 [SMILES: O=C(COc1ccc(CO)cc1)N1CC[C@H](Oc2ccccc2OC(F)(F)F)C1] |
| 36 | 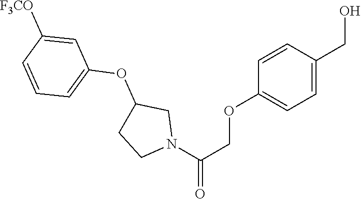 [SMILES: O=C(COc1ccc(CO)cc1)N1CCC(Oc2cccc(OC(F)(F)F)c2)C1] |
| 37 | 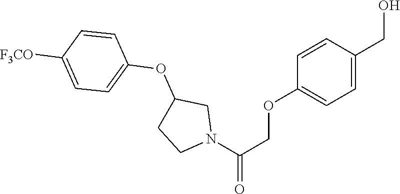 [SMILES: O=C(COc1ccc(CO)cc1)N1CCC(Oc2ccc(OC(F)(F)F)cc2)C1] |
| 38 | 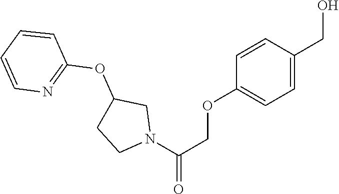 [SMILES: O=C(COc1ccc(CO)cc1)N1CCC(Oc2ccccn2)C1] |
| 39 | 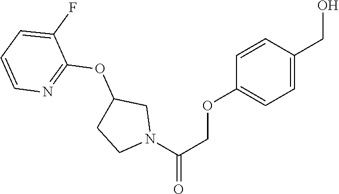 [SMILES: O=C(COc1ccc(CO)cc1)N1CCC(Oc2ncccc2F)C1] |
| 40 | 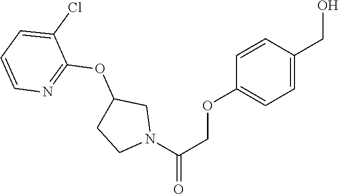 [SMILES: O=C(COc1ccc(CO)cc1)N1CCC(Oc2ncccc2Cl)C1] |
| 41 | 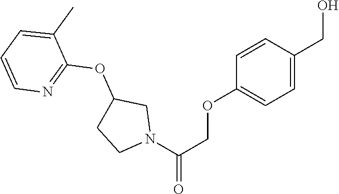 [SMILES: Cc1cccnc1OC1CCN(C(=O)COc2ccc(CO)cc2)C1] |
| 42 | 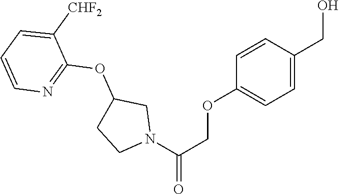 [SMILES: O=C(COc1ccc(CO)cc1)N1CCC(Oc2ncccc2C(F)F)C1] |
| 43 | 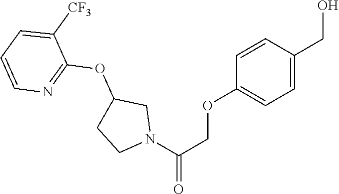 [SMILES: O=C(COc1ccc(CO)cc1)N1CCC(Oc2ncccc2C(F)(F)F)C1] |
| 44 | 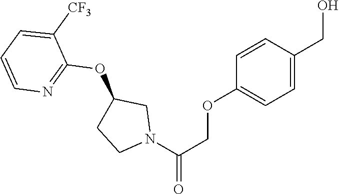 [SMILES: O=C(COc1ccc(CO)cc1)N1CC[C@@H](Oc2ncccc2C(F)(F)F)C1] |
| 45 | 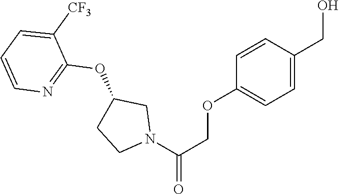 [SMILES: O=C(COc1ccc(CO)cc1)N1CC[C@H](Oc2ncccc2C(F)(F)F)C1] |
| 46 | 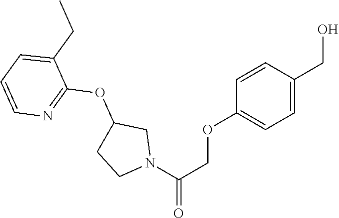 [SMILES: CCc1cccnc1OC1CCN(C(=O)COc2ccc(CO)cc2)C1] |
| 47 | 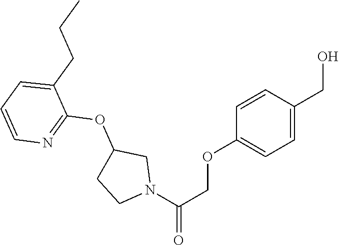 [SMILES: CCCc1cccnc1OC1CCN(C(=O)COc2ccc(CO)cc2)C1] |
| 48 | 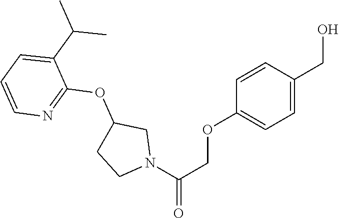 [SMILES: CC(C)c1cccnc1OC1CCN(C(=O)COc2ccc(CO)cc2)C1] |
| 49 | 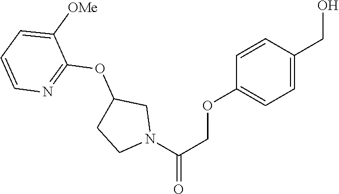 [SMILES: COc1cccnc1OC1CCN(C(=O)COc2ccc(CO)cc2)C1] |
| 50 | 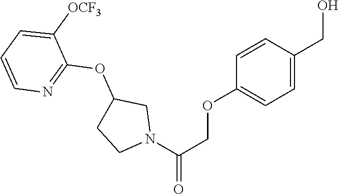 [SMILES: O=C(COc1ccc(CO)cc1)N1CCC(Oc2ncccc2OC(F)(F)F)C1] |
| 51 | 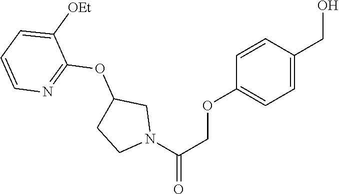 [SMILES: CCOc1cccnc1OC1CCN(C(=O)COc2ccc(CO)cc2)C1] |
| 52 | 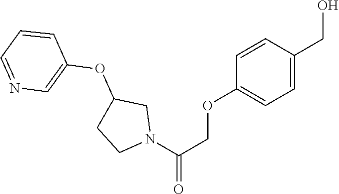 [SMILES: O=C(COc1ccc(CO)cc1)N1CCC(Oc2cccnc2)C1] |
| 53 | 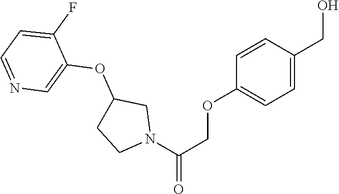 [SMILES: O=C(COc1ccc(CO)cc1)N1CCC(Oc2cnccc2F)C1] |
| 54 | 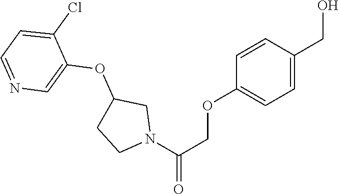 [SMILES: O=C(COc1ccc(CO)cc1)N1CCC(Oc2cnccc2Cl)C1] |
| 55 | 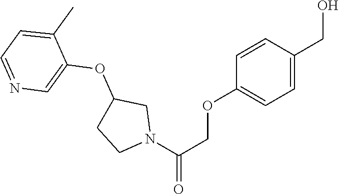 [SMILES: Cc1ccncc1OC1CCN(C(=O)COc2ccc(CO)cc2)C1] |
| 56 | 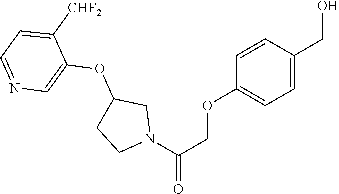 [SMILES: O=C(COc1ccc(CO)cc1)N1CCC(Oc2cnccc2C(F)F)C1] |
| 57 | 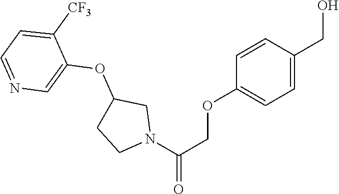 [SMILES: O=C(COc1ccc(CO)cc1)N1CCC(Oc2cnccc2C(F)(F)F)C1] |
| 58 | 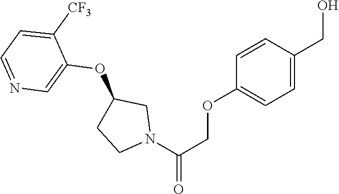 [SMILES: O=C(COc1ccc(CO)cc1)N1CC[C@@H](Oc2cnccc2C(F)(F)F)C1] |
| 59 | 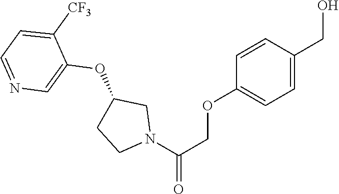 [SMILES: O=C(COc1ccc(CO)cc1)N1CC[C@H](Oc2cnccc2C(F)(F)F)C1] |
| 60 | 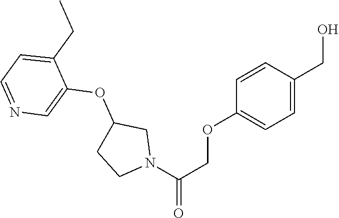 [SMILES: CCc1ccncc1OC1CCN(C(=O)COc2ccc(CO)cc2)C1] |
| 61 | 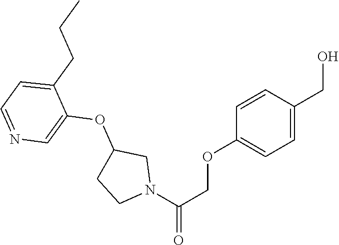 [SMILES: CCCc1ccncc1OC1CCN(C(=O)COc2ccc(CO)cc2)C1] |
| 62 | 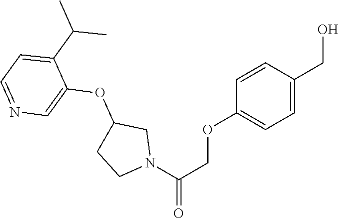 [SMILES: CC(C)c1ccncc1OC1CCN(C(=O)COc2ccc(CO)cc2)C1] |
| 63 | 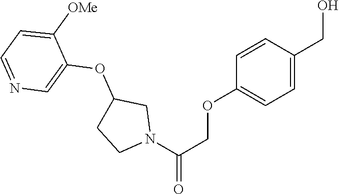 [SMILES: COc1ccncc1OC1CCN(C(=O)COc2ccc(CO)cc2)C1] |
| 64 | 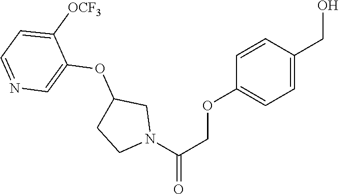 [SMILES: O=C(COc1ccc(CO)cc1)N1CCC(Oc2cnccc2OC(F)(F)F)C1] |
| 65 | 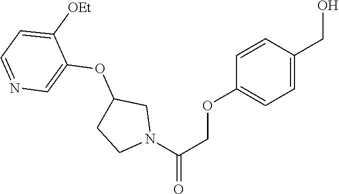 [SMILES: CCOc1ccncc1OC1CCN(C(=O)COc2ccc(CO)cc2)C1] |
| 66 | 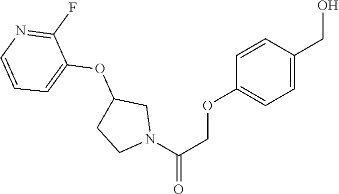 [SMILES: O=C(COc1ccc(CO)cc1)N1CCC(Oc2cccnc2F)C1] |
| 67 | 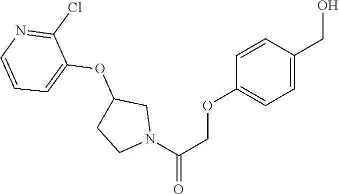 [SMILES: O=C(COc1ccc(CO)cc1)N1CCC(Oc2cccnc2Cl)C1] |
| 68 | 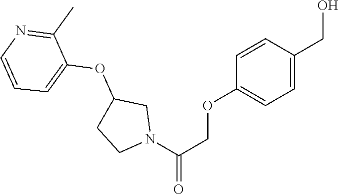 [SMILES: Cc1ncccc1OC1CCN(C(=O)COc2ccc(CO)cc2)C1] |
| 69 | 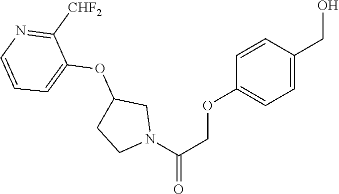 [SMILES: O=C(COc1ccc(CO)cc1)N1CCC(Oc2cccnc2C(F)F)C1] |
| 70 | 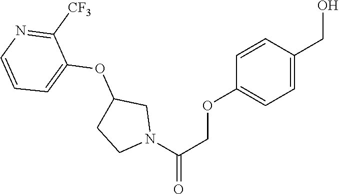 [SMILES: O=C(COc1ccc(CO)cc1)N1CCC(Oc2cccnc2C(F)(F)F)C1] |
| 71 | 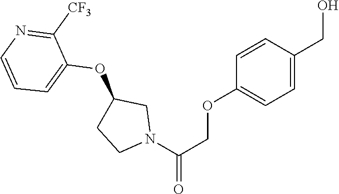 [SMILES: O=C(COc1ccc(CO)cc1)N1CC[C@@H](Oc2cccnc2C(F)(F)F)C1] |
| 72 | 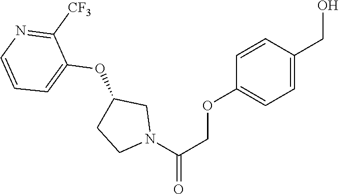 [SMILES: O=C(COc1ccc(CO)cc1)N1CC[C@H](Oc2cccnc2C(F)(F)F)C1] |
| 73 | 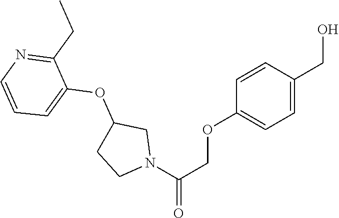 [SMILES: CCc1ncccc1OC1CCN(C(=O)COc2ccc(CO)cc2)C1] |
| 74 | 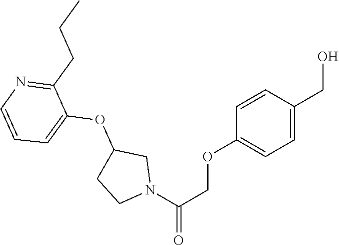 [SMILES: CCCc1ncccc1OC1CCN(C(=O)COc2ccc(CO)cc2)C1] |
| 75 | 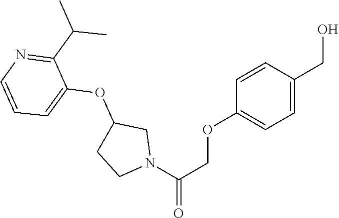 [SMILES: CC(C)c1ncccc1OC1CCN(C(=O)COc2ccc(CO)cc2)C1] |
| 76 | 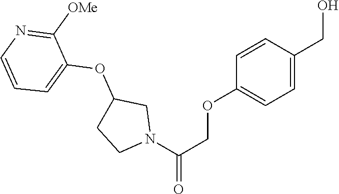 [SMILES: COc1ncccc1OC1CCN(C(=O)COc2ccc(CO)cc2)C1] |
| 77 | 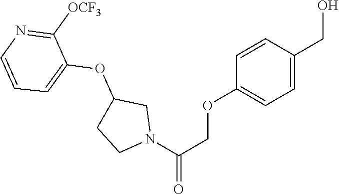 [SMILES: O=C(COc1ccc(CO)cc1)N1CCC(Oc2cccnc2OC(F)(F)F)C1] |
| 78 | 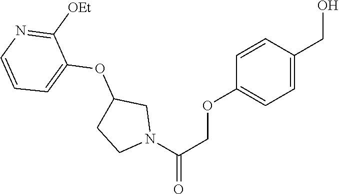 [SMILES: CCOc1ncccc1OC1CCN(C(=O)COc2ccc(CO)cc2)C1] |
| 79 | 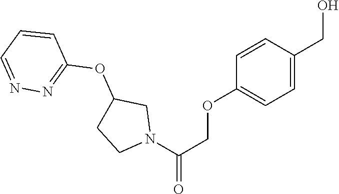 [SMILES: O=C(COc1ccc(CO)cc1)N1CCC(Oc2cccnn2)C1] |
| 80 | 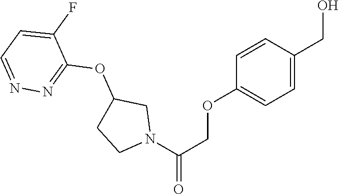 [SMILES: O=C(COc1ccc(CO)cc1)N1CCC(Oc2nnccc2F)C1] |
| 81 | 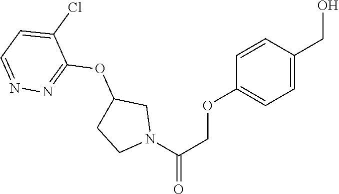 [SMILES: O=C(COc1ccc(CO)cc1)N1CCC(Oc2nnccc2Cl)C1] |
| 82 | 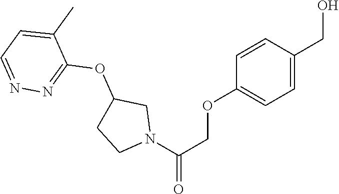 [SMILES: Cc1ccnnc1OC1CCN(C(=O)COc2ccc(CO)cc2)C1] |
| 83 | 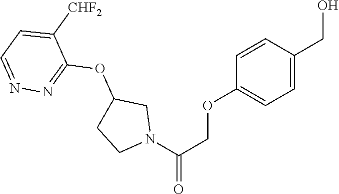 [SMILES: O=C(COc1ccc(CO)cc1)N1CCC(Oc2nnccc2C(F)F)C1] |
| 84 | 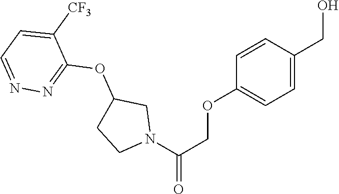 [SMILES: O=C(COc1ccc(CO)cc1)N1CCC(Oc2nnccc2C(F)(F)F)C1] |
| 85 | 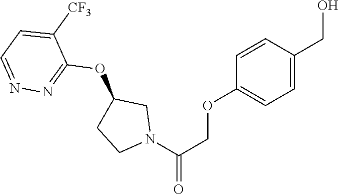 [SMILES: O=C(COc1ccc(CO)cc1)N1CC[C@@H](Oc2nnccc2C(F)(F)F)C1] |
| 86 | 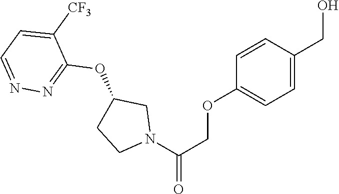 [SMILES: O=C(COc1ccc(CO)cc1)N1CC[C@H](Oc2nnccc2C(F)(F)F)C1] |
| 87 | 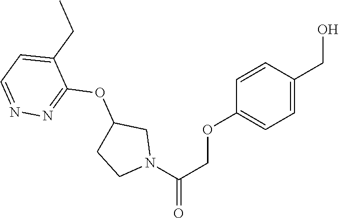 [SMILES: CCc1ccnnc1OC1CCN(C(=O)COc2ccc(CO)cc2)C1] |
| 88 | 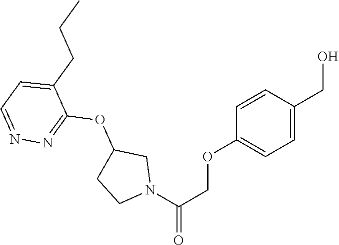 [SMILES: CCCc1ccnnc1OC1CCN(C(=O)COc2ccc(CO)cc2)C1] |
| 89 | 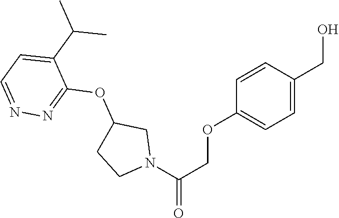 [SMILES: CC(C)c1ccnnc1OC1CCN(C(=O)COc2ccc(CO)cc2)C1] |
| 90 | 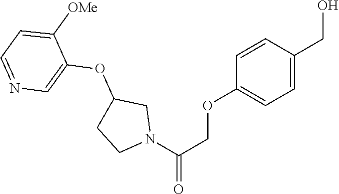 [SMILES: COc1ccncc1OC1CCN(C(=O)COc2ccc(CO)cc2)C1] |
| 91 | 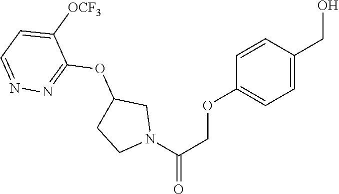 [SMILES: O=C(COc1ccc(CO)cc1)N1CCC(Oc2nnccc2OC(F)(F)F)C1] |
| 92 | 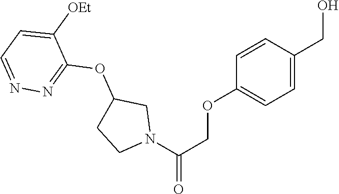 [SMILES: CCOc1ccnnc1OC1CCN(C(=O)COc2ccc(CO)cc2)C1] |
| 93 | 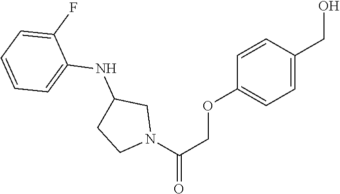 [SMILES: O=C(COc1ccc(CO)cc1)N1CCC(Nc2ccccc2F)C1] |
| 94 | 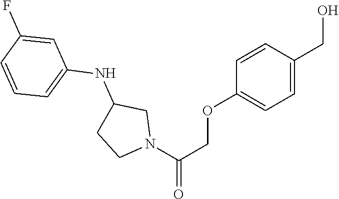 [SMILES: O=C(COc1ccc(CO)cc1)N1CCC(Nc2cccc(F)c2)C1] |
| 95 | 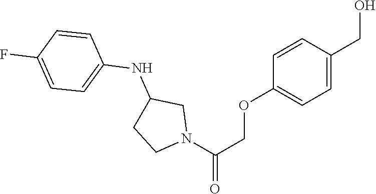 [SMILES: O=C(COc1ccc(CO)cc1)N1CCC(Nc2ccc(F)cc2)C1] |
| 96 | 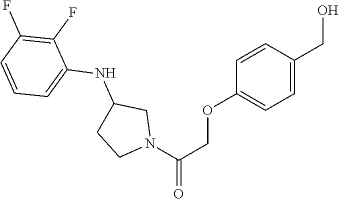 [SMILES: O=C(COc1ccc(CO)cc1)N1CCC(Nc2cccc(F)c2F)C1] |
| 97 | 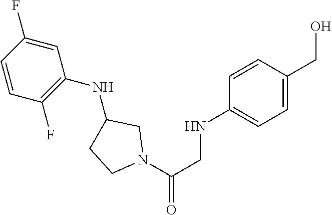 [SMILES: O=C(CNc1ccc(CO)cc1)N1CCC(Nc2cc(F)ccc2F)C1] |
| 98 | 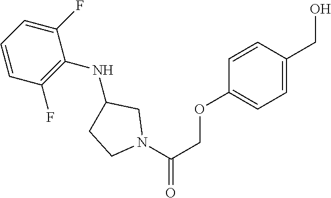 [SMILES: O=C(COc1ccc(CO)cc1)N1CCC(Nc2c(F)cccc2F)C1] |
| 99 | 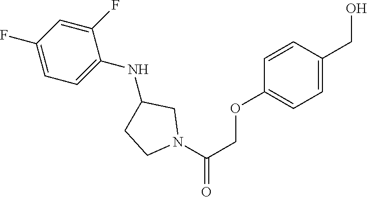 [SMILES: O=C(COc1ccc(CO)cc1)N1CCC(Nc2ccc(F)cc2F)C1] |
| 100 | 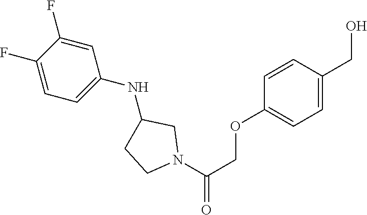 [SMILES: O=C(COc1ccc(CO)cc1)N1CCC(Nc2ccc(F)c(F)c2)C1] |
| 101 | 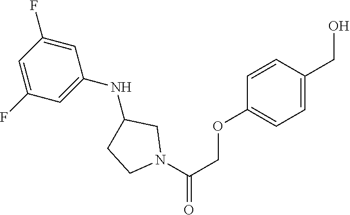 [SMILES: O=C(COc1ccc(CO)cc1)N1CCC(Nc2cc(F)cc(F)c2)C1] |
| 102 | 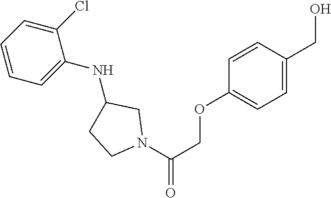 [SMILES: O=C(COc1ccc(CO)cc1)N1CCC(Nc2ccccc2Cl)C1] |
| 103 | 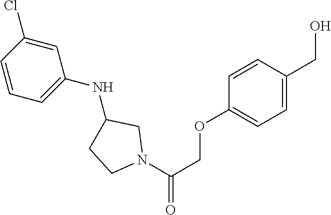 [SMILES: O=C(COc1ccc(CO)cc1)N1CCC(Nc2cccc(Cl)c2)C1] |
| 104 | 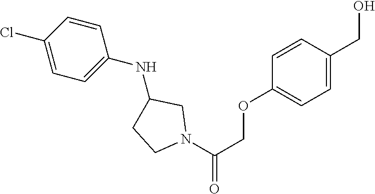 [SMILES: O=C(COc1ccc(CO)cc1)N1CCC(Nc2ccc(Cl)cc2)C1] |
| 105 | 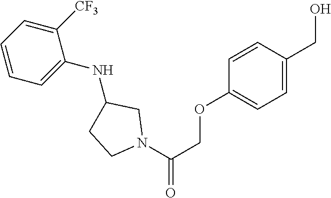 [SMILES: O=C(COc1ccc(CO)cc1)N1CCC(Nc2ccccc2C(F)(F)F)C1] |
| 106 | 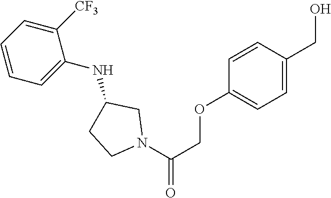 [SMILES: O=C(COc1ccc(CO)cc1)N1CC[C@H](Nc2ccccc2C(F)(F)F)C1] |
| 107 | 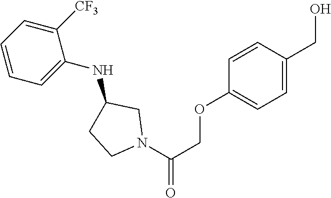 [SMILES: O=C(COc1ccc(CO)cc1)N1CC[C@@H](Nc2ccccc2C(F)(F)F)C1] |
| 108 | 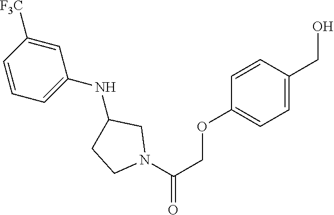 [SMILES: O=C(COc1ccc(CO)cc1)N1CCC(Nc2cccc(C(F)(F)F)c2)C1] |
| 109 | 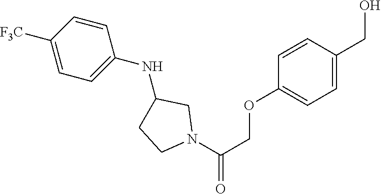 [SMILES: O=C(COc1ccc(CO)cc1)N1CCC(Nc2ccc(C(F)(F)F)cc2)C1] |
| 110 | 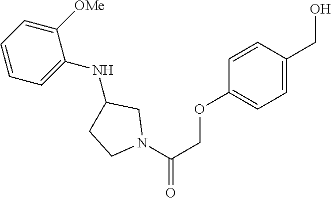 [SMILES: COc1ccccc1NC1CCN(C(=O)COc2ccc(CO)cc2)C1] |
| 111 | 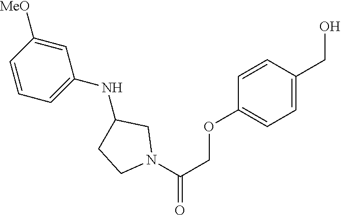 [SMILES: COc1cccc(NC2CCN(C(=O)COc3ccc(CO)cc3)C2)c1] |
| 112 | 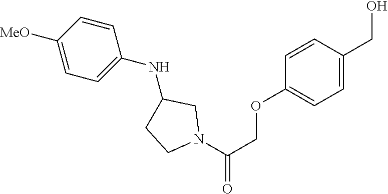 [SMILES: COc1ccc(NC2CCN(C(=O)COc3ccc(CO)cc3)C2)cc1] |
| 113 | 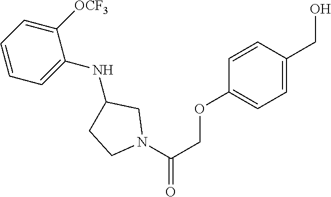 [SMILES: O=C(COc1ccc(CO)cc1)N1CCC(Nc2ccccc2OC(F)(F)F)C1] |
| 114 | 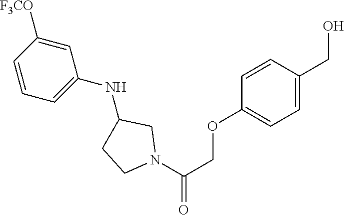 [SMILES: O=C(COc1ccc(CO)cc1)N1CCC(Nc2cccc(OC(F)(F)F)c2)C1] |
| 115 | 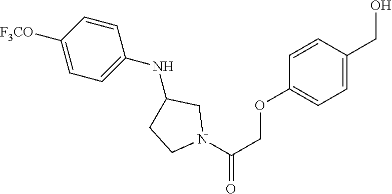 [SMILES: O=C(COc1ccc(CO)cc1)N1CCC(Nc2ccc(OC(F)(F)F)cc2)C1] |
| 116 | 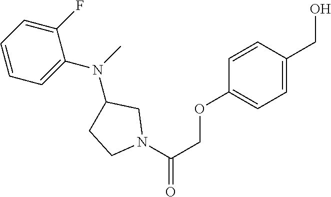 [SMILES: CN(c1ccccc1F)C1CCN(C(=O)COc2ccc(CO)cc2)C1] |
| 117 | 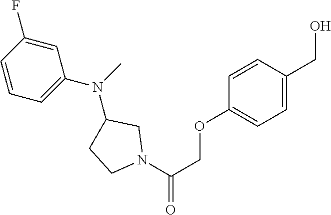 [SMILES: CN(c1cccc(F)c1)C1CCN(C(=O)COc2ccc(CO)cc2)C1] |
| 118 | 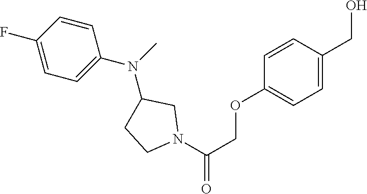 [SMILES: CN(c1ccc(F)cc1)C1CCN(C(=O)COc2ccc(CO)cc2)C1] |
| 119 | 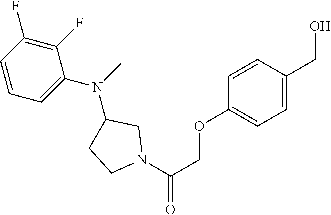 [SMILES: CN(c1cccc(F)c1F)C1CCN(C(=O)COc2ccc(CO)cc2)C1] |
| 120 | 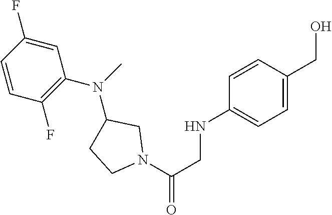 [SMILES: CN(c1cc(F)ccc1F)C1CCN(C(=O)CNc2ccc(CO)cc2)C1] |
| 121 | 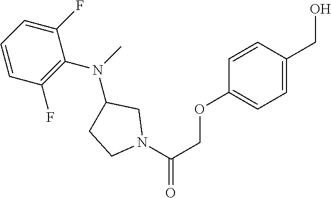 [SMILES: CN(c1c(F)cccc1F)C1CCN(C(=O)COc2ccc(CO)cc2)C1] |
| 122 | 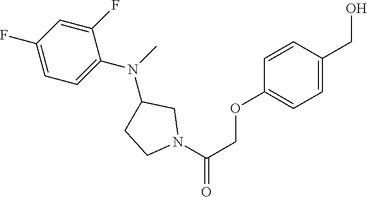 [SMILES: CN(c1ccc(F)cc1F)C1CCN(C(=O)COc2ccc(CO)cc2)C1] |
| 123 | 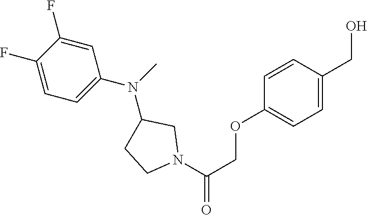 [SMILES: CN(c1ccc(F)c(F)c1)C1CCN(C(=O)COc2ccc(CO)cc2)C1] |
| 124 | 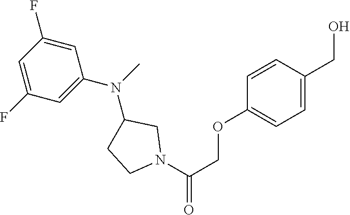 [SMILES: CN(c1cc(F)cc(F)c1)C1CCN(C(=O)COc2ccc(CO)cc2)C1] |
| 125 | 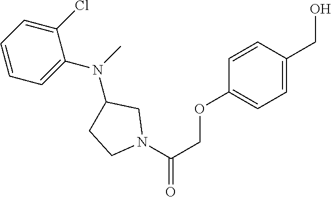 [SMILES: CN(c1ccccc1Cl)C1CCN(C(=O)COc2ccc(CO)cc2)C1] |
| 126 | 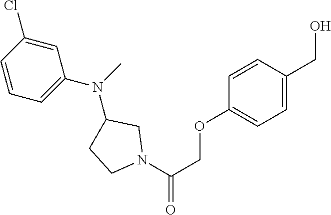 [SMILES: CN(c1cccc(Cl)c1)C1CCN(C(=O)COc2ccc(CO)cc2)C1] |
| 127 | 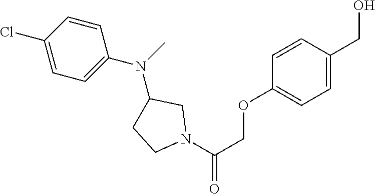 [SMILES: CN(c1ccc(Cl)cc1)C1CCN(C(=O)COc2ccc(CO)cc2)C1] |
| 128 | 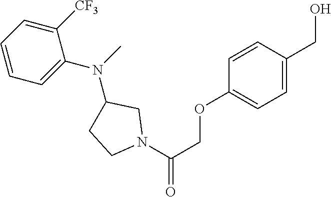 [SMILES: CN(c1ccccc1C(F)(F)F)C1CCN(C(=O)COc2ccc(CO)cc2)C1] |
| 129 | 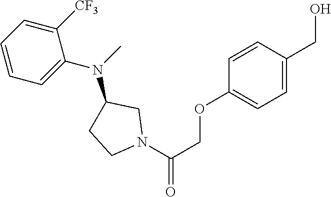 [SMILES: CN(c1ccccc1C(F)(F)F)[C@@H]1CCN(C(=O)COc2ccc(CO)cc2)C1] |
| 130 | 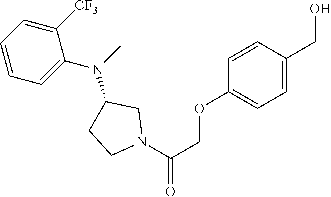 [SMILES: CN(c1ccccc1C(F)(F)F)[C@H]1CCN(C(=O)COc2ccc(CO)cc2)C1] |
| 131 | 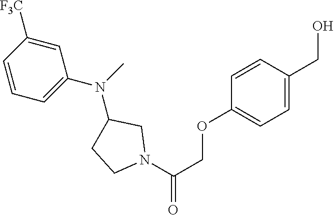 [SMILES: CN(c1cccc(C(F)(F)F)c1)C1CCN(C(=O)COc2ccc(CO)cc2)C1] |
| 132 | 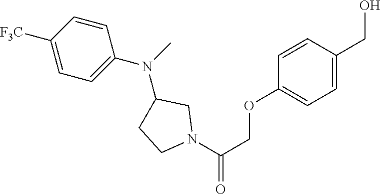 [SMILES: CN(c1ccc(C(F)(F)F)cc1)C1CCN(C(=O)COc2ccc(CO)cc2)C1] |
| 133 | 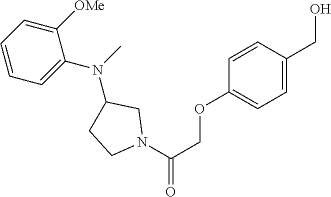 [SMILES: COc1ccccc1N(C)C1CCN(C(=O)COc2ccc(CO)cc2)C1] |
| 134 | 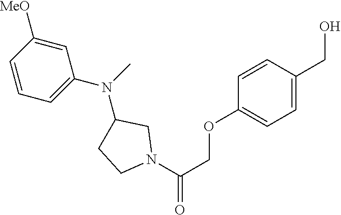 [SMILES: COc1cccc(N(C)C2CCN(C(=O)COc3ccc(CO)cc3)C2)c1] |
| 135 | 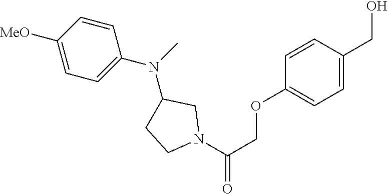 [SMILES: COc1ccc(N(C)C2CCN(C(=O)COc3ccc(CO)cc3)C2)cc1] |
| 136 | 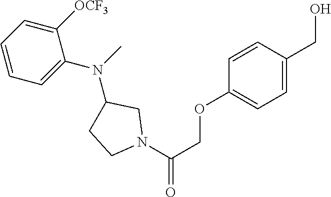 [SMILES: CN(c1ccccc1OC(F)(F)F)C1CCN(C(=O)COc2ccc(CO)cc2)C1] |
| 137 | 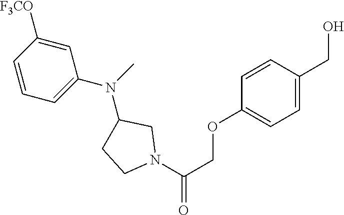 [SMILES: CN(c1cccc(OC(F)(F)F)c1)C1CCN(C(=O)COc2ccc(CO)cc2)C1] |
| 138 | 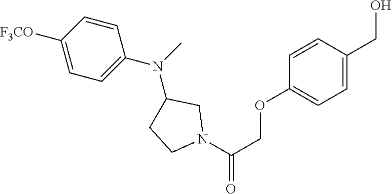 [SMILES: CN(c1ccc(OC(F)(F)F)cc1)C1CCN(C(=O)COc2ccc(CO)cc2)C1] |
| 139 | 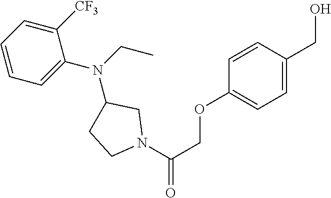 [SMILES: CCN(c1ccccc1C(F)(F)F)C1CCN(C(=O)COc2ccc(CO)cc2)C1] |
| 140 | 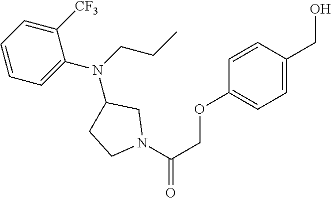 [SMILES: CCCN(c1ccccc1C(F)(F)F)C1CCN(C(=O)COc2ccc(CO)cc2)C1] |
| 141 | 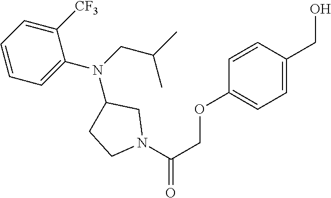 [SMILES: CC(C)CN(c1ccccc1C(F)(F)F)C1CCN(C(=O)COc2ccc(CO)cc2)C1] |
| 142 | 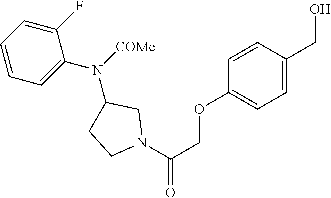 |
| 143 | 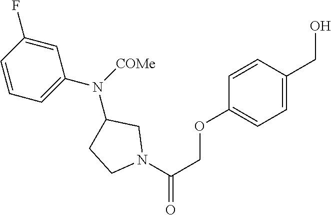 |
| 144 | 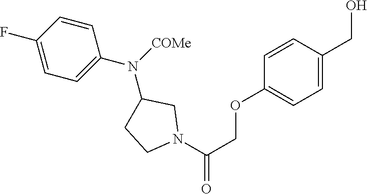 |
| 145 | 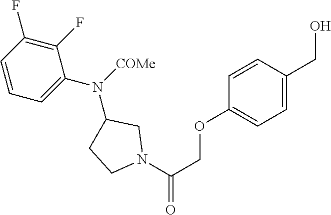 |
| 146 | 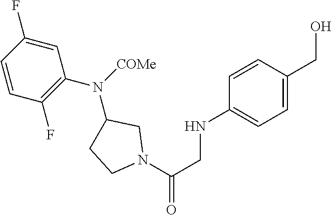 |
| 147 | 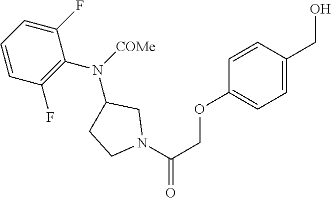 |
| 148 | 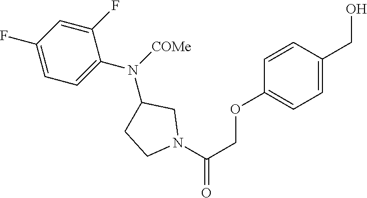 |
| 149 | 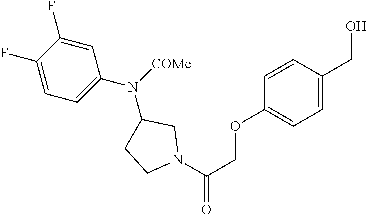 |
| 150 | 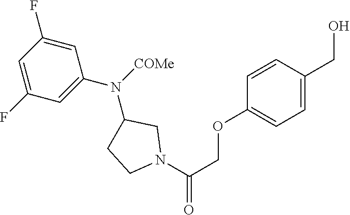 |
| 151 | 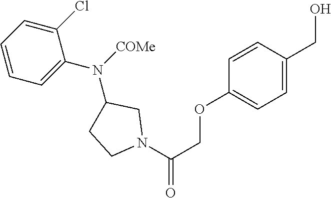 |
| 152 | 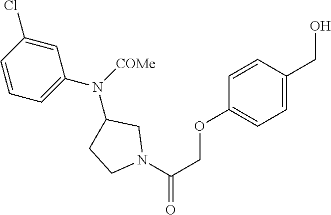 |
| 153 | 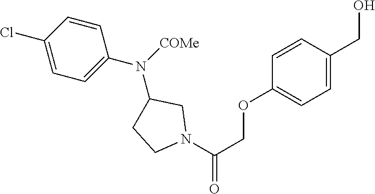 |
| 154 | 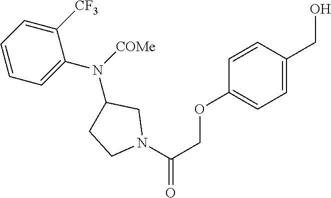 |
| 155 | 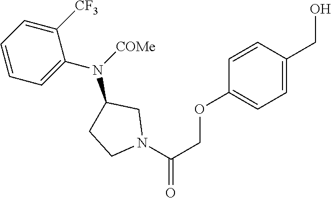 |
| 156 | 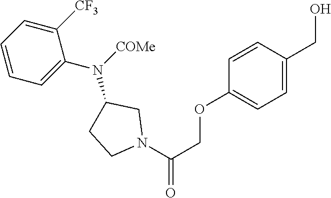 |
| 157 | 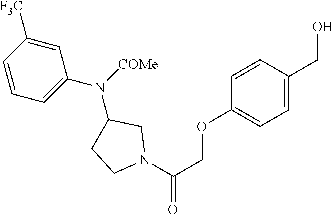 |
| 158 | 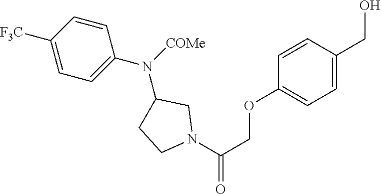 |
| 159 | 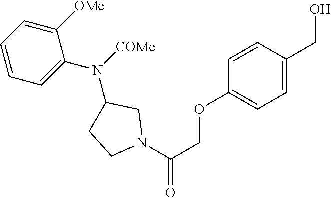 |
| 160 | 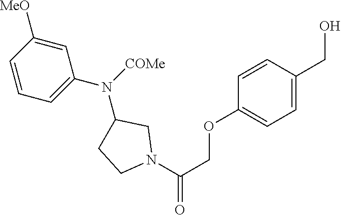 |
| 161 | 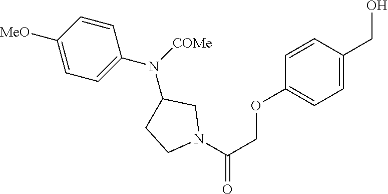 |
| 162 | 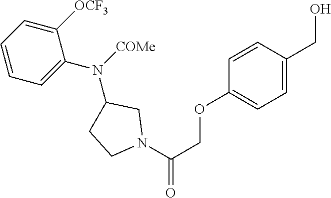 |
| 163 | 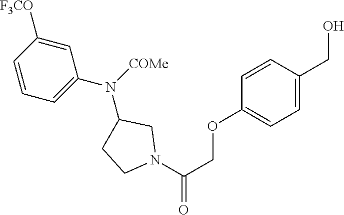 |
| 164 | 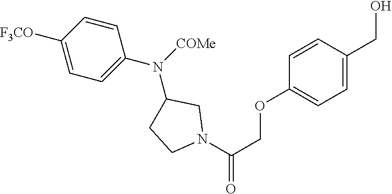 |
| 165 | 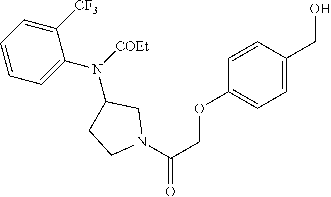 |
| 166 | 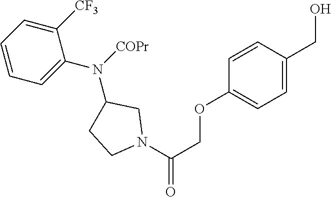 |
| 167 | 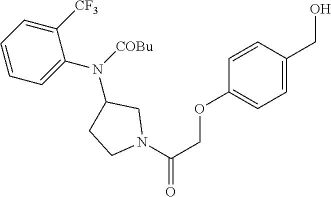 |
| 168 | 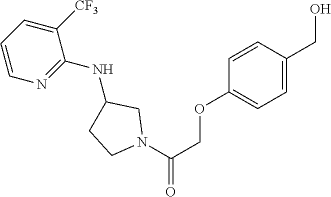 [SMILES: O=C(COc1ccc(CO)cc1)N1CCC(Nc2ncccc2C(F)(F)F)C1] |
| 169 | 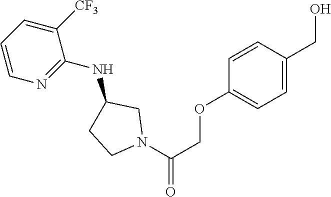 [SMILES: O=C(COc1ccc(CO)cc1)N1CC[C@@H](Nc2ncccc2C(F)(F)F)C1] |
| 170 | 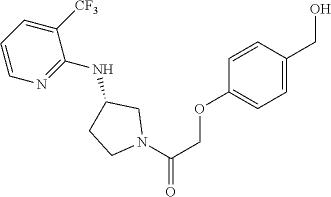 [SMILES: O=C(COc1ccc(CO)cc1)N1CC[C@H](Nc2ncccc2C(F)(F)F)C1] |
| 171 | 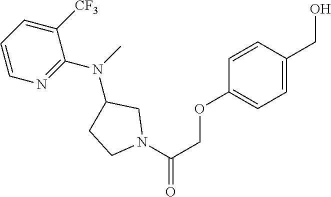 [SMILES: CN(c1ncccc1C(F)(F)F)C1CCN(C(=O)COc2ccc(CO)cc2)C1] |
| 172 | 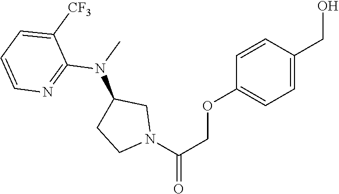 [SMILES: CN(c1ncccc1C(F)(F)F)[C@@H]1CCN(C(=O)COc2ccc(CO)cc2)C1] |
| 173 | 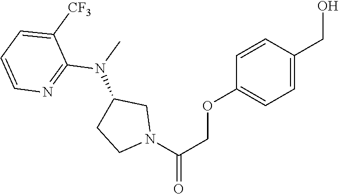 [SMILES: CN(c1ncccc1C(F)(F)F)[C@H]1CCN(C(=O)COc2ccc(CO)cc2)C1] |
| 174 | 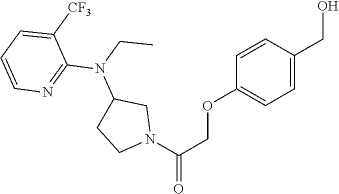 [SMILES: CCN(c1ncccc1C(F)(F)F)C1CCN(C(=O)COc2ccc(CO)cc2)C1] |
| 175 | 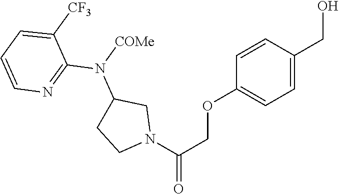 |
| 176 | 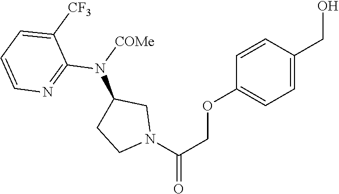 |
| 177 | 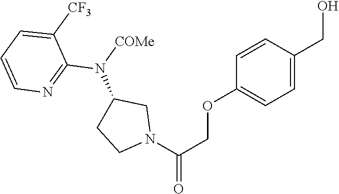 |
| 178 | 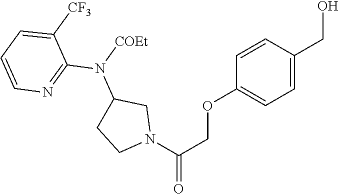 |
| 179 | 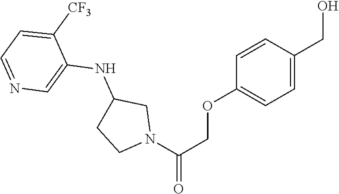 [SMILES: O=C(COc1ccc(CO)cc1)N1CCC(Nc2cnccc2C(F)(F)F)C1] |
| 180 | 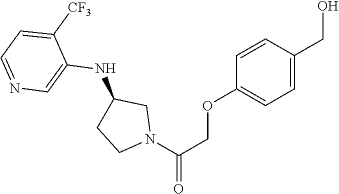 [SMILES: O=C(COc1ccc(CO)cc1)N1CC[C@@H](Nc2cnccc2C(F)(F)F)C1] |
| 181 | 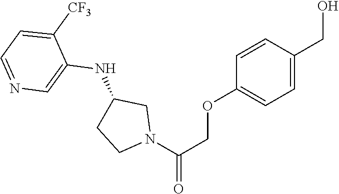 [SMILES: O=C(COc1ccc(CO)cc1)N1CC[C@H](Nc2cnccc2C(F)(F)F)C1] |
| 182 | 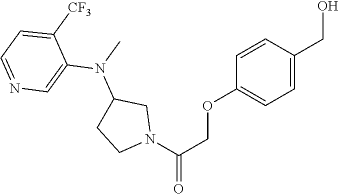 [SMILES: CN(c1cnccc1C(F)(F)F)C1CCN(C(=O)COc2ccc(CO)cc2)C1] |
| 183 | 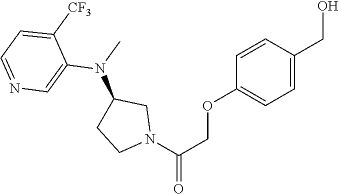 [SMILES: CN(c1cnccc1C(F)(F)F)[C@@H]1CCN(C(=O)COc2ccc(CO)cc2)C1] |
| 184 | 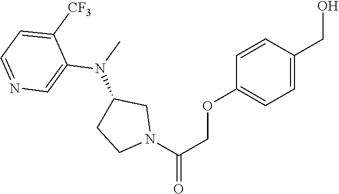 [SMILES: CN(c1cnccc1C(F)(F)F)[C@H]1CCN(C(=O)COc2ccc(CO)cc2)C1] |
| 185 | 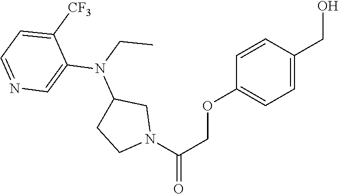 [SMILES: CCN(c1cnccc1C(F)(F)F)C1CCN(C(=O)COc2ccc(CO)cc2)C1] |
| 186 | 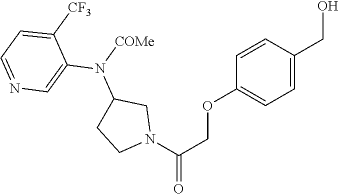 |
| 187 | 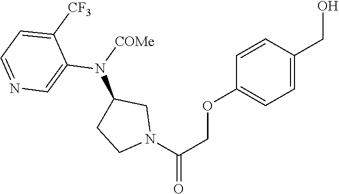 |
| 188 | 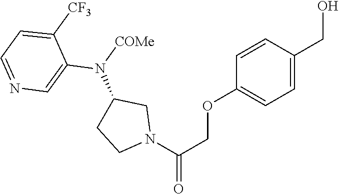 |
| 189 | 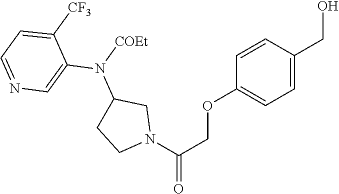 |
| 190 | 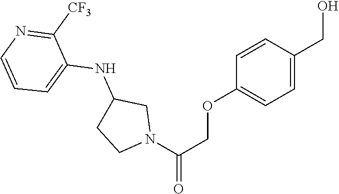 [SMILES: O=C(COc1ccc(CO)cc1)N1CCC(Nc2cccnc2C(F)(F)F)C1] |
| 191 | 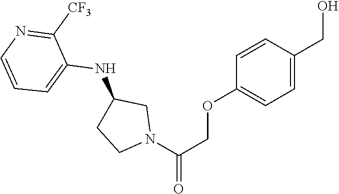 [SMILES: O=C(COc1ccc(CO)cc1)N1CC[C@@H](Nc2cccnc2C(F)(F)F)C1] |
| 192 | 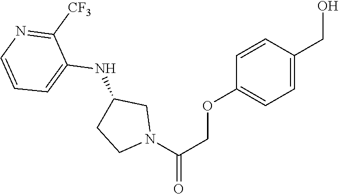 [SMILES: O=C(COc1ccc(CO)cc1)N1CC[C@H](Nc2cccnc2C(F)(F)F)C1] |
| 193 | 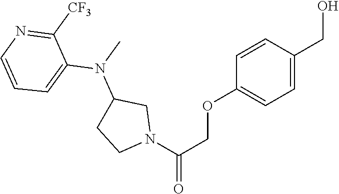 [SMILES: CN(c1cccnc1C(F)(F)F)C1CCN(C(=O)COc2ccc(CO)cc2)C1] |
| 194 | 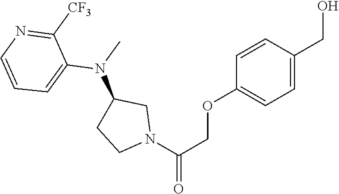 [SMILES: CN(c1cccnc1C(F)(F)F)[C@@H]1CCN(C(=O)COc2ccc(CO)cc2)C1] |
| 195 | 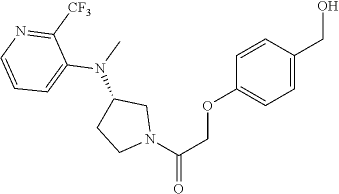 [SMILES: CN(c1cccnc1C(F)(F)F)[C@H]1CCN(C(=O)COc2ccc(CO)cc2)C1] |
| 196 | 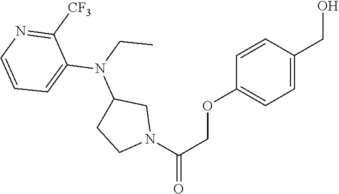 [SMILES: CCN(c1cccnc1C(F)(F)F)C1CCN(C(=O)COc2ccc(CO)cc2)C1] |
| 197 | 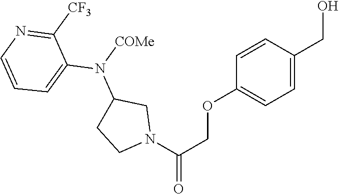 |
| 198 | 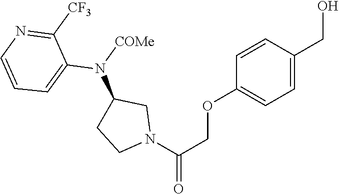 |
| 199 | 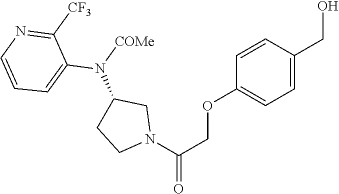 |
| 200 | 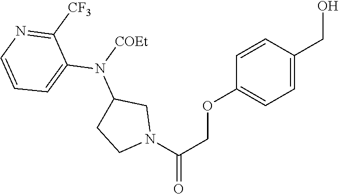 |
| 201 | 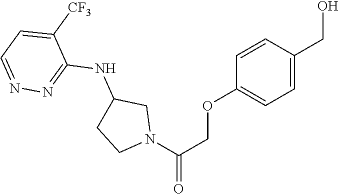 [SMILES: O=C(COc1ccc(CO)cc1)N1CCC(Nc2nnccc2C(F)(F)F)C1] |
| 202 | 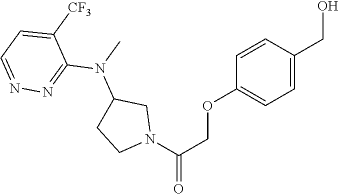 [SMILES: CN(c1nnccc1C(F)(F)F)C1CCN(C(=O)COc2ccc(CO)cc2)C1] |
| 203 | 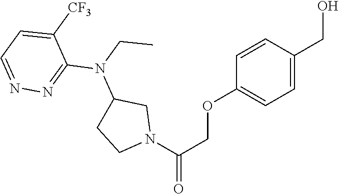 [SMILES: CCN(c1nnccc1C(F)(F)F)C1CCN(C(=O)COc2ccc(CO)cc2)C1] |
| 204 | 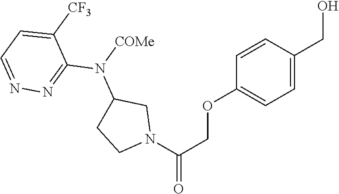 |
| 205 | 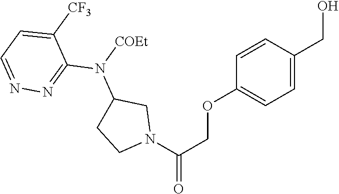 |
| 206 | 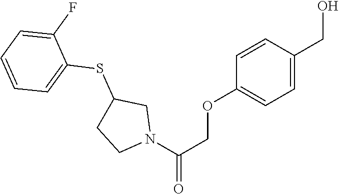 [SMILES: O=C(COc1ccc(CO)cc1)N1CCC(Sc2ccccc2F)C1] |
| 207 | 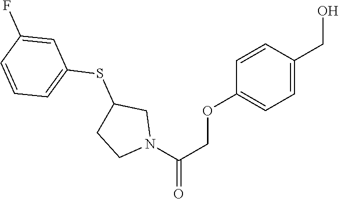 [SMILES: O=C(COc1ccc(CO)cc1)N1CCC(Sc2cccc(F)c2)C1] |
| 208 | 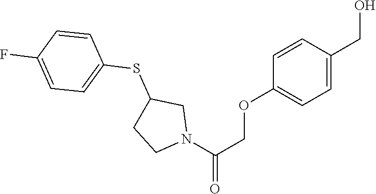 [SMILES: O=C(COc1ccc(CO)cc1)N1CCC(Sc2ccc(F)cc2)C1] |
| 209 | 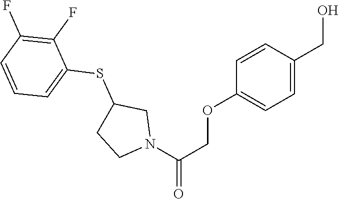 [SMILES: O=C(COc1ccc(CO)cc1)N1CCC(Sc2cccc(F)c2F)C1] |
| 210 | 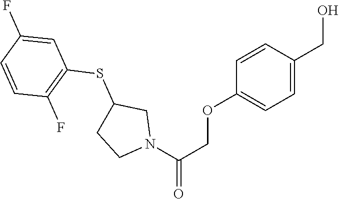 [SMILES: O=C(COc1ccc(CO)cc1)N1CCC(Sc2cc(F)ccc2F)C1] |
| 211 | 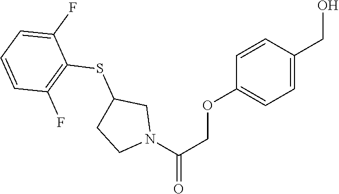 [SMILES: O=C(COc1ccc(CO)cc1)N1CCC(Sc2c(F)cccc2F)C1] |
| 212 | 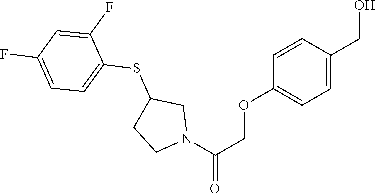 [SMILES: O=C(COc1ccc(CO)cc1)N1CCC(Sc2ccc(F)cc2F)C1] |
| 213 | 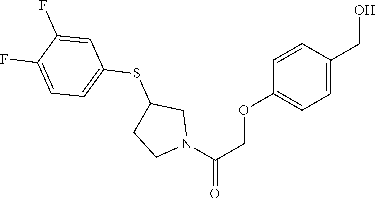 [SMILES: O=C(COc1ccc(CO)cc1)N1CCC(Sc2ccc(F)c(F)c2)C1] |
| 214 | 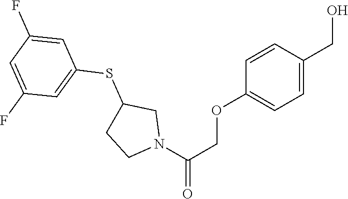 [SMILES: O=C(COc1ccc(CO)cc1)N1CCC(Sc2cc(F)cc(F)c2)C1] |
| 215 | 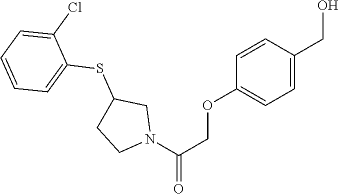 [SMILES: O=C(COc1ccc(CO)cc1)N1CCC(Sc2ccccc2Cl)C1] |
| 216 | 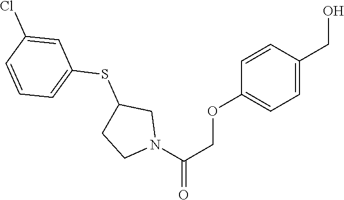 [SMILES: O=C(COc1ccc(CO)cc1)N1CCC(Sc2cccc(Cl)c2)C1] |
| 217 | 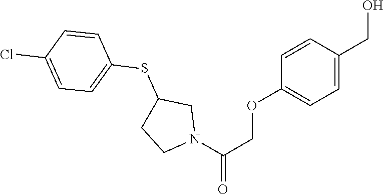 [SMILES: O=C(COc1ccc(CO)cc1)N1CCC(Sc2ccc(Cl)cc2)C1] |
| 218 | 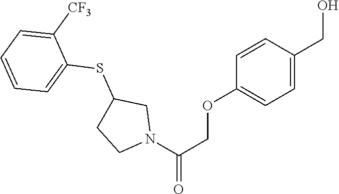 [SMILES: O=C(COc1ccc(CO)cc1)N1CCC(Sc2ccccc2C(F)(F)F)C1] |
| 219 | 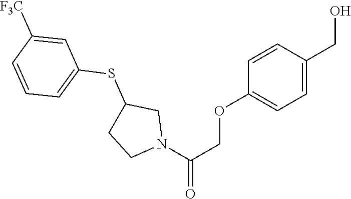 [SMILES: O=C(COc1ccc(CO)cc1)N1CCC(Sc2cccc(C(F)(F)F)c2)C1] |
| 220 | 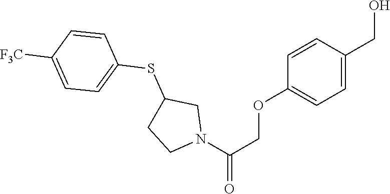 [SMILES: O=C(COc1ccc(CO)cc1)N1CCC(Sc2ccc(C(F)(F)F)cc2)C1] |
| 221 | 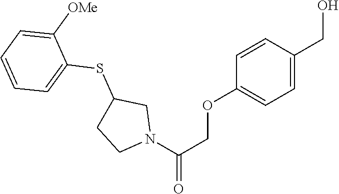 [SMILES: COc1ccccc1SC1CCN(C(=O)COc2ccc(CO)cc2)C1] |
| 222 | 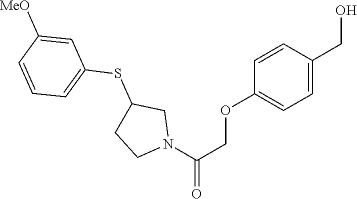 [SMILES: COc1cccc(SC2CCN(C(=O)COc3ccc(CO)cc3)C2)c1] |
| 223 | 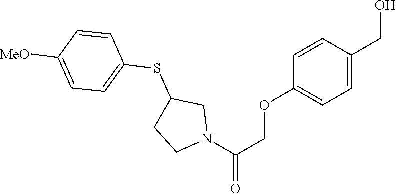 [SMILES: COc1ccc(SC2CCN(C(=O)COc3ccc(CO)cc3)C2)cc1] |
| 224 | 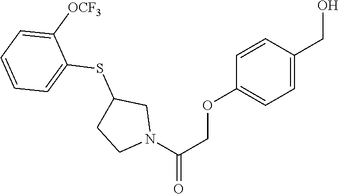 [SMILES: O=C(COc1ccc(CO)cc1)N1CCC(Sc2ccccc2OC(F)(F)F)C1] |
| 225 | 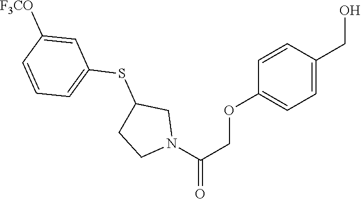 [SMILES: O=C(COc1ccc(CO)cc1)N1CCC(Sc2cccc(OC(F)(F)F)c2)C1] |
| 226 | 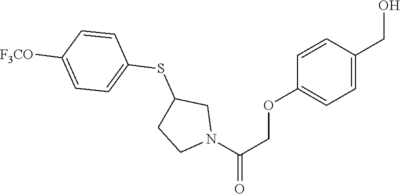 [SMILES: O=C(COc1ccc(CO)cc1)N1CCC(Sc2ccc(OC(F)(F)F)cc2)C1] |
| 227 | 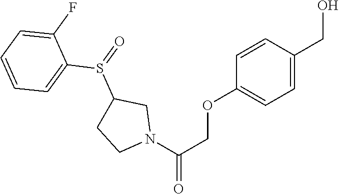 [SMILES: O=C(COc1ccc(CO)cc1)N1CCC(S(=O)c2ccccc2F)C1] |
| 228 | 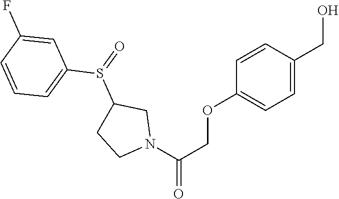 [SMILES: O=C(COc1ccc(CO)cc1)N1CCC(S(=O)c2cccc(F)c2)C1] |
| 229 | 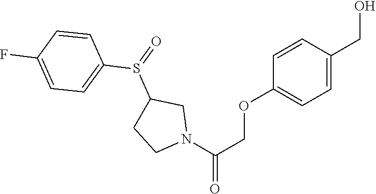 [SMILES: O=C(COc1ccc(CO)cc1)N1CCC(S(=O)c2ccc(F)cc2)C1] |
| 230 | 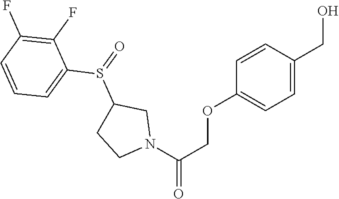 [SMILES: O=C(COc1ccc(CO)cc1)N1CCC(S(=O)c2cccc(F)c2F)C1] |
| 231 | 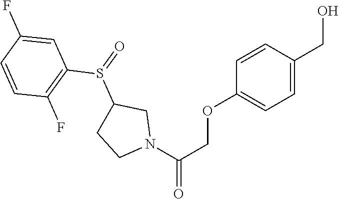 [SMILES: O=C(COc1ccc(CO)cc1)N1CCC(S(=O)c2cc(F)ccc2F)C1] |
| 232 | 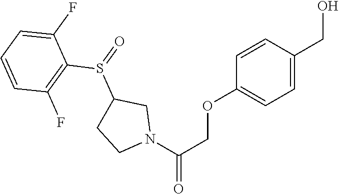 [SMILES: O=C(COc1ccc(CO)cc1)N1CCC(S(=O)c2c(F)cccc2F)C1] |
| 233 | 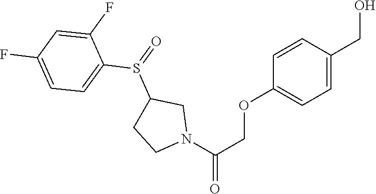 [SMILES: O=C(COc1ccc(CO)cc1)N1CCC(S(=O)c2ccc(F)cc2F)C1] |
| 234 | 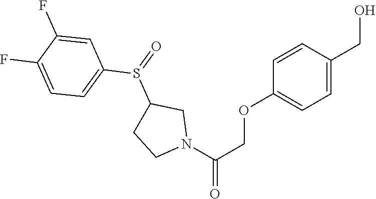 [SMILES: O=C(COc1ccc(CO)cc1)N1CCC(S(=O)c2ccc(F)c(F)c2)C1] |
| 235 | 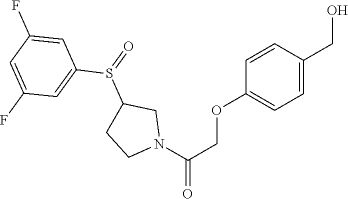 [SMILES: O=C(COc1ccc(CO)cc1)N1CCC(S(=O)c2cc(F)cc(F)c2)C1] |
| 236 | 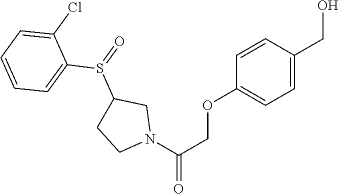 [SMILES: O=C(COc1ccc(CO)cc1)N1CCC(S(=O)c2ccccc2Cl)C1] |
| 237 | 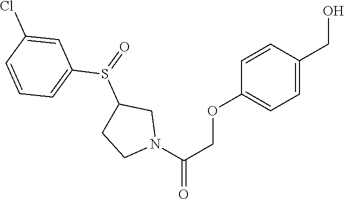 [SMILES: O=C(COc1ccc(CO)cc1)N1CCC(S(=O)c2cccc(Cl)c2)C1] |
| 238 | 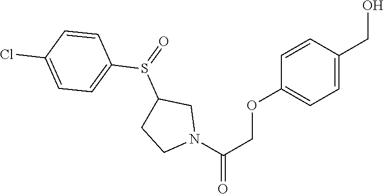 [SMILES: O=C(COc1ccc(CO)cc1)N1CCC(S(=O)c2ccc(Cl)cc2)C1] |
| 239 | 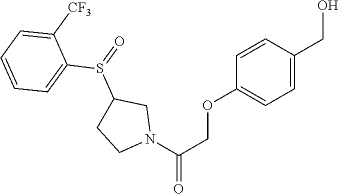 [SMILES: O=C(COc1ccc(CO)cc1)N1CCC(S(=O)c2ccccc2C(F)(F)F)C1] |
| 240 | 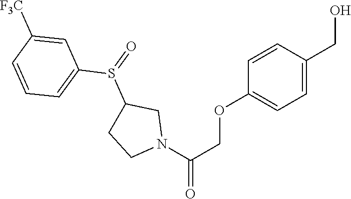 [SMILES: O=C(COc1ccc(CO)cc1)N1CCC(S(=O)c2cccc(C(F)(F)F)c2)C1] |
| 241 | 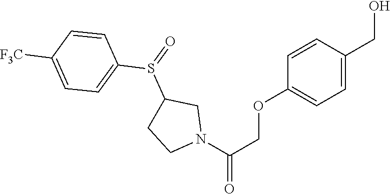 [SMILES: O=C(COc1ccc(CO)cc1)N1CCC(S(=O)c2ccc(C(F)(F)F)cc2)C1] |
| 242 | 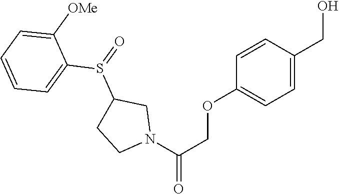 [SMILES: COc1ccccc1S(=O)C1CCN(C(=O)COc2ccc(CO)cc2)C1] |
| 243 | 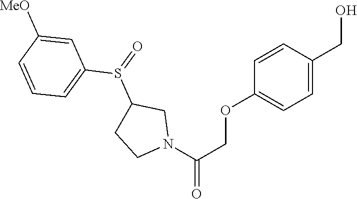 [SMILES: COc1cccc(S(=O)C2CCN(C(=O)COc3ccc(CO)cc3)C2)c1] |
| 244 | 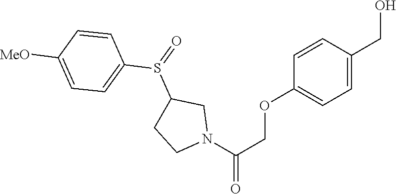 [SMILES: COc1ccc(S(=O)C2CCN(C(=O)COc3ccc(CO)cc3)C2)cc1] |
| 245 | 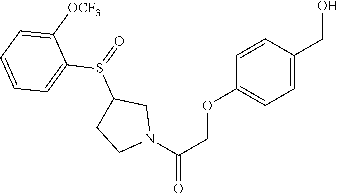 [SMILES: O=C(COc1ccc(CO)cc1)N1CCC(S(=O)c2ccccc2OC(F)(F)F)C1] |
| 246 | 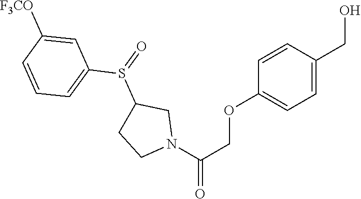 [SMILES: O=C(COc1ccc(CO)cc1)N1CCC(S(=O)c2cccc(OC(F)(F)F)c2)C1] |
| 247 | 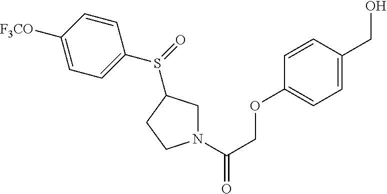 [SMILES: O=C(COc1ccc(CO)cc1)N1CCC(S(=O)c2ccc(OC(F)(F)F)cc2)C1] |
| 248 | 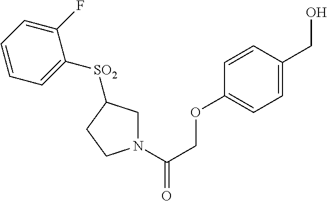 [SMILES: O=C(COc1ccc(CO)cc1)N1CCC(S(=O)(=O)c2ccccc2F)C1] |
| 249 | 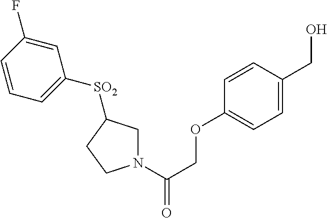 [SMILES: O=C(COc1ccc(CO)cc1)N1CCC(S(=O)(=O)c2cccc(F)c2)C1] |
| 250 | 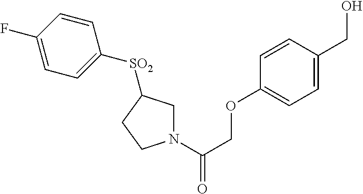 [SMILES: O=C(COc1ccc(CO)cc1)N1CCC(S(=O)(=O)c2ccc(F)cc2)C1] |
| 251 | 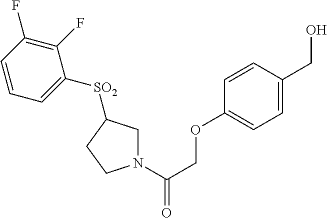 [SMILES: O=C(COc1ccc(CO)cc1)N1CCC(S(=O)(=O)c2cccc(F)c2F)C1] |
| 252 | 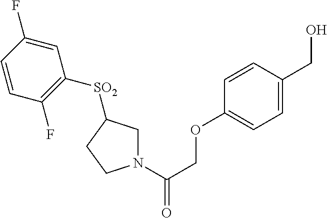 [SMILES: O=C(COc1ccc(CO)cc1)N1CCC(S(=O)(=O)c2cc(F)ccc2F)C1] |
| 253 | 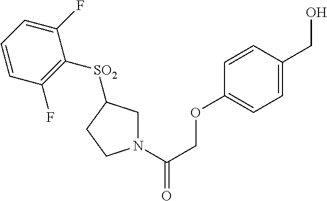 [SMILES: O=C(COc1ccc(CO)cc1)N1CCC(S(=O)(=O)c2c(F)cccc2F)C1] |
| 254 | 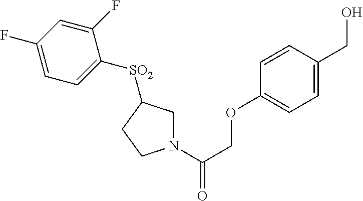 [SMILES: O=C(COc1ccc(CO)cc1)N1CCC(S(=O)(=O)c2ccc(F)cc2F)C1] |
| 255 | 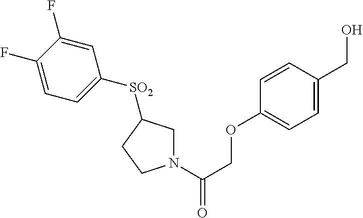 [SMILES: O=C(COc1ccc(CO)cc1)N1CCC(S(=O)(=O)c2ccc(F)c(F)c2)C1] |
| 256 | 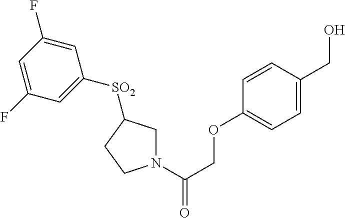 [SMILES: O=C(COc1ccc(CO)cc1)N1CCC(S(=O)(=O)c2cc(F)cc(F)c2)C1] |
| 257 | 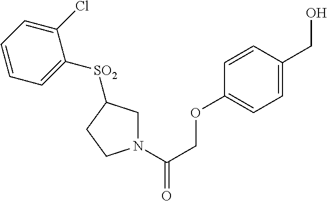 [SMILES: O=C(COc1ccc(CO)cc1)N1CCC(S(=O)(=O)c2ccccc2Cl)C1] |
| 258 | 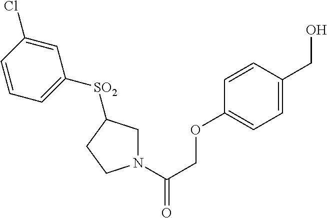 [SMILES: O=C(COc1ccc(CO)cc1)N1CCC(S(=O)(=O)c2cccc(Cl)c2)C1] |
| 259 | 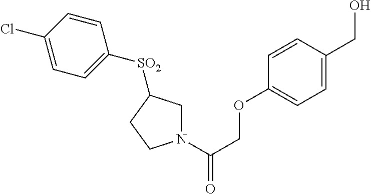 [SMILES: O=C(COc1ccc(CO)cc1)N1CCC(S(=O)(=O)c2ccc(Cl)cc2)C1] |
| 260 | 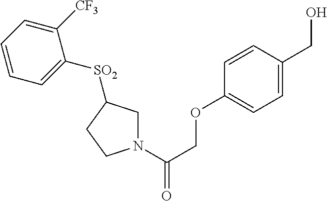 [SMILES: O=C(COc1ccc(CO)cc1)N1CCC(S(=O)(=O)c2ccccc2C(F)(F)F)C1] |
| 261 | 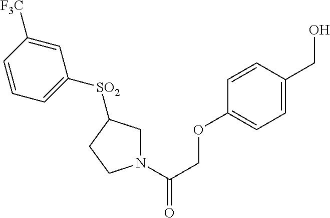 [SMILES: O=C(COc1ccc(CO)cc1)N1CCC(S(=O)(=O)c2cccc(C(F)(F)F)c2)C1] |
| 262 | 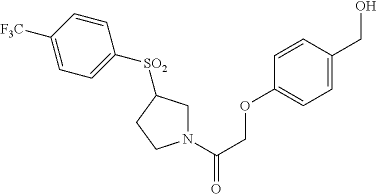 [SMILES: O=C(COc1ccc(CO)cc1)N1CCC(S(=O)(=O)c2ccc(C(F)(F)F)cc2)C1] |
| 263 | 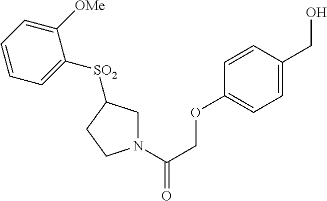 [SMILES: COc1ccccc1S(=O)(=O)C1CCN(C(=O)COc2ccc(CO)cc2)C1] |
| 264 | 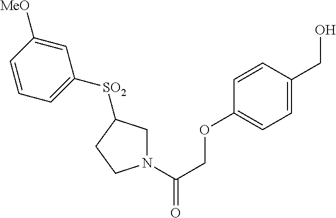 [SMILES: COc1cccc(S(=O)(=O)C2CCN(C(=O)COc3ccc(CO)cc3)C2)c1] |
| 265 | 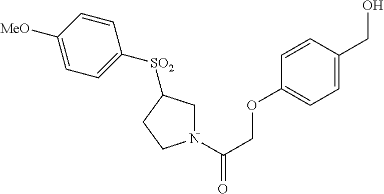 [SMILES: COc1ccc(S(=O)(=O)C2CCN(C(=O)COc3ccc(CO)cc3)C2)cc1] |
| 266 | 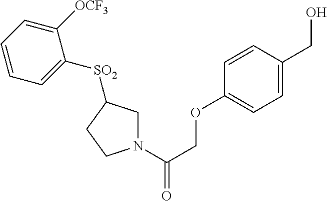 [SMILES: O=C(COc1ccc(CO)cc1)N1CCC(S(=O)(=O)c2ccccc2OC(F)(F)F)C1] |
| 267 | 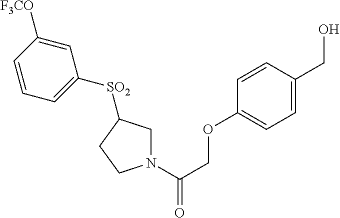 [SMILES: O=C(COc1ccc(CO)cc1)N1CCC(S(=O)(=O)c2cccc(OC(F)(F)F)c2)C1] |
| 268 | 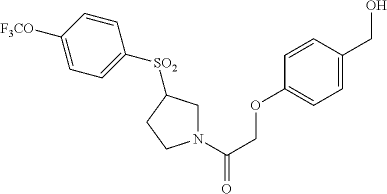 [SMILES: O=C(COc1ccc(CO)cc1)N1CCC(S(=O)(=O)c2ccc(OC(F)(F)F)cc2)C1] |
| 269 | 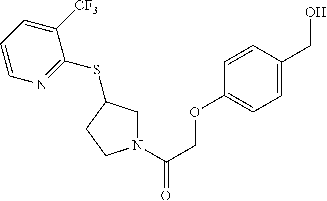 [SMILES: O=C(COc1ccc(CO)cc1)N1CCC(Sc2ncccc2C(F)(F)F)C1] |
| 270 | 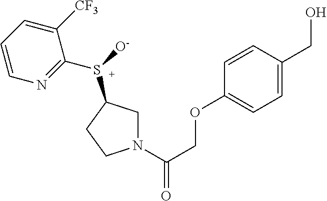 |
| 271 | 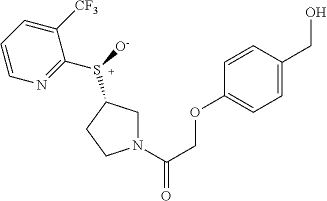 |
| 272 | 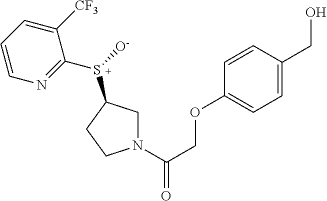 |
| 273 | 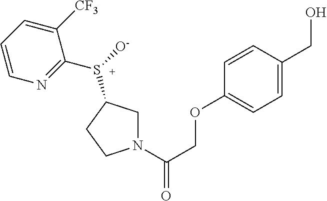 |
| 274 | 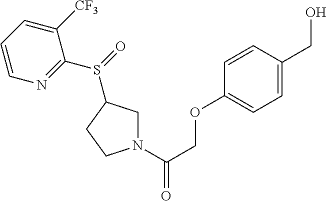 [SMILES: O=C(COc1ccc(CO)cc1)N1CCC(S(=O)c2ncccc2C(F)(F)F)C1] |
| 275 | 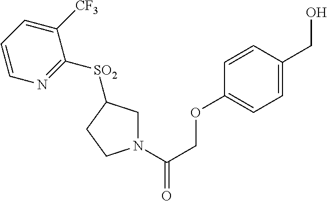 [SMILES: O=C(COc1ccc(CO)cc1)N1CCC(S(=O)(=O)c2ncccc2C(F)(F)F)C1] |
| 276 | 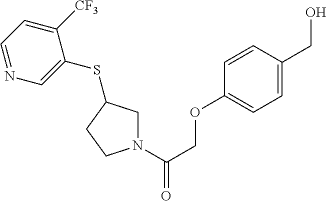 [SMILES: O=C(COc1ccc(CO)cc1)N1CCC(Sc2cnccc2C(F)(F)F)C1] |
| 277 | 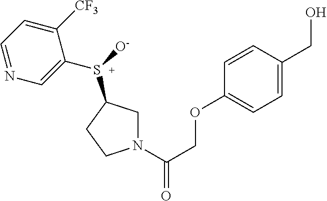 |
| 278 | 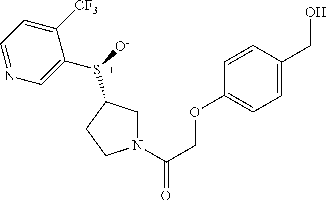 |
| 279 | 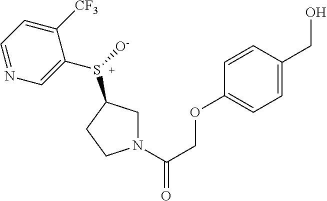 |
| 280 | 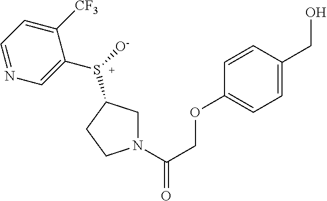 |
| 281 | 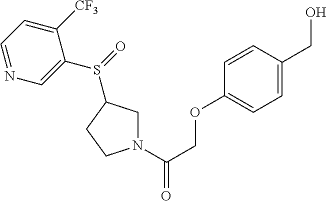 [SMILES: O=C(COc1ccc(CO)cc1)N1CCC(S(=O)c2cnccc2C(F)(F)F)C1] |
| 282 | 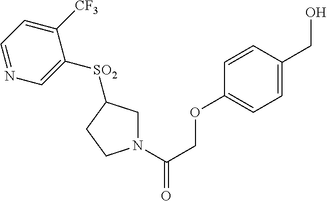 [SMILES: O=C(COc1ccc(CO)cc1)N1CCC(S(=O)(=O)c2cnccc2C(F)(F)F)C1] |
| 283 | 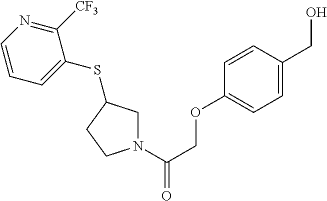 [SMILES: O=C(COc1ccc(CO)cc1)N1CCC(Sc2cccnc2C(F)(F)F)C1] |
| 284 | 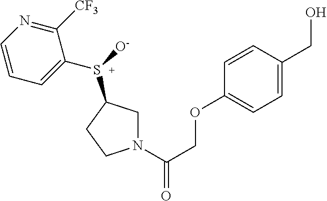 |
| 285 | 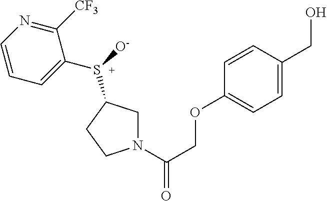 |
| 286 | 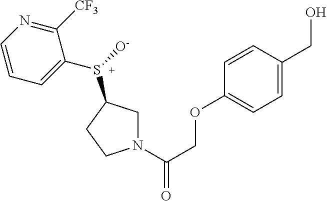 |
| 287 | 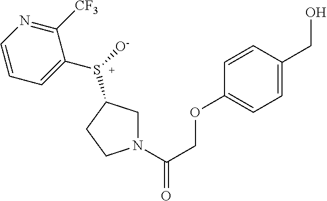 |
| 288 | 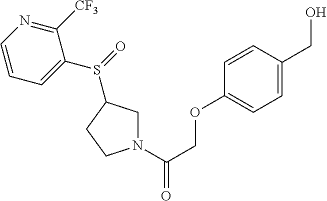 [SMILES: O=C(COc1ccc(CO)cc1)N1CCC(S(=O)c2cccnc2C(F)(F)F)C1] |
| 289 | 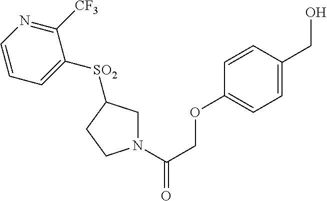 [SMILES: O=C(COc1ccc(CO)cc1)N1CCC(S(=O)(=O)c2cccnc2C(F)(F)F)C1] |
| 290 | 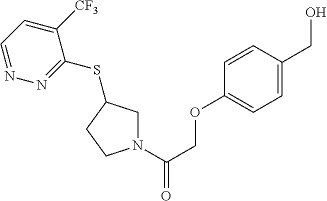 [SMILES: O=C(COc1ccc(CO)cc1)N1CCC(Sc2nnccc2C(F)(F)F)C1] |
| 291 | 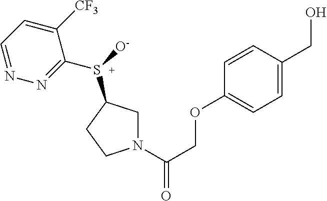 |
| 292 | 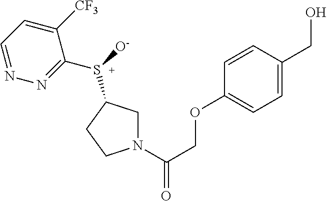 |
| 293 | 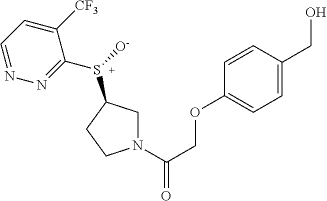 |
| 294 | 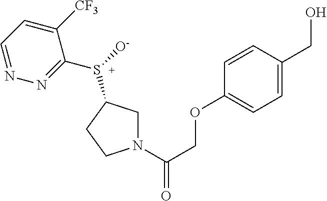 |
| 295 | 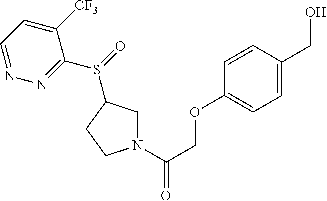 [SMILES: O=C(COc1ccc(CO)cc1)N1CCC(S(=O)c2nnccc2C(F)(F)F)C1] |
| 296 | 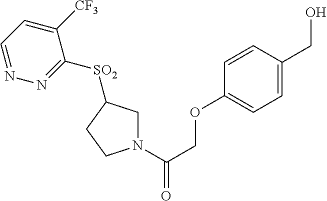 [SMILES: O=C(COc1ccc(CO)cc1)N1CCC(S(=O)(=O)c2nnccc2C(F)(F)F)C1] |

World Intellectual Property Organization, WO/2006/034338, WO/2006/034341, WO/2006/034440, and WO/2006/034441 describe compounds having Formulas II, III, IV, and V and are hereby incorporated by reference in their entirety.

Some embodiments of the present disclosure include compounds of Formula II:

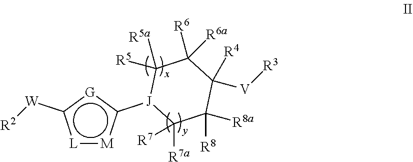

or salts, pharmaceutically acceptable salts, or prodrugs thereof.
wherein variables R², R³, R⁴, R⁵, R^{5a}, R⁶, R^{6a}, R⁷. R^{7a}, R⁸. R^{8a}, G, J, L, M, V, W, x, and y are defined in WO/2006/034338.

Some embodiments of the present disclosure include compounds of Formula III:

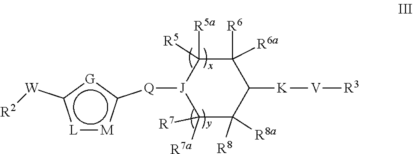

or salts, pharmaceutically acceptable salts, or prodrugs thereof wherein variables R², R³, R⁵, R^{5a}, R⁶, R^{6a}, R⁷. R^{7a}, R⁸. R^{8a}, G, J, K, L, M, Q, V, W, x, and y are defined in WO/2006/034341.

Some embodiments of the present disclosure include compounds of Formula IV:

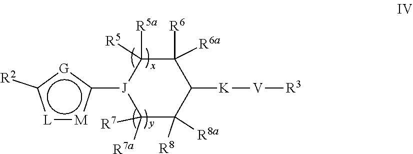

or salts, pharmaceutically acceptable salts, or prodrugs thereof wherein variables R², R³, R⁵, R^{5a}, R⁶, R^{6a}, R⁷. R^{7a}, R⁸. R^{8a}, G, J, K, L, M, V, x, and y are defined in WO/2006/034440.

Some embodiments of the present disclosure include compounds of Formula V:

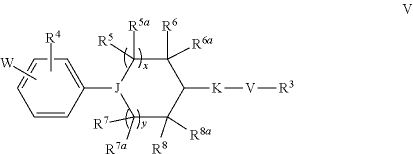

or salts, pharmaceutically acceptable salts, or prodrugs thereof wherein variables R³, R⁴, R⁵, R^{5a}, R⁶, R^{6a}, R⁷. R^{7a}, R⁸. R^{8a}, J, K, V, W, x, and y are defined in WO/2006/034441.

Illustrative compounds of Formulas II, III, IV, and V are shown in Table 2.

TABLE 2

| | |
|---|---|
| 297 | 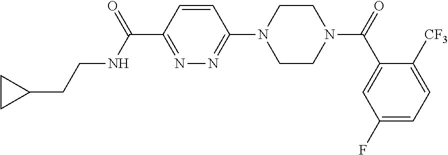 [SMILES: O=C(NCCC1CC1)c1ccc(N2CCN(C(=O)c3cc(F)ccc3C(F)(F)F)CC2)nn1] |
| 298 | 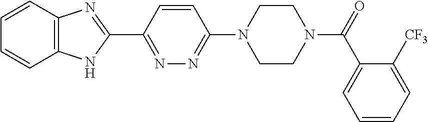 [SMILES: O=C(c1ccccc1C(F)(F)F)N1CCN(c2ccc(-c3nc4ccccc4[nH]3)nn2)CC1] |
| 299 | 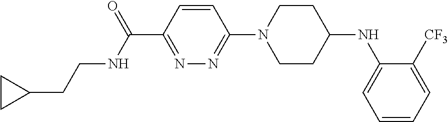 [SMILES: O=C(NCCC1CC1)c1ccc(N2CCC(Nc3ccccc3C(F)(F)F)CC2)nn1] |
| 300 | 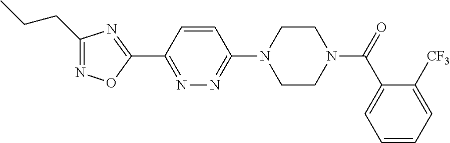 [SMILES: CCCc1noc(-c2ccc(N3CCN(C(=O)c4ccccc4C(F)(F)F)CC3)nn2)n1] |
| 301 | 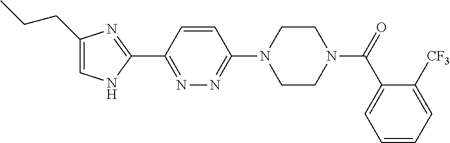 [SMILES: CCCc1c[nH]c(-c2ccc(N3CCN(C(=O)c4ccccc4C(F)(F)F)CC3)nn2)n1] |
| 302 | 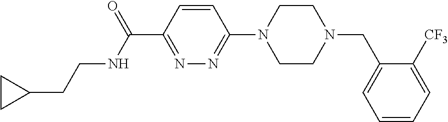 [SMILES: O=C(NCCC1CC1)c1ccc(N2CCN(Cc3ccccc3C(F)(F)F)CC2)nn1] |
| 303 | 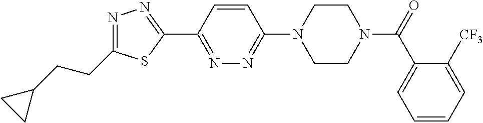 [SMILES: O=C(c1ccccc1C(F)(F)F)N1CCN(c2ccc(-c3nnc(CCC4CC4)s3)nn2)CC1] |
| 304 | 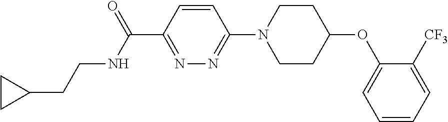 [SMILES: O=C(NCCC1CC1)c1ccc(N2CCC(Oc3ccccc3C(F)(F)F)CC2)nn1] |
| 305 | 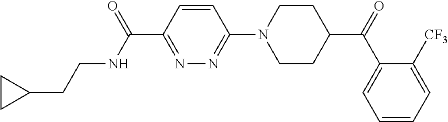 [SMILES: O=C(NCCC1CC1)c1ccc(N2CCC(C(=O)c3ccccc3C(F)(F)F)CC2)nn1] |

World Intellectual Property Organization, WO/2008/024390, WO/2008/036715, WO/2008/074835, WO/2008/127349 and WO/2009/103739 describe compounds having Formulas VI, VII, and VIII and are hereby incorporated by reference in their entirety.

Some embodiments of the present disclosure include compounds of Formula VI:

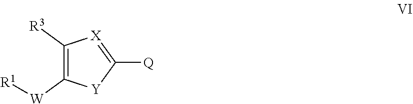

or salts, pharmaceutically acceptable salts, or prodrugs thereof wherein variables R¹, R³, Q, W, X, and Y are defined in WO/2008/024390 and WO/2008/074835.

Some embodiments of the present disclosure include compounds of Formula VII:

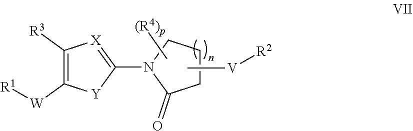

or salts, pharmaceutically acceptable salts, or prodrugs thereof wherein variables R¹, R², R³, R⁴, V, W, X, Y, n, and p are defined in WO/2008/036715.

Some embodiments of the present disclosure include compounds of Formula VIII:

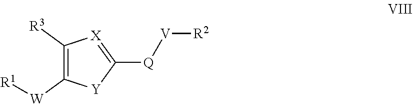

or salts, pharmaceutically acceptable salts, or prodrugs thereof wherein variables R¹, R², R³, Q, V, W, X, and Y are defined in WO/2008/127349 and WO/2009/103739.

Illustrative compounds of Formulas VI, VII, and VIII are shown in Table 3.

TABLE 3

| | |
|---|---|
| 306 | 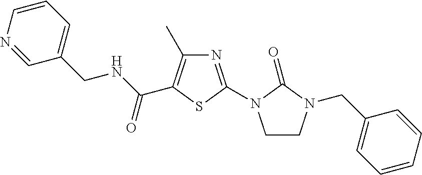 [SMILES: Cc1nc(N2CCN(Cc3ccccc3)C2=O)sc1C(=O)NCc1cccnc1] |
| 307 | 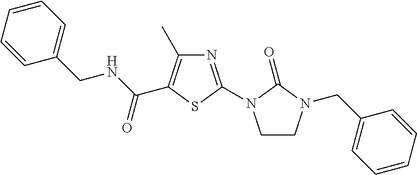 [SMILES: Cc1nc(N2CCN(Cc3ccccc3)C2=O)sc1C(=O)NCc1ccccc1] |
| 308 | 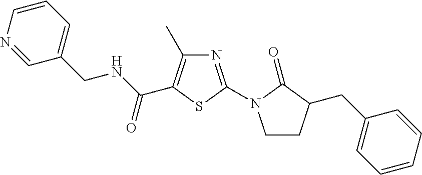 [SMILES: Cc1nc(N2CCC(Cc3ccccc3)C2=O)sc1C(=O)NCc1cccnc1] |
| 309 | 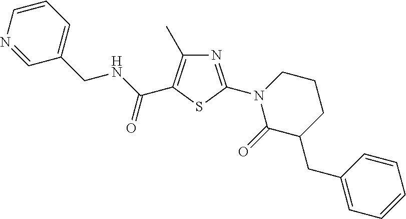 [SMILES: Cc1nc(N2CCCC(Cc3ccccc3)C2=O)sc1C(=O)NCc1cccnc1] |
| 310 | 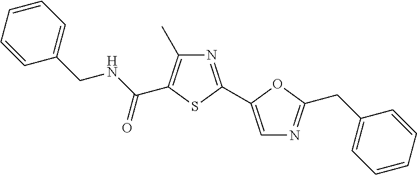 [SMILES: Cc1nc(-c2cnc(Cc3ccccc3)o2)sc1C(=O)NCc1ccccc1] |
| 311 | 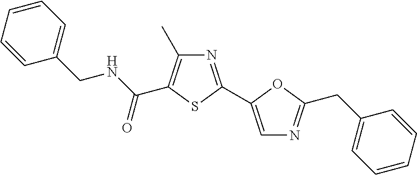 [SMILES: Cc1nc(-c2cnc(Cc3ccccc3)o2)sc1C(=O)NCc1ccccc1] |
| 312 | 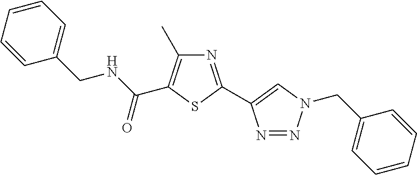 [SMILES: Cc1nc(-c2cn(Cc3ccccc3)nn2)sc1C(=O)NCc1ccccc1] |
| 313 | 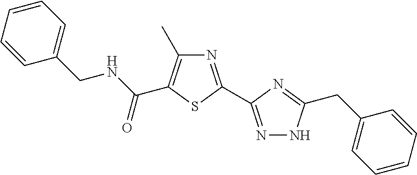 [SMILES: Cc1nc(-c2n[nH]c(Cc3ccccc3)n2)sc1C(=O)NCc1ccccc1] |
| 314 | 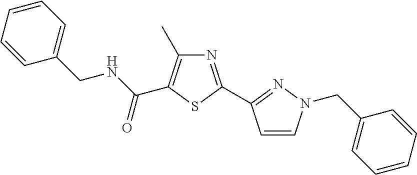 [SMILES: Cc1nc(-c2ccn(Cc3ccccc3)n2)sc1C(=O)NCc1ccccc1] |
| 315 | 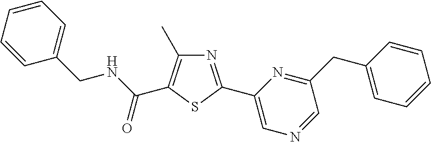 [SMILES: Cc1nc(-c2cncc(Cc3ccccc3)n2)sc1C(=O)NCc1ccccc1] |

World Intellectual Property Organization, WO/2009/156484, WO/2010/112520, and WO/2011/039358, and publication, Bioorganic & Medicinal Chemistry (2015), 23(3), 455-465 describe compounds having Formulas IX, X, and XI and are hereby incorporated by reference in their entirety.

Some embodiments of the present disclosure include compounds of Formula IX:

R¹—W—P-Q-R² IX

or salts, pharmaceutically acceptable salts, or prodrugs thereof wherein variables R², R², P, Q, and W are defined in WO/2009/156484.

Some embodiments of the present disclosure include compounds of Formula X:

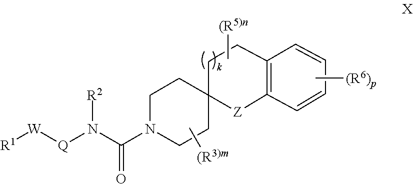

or salts, pharmaceutically acceptable salts, or prodrugs thereof wherein variables R¹, R², R³, R⁵, R⁶, Q, W, Z, k, m, n and p are defined in WO/2010/112520.

Some embodiments of the present disclosure include compounds of Formula XI:

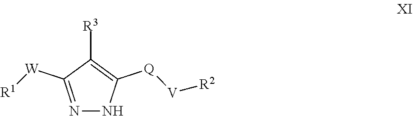

or salts, pharmaceutically acceptable salts, or prodrugs thereof.
wherein variables R¹, R², R³, Q, V, and W are defined in WO/2011/039358.

Illustrative compounds of Formulas IX, X, and XI are shown in Table 4.

TABLE 4

| | |
|---|---|
| 316 | 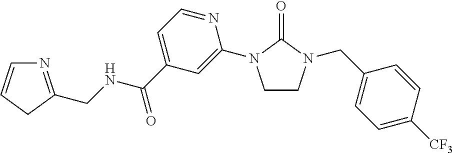 [SMILES: O=C(NCC1=NC=CC1)c1ccnc(N2CCN(Cc3ccc(C(F)(F)F)cc3)C2=O)c1] |
| 317 | 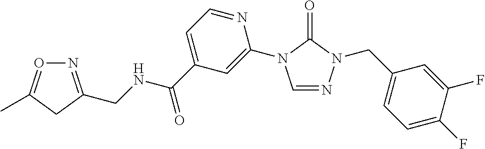 |
| 318 | 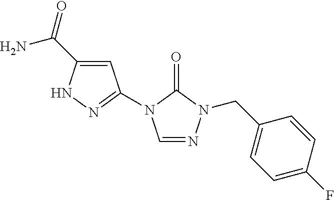 [SMILES: NC(=O)c1cc(-n2cnn(Cc3ccc(F)cc3)c2=O)n[nH]1] |
| 319 | 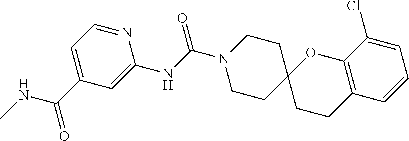 [SMILES: CNC(=O)c1ccnc(NC(=O)N2CCC3(CCc4cccc(Cl)c4O3)CC2)c1] |
| 320 | 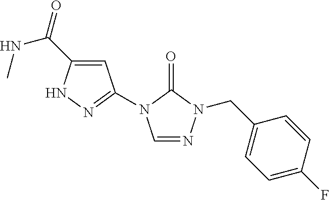 [SMILES: CNC(=O)c1cc(-n2cnn(Cc3ccc(F)cc3)c2=O)n[nH]1] |
| 321 | 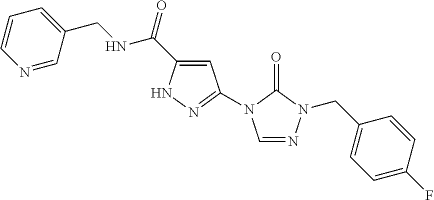 [SMILES: O=C(NCc1cccnc1)c1cc(-n2cnn(Cc3ccc(F)cc3)c2=O)n[nH]1] |

World Intellectual Property Organization, WO/2006/130986, describe compounds having Formula XII and is hereby incorporated by reference in its entirety.

Some embodiments of the present disclosure include compounds of Formula XII:

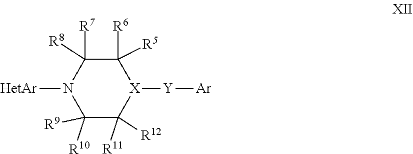

or salts, pharmaceutically acceptable salts, or prodrugs thereof.
wherein variables R⁵, R⁶, R⁷. R⁸. R⁹, R¹⁰, R¹¹, R¹², X, Y, Ar, and HetAr are defined in WO/2006/130986.

Some embodiments of the present disclosure include compounds of Formula XIII:

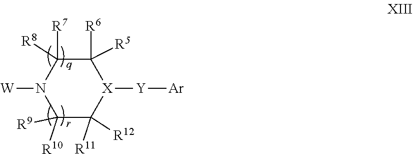

or salts, pharmaceutically acceptable salts, or prodrugs thereof.

wherein variables R⁵, R⁶, R⁷. R⁸. R⁹, R¹⁰, R¹¹, R¹², X, Y, Ar, W, q, and r are defined in WO/2008/064474.

Illustrative compounds of Formulas XII and XIII are shown in Table 5.

TABLE 5

| | |
|---|---|
| 322 | 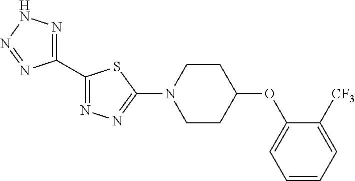 [SMILES: FC(F)(F)c1ccccc1OC1CCN(c2nnc(-c3nn[nH]n3)s2)CC1] |
| 323 | 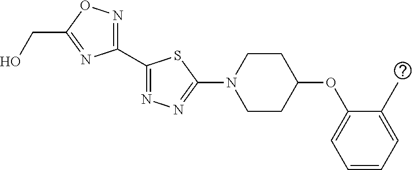 [SMILES: Cc1ccccc1OC1CCN(c2nnc(-c3noc(CO)n3)s2)CC1] |
| 324 | 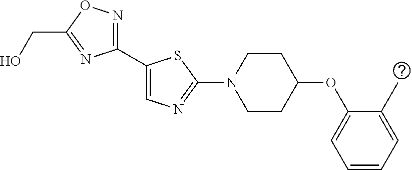 [SMILES: Cc1ccccc1OC1CCN(c2ncc(-c3noc(CO)n3)s2)CC1] |
| 325 | 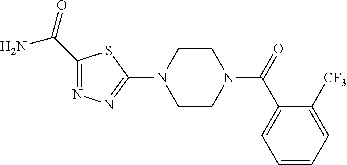 [SMILES: NC(=O)c1nnc(N2CCN(C(=O)c3ccccc3C(F)(F)F)CC2)s1] |
| 326 | 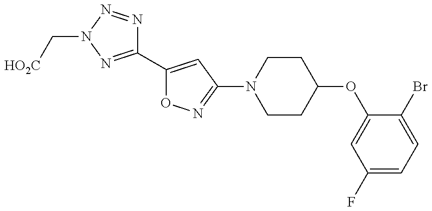 [SMILES: O=C(O)Cn1nnc(-c2cc(N3CCC(Oc4cc(F)ccc4Br)CC3)no2)n1] |

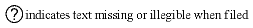

World Intellectual Property Organization, WO/2007/009236, Bioorganic & Medicinal Chemistry Letters (2010), 20, 499-502; and Bioorganic & Medicinal Chemistry Letters (2011), 21, 479-483 describe compounds having Formula XIV and are hereby incorporated by reference in its entirety.

Some embodiments of the present disclosure include compounds of Formula XIV:

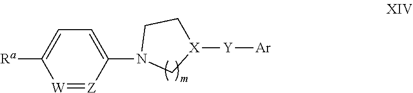

or salts, pharmaceutically acceptable salts, or prodrugs thereof wherein variables R^{a}, X, X, Y, Ar, and m are defined in WO/2007/009236.

Illustrative compounds of Formula (XIV) are shown in Table 6.

TABLE 6

| | |
|---|---|
| 327 | 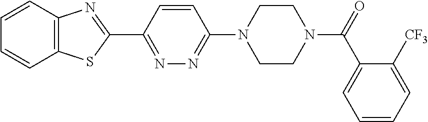 [SMILES: O=C(c1ccccc1C(F)(F)F)N1CCN(c2ccc(-c3nc4ccccc4s3)nn2)CC1] |
| 328 | 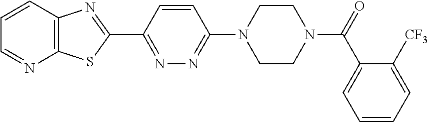 [SMILES: O=C(c1ccccc1C(F)(F)F)N1CCN(c2ccc(-c3nc4cccnc4s3)nn2)CC1] |
| 329 | 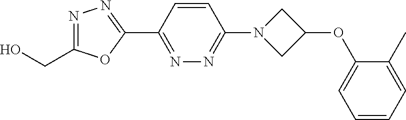 [SMILES: Cc1ccccc1OC1CN(c2ccc(-c3nnc(CO)o3)nn2)C1] |
| 330 | 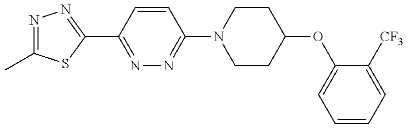 [SMILES: Cc1nnc(-c2ccc(N3CCC(Oc4ccccc4C(F)(F)F)CC3)nn2)s1] |

Compound 331 and analogs thereof are described in Bioorganic & Medicinal Chemistry Letters (2010), 20, 6366-6369 and Compound 332 and analogs thereof are described in Bioorganic & Medicinal Chemistry Letters (2011), 21, 5692-5696 and are hereby incorporated by reference in their entirety.

TABLE 7

| | |
|---|---|
| 331 | 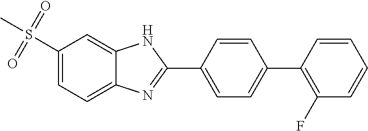 [SMILES: CS(=O)(=O)c1ccc2nc(-c3ccc(-c4ccccc4F)cc3)[nH]c2c1] |
| 332 | 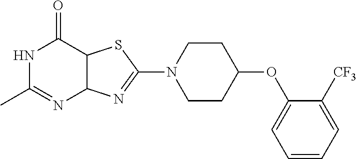 [SMILES: CC1=NC2N=C(N3CCC(Oc4ccccc4C(F)(F)F)CC3)SC2C(=O)N1] |

World Intellectual Property Organization, WO/2007/143824, describe compounds having Formula XV and is hereby incorporated by reference in its entirety.

Some embodiments of the present disclosure include compounds of Formula XV:

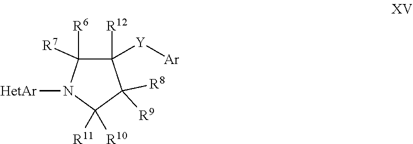

or salts, pharmaceutically acceptable salts, or prodrugs thereof wherein variables R⁶, R⁷. R⁸. R⁹, R¹⁰, R¹¹, R¹², Y, Ar, and HetAr are defined in WO/2007/143824.

An illustrative compound of Formula (XV) is shown in Table

TABLE 8

| | |
|---|---|
| 333 | 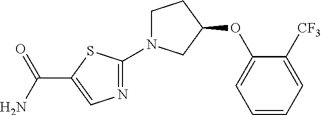 [SMILES: NC(=O)c1cnc(N2CC[C@@H](Oc3ccccc3C(F)(F)F)C2)s1] |

Compounds 334, 335 and analogs thereof are described in Bioorganic & Medicinal Chemistry Letters (2007), 17(12), 3388-3391, compound 336 and analogs thereof are described in Journal of Medicinal Chemistry (2007), 50(13), 3086-3100, and compound 337 and analogs thereof are described in Bioorganic & Medicinal Chemistry Letters (2008), 18(15), 4298-4302, and are hereby incorporated by reference in their entirety.

TABLE 9

| | |
|---|---|
| 334 | 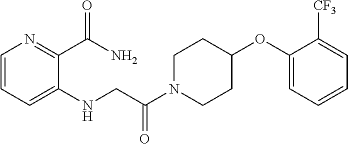 [SMILES: NC(=O)c1ncccc1NCC(=O)N1CCC(Oc2ccccc2C(F)(F)F)CC1] |
| 335 | 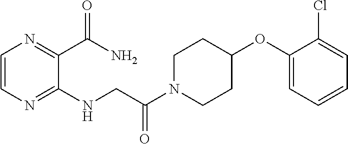 [SMILES: NC(=O)c1nccnc1NCC(=O)N1CCC(Oc2ccccc2Cl)CC1] |
| 336 | 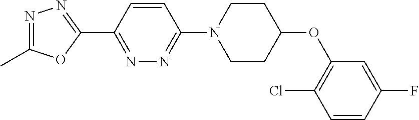 [SMILES: Cc1nnc(-c2ccc(N3CCC(Oc4cc(F)ccc4Cl)CC3)nn2)o1] |
| 337 | 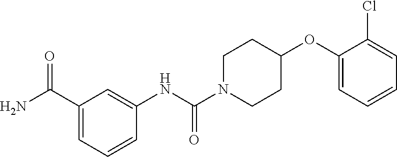 [SMILES: NC(=O)c1cccc(NC(=O)N2CCC(Oc3ccccc3Cl)CC2)c1] |

World Intellectual Property Organization, WO/2008/135141, WO/2010/028761, European Journal of Pharmacology (2013), 707(1-3), 140-146 describe compounds having Formula XVI and are hereby incorporated by reference in its entirety.

Some embodiments of the present disclosure include compounds of Formula XVI:

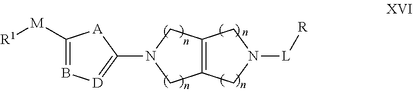

or salts, pharmaceutically acceptable salts, or prodrugs thereof wherein variables R, R¹, A, B, D, L, M, and n are defined in WO/2008/135141 and WO/2010/028761.

Illustrative compounds of Formula (XVI) are shown in Table 10.

TABLE 10

| | |
|---|---|
| 338 | 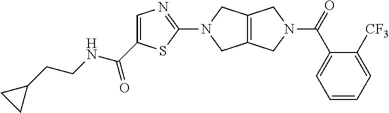 [SMILES: O=C(NCCC1CC1)c1cnc(N2CC3=C(CN(C(=O)c4ccccc4C(F)(F)F)C3)C2)s1] |
| 339 | 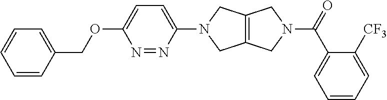 [SMILES: O=C(c1ccccc1C(F)(F)F)N1CC2=C(C1)CN(c1ccc(OCc3ccccc3)nn1)C2] |

World Intellectual Property Organization, WO/2009/024287 describe compounds having Formula XVII and is hereby incorporated by reference in its entirety.

Some embodiments of the present disclosure include compounds of Formula XVII:

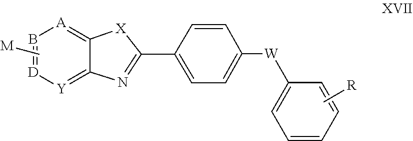

or salts, pharmaceutically acceptable salts, or prodrugs thereof wherein variables R, A, B, D, M, W, X, and Y are defined in WO/2009/024287.

Illustrative compounds of Formula (XVII) are shown in Table 11.

TABLE 11

| | |
|---|---|
| 340 | 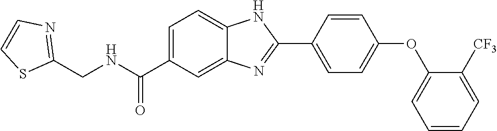 [SMILES: O=C(NCc1nccs1)c1ccc2[nH]c(-c3ccc(Oc4ccccc4C(F)(F)F)cc3)nc2c1] |
| 341 | 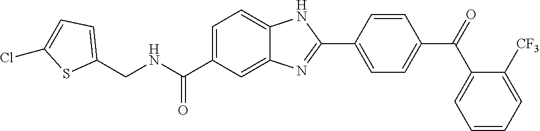 [SMILES: O=C(NCc1ccc(Cl)s1)c1ccc2[nH]c(-c3ccc(C(=O)c4ccccc4C(F)(F)F)cc3)nc2c1] |

World Intellectual Property Organization, WO/2008/157844, WO/2009/117659, WO/2009/117676 and U.S. application No. 20100160323, describe compounds having Formulas XVIII, XIX, XX, and XXI and are hereby incorporated by reference in their entirety.

Some embodiments of the present disclosure include compounds of Formula XVIII:

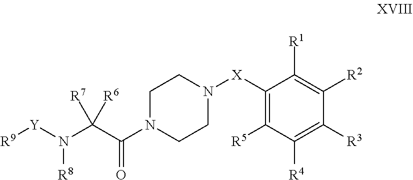

or salts, pharmaceutically acceptable salts, or prodrugs thereof.
wherein variables R¹, R², R³, R⁴, R⁵, R⁶, R⁷, R⁸, R⁹, X, and Y are defined in WO/2008/157844.

Some embodiments of the present disclosure include compounds of Formula XIX:

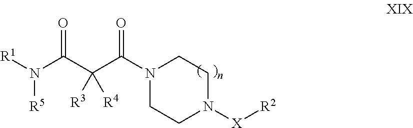

or salts, pharmaceutically acceptable salts, or prodrugs thereof wherein variables R¹, R², R³, R⁴, R⁵, X, and n are defined in WO/2009/117659.

Some embodiments of the present disclosure include compounds of Formula XX:

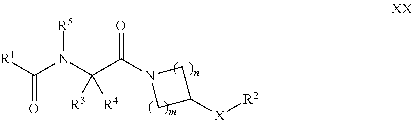

or salts, pharmaceutically acceptable salts, or prodrugs thereof wherein variables R¹, R², R³, R⁴, R⁵, X, m, and n are defined in WO/2009/117676.

Some embodiments of the present disclosure include compounds of Formula XXI:

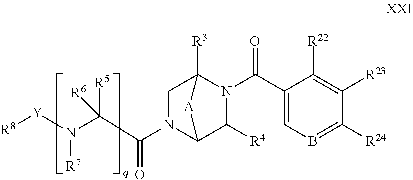

or salts, pharmaceutically acceptable salts, or prodrugs thereof wherein variables R³, R⁴, R⁵, R⁶, R⁷, R⁸, A, B, Y, and q are defined in US20100160323.

Illustrative compounds of Formulas XVIII, XIX, XX, and XXI are shown in Table 12.

TABLE 12

| | |
|---|---|
| 342 | 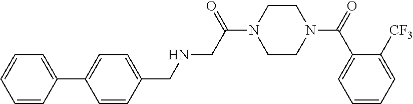 [SMILES: O=C(CNCc1ccc(-c2ccccc2)cc1)N1CCN(C(=O)c2ccccc2C(F)(F)F)CC1] |
| 343 | 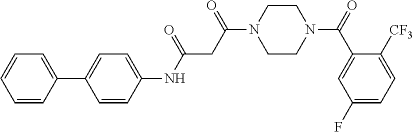 [SMILES: O=C(CC(=O)N1CCN(C(=O)c2cc(F)ccc2C(F)(F)F)CC1)Nc1ccc(-c2ccccc2)cc1] |
| 344 | 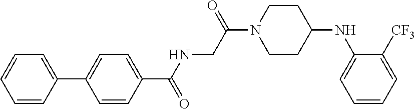 [SMILES: O=C(NCC(=O)N1CCC(Nc2ccccc2C(F)(F)F)CC1)c1ccc(-c2ccccc2)cc1] |
| 345 | 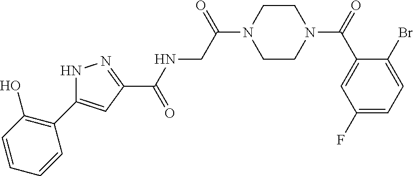 [SMILES: O=C(NCC(=O)N1CCN(C(=O)c2cc(F)ccc2Br)CC1)c1cc(-c2ccccc2O)[nH]n1] |
| 346 | 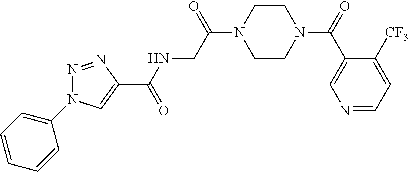 [SMILES: O=C(NCC(=O)N1CCN(C(=O)c2cnccc2C(F)(F)F)CC1)c1cn(-c2ccccc2)nn1] |
| 347 | 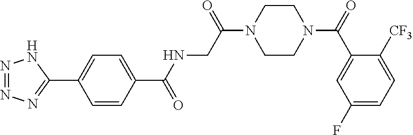 [SMILES: O=C(NCC(=O)N1CCN(C(=O)c2cc(F)ccc2C(F)(F)F)CC1)c1ccc(-c2nnn[nH]2)cc1] |

World Intellectual Property Organization, WO/2008/074832, WO/2008/104524, WO/2009/010560, WO/2009/016216, WO/2009/056556, WO/2009/060053, WO/2009/060054, WO/2009/150196, and Clinical Pharmacology in Drug Development (2019), 8(3), 270-280, describe compounds having Formulas XXII, XXIII, XXIV, XXV, XXVI, and XXVII and are hereby incorporated by reference in their entirety.

Some embodiments of the present disclosure include compounds of Formula XXII:

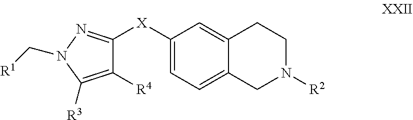

or salts, pharmaceutically acceptable salts, or prodrugs thereof.
wherein variables R¹, R², R³, R⁴, and X are defined in WO/2008/074832.

Some embodiments of the present disclosure include compounds of Formula XXIII:

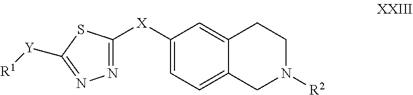

or salts, pharmaceutically acceptable salts, or prodrugs thereof.
wherein variables R¹, R², X, and Y are defined in WO/2008/104524.

Some embodiments of the present disclosure include compounds of Formula XXIV:

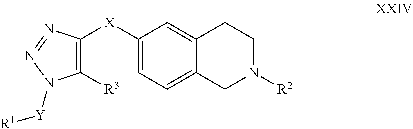

or salts, pharmaceutically acceptable salts, or prodrugs thereof.
wherein variables R¹, R², R³, X, and Y are defined in WO/2009/016216.

Some embodiments of the present disclosure include compounds of Formula XXV:

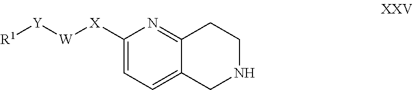

or salts, pharmaceutically acceptable salts, or prodrugs thereof.
wherein variables R¹, W, X, and Y are defined in WO/2009/056556.

Some embodiments of the present disclosure include compounds of Formula XXVI:

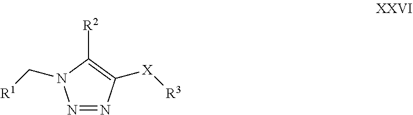

or salts, pharmaceutically acceptable salts, or prodrugs thereof.

wherein variables R¹, R², R³, and X are defined in WO/2009/060053 and WO/2009/060054.

Some embodiments of the present disclosure include compounds of Formula XXVII:

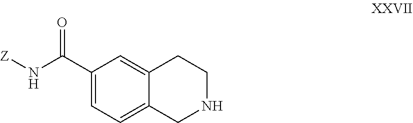

or salts, pharmaceutically acceptable salts, or prodrugs thereof.

wherein variable Z is defined in WO/2009/150196.

Illustrative compounds of Formulas XXII, XXIII, XXIV, XV, XXVI, and XXVII are shown in Table 13.

TABLE 13

| | |
|---|---|
| 348 | 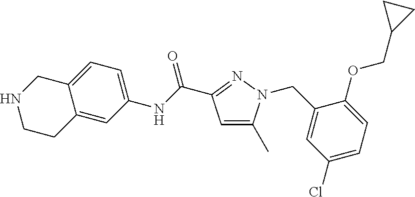 [SMILES: Cc1cc(C(=O)Nc2ccc3c(c2)CCNC3)nn1Cc1cc(Cl)ccc1OCC1CC1] |
| 349 | 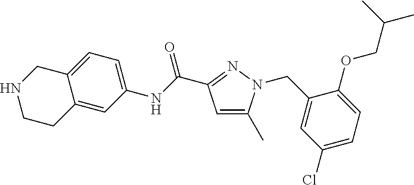 [SMILES: Cc1cc(C(=O)Nc2ccc3c(c2)CCNC3)nn1Cc1cc(Cl)ccc1OCC(C)C] |
| 350 | 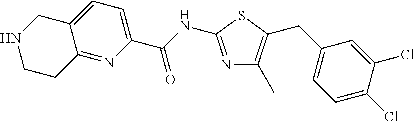 [SMILES: Cc1nc(NC(=O)c2ccc3c(n2)CCNC3)sc1Cc1ccc(Cl)c(Cl)c1] |
| 351 | 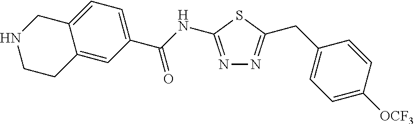 [SMILES: O=C(Nc1nnc(Cc2ccc(OC(F)(F)F)cc2)s1)c1ccc2c(c1)CCNC2] |
| 352 | 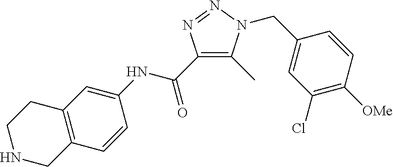 [SMILES: COc1ccc(Cn2nnc(C(=O)Nc3ccc4c(c3)CCNC4)c2C)cc1Cl] |
| 353 | 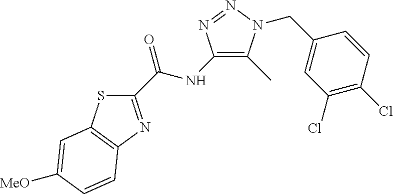 [SMILES: COc1ccc2nc(C(=O)Nc3nnn(Cc4ccc(Cl)c(Cl)c4)c3C)sc2c1] |
| 354 | 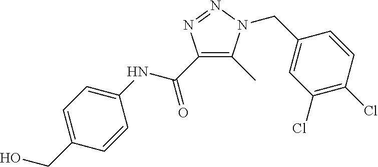 [SMILES: Cc1c(C(=O)Nc2ccc(CO)cc2)nnn1Cc1ccc(Cl)c(Cl)c1] |

World Intellectual Property Organization, WO/2008/044767 and WO/2008/096746 describe compounds having Formulas XXVIII and XXIX and are hereby incorporated by reference in their entirety.

Some embodiments of the present disclosure include compounds of Formula XXVIII:

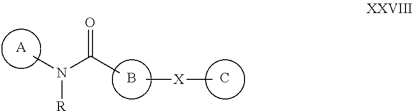

or salts, pharmaceutically acceptable salts, or prodrugs thereof.
wherein variables R, A, B, C, and x are defined in WO/2008/044767.

Some embodiments of the present disclosure include compounds of Formula XXIX:

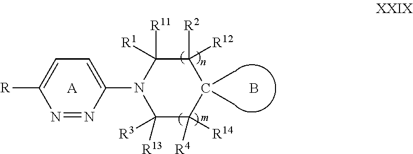

or salts, pharmaceutically acceptable salts, or prodrugs thereof.
wherein variables R¹, R², R³, R⁴, R, R¹², R¹³, R¹⁴. A, B, m, and n are defined in WO/2008/096746.

Illustrative compounds of Formulas XXVIII and XXIX are shown in Table 14.

TABLE 14

| | |
|---|---|
| 355 | 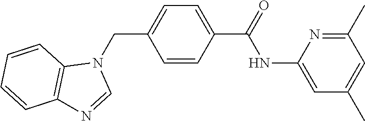 [SMILES: Cc1cc(C)nc(NC(=O)c2ccc(Cn3cnc4ccccc43)cc2)c1] |
| 356 | 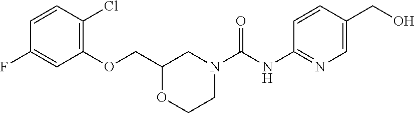 [SMILES: O=C(Nc1ccc(CO)cn1)N1CCOC(COc2cc(F)ccc2Cl)C1] |
| 357 | 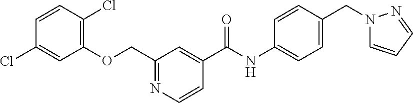 [SMILES: O=C(Nc1ccc(Cn2cccn2)cc1)c1ccnc(COc2cc(Cl)ccc2Cl)c1] |
| 358 | 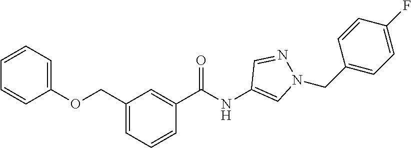 [SMILES: O=C(Nc1cnn(Cc2ccc(F)cc2)c1)c1cccc(COc2ccccc2)c1] |
| 359 | 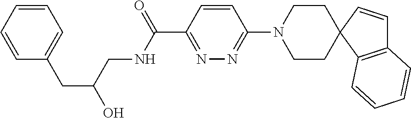 [SMILES: O=C(NCC(O)Cc1ccccc1)c1ccc(N2CCC3(C=Cc4ccccc43)CC2)nn1] |
| 360 | 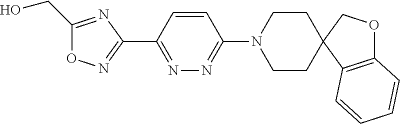 [SMILES: OCc1nc(-c2ccc(N3CCC4(CC3)COc3ccccc34)nn2)no1] |
| 361 | 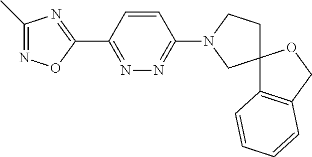 [SMILES: Cc1noc(-c2ccc(N3CCC4(C3)OCc3ccccc34)nn2)n1] |
| 362 | 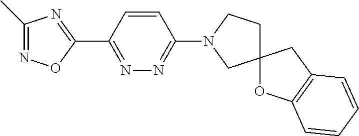 [SMILES: Cc1noc(-c2ccc(N3CCC4(Cc5ccccc5O4)C3)nn2)n1] |

Bioorganic & Medicinal Chemistry Letters (2011), 21(6), 1621-1625 describe compounds 363-365 shown in Table 15 and are hereby incorporated by reference in their entirety.

TABLE 15

| | |
|---|---|
| 363 | 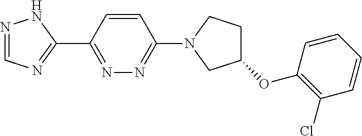 [SMILES: Clc1ccccc1O[C@H]1CCN(c2ccc(-c3ncn[nH]3)nn2)C1] |
| 364 | 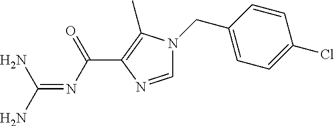 [SMILES: Cc1c(C(=O)N=C(N)N)ncn1Cc1ccc(Cl)cc1] |
| 365 | 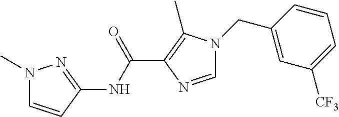 [SMILES: Cc1c(C(=O)Nc2ccn(C)n2)ncn1Cc1cccc(C(F)(F)F)c1] |

World Intellectual Property Organization, WO/2008/029266, WO/2008/062276, WO/2009037542, and WO/2010/007482 describe compounds having Formulas XXX, XXXI, XXXII, and XXXIII and are hereby incorporated by reference in their entirety.

Some embodiments of the present disclosure include compounds of Formula XXX:

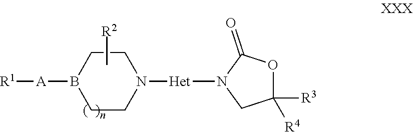

or salts, pharmaceutically acceptable salts, or prodrugs thereof wherein variables R¹, R², R³, R⁴, A, B, Het, and n are defined in WO/2008/029266.

Some embodiments of the present disclosure include compounds of Formula XXXI:

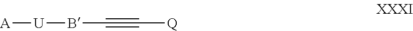

or salts, pharmaceutically acceptable salts, or prodrugs thereof.
wherein variables A, B′, Q, and U are defined in WO/2008/062276.

Some embodiments of the present disclosure include compounds of Formula XXXII:

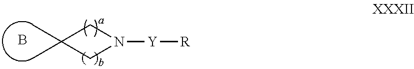

or salts, pharmaceutically acceptable salts, or prodrugs thereof wherein variables R, B, Y, a, and b are defined in WO/2009037542.

Some embodiments of the present disclosure include compounds of Formula XXXIII:

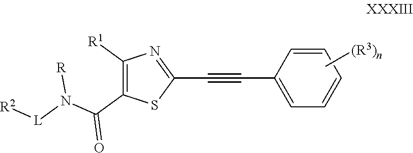

or salts, pharmaceutically acceptable salts, or prodrugs thereof wherein variables R, R¹, R², R³, L, and n are defined in WO/2010/007482.

Illustrative compounds of Formulas XXX, XXXI, XXXII, and XXXIII are shown in Table 16.

TABLE 16

| | |
|---|---|
| 366 | 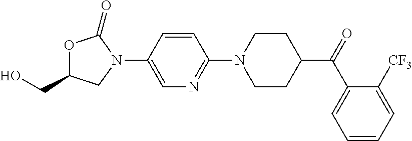 [SMILES: O=C(c1ccccc1C(F)(F)F)C1CCN(c2ccc(N3C[C@@H](CO)OC3=O)cn2)CC1] |
| 367 | 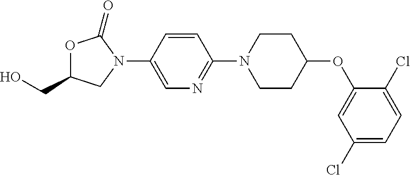 [SMILES: O=C1O[C@H](CO)CN1c1ccc(N2CCC(Oc3cc(Cl)ccc3Cl)CC2)nc1] |
| 368 | 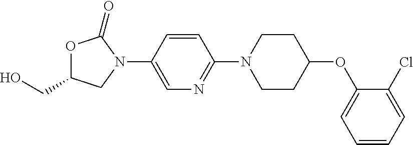 [SMILES: O=C1O[C@@H](CO)CN1c1ccc(N2CCC(Oc3ccccc3Cl)CC2)nc1] |
| 369 | 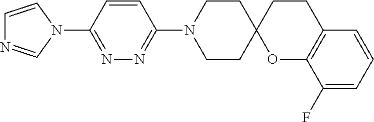 [SMILES: Fc1cccc2c1OC1(CC2)CCN(c2ccc(-n3ccnc3)nn2)CC1] |
| 370 | 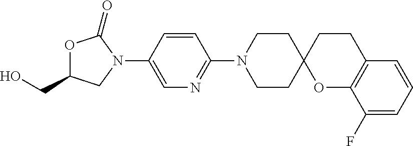 [SMILES: O=C1O[C@H](CO)CN1c1ccc(N2CCC3(CCc4cccc(F)c4O3)CC2)nc1] |
| 371 | 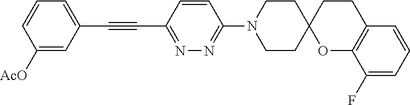 [SMILES: CC(=O)Oc1cccc(C#Cc2ccc(N3CCC4(CCc5cccc(F)c5O4)CC3)nn2)c1] |
| 372 | 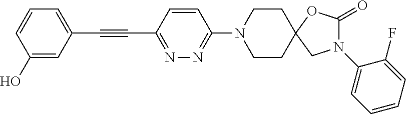 [SMILES: O=C1OC2(CCN(c3ccc(C#Cc4cccc(O)c4)nn3)CC2)CN1c1ccccc1F] |
| 373 | 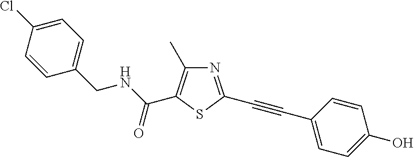 [SMILES: Cc1nc(C#Cc2ccc(O)cc2)sc1C(=O)NCc1ccc(Cl)cc1] |

World Intellectual Property Organization, WO/2008/062276, WO/2008/123891, and WO/2010/045374 describe compounds having Formulas XXXIV, XXXV, and XXXVI and are hereby incorporated by reference in their entirety.

Some embodiments of the present disclosure include compounds of Formula XXXIV:

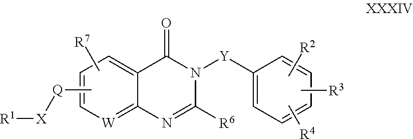

or salts, pharmaceutically acceptable salts, or prodrugs thereof wherein variables R¹, R², R³, R⁴, R⁵, R⁶, R⁷, Q, X, and Y are defined in WO/2008/062276.

Some embodiments of the present disclosure include compounds of Formula XXXV:

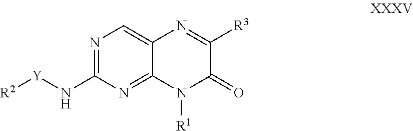

or salts, pharmaceutically acceptable salts, or prodrugs thereof wherein variables R¹, R², R³, and Y are defined in WO/2008/123891.

Some embodiments of the present disclosure include compounds of Formula XXXVI:

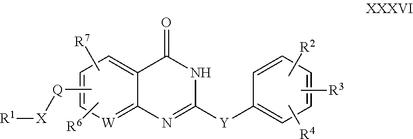

or salts, pharmaceutically acceptable salts, or prodrugs thereof.
wherein variables R¹, R², R³, R⁴, R⁶, R⁷, Q, X, and Y are defined in WO/2010/045374.

Illustrative compounds of Formulas XXXIV, XXXV, and XXXVI are shown in Table 17.

TABLE 17

| | |
|---|---|
| 374 | 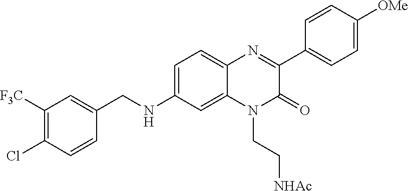 [SMILES: COc1ccc(-c2nc3ccc(NCc4ccc(Cl)c(C(F)(F)F)c4)cc3n(CCNC(C)=O)c2=O)cc1] |
| 375 | 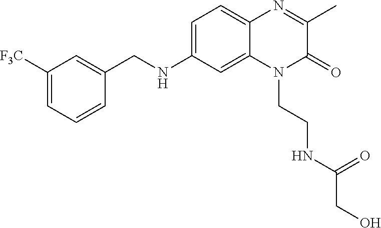 [SMILES: Cc1nc2ccc(NCc3cccc(C(F)(F)F)c3)cc2n(CCNC(=O)CO)c1=O] |
| 376 | 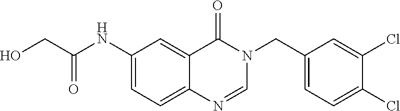 [SMILES: O=C(CO)Nc1ccc2ncn(Cc3ccc(Cl)c(Cl)c3)c(=O)c2c1] |

World Intellectual Property Organization, WO/2008/120744, WO/2008/120759, and WO/2008/123469 describe compounds having Formulas XXXVII, XXXVIII, and XXXIX and are hereby incorporated by reference in their entirety.

Some embodiments of the present disclosure include compounds of Formula XXXVII:

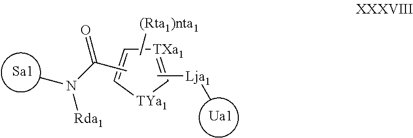

or salts, pharmaceutically acceptable salts, or prodrugs thereof wherein variables Sa1, Ua1, Rda₁, TYa₁, TXa₁, Lja₁, and (Rta₁)nta₁ are defined in WO/2008/120744.

Some embodiments of the present disclosure include compounds of Formula XXXVIII:

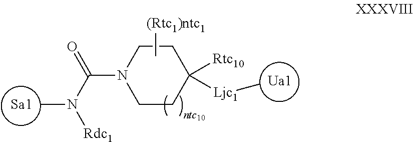

or salts, pharmaceutically acceptable salts, or prodrugs thereof wherein variables Sa1, Ua1, Rdc₁, Rtc₁₀, Ljc₁, (Rtc₁)ntc₁, and ntc₁₀ are defined in WO/2008/120759.

Some embodiments of the present disclosure include compounds of Formula XXXIX:

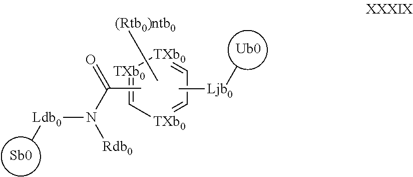

or salts, pharmaceutically acceptable salts, or prodrugs thereof wherein variables Sb0, Ub0, Rdb₀, Ldb₀, Ljb₀, TXb₀, and (Rtb₀)ntb₀ are defined in WO/2008/123469.

Illustrative compounds of Formulas XXXVII, XXXVIII, and XXXIX are shown in Table 18.

TABLE 18

| | |
|---|---|
| 377 | 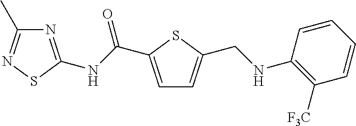 [SMILES: Cc1nsc(NC(=O)c2ccc(CNc3ccccc3C(F)(F)F)s2)n1] |
| 378 | 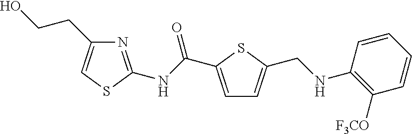 [SMILES: O=C(Nc1nc(CCO)cs1)c1ccc(CNc2ccccc2OC(F)(F)F)s1] |
| 379 | 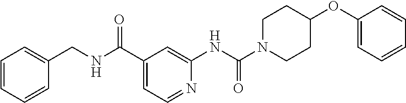 [SMILES: O=C(NCc1ccccc1)c1ccnc(NC(=O)N2CCC(Oc3ccccc3)CC2)c1] |
| 380 | 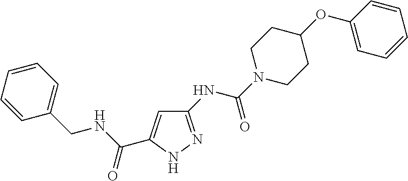 [SMILES: O=C(NCc1ccccc1)c1cc(NC(=O)N2CCC(Oc3ccccc3)CC2)n[nH]1] |
| 381 | 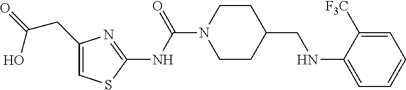 [SMILES: O=C(O)Cc1csc(NC(=O)N2CCC(CNc3ccccc3C(F)(F)F)CC2)n1] |
| 382 | 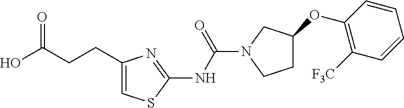 [SMILES: O=C(O)CCc1csc(NC(=O)N2CC[C@H](Oc3ccccc3C(F)(F)F)C2)n1] |
| 383 | 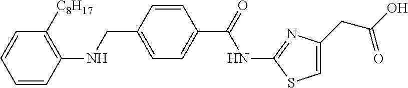 |
| 384 | 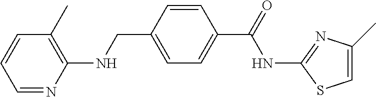 [SMILES: Cc1csc(NC(=O)c2ccc(CNc3ncccc3C)cc2)n1] |

World Intellectual Property Organization, WO/2010/006962, WO/2011/015629, and WO/2011/131593 describe compounds having Formulas XL, XLI, and XLII and are hereby incorporated by reference in their entirety.

Some embodiments of the present disclosure include compounds of Formula XL:

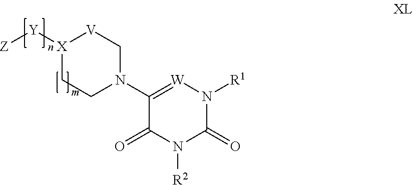

or salts, pharmaceutically acceptable salts, or prodrugs thereof wherein variables R¹, R², V, W, X, Y, Z, m, and n are defined in WO/2010/006962.

Some embodiments of the present disclosure include compounds of Formula XLI:

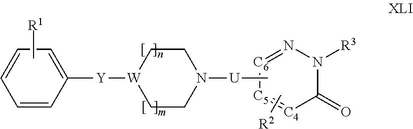

or salts, pharmaceutically acceptable salts, or prodrugs thereof.
wherein variables R¹, R², R³, U, V, W, Y, m, and n are defined in WO/2011/015629.

Some embodiments of the present disclosure include compounds of Formula XLII:

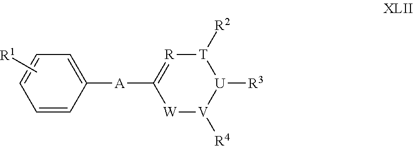

or salts, pharmaceutically acceptable salts, or prodrugs thereof wherein variables R¹, R², R³, R⁴, A, R, T, U, V, and W are defined in WO/2011/131593.

Illustrative compounds of Formulas XL, XLI, and XLII are shown in Table 19.

TABLE 19

| | |
|---|---|
| 385 | 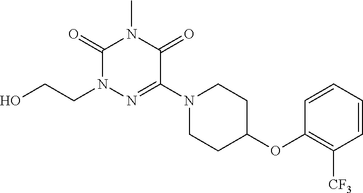 [SMILES: Cn1c(=O)c(N2CCC(Oc3ccccc3C(F)(F)F)CC2)nn(CCO)c1=O] |
| 386 | 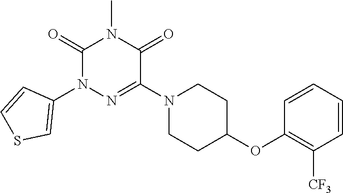 [SMILES: Cn1c(=O)c(N2CCC(Oc3ccccc3C(F)(F)F)CC2)nn(-c2ccsc2)c1=O] |
| 387 | 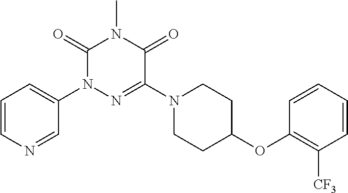 [SMILES: Cn1c(=O)c(N2CCC(Oc3ccccc3C(F)(F)F)CC2)nn(-c2cccnc2)c1=O] |
| 388 | 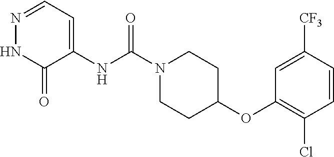 [SMILES: O=C(Nc1ccn[nH]c1=O)N1CCC(Oc2cc(C(F)(F)F)ccc2Cl)CC1] |
| 389 | 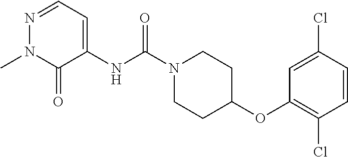 [SMILES: Cn1nccc(NC(=O)N2CCC(Oc3cc(Cl)ccc3Cl)CC2)c1=O] |
| 390 | 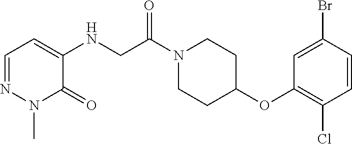 [SMILES: Cn1nccc(NCC(=O)N2CCC(Oc3cc(Br)ccc3Cl)CC2)c1=O] |
| 391 | 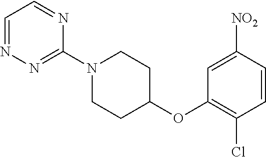 [SMILES: O=[N+]([O-])c1ccc(Cl)c(OC2CCN(c3nccnn3)CC2)c1] |
| 392 | 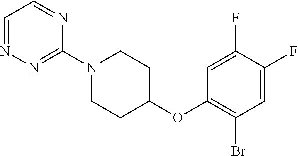 [SMILES: Fc1cc(Br)c(OC2CCN(c3nccnn3)CC2)cc1F] |
| 393 | 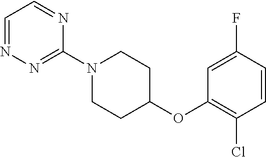 [SMILES: Fc1ccc(Cl)c(OC2CCN(c3nccnn3)CC2)c1] |

U.S. application No. 20170015654, describe compounds having Formula XLIII and is hereby incorporated by reference in their entirety.

Some embodiments of the present disclosure include compounds of Formula XLIII:

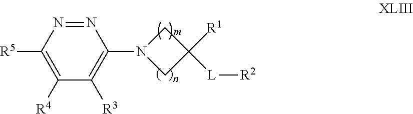

or salts, pharmaceutically acceptable salts, or prodrugs thereof.
wherein variables R¹, R², R³, R⁴, R⁵, L, m, and n are defined in US 20170015654.

Illustrative compounds of Formula XLIII are shown in Table 20.

TABLE 20

| | |
|---|---|
| 394 | 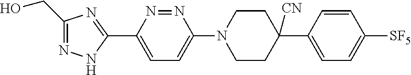 |
| 395 | 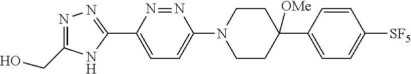 |
| 396 | 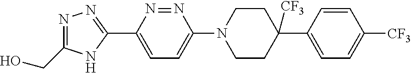 [SMILES: OCc1nnc(-c2ccc(N3CCC(c4ccc(C(F)(F)F)cc4)(C(F)(F)F)CC3)nn2)[nH]1] |

World Intellectual Property Organization, WO/1999/52932, WO/2015/083164 and WO/2020/095293 describe compounds having Formula XLIV and are hereby incorporated by reference in their entirety.

Some embodiments of the present disclosure include compounds of Formula XLIV:

W—X-G XLIV

and pharmaceutically acceptable salts, and prodrugs thereof, wherein variables W, X, and G as defined in published international application WO/1999/52932, including the meglumine and other salts of formula XLIV e.g., Aramchol. Such compounds are described in US patents 11,197.870, U.S. Pat. Nos. 11,166,964 and 10,849,911, which are hereby incorporated by reference in their entirety.

An illustrative compound of Formula XLIV is shown in Table 21.

TABLE 21

| | |
|---|---|
| 397 | 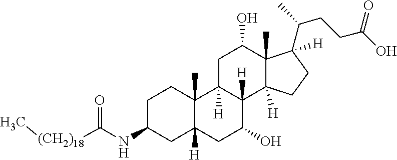 |

### Compound Preparation

The starting materials used in preparing the compounds of the disclosure are known, made by known methods, or are commercially available. It will be apparent to the skilled artisan that methods for preparing precursors and functionality related to the compounds claimed herein are generally described in the literature. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March's Advanced Organic Chemistry: Reactions, Mechanisms, and Structure 7^{th} Ed., John Wiley & Sons (2013), Carey and Sundberg, Advanced Organic Chemistry 5^{th} Ed., Springer (2007), Comprehensive Organic Transformations: A Guide to Functional Group Transformations, 2^{nd} Ed., John Wiley & Sons (1999) (incorporated herein by reference in its entirety) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in P. Wuts Greene's Protective Groups in Organic Synthesis, 5th Ed., John Wiley & Sons (2014), incorporated herein by reference in its entirety.

The following example schemes are provided for the guidance of the reader, and collectively represent an example method for making the compounds provided herein. Furthermore, other methods for preparing compounds of the disclosure will be readily apparent to the person of ordinary skill in the art in light of the following reaction schemes and examples. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application. Unless otherwise indicated, all variables are as defined above.

### General Procedures

General synthetic schemes for preparing compounds of Formula I are set forth below in Scheme 1 and Scheme 2.

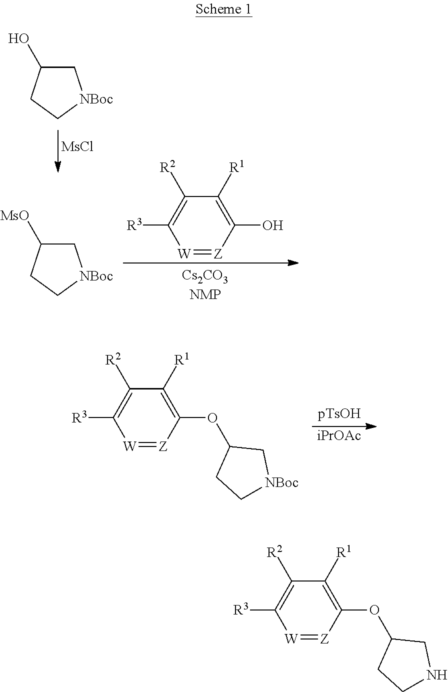

In Scheme 1 reacting the Boc protected pyrrolidine with mesyl chloride followed by SN2 type displacement with the anion derived from the suitable aromatic alcohol/phenol in the presence of a suitable base such as Cesium Carbonate affords the Boc protected derivative shown. In a separate transformation this can be readily hydrolyzed with p-toluene sulfonic acid in isopropyl acetate to the corresponding 3-substituted pyrrolidine. These transformations can be carried out with racemic materials, partially optically enriched materials and optically pure materials.

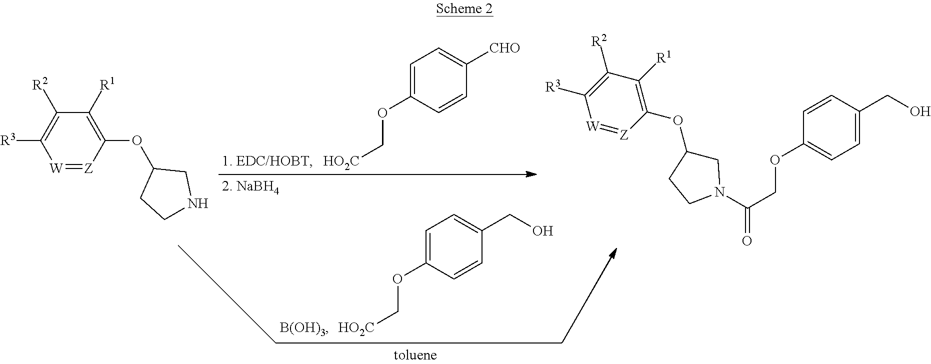

Scheme 2 outlines the two general synthetic routes leading to representative compounds. In particular, this synthesis exploits relatively mild amide bond forming reactions such as with carbodiimides. The 1^{st} route is completed by a sodium borohydride reduction of the side chain aldehyde to the alcohol wherein the 2^{nd} a route utilizes an acid already containing the methyl alcohol. Again, these transformations can be carried out with racemic materials, partially optically enriched materials and optically pure materials.

### Administration and Pharmaceutical Compositions

Some embodiments include pharmaceutical compositions comprising: (a) a therapeutically effective amount of a compound provided herein, or its corresponding enantiomer, diastereoisomer or tautomer, or pharmaceutically acceptable salt; and (b) a pharmaceutically acceptable carrier.

In some embodiments of the method described herein, an SCD-1 inhibitor is a compound of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV, and may be administered as a lotion, a spray, an ointment, a cream, a gel, a paste, and a patch. In some embodiments, SCD-1 inhibitor compounds disclosed herein are produced as a pharmaceutical composition suitable for compositions for topical or local administration.

For purposes of the method described herein, the SCD-1 inhibitor compound is (S)-2-(4-(hydroxymethyl)phenoxy)-1-(3-(2-(trifluoromethyl)phenoxy)pyrrolidin-1-yl)ethan-1-one (25) and may be administered as a lotion, a spray, an ointment, a cream, a gel, a paste, and a patch. In some embodiments, SCD-1 inhibitor compound 25 is produced as a pharmaceutical composition suitable for compositions for topical or local administration.

Topical formulations are ointments, creams, milks, pomades, powders, impregnated pads, films, solutions, gels, sprays, lotions or suspensions. They may also be in the form of microspheres or nanospheres or lipid or polymer vesicles or polymer patches and hydrogels allowing controlled release.

In some embodiments, the active pharmaceutical ingredient is a salt of an SCD-1 inhibitor compound. In some embodiments, the cation is selected from the group consisting of ingoganic ions such as sodium, potassium, lithium, ammonium, calcium, magnesium, iron, zinc, copper, manganese, aluminum, and beryllium. The cation may also be protonated organic amines, or amino acids.

In some embodiments, the active pharmaceutical ingredient is not a salt of an SCD-1 inhibitor compound. In some embodiments, the composition is a stable, water-soluble formulation.

The compounds can be administered either alone or in combination with a conventional pharmaceutical carrier, excipient or the like. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-$-cyclodextrins, or other solubilized derivatives can also be used to enhance delivery of compounds described herein. Actual methods of preparing dosage forms are known, or will be apparent, to those skilled in this art; for example, see Remington: The Science and Practice of Pharmacy, 22^{nd} a Edition (Pharmaceutical Press, London, U K. 2012).

Liquid pharmaceutically administrable compositions can, for example, be prepared by dissolving, dispersing, etc. a compound provided herein and optional pharmaceutical adjuvants in a carrier (e.g., water, saline, aqueous dextrose, glycerol, glycols, ethanol or the like) to form a solution, colloid, liposome, emulsion, complexes, coacervate or suspension. If desired, the pharmaceutical composition can also contain minor amounts of nontoxic auxiliary substances such as wetting agents, emulsifying agents, co-solvents, solubilizing agents, pH buffering agents and the like (e.g., sodium acetate, sodium citrate, cyclodextrin derivatives, sorbitan monolaurate, triethanolamine acetate, triethanolamine oleate, and the like).

In some embodiments, the pharmaceutically acceptable carrier which provides an environment of physical and chemical stability comprises a combination of one or more antioxidant, one or more chelator and a vehicle base comprising water and one or more pharmaceutically acceptable non-aqueous solvents, one or more absorption enhancers, one or more humectant, one or more gelling agents and one or more pH buffering agent. The antioxidant is selected from the group consisting of alpha tocopherol, beta tocopherol, delta tocopherol, gamma tocopherol, tocopherols, ascorbic acid, ascorbyl palmitate, butylated hydroxyanisol (BHA), butylated hydroxytoluene (BHT), citric acid, Coenzyme Q10 (CoQlO), epigallocatechin 3-gallate (EGCG), fumaric acid, idebenone, lycopene, malic acid, methionine, propyl gallate, silymarin sodium ascorbate, sodium metabisulfate, sodium thiosulfate, and sodium bisulfate.

In some embodiments, the antioxidant is butylated hydroxytoluene (BHT) at a concentration of least 0.05%. In another embodiment the butylated hydroxytoluene (BHT) is at a concentration of at least 0.1%.

In some embodiments, the topical formulation is substantially free of oxygen.

In some embodiments, the chelator is selected from ethylenediamine tetraacetic acid (EDTA) and its sodium, potassium and calcium salts, sodium pyrophosphate, citric acid, gluconic acid, catechol and various thiol derivatives.

In some embodiments, the chelator is di-sodium EDTA at a concentration of least 0.001%. In another embodiment the di-sodium EDTA is at a concentration of at least 0.005%.

In some embodiments, the one or more non-aqueous solvents is selected from ethanol, acetone, benzyl alcohol, 2-(2-ethoxyethoxy)ethanol, diethylene glycol monoethyl ether, glycerin, propylene glycol, propylene carbonate, acetone, hexylene glycol, isopropyl alcohol, polyethylene glycols (PEGs), methoxypolyethylene glycols, diethyl sebacate, dimethyl isosorbide, propylene carbonate, dimethyl sulfoxide (DMSO), diisopropyl adipate, isopropyl myristate, vegetable oils, a mineral oil, and isopropyl palmitate. Preferred non-aqueous solvents are ethanol, phenoxyethanol, diethylene glycol monoethyl ether (otherwise known as DEGEE or Transcutol P®), propylene glycol or PEG400.

In some embodiments, the one or more solvents or penetration enhancers include includes a fatty alcohol. As used herein, the term “fatty alcohol” refers to an aliphatic alcohol that is straight or branched and saturated or unsaturated. In some embodiments, the fatty alcohol is in a mixture of different fatty alcohols. In some embodiments, the fatty alcohol has between about 12-20, 14-20, 12-18, 14-18, or 16-18 carbons on average. Suitable fatty alcohols include, but are not limited to, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, oleyl alcohol, nonadecyl alcohol, arachidyl alcohol, heneicosyl alcohol, behenyl alcohol, erucyl alcohol, lignoceryl alcohol, or mixtures thereof. In some embodiments, the solvent or penetration enhancer includes one or more fatty alcohols selected from capric alcohol, lauryl alcohol, myristyl alcohol, cetyl alcohol, palmitoleyl alcohol, stearyl alcohol, oleyl alcohol, arachidyl alcohol, heneicosyl alcohol, behenyl alcohol, erucyl alcohol, and lignoceryl alcohol. In some embodiments, the one or more solvents or penetration enhancers include oleyl alcohol.

In some embodiments, fatty alcohol is present in an amount of about 0.5% to about 20% by weight of the base formulation. In some embodiments, fatty alcohol is present in an amount of from 1% to 20%, from 1% to 15%, 1% to 10%, from 5% to 20%, from 5% to 15%, or from 5% to 10% by weight of the base formulation. In some embodiments, fatty alcohol is present in an amount of about 10% by weight of the base formulation. In some embodiments, oleyl alcohol is present in an amount of from 0.5% to 20%, from 1% to 20%, from 1% to 15%, 1% to 10%, from 5% to 20%, from 5% to 15%, or from 5% to 10% by weight of the base formulation. In some embodiments, oleyl alcohol is present in an amount of about 10% by weight of the base formulation.

In some embodiments, the one or more solvents or penetration enhancers include a fatty acid. In some embodiments, the fatty acid is in a mixture of different fatty acids. In some embodiments, the fatty acid has between about 8 to about 30 carbons on average. In some embodiments, the fatty acid has about 12-20, 14-20, 12-18, 14-18, or 16-18 carbons on average. Suitable fatty acids include, but are not limited to, capric acid, neodecanoic acid, undecylic acid, lauric acid, tridecylic acid, myristic acid, pentadecylic acid, palmitic acid, margaric acid, stearic acid, isostearic acid, nonadecylic acid, arachidic acid, heneicosylic acid, behenic acid, tricosylic acid, lignoceric acid, caproleic acid, lauroleic acid, myristoleic acid, palmitoleic acid, sapienic acid, oleic acid, elaidic acid, vaccenic acid, gadoleic acid, eicosenoic acid, erucic acid, brassidic acid, nervonic acid, linoleic acid, eicosadienoic acid, docosadienoic acid, alpha-linolenic acid, gamma-linolenic acid, columbinic acid, pinolenic acid, alpha-eleostearic acid, beta-eleostearic acid, mead acid, dihomo-y-linolenic acid, eicosatrienoic acid, stearidonic acid, arachidonic acid, eicosapentaenoic acid, docosapentaenoic acid, docosahexaenoic acid, or mixtures thereof. In some embodiments, the fatty acid is selected from capric acid, neodecanoic acid, lauric acid, myristic acid, palmitic acid, stearic acid, isostearic acid, behenic acid, caproleic acid, lauroleic acid, myristoleic acid, palmitoleic acid, oleic acid, erucic acid, linoleic acid, linolenic acid, hydroxystearic acid, 12-hydroxy stearic acid, cetostearic acid, isostearic acid, sesquioleic acid, sesqui-9-octadecanoic acid, sesquisooctadecanoic acid, behenic acid, isobehenic acid, arachidonic acid, and combinations thereof. In some embodiments, the one or more solvents or penetration enhancers include one or more fatty acids selected from neodecanoic acid, isostearic acid, caproleic acid, lauroleic acid, myristoleic acid, palmitoleic acid, oleic acid, linoleic acid, and linolenic acid. In some embodiments, the one or more solvents or penetration enhancers include one or more fatty acids selected from neodecanoic acid, isostearic acid, and oleic acid. In some embodiments, the one or more solvents or penetration enhancers include neodecanoic acid. In some embodiments, the one or more solvents or penetration enhancers include isostearic acid. In some embodiments, the one or more solvents or penetration enhancers include oleic acid. In some embodiments, the one or more solvents or penetration enhancers include linoleic acid. In some embodiments, the one or more solvents or penetration enhancers include linolenic acid.

In one embodiment, the non-aqueous solvent is selected from ethanol in the range of 1.0-20.0% w/w, phenoxyethanol in the range 0.1-5.0% w/w, diethylene glycol monoethyl ether (otherwise known as DEGEE or Transcutol P®) in the range 5.0-40.0% w/w, propylene glycol in the range 5.0-40.0% w/w or PEG400 in the range 5.0-40.0% w/w. In a further embodiment, the pharmaceutical composition comprises three or more, four or more, or all of: ethanol in the range of 1.0-20.0% w/w, phenoxyethanol in the range 0.1-5.0% w/w, diethylene glycol monoethyl ether (otherwise known as DEGEE or Transcutol P®) in the range 5.0-40.0% w/w, propylene glycol in the range 5.0-40.0% w/w or PEG400 in the range 5.0-40.0% w/w. In yet further embodiments, ethanol is in the range 5.0-15.0% w/w, phenoxyethanol in the range 0.5-2.0% w/w, diethylene glycol monoethyl ether (otherwise known as DEGEE or Transcutol P®) in the range 20.0-30.0% w/w, propylene glycol in the range 15.0-25.0% w/w and/or PEG400 in the range 15.0-25.0% w/w. One or more pharmaceutically acceptable non-aqueous solvent which can also act as a topical absorption (permeation) enhancer is selected from ethanol, benzyl alcohol, propylene glycol, 2-(2ethoxyethoxy)ethanol, hexylene glycol, PEG400, diisopropyl adipate, diethylene glycol monoethyl ether (DEGEE or Transcutol P®), dimethylsulfoxide (DMSO), decylmethylsulfoxide, N,N-dimethyl acetamide, N,N-dimethyl formamide, 2-pyrrolidone, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, propylene glycol, ethanol, isopropanol, oleic acid, laurocapram (AZone), limonene, cineole, diethyl-m-toluamide (DEET), sodium dodecylsulfate, di-methyl Isosorbide, triethyl citrate, tetrahydrofurfuryl alcohol, glycerol monolaurate, methyl oleate, propylene glycol monolaurate, and oleyl alcohol.

In some embodiments, the one or more solvents or penetration enhancers are selected from the group consisting of DMSO, oleic acid, 2-(2-ethoxyethoxy)ethanol, dipropylene glycol, and PEG400.

In some embodiments, the one or more solvents or penetration enhancers do not include DMSO. In some embodiments, the one or more solvents or penetration enhancers include DMSO. In some embodiments, DMSO is present in an amount of less than 50%, less than 40%, less than 30%, or less than 20% by weight of the base formulation. In some embodiments, DMSO is present in an amount of from 30% to 50%, from 20% to 50%, from 10% to 50%, from 30% to 40%, from 20% to 40%, from 10% to 40%, from 20% to 30%, from 10% to 30%, or from 10% to 20% by weight of the base formulation. In some embodiments, DMSO is present in an amount of from 30% to 50%, from 20% to 50%, from 30% to 40%, from 20% to 40%, or from 20% to 30% by weight of the base formulation. In some embodiments, DMSO is present in an amount of from 20% to 40% or from 20% to 30% by weight of the base formulation. In some embodiments, DMSO is present in an amount of about 30% by weight of the base formulation.

In some embodiments, the topical absorption (permeation) enhancer is selected from diethylene glycol monoethyl ether (DEGEE or Transcutol P®), propylene glycol and ethanol. In one embodiment, at least one topical absorption (permeation) enhancer is selected from diethylene glycol monoethyl ether (DEGEE or Transcutol P®) in the range 5.0-40.0% w/w, propylene glycol in the range 5.0-40.0% w/w and ethanol in the range of 1.0-20.0% w/w. One or more humectant is selected from the groups consisting of hexylene glycol, glycerin, propylene glycol, sorbitol, lactic acid, sodium lactate, mannitol, butylene glycol, panthenol, hyaluronic acid, urea, chitosan, polyols, methyl gluceth-10, methyl gluceth-20, and polyethylene glycols.

In some embodiments, one or more humectants are selected from propylene glycol, polyethylene glycols and hexylene glycol. In one embodiment, one or more humectant is selected from propylene glycol, polyethylene glycols and hexylene glycol in the range 5.0-40.0% w/w.

In some embodiments, water is included at 5-40% v/v together with one or more pH buffering agents are selected from Trolamine or Sodium hydroxide or other suitable aqueous base in order to solubilize the gelling agents and stabilize the resulting semi-solid formulation. In one embodiment, the Trolamine or Sodium hydroxide provides an apparent pH in the range 6.50 to 7.50.

Hydrophilic polymer thickeners (gelling agents) that may be used in the invention include those known to one skilled in the art, such as hydrophilic and hydroalcoholic gelling agents frequently used in the cosmetic and pharmaceutical industries. In some embodiments, the one or more hydrophilic and hydrophobic gelling agents are Carbopols (now known as carbomers), carboxymethyl cellulose, ethylcellulose, gelatin, hydroxyethyl cellulose, hydroxypropyl cellulose, magnesium aluminum silicate (Veegum), methylcellulose, poloxamers (Pluronics), polyvinyl alcohol, sodium alginate, tragacanth, xanthan gum, or combinations thereof. In some embodiments, the one or more gelling agents include hydroxypropyl cellulose. In some embodiments, the hydroxypropyl cellulose has a molecular weight selected from the group consisting of 40,000 Da, 80,000 Da, 100,000 Da, 140,000 Da, 180,000 Da, 280,000 Da, 370,000 Da, 700,000 Da, 850,000 Da, 1,000,000 Da, 1,150,000 Da, and 2,500,000 Da. In some embodiments, the hydroxypropyl cellulose has the molecular weight selected from the group consisting of 140,000 Da, 180,000 Da, 280,000 Da, 370,000 Da, 700,000 Da, 850,000 Da, 1,000,000 Da, and 1,150,000 Da. In some embodiments, the hydroxypropyl cellulose has the molecular weight selected from the group consisting of 700,000 Da, 850,000 Da, 1,000,000 Da, and 1,150,000 Da.

In some embodiments, one or more gelling agents are selected from hydroxypropylcellulose, hydroxyethylcellulose, hydroxypropyl methyl cellulose, polyvinyl alcohol, povidone, fatty alcohols, cetylalcohol, stearyl alcohol, cetostearyl alcohol, and myristyl alcohol, carbomer, carboxymethylcellulose, xanthan gum, guar gum, chitosan, carrageenan and alginic acid.

When the one or more gelling agents are present, in some embodiments, the topical formulation has a viscosity of 5,000 to 100,000 cP. When the one or more gelling agents are present, in some embodiments, the topical formulation has a viscosity of 5,000 to 50,000 cP. When the one or more gelling agents are present, in some embodiments, the topical formulation has a viscosity of 5,000 to 15,000 cP. When the hydroxypropyl cellulose is present, in some embodiments, the topical formulation has a viscosity of 5,000 to 100,000 cP. When the hydroxypropyl cellulose is present, in some embodiments, the topical formulation has a viscosity of 5,000 to 50,000 cP. When the hydroxypropyl cellulose is present, in some embodiments, the topical formulation has a viscosity of 5,000 to 15,000 cP.

In some embodiments, the one or more gelling agents are present in an amount of from about 0.5% to about 30% by weight of the base formulation, while the topical formulation has a viscosity of 5,000 to 100,000 cP. In some embodiments, the one or more gelling agents are present in an amount of from about 0.5% to about 30% by weight of the base formulation, while the topical formulation has a viscosity of 5,000 to 50,000 cP. In some embodiments, the one or more gelling agents are present in an amount of from about 0.5% to about 30% by weight of the base formulation, while the topical formulation has a viscosity of 5,000 to 15,000 cP. In some embodiments, the hydroxypropyl cellulose is present in an amount of from about 0.5% to about 5%, from about 5% to about 10%, from about 10% to about 20%, or from about 20% to about 30% by weight of the base formulation, while the topical formulation has a viscosity of 5,000 to 100,000 cP. When a hydroxypropyl cellulose having an average molecular weight of less than 700,000 Da is used, in some embodiments, the hydroxypropyl cellulose is present in an amount of about 5% to about 30% by weight of the base formulation, while the topical formulation has a viscosity of 5,000 to 100,000 cP. In some embodiments, the hydroxypropyl cellulose is present in an amount of from 0.5% to 4%, from 0.5% to 3%, from 0.5% to 2%, from 1% to 5%, from 1% to 4%, from 1% to 3%, from 1% to 2%, or from 2% to 5% by weight of the base formulation, while the topical formulation has a viscosity of 5000 to 15000 cP. In some embodiments, the hydroxypropyl cellulose having an average molecular weight selected from 700,000 Da and 1,150,000 Da is present in an amount of about 2% by weight of the base formulation. In some embodiments, the hydroxypropyl cellulose having an average molecular weight selected from 700,000 Da and 1,150,000 Da is present in an amount of about 1% by weight of the base formulation.

In some embodiments, the gelling agents is a carbomer such as carbomer homopolymer type C980. In one embodiment, the carbomer homopolymer type C980 is in the range of 0.5 to 2.0% w/w.

In a further embodiment, the pharmaceutical composition comprises two or more of: (i) butylated hydroxytoluene (BHT) at a concentration of least 0.05%; (ii) di-sodium EDTA at a concentration of least 0.001%; and (iii) Trolamine to provide an apparent pH in the range 6.50 to 7.50.

In certain embodiments, the pharmaceutical composition comprises both (i) butylated hydroxytoluene (BHT) at a concentration of least 0.05%; and (ii) di-sodium EDTA at a concentration of least 0.001%.

In another embodiment, the pharmaceutical composition comprises each of (i) butylated hydroxytoluene (BHT) at a concentration of least 0.05%; (ii) di-sodium EDTA at a concentration of least 0.001%; and (iii) Trolamine to provide an apparent pH in the range 6.50 to 7.50.

In some embodiment, the pharmaceutical composition comprises: (i) ethanol in the range of 1.0-20.0% w/w; (ii) phenoxyethanol in the range 0.1-5.0% w/w; (iii) diethylene glycol monoethyl ether (otherwise known as DEGEE or Transcutol P®) in the range 5.0-40.0% w/w; (iv) propylene glycol in the range 5.0-40.0% w/w; (v) PEG400 in the range 5.0-40.0% w/w; and (vi) a carbomer such as carbomer homopolymer type C980 in the range of 0.5 to 2.0% w/w.

In another embodiment, the pharmaceutical composition comprises: (i) ethanol in the range of 1.0-20.0% w/w; (ii) phenoxyethanol in the range 0.1-5.0% w/w; (iii) diethylene glycol monoethyl ether (otherwise known as DEGEE or Transcutol P®) in the range 5.0-40.0% w/w; (iv) propylene glycol in the range 5.0-40.0% w/w; (v) PEG400 in the range 5.0-40.0% w/w; (vi) a carbomer such as carbomer homopolymer type C980 in the range of 0.5 to 2.0% w/w; (vii) butylated hydroxytoluene (BHT) at a concentration of least 0.05%; (viii) di-sodium EDTA at a concentration of least 0.001%; and (ix) Trolamine to provide an apparent pH in the range 6.50 to 7.50.

In another embodiment, the pharmaceutical composition comprises: (i) ethanol in the range of 5.0-15.0% w/w; (ii) phenoxyethanol in the range 0.5-2.0% w/w; (iii) diethylene glycol monoethyl ether (otherwise known as DEGEE or Transcutol P®) in the range 20.0-30.0% w/w; (iv) propylene glycol in the range 15.0-25.0% w/w; (v) PEG400 in the range 15.0-25.0% w/w; and (vi) a carbomer such as carbomer homopolymer type C980 in the range of 0.75 to 1.5% w/w.

In another embodiment, the pharmaceutical composition comprises: (i) ethanol in the range of 5.0-15.0% w/w; (ii) phenoxyethanol in the range 0.5-2.0% w/w; (iii) diethylene glycol monoethyl ether (otherwise known as DEGEE or Transcutol P®) in the range 20.0-30.0% w/w; (iv) propylene glycol in the range 15.0-25.0% w/w; (v) PEG400 in the range 15.0-25.0% w/w; (vi) a carbomer such as carbomer homopolymer type C980 in the range of 0.75 to 1.5% w/w. (vii) butylated hydroxytoluene (BHT) at a concentration of least 0.1%; (viii) di-sodium EDTA at a concentration of least 0.005%; and (ix) Trolamine to provide an apparent pH in the range 6.50 to 7.50.

In another embodiments, the pharmaceutical composition comprises: (i) ethanol at a concentration of 10.0% w/w; (ii) phenoxyethanol at a concentration of 1% w/w; (iii) diethylene glycol monoethyl ether (otherwise known as DEGEE or Transcutol P®) at a concentration of 25.0% w/w; (iv) propylene glycol at a concentration of 20.0% w/w; (v) PEG400 at a concentration of 21.0% w/w; (vi) carbomer homopolymer type C980 at a concentration of 1.0% w/w; and (vii) water at a concentration of 19.5-22% w/w.

In yet other specific embodiments, the pharmaceutical composition comprises: (i) ethanol at a concentration of 10.0% w/w; (ii) phenoxyethanol at a concentration of 1% w/w; (iii) diethylene glycol monoethyl ether (otherwise known as DEGEE or Transcutol P®) at a concentration of 25.0% w/w; (iv) propylene glycol at a concentration of 20.0% w/w; (v) PEG400 at a concentration of 21.0% w/w; (vi) carbomer homopolymer type C980 at a concentration of 1.0% w/w; (vii) butylated hydroxytoluene (BHT) at a concentration of 0.1% w/w; (viii) di-sodium EDTA at a concentration of 0.005% w/w; (ix) Trolamine at a concentration of 0.375% w/w; and (x) water at a concentration of 19.02-21.52% w/w.

In yet other specific embodiments, the pharmaceutical composition of either of the above two embodiments wherein the compound is (S)-2-(4-(hydroxymethyl)phenoxy)-1-(3-(2-(trifluoromethyl)phenoxy)pyrrolidin-1-yl)ethan-1-one (25) at a concentration up to 2.50% w/w, particularly at a concentration of 0.25%, 0.75% or 1.75%. In yet further embodiments the pharmaceutically acceptable carrier is a cream or a lotion, which provides an environment of physical and chemical stability, comprising a combination of one or more antioxidant, one or more chelator and a vehicle base comprising water and one or more pharmaceutically acceptable non-aqueous solvents, one or more oil, one or more structural lipids, one or more absorption enhancers, one or more aqueous emulsifier surfactants, one or more emollients, one or more humectant, one or more gelling agents and one or more pH buffering agent.

In another embodiments, one or more oils are selected from hydrogenated castor oil, liquid paraffin, white soft paraffin, corn oil, cottonseed oil, ethyl oleate, petrolatum, sesame oil, peanut oil, soybean oil, safflower oil, olive oil, almond oil, coconut oil, walnut oil, avocado nut oil.

In another embodiments, the combination of oils is liquid paraffin at not less than 2% and white soft paraffin at not less than 1%.

In further embodiments, one or more antioxidant is selected from the group consisting of alpha tocopherol, beta tocopherol, delta tocopherol, gamma tocopherol, tocopherols, ascorbic acid, ascorbyl palmitate, butylated hydroxyanisol (BHA), butylated hydroxytoluene (BHT), fumaric acid, malic acid, methionine, propyl gallate, sodium ascorbate, sodium metabisulfate, sodium thiosulfate, sodium bisulfate.

In another embodiments, the antioxidant is butylated hydroxytoluene (BHT) at a concentration of least 0.05%. In other embodiments one or more structural lipids are selected from stearic acid, stearyl alcohol, cetostearyl alcohol, cetrimide, cetyl alcohol, cetyl esters wax, lanolin, lanolin alcohols, emulsifying wax, microcrystalline wax, white wax, yellow wax, hydrogenated castor oil.

In another embodiments, the structural lipid is cetostearyl alcohol at not less than 1%.

In other embodiments, one or more oil and aqueous emulsifier surfactants are selected from medium chain triglycerides, Tween 60, Tween 80, Span 60, Brij 721, Brij 72, Aracel 165, Polyoxyethylene castor oil derivatives, Cetomacrogol 1000, Polyoxyethylene stearates. A preferred combination of surfactants is Brij 721 at not less than 1% with Brij 72 at not less than 2%.

In other embodiments, one or more emollients are selected from diisopropyl adipate, isopropyl myristate, isopropyl palmitate, cetearyl octonoate, isopropyl isostearate, myristyl lactate, octyldodecanol, oleyl alcohol, mineral oil, petrolatum, vegetable/plant oils (e.g., peanut, soybean, safflower, olive, almond, coconut), PPG-15 stearyl ether, PPG-26 oleate, PEG-4 dilaurate, lecithin, lanolin, lanolin alcohol, polyoxyl 75 lanolin, cholesterol, cetyl esters wax, cetostearyl alcohol, glyceryl monostearate, medium chain triglycerides, dimethicone, and cyclomethicone.

In another embodiments, the emollient combination is cetostearyl alcohol at not less than 1% and Crodamoi GTCC medium chain triglydcerides at not less than 6%

In other embodiments, one or more pharmaceutically acceptable non-aqueous solvents which can also act as absorption enhancers are selected from propylene glycol, 2-(2ethoxyethoxy)ethanol, hexylene glycol, PEG400, diisopropyl adipate, diethylene glycol monoethyl ether (DEGEE or Transcutol P®),Dimethylsulfoxide (DMSO), decylmethylsulfoxide, N,N-dimethyl acetamide, N,N-dimethyl formamide, 2-pyrrolidone, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, propylene glycol, ethanol, isopropanol, oleic acid, laurocapram (AZone), limonene, cineole, diethyl-m-toluamide (DEET), sodium dodecylsulfate, di-methyl isosorbide, triethyl citrate, tetrahydrofurfuryl alcohol, glycerol monolaurate, methyl oleate, propylene glycol monolaurate, and oleyl alcohol, alcohol (ethanol), acetone, benzyl alcohol, phenoxyethanol, diethylene glycol monoethyl ether (Transcutol P), glycerin, hexylene glycol, propylene glycol, isopropyl alcohol, polyethylene glycols (PEGs), methoxypolyethylene glycols, diethyl sebacate, dimethyl isosorbide, propylene carbonate, and dimethyl sulfoxide. A preferred non-aqueous solvent combination is ethanol at not less than 8%, PEG400 at not less than 20%, phenoxyethanol at not less than 1%, diethylene glycol monoethyl ether (Transcutol P) at not less than 12% and glycerol at not less than 8%. In further embodiments one or more pH buffering agents are selected from sodium citrate, monosodium phosphate, sodium acetate, sodium lactate, sodium tartrate, sodium fumarate at or around pH 5.5 to pH 6. A preferred buffer system is sodium citrate at 0.01M adjusted to pH 5.5.

In another embodiments, one or more humectants are selected from glycerol, hexylene glycol, propylene glycol, sorbitol, lactic acid, sodium lactate, mannitol, butylene glycol, panthenol, hyaluronic acid, urea, chitosan, polyols, methyl gluceth-10, methyl gluceth-20, and polyethylene glycols (PEG's).

In another embodiments, the humectants are glycerol at not less than 8% and PEG 400 at not less than 20%.

In other embodiments, one or more gelling agents are selected from hydroxypropylcellulose, hydroxyethylcellulose, hydroxypropyl methyl cellulose, polyvinyl alcohol, povidone, fatty alcohols, cetylalcohol, stearyl alcohol, cetostearyl alcohol, and myristyl alcohol, carbomer, carboxymethylcellulose, xanthan gum, guar gum, chitosan, carrageenan and alginic acid.

In another embodiments, the gelling agent is a carbomer such as carbomer homopolymer type C980 at not less than 0.25%. In further embodiments, a compound of Formulas I, II, III, IV, V, VI, VII, VIII, IX, X, XI, XII, XIII, XIV, XV, XVI, XVII, XVIII, XIX, XX, XXI, XXII, XXIII, XIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, and XLIV is present at a concentration between about 0.005% and about 5% by weight. In certain embodiments the compound is present in the pharmaceutical composition at a concentration between about 0.01% and about 2.5% w/w, and in specific alternative embodiments the pharmaceutical composition is at a concentration of 0.25%, 0.75% or 1.75% w/w. In yet further embodiments, the compound is a racemic mixture, a diastereoisomeric mixture, a single enantiomer, an enantiomeric diastereomer, a meso compound, a pure epimer, or a mixture of epimers thereof.

SCD-1 inhibitor compounds provided herein intended for pharmaceutical use may be administered as crystalline or amorphous products which may also be salts, complexes or co-crystals. Another desirable characteristic for the liquid solvent is that it is sterile. An aqueous liquid would be subject to the risk of considerable microbiological contamination and growth if no measures were taken to ensure sterility. In order to provide a substantially sterile liquid, an effective amount of an acceptable antimicrobial agent or preservative can be incorporated or the liquid can be sterilized prior to providing it and to seal it with an air-tight seal. In one embodiment, the liquid is a sterilized liquid free of preservatives and provided in an appropriate air-tight container.

In some embodiments, the composition may be in the form of a patch. In some embodiments, each patch contains a single unit dose for administration to a subject.

It is to be noted that concentrations and dosage values may also vary depending on the specific compound and the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Also provided herein are kits. Typically, a kit includes one or more compounds or compositions as described herein. In certain embodiments, a kit can include one or more delivery systems, e.g., for delivering or administering a compound as provided herein, and directions for use of the kit (e.g., instructions for treating a patient). In another embodiment, the kit can include a compound or composition as described herein and a label that indicates that the contents are to be administered to a patient.

The compositions of the disclosure that include SCD-1 inhibitor compounds may be applied to different areas of the skin of a mammal directly or indirectly in any convenient or desired manner, such as by a lotion, a spray, an ointment, a cream, a gel, a paste, or a patch. These compositions may be applied to any sized area of the skin including, but not limited to, an area the size of a pinpoint (or smaller) to an area that spans one or a plurality of square centimeters, inches or even feet (or larger).

In some embodiments, the topical treatment containing a SCD-1 inhibitor compound was applied to <1% of the total body surface area (BSA) of the skin, in some embodiments, the topical treatment was applied to about 2% of the total BSA of the skin, in some embodiments, the topical treatment was applied to about 3% of the total BSA of the skin, in some embodiments, the topical treatment was applied to about 4% of the total BSA of the skin, in some embodiments, the topical treatment was applied to about 5% of the total BSA of the skin, in some embodiments, the topical treatment was applied to about 6% of the total BSA of the skin, in some embodiments, the topical treatment was applied to about 7% of the total BSA of the skin, in some embodiments, the topical treatment was applied to about 8% of the total BSA of the skin, in some embodiments, the topical treatment was applied to about 9% of the total BSA of the skin, in some embodiments, the topical treatment was applied to about 10% of the total BSA of the skin, in some embodiments, the topical treatment was applied to about 15% of the total BSA of the skin, in some embodiments, the topical treatment was applied to about 20% of the total BSA of the skin, in some embodiments, the topical treatment was applied to about 25% of the total BSA of the skin, in some embodiments, the topical treatment was applied to about 30% of the total BSA of the skin, and in some embodiments, the topical treatment was applied to about 35% of the total BSA of the skin.

In some embodiments, the topical treatment containing a SCD-1 inhibitor compound was applied on a continuous daily dosing schedule, once a day, twice a day, three times a day, once every two days, once every three days, once a week, once every two weeks, once a month, or once every two months.

In some embodiments, the topical treatment containing a SCD-1 inhibitor compound was applied for 1 week; in some embodiments, for 2 weeks; in some embodiments, for 3 weeks; in some embodiments, for 4 weeks; in some embodiments, for 5 weeks; in some embodiments, for 6 weeks; in some embodiments, for 7 weeks; in some embodiments, for 8 weeks; in some embodiments, for 9 weeks; in some embodiments, for 10 weeks; in some embodiments, for 11 weeks; in some embodiments, for 12 weeks; in some embodiments, for 13 weeks; in some embodiments, for 14 weeks; in some embodiments, for 15 weeks; in some embodiments, for 16 weeks; in some embodiments, for 17 weeks; in some embodiments, for 18 weeks; in some embodiments, for 19 weeks; in some embodiments, for 20 weeks; in some embodiments, for 21 weeks; in some embodiments, for 22 weeks; in some embodiments, for 23 weeks; in some embodiments, for 24 weeks; in some embodiments, for 25 weeks; in some embodiments, for 26 weeks; in some embodiments, for 1 month; in some embodiments, for 2 months; in some embodiments, for 3 months; in some embodiments, for 4 months; in some embodiments, for 5 months; in some embodiments, for 6 months; in some embodiments, for 7 months; in some embodiments, for 8 months; in some embodiments, for 9 months; in some embodiments, for 10 months; in some embodiments, for 11 months; and in some embodiments, for 12 months.

In certain embodiments, each administration of the SCD-1 inhibitor compounds is applied to the same location, or to several different locations on the individual. When applied to different locations, the doses for each location may be the same, or be adjusted based on factors such as skin thickness and differences in drug penetration (if any). In certain embodiments, the formulations can be applied to any skin surface, including for example, the skin of the abdomen, hands, neck, chest, back, arms, or legs.

### Methods of Treatment

The compounds and compositions described herein can be used as anti-fibrotic agents. In another aspect, the disclosure provides methods of reducing fibrosis in cells or tissues. In addition, the compounds can be used as inhibitors of SCD-1.

These methods can be carried out in vitro or in vivo. In one aspect, the method is carried out in vivo, for example, in a mammalian subject (e.g., an animal model). In one aspect the subject is a human. In some aspects, reducing fibrosis includes: (a) reducing or inhibiting the formation or deposition of tissue fibrosis; (b) reducing the size, cellularity (e.g., fibroblast or immune cell numbers), composition; or cellular content, of a fibrotic lesion; (c) reducing the collagen or hydroxyproline content, of a fibrotic lesion; (d) reducing expression or activity of one or more fibrogenic proteins; and/or (e) reducing fibrosis associated with an inflammatory response. In some aspects, reducing fibrosis includes: (a) reducing or inhibiting the formation or deposition of tissue fibrosis; (b) reducing the size, cellularity (e.g., fibroblast or immune cell numbers), composition; or cellular content, of a fibrotic lesion; (c) reducing the collagen or hydroxyproline content, of a fibrotic lesion; (d) reducing expression or activity of one or more fibrogenic proteins; and/or (e) reducing fibrosis associated with inflammation.

In some aspects, the disclosure provides methods of reducing the loss of, or improving, hepatic function. In some aspects, the method results in: (a) preventing or slowing the formation or deposition of tissue fibrosis in the corresponding organ; (b) reducing the size, cellularity (e.g., fibroblast or immune cell numbers), composition; or cellular content, of a fibrotic lesion in the corresponding organ; (c) reducing the collagen or hydroxyproline content or degree of cross-linking, of a fibrotic lesion in the corresponding organ; (d) reducing expression or activity of one or more fibrogenic proteins (e.g., fibrinogen and collagen) in the corresponding organ; (d) reducing expression extracellular matrix and/or EMT in the corresponding organ; (e) reducing fibrosis associated with an inflammatory response in the corresponding organ; and/or (f) reducing hepatic stellate cells in the liver.

The disclosure also provides methods of treating and/or ameliorating fibrotic condition(s) of the liver. In some aspects, the method comprises administering a SCD-1 inhibitor or an effective amount of a pharmaceutical composition comprising a SCD-1 inhibitor to a subject having or at risk of developing, a fibrotic condition of the liver. Further provided is use of an SCD-1 inhibitor as provided herein in the manufacture of a medicament for the treatment or amelioration of a fibrotic condition of the liver. Fibrotic conditions of the liver that can be treated using SCD-1 inhibitors provided herein include one or more members of the group consisting of: steatosis (e.g., nonalcoholic steatohepatitis (NASH), fatty liver disease, cholestatic liver disease (e.g., primary biliary cirrhosis (PBC)), liver cirrhosis, alcohol induced liver fibrosis, infection-induced liver fibrosis, biliary duct injury, biliary fibrosis, congenital hepatic fibrosis, autoimmune hepatitis, and a cholangiopathy. In further aspects, the infection-induced liver fibrosis is bacterial-induced or viral-induced.

In an additional aspect, the fibrotic condition of the liver that can be treated with a SCD-1 inhibitor provided herein is one or more members of the group consisting of: hepatic fibrosis associated with viral infection (e.g., hepatitis (hepatitis C, B and D) and COVID-19), autoimmune hepatitis, non-alcoholic fatty liver disease (NAFLD), progressive massive fibrosis, alcoholism, and exposure to toxins or irritants (e.g., alcohol, pharmaceutical drugs and environmental toxins).

According to some aspects, the disclosure provides methods of reducing the loss of hepatic function in a subject. In some aspects, the method comprises administering a SCD-1 inhibitor topically to a subject in need thereof. In some aspects the method reduces the loss of hepatic function in a subject. In further aspects, the method reduces the loss of hepatic function in a subject through reducing the progression of hepatic fibrosis.

In some embodiments, the disclosure provides a method of treating or preventing or slowing the rate of fibrosis progression associated with NASH.

In addition to fibrosis associated with NASH, SCD-1 inhibitor compounds may be useful in the treatment of fibrosis associated with inflammation. Such inflammation may or may not be caused by accumulation of hepatic fat and or fat throughout the body.

Some embodiments provided herein relate to a method for preventing fatty liver that develops in recipients after liver transplants and ultimately impacts the efficacy of the transplant (World Journal of Gastroenterology (2018), 24(14), 1491-1506).

Patients with NASH cirrhosis are at significant risk for complications related to portal hypertension (Gastroenterology (2020), 158(5),1334-1345) because the fibrosis interferes with the normal hepatic metabolic function and hampers hepatic portal blood flow which increases intrahepatic vascular resistance (IHVR) (Scientific Reports (2016), 6, 33453).

Some embodiments provided herein relate to a method for preventing or treating portal hypertension and its complications such as variceal bleeding, ascites with bacterial peritonitis and hepatic encephalopathy

In some embodiments the present disclosure relates to a SCD-1 inhibitor for use in the prevention and/or treatment of NASH, wherein said use prevents and/or delays increase in the relative liver weight, plasma alanine aminotransferase levels, liver triglyceride content and/or liver cholesterol. The relative liver weight is defined as liver weight as percentage of total body weight. In some embodiments said use reduces the relative liver weight, plasma alanine aminotransferase levels, liver triglyceride content and/or liver cholesterol.

In some embodiments the present disclosure relates to a SCD-1 inhibitor for use in the prevention and/or treatment of NASH, wherein said use prevents, delays and/or reduces any histopathological signs of steatosis.

In some embodiments the present disclosure relates to a SCD-1 inhibitor for use in the prevention and/or treatment of NASH, wherein said use prevents, delays and/or reduces inflammation in the liver.

In some embodiments the present disclosure relates to a SCD-1 inhibitor for use in the prevention and/or treatment of NASH, wherein said use prevents or slows the rate of fibrosis progression.

In some embodiments the present disclosure relates to a SCD-1 inhibitor for use in the prevention and/or treatment of NASH, wherein said SCD-1 inhibitor is administered in a therapeutically effective amount to a subject in need thereof. In some embodiments said subject is obese and/or has diabetes. In some embodiments said subject suffers from obesity, hyperglycemia, type 2 diabetes, impaired glucose tolerance and/or type 1 diabetes.

BMI (body mass index) is a measure of body fat based on height and weight. The formula for calculation is BMI=(weight in kilograms)/(height in meters)². In some embodiments the present disclosure relates to a SCD-1 inhibitor use in the prevention and/or treatment of NASH, wherein said compound is administered in a therapeutically effective amount to a subject in need thereof, wherein said subject has a BMI of at least 25 kg/m². In some embodiments said subject has a BMI of at least 30 kg/m². In some embodiments said subject has a BMI between 30-50 kg/m².

### Characterization of Liver Fibrosis

Liver fibrosis consists of many different disease-states, which may be characterized by multiple invasive and non- or minimally invasive tests (Cells (2020), 9(4), 1005) (Wong, V. W-S; Chan, H. L-Y. (2013), Non-invasive methods to determine the severity of NAFLD and NASH. In G. C. Farrell, A. J. McCullough, and C. P. Day (Ed.) Non-Alcoholic Fatty Liver Disease: A Practical Guide (First Edition, pp. 112-131). Chichester, West Sussex, UK: John Wiley & Sons, Ltd). Liver biopsy is the current gold standard in diagnosis and prognosis. Characterization can be performed by liver needle biopsy, hepatic ultrasound, transient ultrasound elastography, magnetic resonance spectroscopy (MRS), magnetic resonance imaging (MRI), magnetic resonance elastography (MRE), and/or one of many different scoring systems including the Child-Turcotte-Pugh score (Journal of Hepatology (2006), 44(1), 217-231), Scheuer, Batts-Ludwig (The American Journal of Surgical Pathology (1995), 19(12), 1409-1417), Knodell, Ishak, METAVIR, International Association for Study of the Liver, FibroSure, HepaScore, Forns Index, FIB-4, Fibrolndex, aspartate aminotransferase-to-platelet ration index and/or other tests. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase (Journal of Hepatology (2007), 47(4), 598-607).

Multiparametric magnetic resonance imaging (MRI) as a non-invasive tool to evaluate and monitor liver disease has recently been shown to correctly identified patients with both steatosis and fibrosis, regardless of etiology (Journal of Hepatology (2014), 60(1), P69-77). The novel scanning method provides high diagnostic accuracy for the assessment of liver fibrosis, steatosis and hemosiderosis and could potentially replace liver biopsy for many indications. The novel magnetic resonance (MR) protocol (LiverMultiScan (Perspectum Ltd, UK)) quantifies iron-corrected T1 (cT1)20, by removing the effect of elevated iron on liver T1. Liver cT1 has been shown to correlate with the hallmarks of fibro-inflammatory disease and shows promise as a risk stratification tool in NASH. The proton density fat fraction (PDFF) distinguishes the proportion of the MR-visible protons due to fat from all MR-visible protons (attributable to both fat and water) expressed as a percent, and correlates very well with histologically graded liver steatosis (Scientific Reports (2020), 10, 15308). Utilizing paired MRI-PDFF and liver histology data, a relative reduction of 29% in liver fat on MRI-PDFF is associated with a histologic response in NASH (Therapeutic Advances in Gastroenterology (2016), 9(5), 692-701). Moreover, MRI-PDFF has been validated against liver histology and shown to be more sensitive in detecting changes in hepatic fat content and treatment response in clinical trials (Cells (2020), 9(4), 1005).

Magnetic resonance elastography (MRE) is a non-invasive medical imaging technique that measures the stiffness of soft tissues by generating shear waves in tissue, imaging their propagation using MRI, and processing the images to generate a stiffness map. MRE is the most accurate method of liver stiffness measurement (LSM) in nonalcoholic fatty liver disease (NAFLD). Liver stiffness has been associated with disease severity including fibrosis, inflammation, and ballooning (European radiology (2019), 29(11), 5823-5831). MRE was significantly correlated with fibrosis stage. Imaging biomarkers from multiparametric hepatic MRI/MRE can provide an accurate, noninvasive prediction of the NAFLD activity score.

Total NAS score represents the sum of scores for steatosis, lobular inflammation, cytological ballooning, and fibrosis (disease stage), and ranges from 0-8. The semiquantitative scoring system was validated by the National Institutes of Health-sponsored Nonalcoholic Steatohepatitis Clinical Research Network (NASH CRN). In the reference study, NAS scores of 0-2 occurred in cases largely considered not diagnostic of NASH, scores of 3-4 were evenly divided among those considered not diagnostic, borderline, or positive for NASH. Scores of 5-8 occurred in cases that were largely considered diagnostic of NASH (Hepatology (2005), 41(6), 1313-1321). FDA has accepted as critical inclusion criteria in NASH trials a NASH activity score (NAS) greater than or equal to 4 with at least 1 point each in inflammation and ballooning along with a NASH Clinical Research Network (CRN) fibrosis score greater than stage 1 fibrosis but less than stage 4 fibrosis.

In some embodiments, there is no significantly change in histologic score at 1 week; in some embodiments, for 2 weeks; in some embodiments, for 3 weeks; in some embodiments, for 4 weeks; in some embodiments, for 5 weeks; in some embodiments, for 6 weeks; in some embodiments, for 7 weeks; in some embodiments, for 8 weeks; in some embodiments, for 9 weeks; in some embodiments, for 10 weeks; in some embodiments, for 11 weeks; in some embodiments, for 12 weeks; in some embodiments, for 13 weeks; in some embodiments, for 14 weeks; in some embodiments, for 15 weeks; in some embodiments, for 16 weeks; in some embodiments, for 17 weeks; in some embodiments, for 18 weeks; in some embodiments, for 19 weeks; in some embodiments, for 20 weeks; in some embodiments, for 21 weeks; in some embodiments, for 22 weeks; in some embodiments, for 23 weeks; in some embodiments, for 24 weeks; in some embodiments, for 25 weeks; in some embodiments, for 26 weeks; in some embodiments, for 40 weeks; in some embodiments, for 52 weeks; in some embodiments, for 65 weeks; in some embodiments, for 72 weeks; in some embodiments, for 96 weeks; in some embodiments, for 1 month; in some embodiments, for 2 months; in some embodiments, for 3 months; in some embodiments, for 4 months; in some embodiments, for 5 months; in some embodiments, for 6 months; in some embodiments, for 7 months; in some embodiments, for 8 months; in some embodiments, for 9 months; in some embodiments, for 10 months; in some embodiments, for 11 months; in some embodiments, for 12 months; in some embodiments, for 1 year; in some embodiments, for 2 years; in some embodiments, for 3 years; in some embodiments, for 4 years; from the commencement of administration of a SCD-1 inhibitor compound.

In some embodiments, there is a reduction in histologic score at 1 week; in some embodiments, for 2 weeks; in some embodiments, for 3 weeks; in some embodiments, for 4 weeks; in some embodiments, for 5 weeks; in some embodiments, for 6 weeks; in some embodiments, for 7 weeks; in some embodiments, for 8 weeks; in some embodiments, for 9 weeks; in some embodiments, for 10 weeks; in some embodiments, for 11 weeks; in some embodiments, for 12 weeks; in some embodiments, for 13 weeks; in some embodiments, for 14 weeks; in some embodiments, for 15 weeks; in some embodiments, for 16 weeks; in some embodiments, for 17 weeks; in some embodiments, for 18 weeks; in some embodiments, for 19 weeks; in some embodiments, for 20 weeks; in some embodiments, for 21 weeks; in some embodiments, for 22 weeks; in some embodiments, for 23 weeks; in some embodiments, for 24 weeks; in some embodiments, for 25 weeks; in some embodiments, for 26 weeks; in some embodiments, for 40 weeks; in some embodiments, for 52 weeks; in some embodiments, for 65 weeks; in some embodiments, for 72 weeks; in some embodiments, for 96 weeks; in some embodiments, for 1 month; in some embodiments, for 2 months; in some embodiments, for 3 months; in some embodiments, for 4 months; in some embodiments, for 5 months; in some embodiments, for 6 months; in some embodiments, for 7 months; in some embodiments, for 8 months; in some embodiments, for 9 months; in some embodiments, for 10 months; in some embodiments, for 11 months; in some embodiments, for 12 months; in some embodiments, for 1 year; in some embodiments, for 2 years; in some embodiments, for 3 years; in some embodiments, for 4 years; from the commencement of administration of a SCD-1 inhibitor compound.

The FDA currently recommends considering the following liver histological improvements as endpoints reasonably likely to predict clinical benefit. Resolution of steatohepatitis on overall histopathological reading and no worsening of liver fibrosis on NASH CRN fibrosis score. Resolution of steatohepatitis is defined as absent fatty liver disease or isolated or simple steatosis without steatohepatitis and a NAS score of 0-1 for inflammation, 0 for ballooning, and any value for steatosis. Improvement in liver fibrosis greater than or equal to one stage (NASH CRN fibrosis score) and no worsening of steatohepatitis (defined as no increase in NAS for ballooning, inflammation, or steatosis). Both resolution of steatohepatitis and improvement in fibrosis.

The surrogate markers currently accepted by the FDA are histological in nature. One would either need to demonstrate that treatment resulted in a significant proportion of patients that achieve >1 stage improvement in fibrosis without worsening of NASH or a significant proportion of patients who achieve NASH resolution without worsening of liver fibrosis. NASH resolution is defined as the presence of any grade of steatosis, no ballooning, and only minimal (grade 1) lobular inflammation.

Other non-invasive potential surrogate endpoints could include circulating biomarkers and/or imaging techniques for liver fat, inflammation and fibrosis. These are being shown to correlate with histological improvement by paired biopsy and may one day in agreement with the agency replace the need for biopsy as an endpoint in clinical trials. SCD-1 inhibitors that improve histological NASH would similarly be conceived as improving these novel surrogate endpoints.

### Evaluation of Biological Activity

Various SCD-1 assay methods have been described in the art (Journal of Medicinal Chemistry (2014), 57(12), 5039-5056; Bioorganic & Medicinal Chemistry Letters (2009), 19(15), 4159-4166; and US 20110184027).

The ability of the compound to inhibit isolated SCD-1 enzyme activity can be evaluated in rat and human microsomes as described below (Oncotarget (2018), 9, 3-20). The compound is added as various concentrations to the liver microsomes and incubated for 5 minutes with ¹³C-labelled Stearoyl CoA, which is the substrate for SCD-1. The activity of SCD-1 is then determined by the conversion of ¹³C-Steoryl CoA to ¹³C-Oleoyl CoA using an LC/MS/MS method. The ability of the compound to inhibit SCD-1 activity is expressed as an IC₅₀.

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples.

### EXAMPLES

### Example 1

Compound 25 was evaluated for inhibition of SCD-1 activity in rat and human liver microsomes as described below.

Compound 25 is dissolved in DMSO to prepare a 10 mM stock solution.

Rat liver microsomes (RLM, BiolVT) and human liver microsome (HLM, InVitroCYP™ H-class, Pool of 25 donors BiolVT) solutions are prepared by adding the microsomes to 100 mM sodium phosphate, pH 7.4, at a protein concentration of 1.00 mg/mL.

Compound 25 stock solution is then added to the RLM and HLM solution at an initial concentration of 0.2 μM for direct inhibition. Each LM/Inhibitor Solution is added to blank LM solution to give 8 concentrations prepared by a 3-fold serial dilution. These diluted LM/inhibitor solutions are then added to a reaction buffer containing 2 mM NADH, 120 μM Coenzyme A, 2 mM ATP, 2 mM DTT, and 10 mM MgCl₂ in 100 mM sodium phosphate, pH 7.4.

A Stearoyl-¹³Cis-CoA solution in 10% methanol in water is prepared at a final concentration of 3.00 mM. The reactions are initiated by adding 4.00 μL of the 3.00 mM Stearoyl-¹³C₁₈-CoA to 200 μL of the reaction mixture and incubating at 37° C. for 5 minutes. The analysis of Oleoyl-¹³C₁₈-CoA resulting from the incubations is performed by liquid chromatography in conjunction with tandem mass spectrometry (LC/MS/MS).

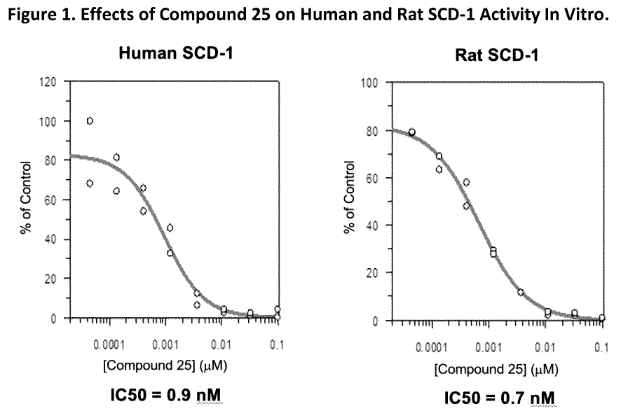

FIG. 1 shows the concentration-dependent inhibition of both rat and human SCD-1 activity by Compound 25 and the IC50s determined for inhibition of rat and human SCD-1

### Example 2

SCD-1 inhibitor compounds can be tested in vivo in a rodent diet-induced model of biopsy-confirmed NASH. In this experiment, the NASH phenotype is induced by maintaining mice upon a GAN diet for 33 weeks prior to drug administration (BMC Gastroenterology (2020), 20(1), 210; World Journal of Gastroenterology (2019), 25(33), 4904-4920). A biopsy is performed prior to drug treatment to eliminate any mice that have not developed the NASH phenotype and to mirror clinical trial design where liver biopsies prior to and at the end of treatment are compared. In addition to pre- and post-treatment histological analyses, metabolic assessments such as body weight, body composition, food intake and clinical chemistries are measured during and following the 12-week study dosing period.

Mice were dosed topically to 10% of the total BSA of the skin with either a placebo gel or a gel containing compound 25 (at a concentration of 0.05%, 0.25%, 0.75%, or 2.5%). (World Journal of Gastroenterology (2018), 24(2), 179-194).

TABLE 22
Gel Formulation Compositions

| Components | % w/w |
|---|---|
| Compound 25 | 0, 0.05, 0.25, |
| | 0.75. 2.5 |
| Propylene glycol, USP | 20.0 |
| Polyethylene glycol 400, NF | 21.0 |
| Diethylene glycol monoethyl ether | 25.0 |
| (Transcutol ® P), NF | |
| Dehydrated alcohol, USP | 10.0 |
| Phenoxyethanol, NF | 1.0 |
| Butylated hydroxytoluene, NF | 0.1 |
| Edetate disodium, dihydrate, USP | 0.005 |
| Carbopol 980, NF | 1.0 |
| Trolamine, NF | 0.375 |
| Purified Water NF | q.s. to 100 |

Animals and Diet: The Danish Animal Experiments Inspectorate approved all experiments which were conducted using internationally accepted principles for the use of laboratory animals under the personal license #2013-15-2934-00784. B6.V-Lepob/JRj (ob/ob) mice (6 weeks old) were from Janvier Labs (Le Genest Saint Isle, France) and housed in a controlled environment (12h light/dark cycle, lights on at 3 AM, 21+2° C., humidity 50+10%). Each animal was identified by an implantable subcutaneous microchip (PetID Microchip, E-vet, Haderslev, Denmark). Mice had ad libitum access to tap water and the Gubra Amylin NASH diet [GAN diet; 40 kcal-% fat (of these 0% trans-fat and 46% saturated fatty acids by weight), 22% fructose, 10% sucrose, 2% cholesterol; D09100310, Research Diets]. Mice were maintained on the GAN diet for 33 weeks prior to study initiation to induce NASH and remained on the GAN diet during the 12 weeks of drug administration. Mice were weighed daily during the study and body composition was measured by non-invasive EchoMRI scanning using EchoMRI-900 (EchoMRI, Houston, TX).

Liver Biopsy: After 33 weeks of GAN diet feeding, mice were anesthetized with isoflurane (2%-3%, in 100% oxygen), a small abdominal incision in the midline was made, and the left lateral lobe of the liver was exposed. A cone-shaped wedge of liver tissue (50-100 mg) was excised from the distal part of the lobe. The cut surface of the liver was closed by electrosurgical bipolar coagulation using an electrosurgical unit (ERBE VIO® 100C, ERBE, Marietta, GA, United States). The liver was returned to the abdominal cavity, the abdominal wall was sutured and skin stapled. Carprofen (5 mg/kg, i.p.) was administered at the time of surgery and at post-operative day one and two.

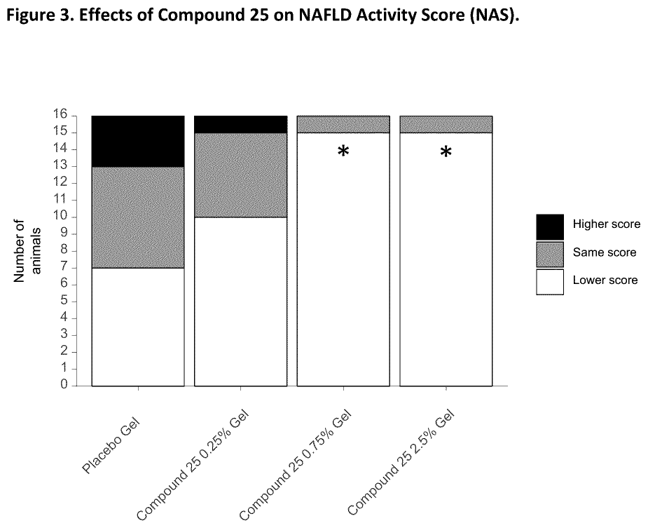

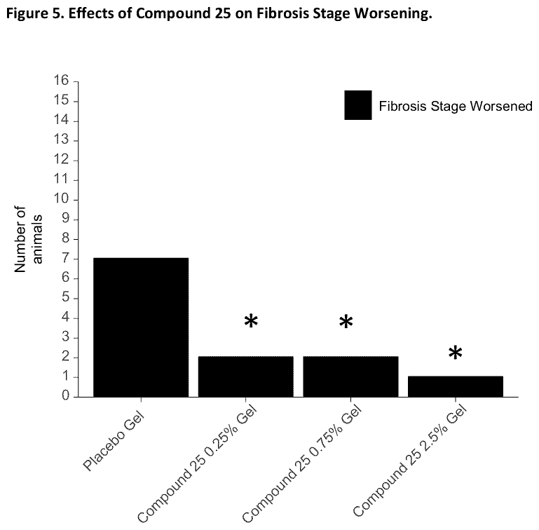

Histology and Digital Image Analyses: Biopsy and terminal liver samples (both from the left lateral lobe) were fixed overnight in 4% paraformaldehyde. Liver tissue was paraffin-embedded and sectioned (3 μm thickness). Sections were stained with hematoxylin-eosin (HE, Dako, Glostrup, Denmark), Picro-Sirius red (Sigma-Aldrich, Broendby, Denmark), anti-galectin-3 (cat. 125402, Biolegend, San Diego, CA, United States), anti-type I collagen (Collal; cat. 1310-01, Southern Biotech, Birmingham, AL, United States) or anti-α-SMA (AbCam, cat. no. abl24964) using previously established procedures (World Journal of Hepatology (2016), 8(16), 673-684). The NAS (FIG. 3) and fibrosis staging system (FIG. 5) was applied to liver pre-biopsies for scoring as outlined by Kleiner et al (Hepatology (2005), 41(6), 1313-1321). Quantitative histomorphometry was analyzed using digital imaging software (VIS Software, Visiopharm, Horsholm, Denmark). All histological assessments were performed by histologists blinded to the experimental groups.

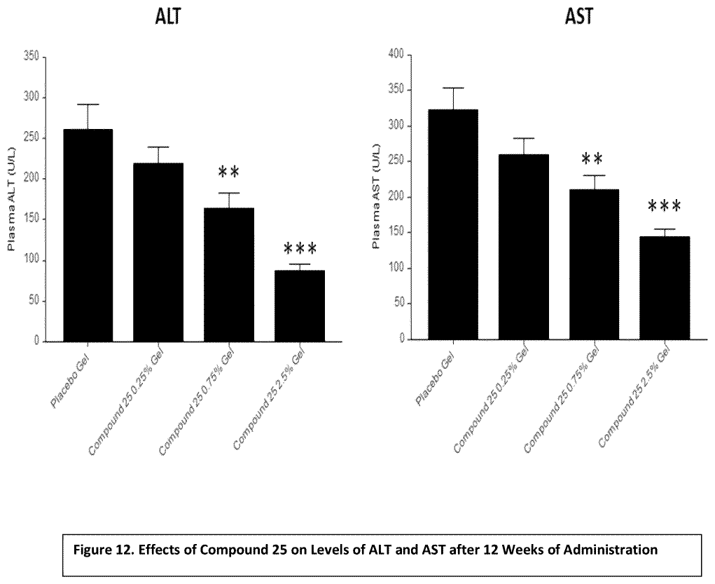

Plasma Biochemistry Analysis: Blood samples are collected in heparinized tubes and plasma is separated and stored at −80° C. until analysis. Alanine aminotransferase and aspartate aminotransferase (FIG. 12) were measured using commercial kits (Roche Diagnostics) on the Cobas c 501 autoanalyzer according to the manufacturer's instructions.

Terminal Hepatic Triglyceride Content: A sacrifice of the animals, a small liver piece (˜100 mg) was collected in FastPrep tubes and snap-frozen in liquid nitrogen. One milliliter 5% NH-40/ddH2O solution was added to the tube and the samples were homogenized. After homogenization the samples were slowly heated to 80° C.-100° C. for three minutes and then allowed to return to room temperature prior to a second round of heating. Next, samples were centrifuged for two minutes to remove any insoluble material. Triglyceride content in liver homogenates was measured using an auto analyzer Cobas C-111 with commercial kit (Roche Diagnostics, Germany) according to manufacturer's instructions.

Statistical Analysis: Comparison of data and significance differences for multiple testing was visualized on the graph when p<0.05. For single-timepoint continuous data, the data were fitted to a one-factor linear regression model with the treatment groups as categorical, independent (predictor) variables and Dunnett's test was used to compare treatments to control. Continuous data with repeated sample collection over time, the data is fitted to a two-factor linear regression model with the treatment groups and time points as categorical independent (predictor) variables with interaction. Dunnett's test was used to compare treatments to control for each timepoint. Data from categorical endpoints, such as histopathological scoring values, were partitioned into a 2×2 contingency table containing responders and non-responders in control and treatment groups and either a Fisher's or Chi-Square test was applied.

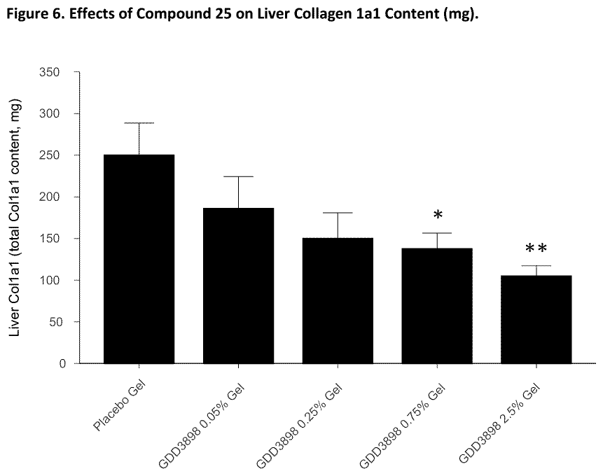

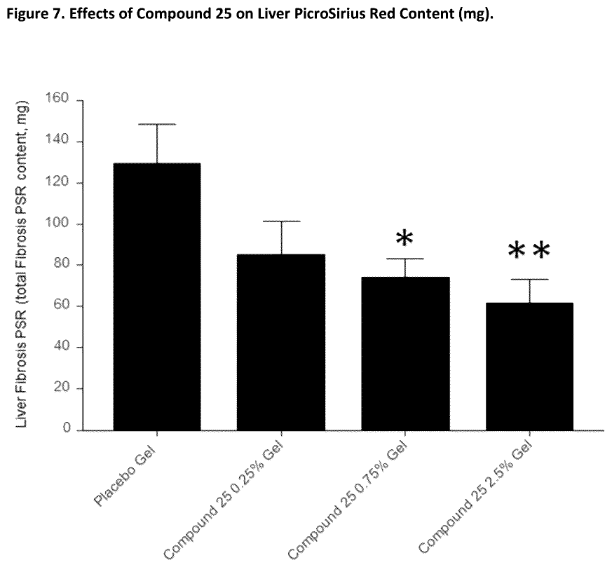

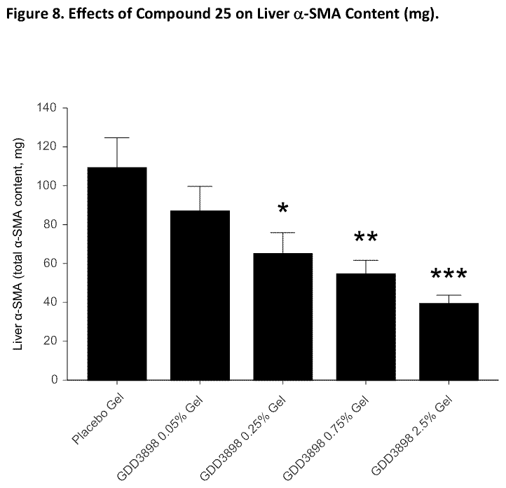

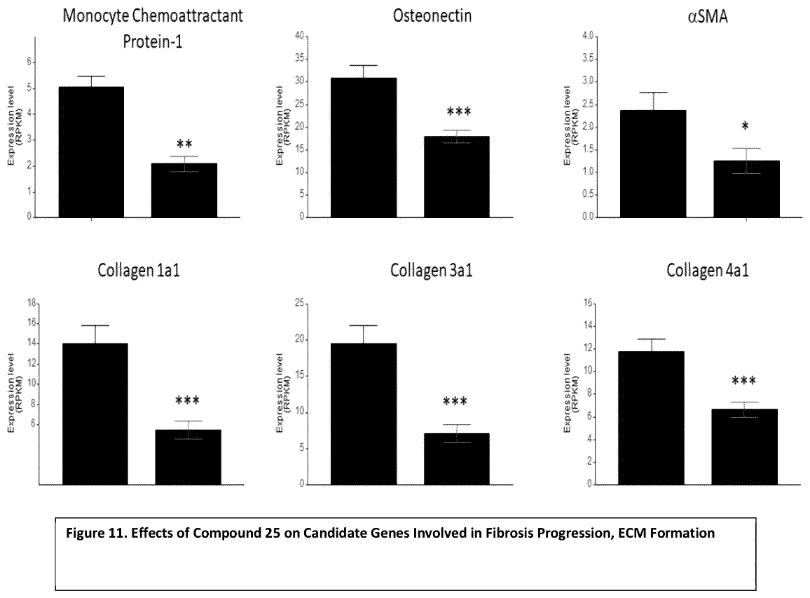

Conclusion: In a diet-induced mouse model of biopsy-confirmed NASH, topical dosing of Compound 25 to just 10% of the BSA of the skin improved multiple features of NASH. Convergent lines of evidence suggest that Compound 25 can prevent and slow the rate of fibrosis progression. Compound 25 improved overall NAFLD Activity Score or NAS (FIG. 3) and very unexpectedly prevented worsening of fibrosis stage (FIG. 5). Multiple quantitative markers of collagen (Collagen 1a1 immunohistochemistry; PSR histochemical staining and Collagen 1a1, Collagen 3a1 and Collagen 4a1 mRNA expression; FIG. 6, FIG. 7 and FIG. 11, respectively) were shown to be decreased by compound 25. At least in part, this decrease may be due to the reduced activation of the stellate cells indicated by decreased α-SMA expression (FIG. 8). These changes are also clearly observed at doses of compound 25 that had no or minimal effect on either total body weight or body fat, strongly suggesting the effects on fibrosis are not simply secondary consequences to reduction of fat. Compound 25 also improved hepatic steatosis and decreased inflammation which are known to promote fibrosis progression. This is consistent with the marked decrease in serum ALT and AST after topical dosing. Additionally, compound 25 favorably regulated several candidate genes whose expression may be predictive for poor outcomes like liver cirrhosis and cancer. Collectively, these findings suggest that compound 25 exerts therapeutic effects to prevent, reduce the incidence of, delay the onset of, reduce the severity of, or treat liver fibrosis in a subject.

What is claimed is:

1. A method of treating a patient with non-alcoholic steatohepatitis (NASH) but without fibrosis so as to prevent the development of fibrosis which comprises topically applying to the patient's skin an amount of an SCD-1 inhibitor compound effective to prevent the development of fibrosis in the patient.

2. The method of claim 1, wherein the patient with NASH but without fibrosis is characterized by a NASH CRN fibrosis score of 0, and the prevention of the development of fibrosis is characterized by no increase in the NASH CRN fibrosis score in the patient.

3. A method of treating a patient with NASH and with early-stage fibrosis so as to prevent, or slow the rate of progression of, the patient's fibrosis to a later-stage fibrosis which comprises topically applying to the patient's skin an amount of an SCD-1 inhibitor compound effective to prevent, or slow the rate of progression, of the fibrosis to a later-stage fibrosis in the patient.

4. The method of claim 3, wherein the patient with NASH and with early-stage fibrosis is characterized by a NASH CRN fibrosis score of 1-2 and the prevention of the patient's early stage fibrosis to a later-stage fibrosis is characterized by no increase in the NASH CRN fibrosis score in said patient or the slowing of the rate of progression of the patient's early stage fibrosis to an later-stage fibrosis is characterized by a reduced rate of increase in the NASH CRN fibrosis score in the patient.

5. A method of treating a patient with NASH and with later-stage fibrosis, so as to reduce the extent of the patient's fibrosis which comprises topically applying to the patient's skin an amount of an SCD-1 inhibitor compound effective to reduce the extent of fibrosis in the patient.

6. The method of claim 5, wherein the patient with NASH and with later-stage fibrosis is characterized by a NASH CRN fibrosis score of 3-4, and the extent of fibrosis is characterized by a decrease in the NASH CRN fibrosis score in said patient.

7. The method of claim 4, wherein the patient has a NASH CRN fibrosis score of 1.

8. The method of claim 4, wherein the patient has a NASH CRN fibrosis score of 2.

9. The method of claim 6, wherein the patient has a NASH CRN fibrosis score of 3.

10. The method of claim 6, wherein the patient has a NASH CRN fibrosis score of 4.

11. The method of any one of claims 1-10, wherein the method which further comprises treating the patient with an amount of an anti-NASH compound effective to inhibit progression of, or effect resolution of, NASH.

12. The method of claim 2 or 4, wherein the no increase in, or slowed rate of increase in the NASH CRN fibrosis score is observed at 2, 4, 8, 24, 40, 52, 65, 72, or 96 weeks; or at 2, 3, or 4 years after commencement of administration of the SCD-1 inhibitor compound.

13. The method of claim 6, wherein the decrease in the NASH CRN fibrosis score is observed at 2, 4, 8, 24, 40, 52, 65, 72, or 96 weeks; or at 2, 3, or 4 years after commencement of administration of the SCD-1 inhibitor compound.

14. The method of any one of claims 1-13, wherein the method results in an improvement in the patient's liver (as determined by histology.)

15. The method of any one of claims 1-13, wherein the method results in no worsening of the patient's liver (as determined by histology.)

16. The method of any one of claims 1-13, wherein the method prevents, reduces incidence of, delays onset of, or reduces severity of, conditions associated with increased levels of liver triglycerides in the patient.

17. The method of any one of claims 1-13, wherein the method prevents, reduces incidence of, delays onset of, or reduce severity of, conditions associated with changes in the ratio of lipid-containing to non-lipid containing hepatocytes in the patient.

18. The method of any one of claims 1-13, wherein the method prevents, reduces incidence of, delays onset of, or reduce severity of conditions associated with changes in the level of hepatic inflammation in the patient.

19. The method of any one of claims 1-13, wherein the method prevents, reduced incidence of, delays onset of, or reduce severity of conditions associated with fibrillar and matrix forming collagens in the patient.

20. The method of any one of claims 1-13, wherein the method prevents, reduces incidence of, delays onset of, or reduces severity of, conditions associated with hepatic stellate cell activation in the patient.

21. The method of any one of claims 1-13, wherein the method prevents, reduces incidence of, delays onset of, or reduces severity of hepatocellular ballooning in the patient.

22. The method of any one of claims 1-13, wherein the method prevents, reduces incidence of, delays onset of, or reduces severity of hepatic steatosis in the patient.

23. The method of any one of claims 1-22, wherein the SCD-1 inhibitor compound is topically applied to at least 30% of the patient's body surface area (BSA), 20% of the patient's BSA, 10% of the patient's BSA, 5% of the patient's BSA, 2% of the patient's BSA, or 1% of the patient's BSA.

24. The method according to any one of claims 1-22, wherein the SCD-1 inhibitor compound is topically applied to 1-2% of the patient's BSA.

25. The method of any one of claims 1-24, wherein the SCD-1 inhibitor compound is topically applying to the patient's skin once a day, twice a day, once every two days, once every week, or once every two weeks.

26. The method of any one of claims 1-25, wherein the SCD-1 inhibitor compound has the structure of Formula I: 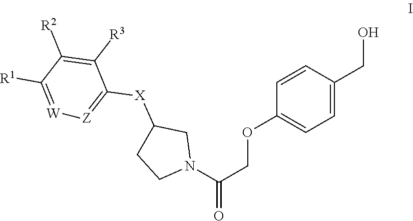
or pharmaceutically acceptable salts thereof,
wherein:
X is selected from the group consisting of O, NH, N-alkyl or N-acyl, S, SO and SO₂;
W is independently CR⁴ or N;
Z is independently CR⁵ or N;
each R¹, R², R³, R⁴ and R⁵ are independently selected from the group consisting of H, OH, F, Cl, Br, I, C₁ to C₆ straight chain or branched chain alkyl, CH₂F, CHF₂, CF₃, CH₂CH₂F, CH₂CHF₂, CH₂CF₃, CHFCH₂F, CHFCHF₂, CHFCF₃, CF₂CH₂F, CF₂CHF₂, CF₂CF₃, 0-alkyl, 0-cycloalkyl, 0-alkylcycloalkyl, OCH₂F, OCHF₂, OCF₃, OCH₂CH₂F, OCH₂CHF₂, OCH₂CF₃, OCHFCH₂F, OCHFCHF₂, OCHFCF₃, OCF₂CH₂F, OCF₂CHF₂, OCF₂CF₃, O—(CO)—R⁶, O—(CNH)—R⁶, O—(CNR⁶)—R⁷, SO₃H or a ester thereof, CO₂H or a ester thereof, PO₂(OCH₃)H or a phosphonate thereof, NO₂, NH₂, NHCH(O), NR⁶CH(O), NHC(O)R⁶, NR⁶C(O)R⁷, C(O)NR⁶R⁷, C(NH)NR⁶R⁷, C(NH)NR⁶OH, C(NH)NR⁶NO₂, and C(NR⁶)NR⁷C(NR⁸)NR⁹R¹⁰;
wherein adjacent substituents R¹, R², R³, R⁴ and R⁵ may form a saturated or unsaturated 5-membered or 6-membered carbocyclic or heterocyclic ring;
each R⁶, R⁷, R⁸, R⁹ and R¹⁰ are independently selected from the group consisting of H, OH, O-Rx, optionally substituted alkyl, cycloalkyl, heterocycloalkyl, alkylheterocycloalkyl, optionally substituted alkenyl, optionally substituted alkynyl, optionally substituted aryl, optionally substituted alkylaryl, optionally substituted heteroaryl, and optionally substituted alkylheteroaryl; and
Rx is selected from the group consisting of alkyl, cycloalkyl, alkylcycloalkyl, acyl, ester, or thioester.

27. The method of any one of claims 1-26, wherein the SCD-1 inhibitor of Formula I is selected from the group consisting of: 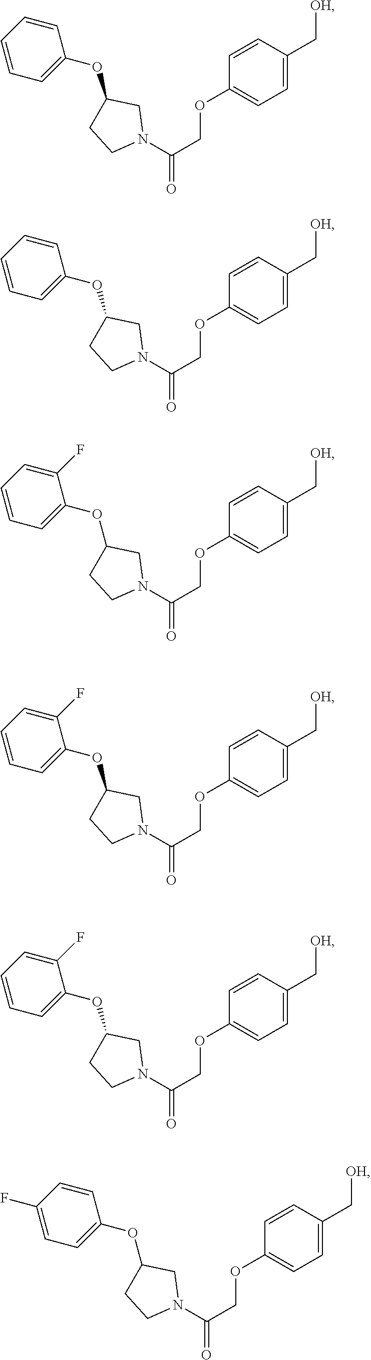 [SMILES: O=C(COc1ccc(CO)cc1)N1CC[C@@H](Oc2ccccc2)C1 ; O=C(COc1ccc(CO)cc1)N1CC[C@H](Oc2ccccc2)C1 ; O=C(COc1ccc(CO)cc1)N1CCC(Oc2ccccc2F)C1 ; O=C(COc1ccc(CO)cc1)N1CC[C@@H](Oc2ccccc2F)C1 ; O=C(COc1ccc(CO)cc1)N1CC[C@H](Oc2ccccc2F)C1 ; O=C(COc1ccc(CO)cc1)N1CCC(Oc2ccc(F)cc2)C1] 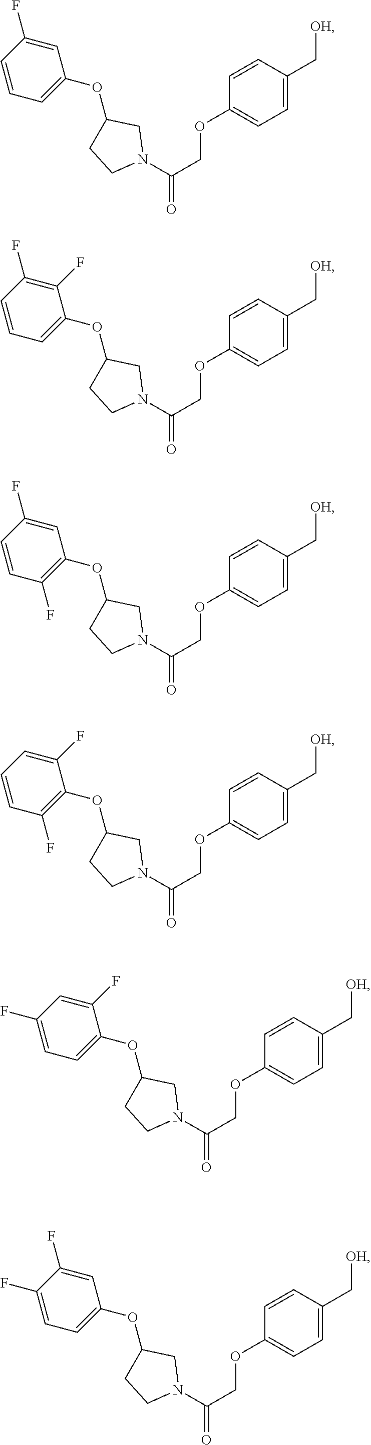 [SMILES: O=C(COc1ccc(CO)cc1)N1CCC(Oc2cccc(F)c2)C1 ; O=C(COc1ccc(CO)cc1)N1CCC(Oc2cccc(F)c2F)C1 ; O=C(COc1ccc(CO)cc1)N1CCC(Oc2cc(F)ccc2F)C1 ; O=C(COc1ccc(CO)cc1)N1CCC(Oc2c(F)cccc2F)C1 ; O=C(COc1ccc(CO)cc1)N1CCC(Oc2ccc(F)cc2F)C1 ; O=C(COc1ccc(CO)cc1)N1CCC(Oc2ccc(F)c(F)c2)C1] 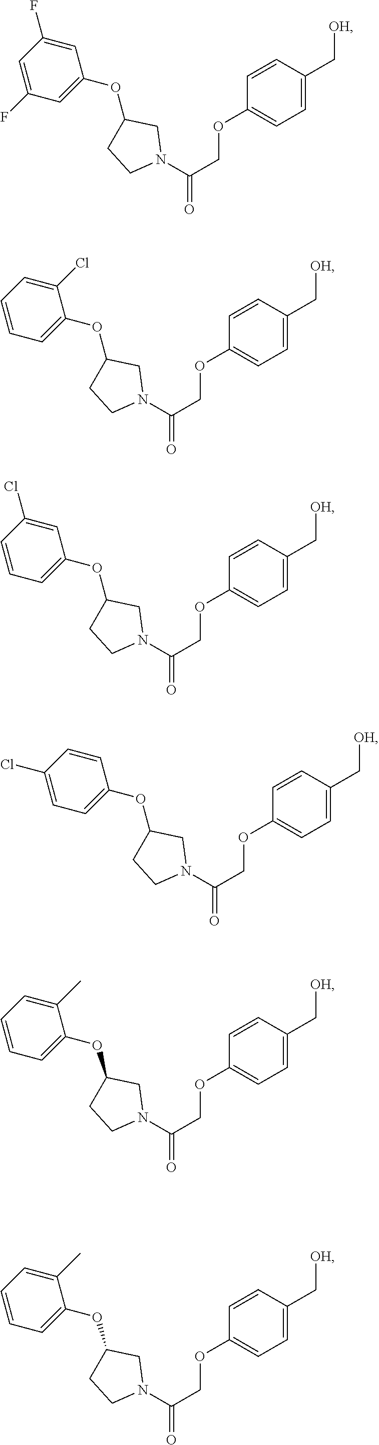 [SMILES: O=C(COc1ccc(CO)cc1)N1CCC(Oc2cc(F)cc(F)c2)C1 ; O=C(COc1ccc(CO)cc1)N1CCC(Oc2ccccc2Cl)C1 ; O=C(COc1ccc(CO)cc1)N1CCC(Oc2cccc(Cl)c2)C1 ; O=C(COc1ccc(CO)cc1)N1CCC(Oc2ccc(Cl)cc2)C1 ; Cc1ccccc1O[C@@H]1CCN(C(=O)COc2ccc(CO)cc2)C1 ; Cc1ccccc1O[C@H]1CCN(C(=O)COc2ccc(CO)cc2)C1] 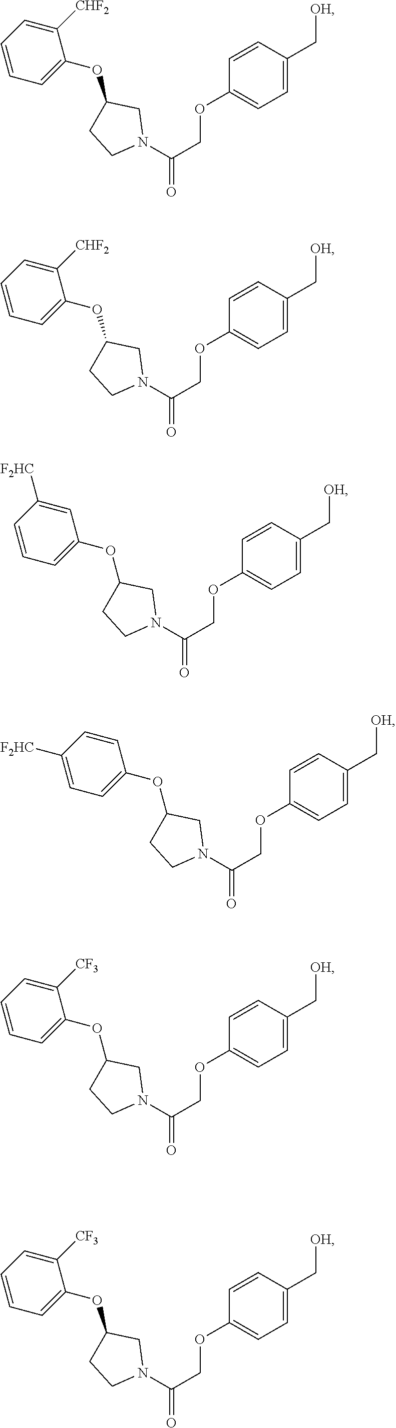 [SMILES: O=C(COc1ccc(CO)cc1)N1CC[C@@H](Oc2ccccc2C(F)F)C1 ; O=C(COc1ccc(CO)cc1)N1CC[C@H](Oc2ccccc2C(F)F)C1 ; O=C(COc1ccc(CO)cc1)N1CCC(Oc2cccc(C(F)F)c2)C1 ; O=C(COc1ccc(CO)cc1)N1CCC(Oc2ccc(C(F)F)cc2)C1 ; O=C(COc1ccc(CO)cc1)N1CCC(Oc2ccccc2C(F)(F)F)C1 ; O=C(COc1ccc(CO)cc1)N1CC[C@@H](Oc2ccccc2C(F)(F)F)C1] 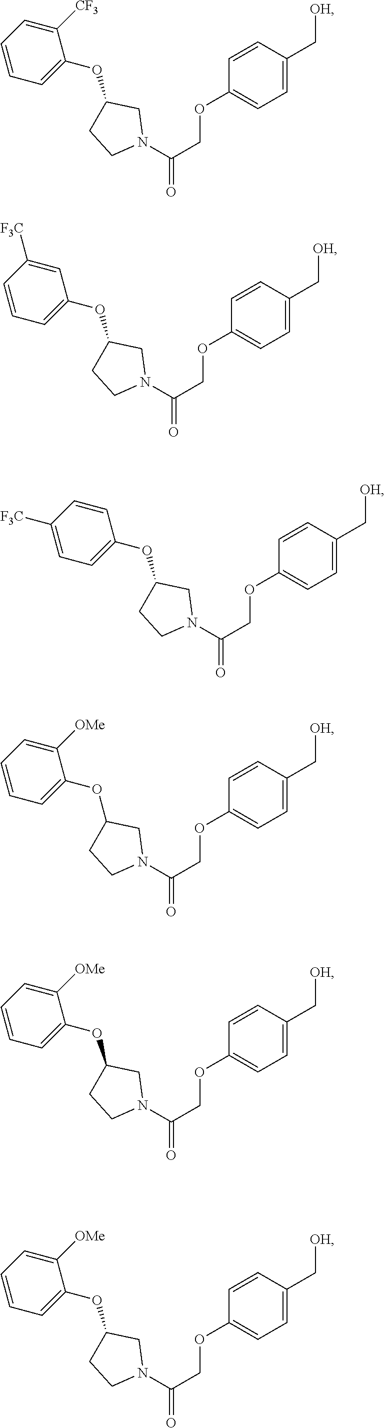 [SMILES: O=C(COc1ccc(CO)cc1)N1CC[C@H](Oc2ccccc2C(F)(F)F)C1 ; O=C(COc1ccc(CO)cc1)N1CC[C@H](Oc2cccc(C(F)(F)F)c2)C1 ; O=C(COc1ccc(CO)cc1)N1CC[C@H](Oc2ccc(C(F)(F)F)cc2)C1 ; COc1ccccc1OC1CCN(C(=O)COc2ccc(CO)cc2)C1 ; COc1ccccc1O[C@@H]1CCN(C(=O)COc2ccc(CO)cc2)C1 ; COc1ccccc1O[C@H]1CCN(C(=O)COc2ccc(CO)cc2)C1] 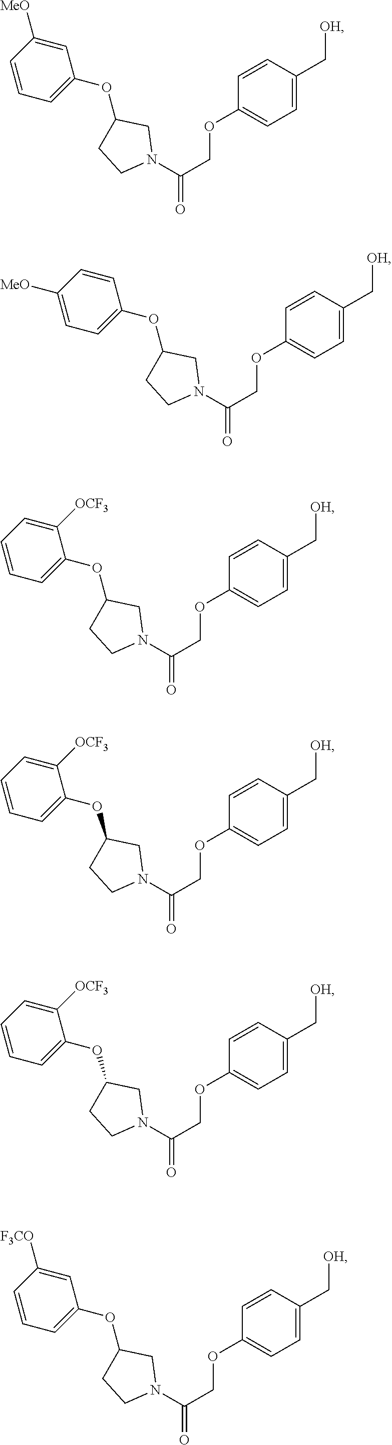 [SMILES: COc1cccc(OC2CCN(C(=O)COc3ccc(CO)cc3)C2)c1 ; COc1ccc(OC2CCN(C(=O)COc3ccc(CO)cc3)C2)cc1 ; O=C(COc1ccc(CO)cc1)N1CCC(Oc2ccccc2OC(F)(F)F)C1 ; O=C(COc1ccc(CO)cc1)N1CC[C@@H](Oc2ccccc2OC(F)(F)F)C1 ; O=C(COc1ccc(CO)cc1)N1CC[C@H](Oc2ccccc2OC(F)(F)F)C1 ; O=C(COc1ccc(CO)cc1)N1CCC(Oc2cccc(OC(F)(F)F)c2)C1] 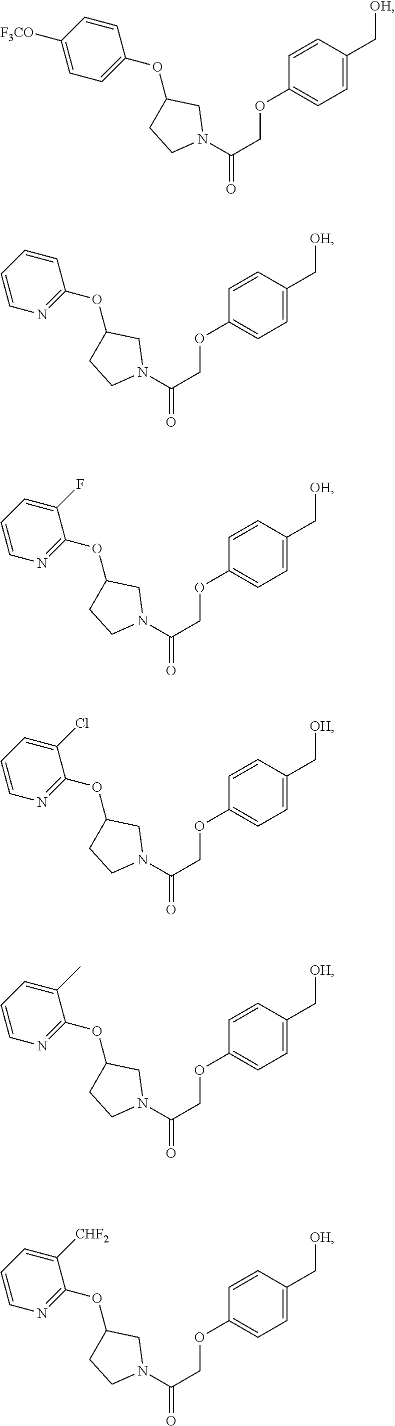 [SMILES: O=C(COc1ccc(CO)cc1)N1CCC(Oc2ccc(OC(F)(F)F)cc2)C1 ; O=C(COc1ccc(CO)cc1)N1CCC(Oc2ccccn2)C1 ; O=C(COc1ccc(CO)cc1)N1CCC(Oc2ncccc2F)C1 ; O=C(COc1ccc(CO)cc1)N1CCC(Oc2ncccc2Cl)C1 ; Cc1cccnc1OC1CCN(C(=O)COc2ccc(CO)cc2)C1 ; O=C(COc1ccc(CO)cc1)N1CCC(Oc2ncccc2C(F)F)C1] 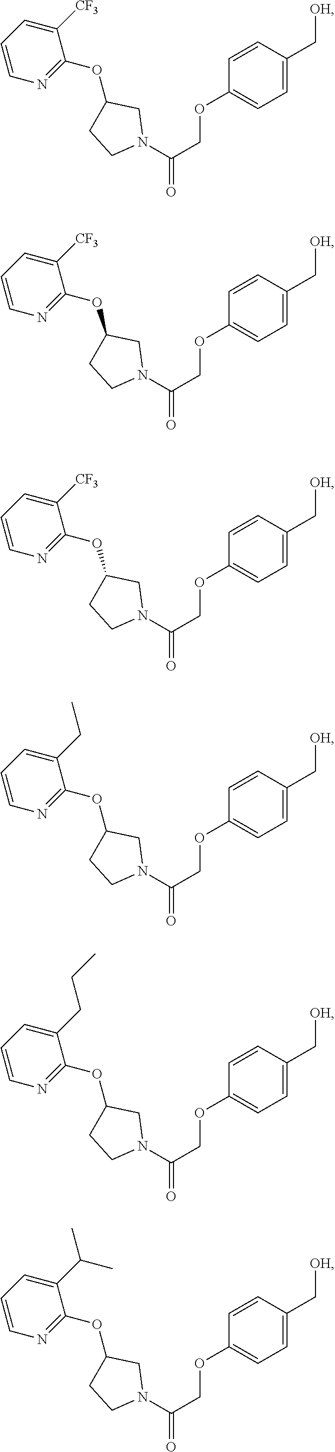 [SMILES: O=C(COc1ccc(CO)cc1)N1CCC(Oc2ncccc2C(F)(F)F)C1 ; O=C(COc1ccc(CO)cc1)N1CC[C@@H](Oc2ncccc2C(F)(F)F)C1 ; O=C(COc1ccc(CO)cc1)N1CC[C@H](Oc2ncccc2C(F)(F)F)C1 ; CCc1cccnc1OC1CCN(C(=O)COc2ccc(CO)cc2)C1 ; CCCc1cccnc1OC1CCN(C(=O)COc2ccc(CO)cc2)C1 ; CC(C)c1cccnc1OC1CCN(C(=O)COc2ccc(CO)cc2)C1] 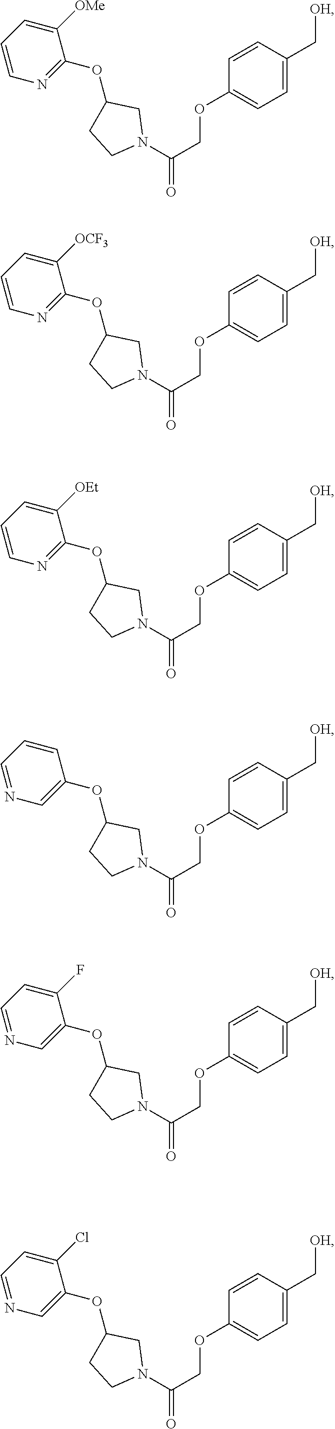 [SMILES: COc1cccnc1OC1CCN(C(=O)COc2ccc(CO)cc2)C1 ; O=C(COc1ccc(CO)cc1)N1CCC(Oc2ncccc2OC(F)(F)F)C1 ; CCOc1cccnc1OC1CCN(C(=O)COc2ccc(CO)cc2)C1 ; O=C(COc1ccc(CO)cc1)N1CCC(Oc2cccnc2)C1 ; O=C(COc1ccc(CO)cc1)N1CCC(Oc2cnccc2F)C1 ; O=C(COc1ccc(CO)cc1)N1CCC(Oc2cnccc2Cl)C1] 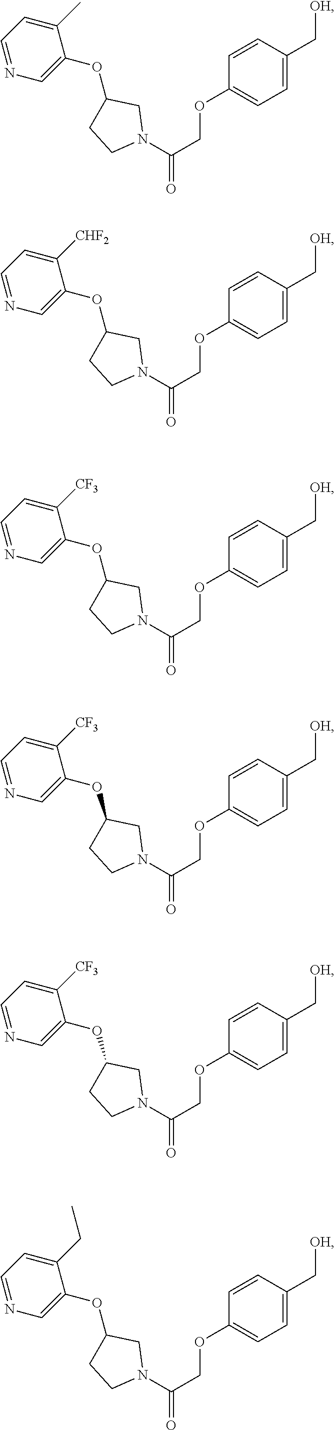 [SMILES: Cc1ccncc1OC1CCN(C(=O)COc2ccc(CO)cc2)C1 ; O=C(COc1ccc(CO)cc1)N1CCC(Oc2cnccc2C(F)F)C1 ; O=C(COc1ccc(CO)cc1)N1CCC(Oc2cnccc2C(F)(F)F)C1 ; O=C(COc1ccc(CO)cc1)N1CC[C@@H](Oc2cnccc2C(F)(F)F)C1 ; O=C(COc1ccc(CO)cc1)N1CC[C@H](Oc2cnccc2C(F)(F)F)C1 ; CCc1ccncc1OC1CCN(C(=O)COc2ccc(CO)cc2)C1] 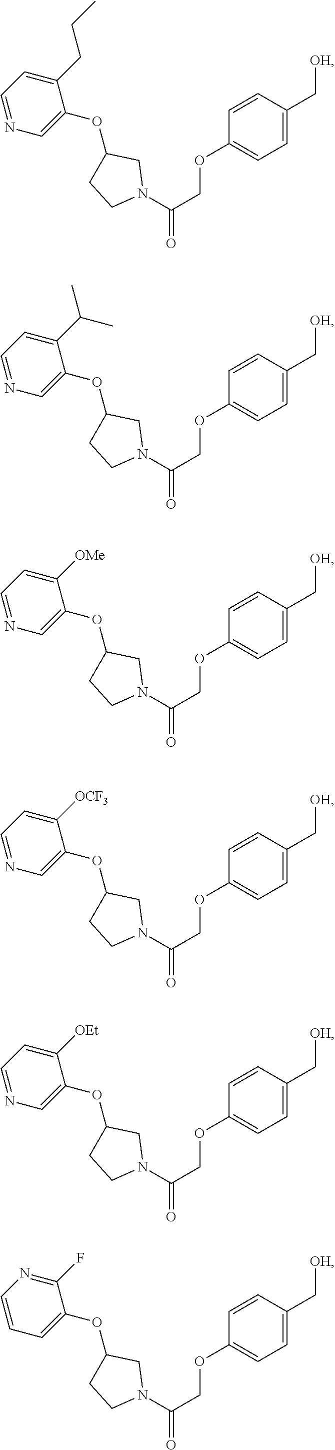 [SMILES: CCCc1ccncc1OC1CCN(C(=O)COc2ccc(CO)cc2)C1 ; CC(C)c1ccncc1OC1CCN(C(=O)COc2ccc(CO)cc2)C1 ; COc1ccncc1OC1CCN(C(=O)COc2ccc(CO)cc2)C1 ; O=C(COc1ccc(CO)cc1)N1CCC(Oc2cnccc2OC(F)(F)F)C1 ; CCOc1ccncc1OC1CCN(C(=O)COc2ccc(CO)cc2)C1 ; O=C(COc1ccc(CO)cc1)N1CCC(Oc2cccnc2F)C1] 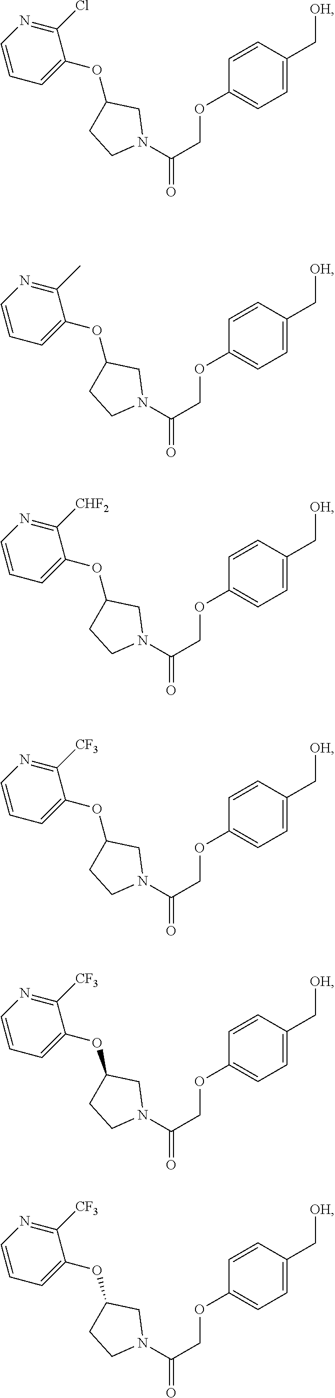 [SMILES: O=C(COc1ccc(CO)cc1)N1CCC(Oc2cccnc2Cl)C1 ; Cc1ncccc1OC1CCN(C(=O)COc2ccc(CO)cc2)C1 ; O=C(COc1ccc(CO)cc1)N1CCC(Oc2cccnc2C(F)F)C1 ; O=C(COc1ccc(CO)cc1)N1CCC(Oc2cccnc2C(F)(F)F)C1 ; O=C(COc1ccc(CO)cc1)N1CC[C@@H](Oc2cccnc2C(F)(F)F)C1 ; O=C(COc1ccc(CO)cc1)N1CC[C@H](Oc2cccnc2C(F)(F)F)C1] 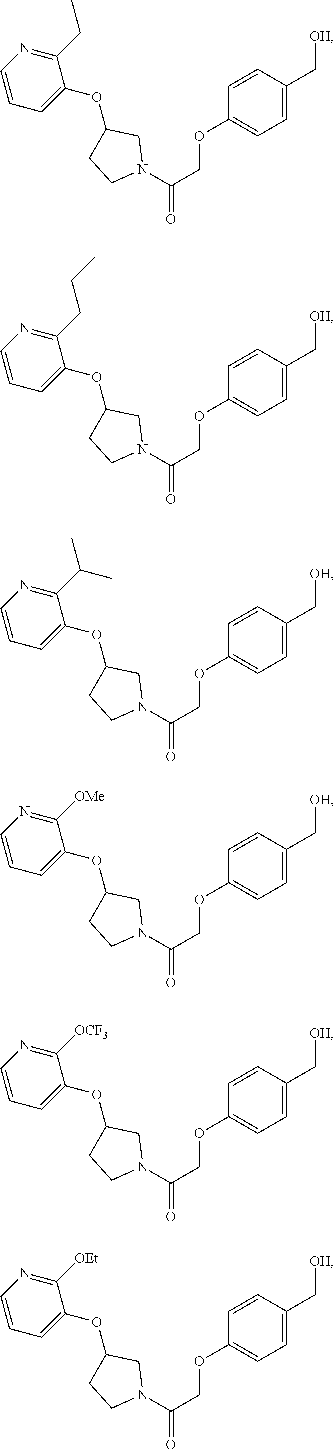 [SMILES: CCc1ncccc1OC1CCN(C(=O)COc2ccc(CO)cc2)C1 ; CCCc1ncccc1OC1CCN(C(=O)COc2ccc(CO)cc2)C1 ; CC(C)c1ncccc1OC1CCN(C(=O)COc2ccc(CO)cc2)C1 ; COc1ncccc1OC1CCN(C(=O)COc2ccc(CO)cc2)C1 ; O=C(COc1ccc(CO)cc1)N1CCC(Oc2cccnc2OC(F)(F)F)C1 ; CCOc1ncccc1OC1CCN(C(=O)COc2ccc(CO)cc2)C1] 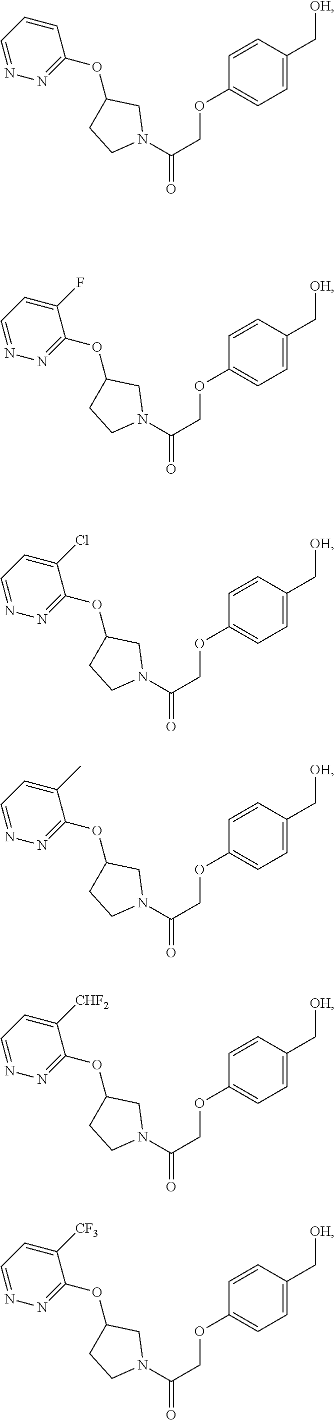 [SMILES: O=C(COc1ccc(CO)cc1)N1CCC(Oc2cccnn2)C1 ; O=C(COc1ccc(CO)cc1)N1CCC(Oc2nnccc2F)C1 ; O=C(COc1ccc(CO)cc1)N1CCC(Oc2nnccc2Cl)C1 ; Cc1ccnnc1OC1CCN(C(=O)COc2ccc(CO)cc2)C1 ; O=C(COc1ccc(CO)cc1)N1CCC(Oc2nnccc2C(F)F)C1 ; O=C(COc1ccc(CO)cc1)N1CCC(Oc2nnccc2C(F)(F)F)C1] 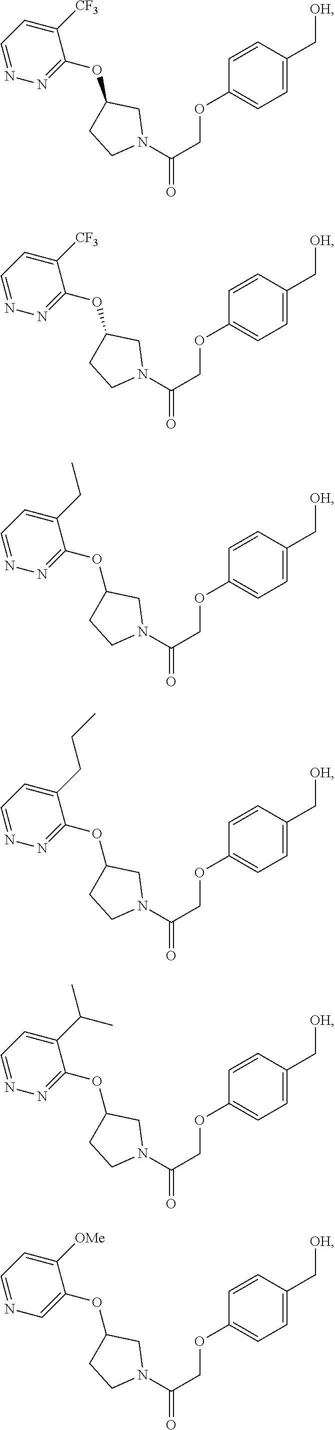 [SMILES: O=C(COc1ccc(CO)cc1)N1CC[C@@H](Oc2nnccc2C(F)(F)F)C1 ; O=C(COc1ccc(CO)cc1)N1CC[C@H](Oc2nnccc2C(F)(F)F)C1 ; CCc1ccnnc1OC1CCN(C(=O)COc2ccc(CO)cc2)C1 ; CCCc1ccnnc1OC1CCN(C(=O)COc2ccc(CO)cc2)C1 ; CC(C)c1ccnnc1OC1CCN(C(=O)COc2ccc(CO)cc2)C1 ; COc1ccncc1OC1CCN(C(=O)COc2ccc(CO)cc2)C1] 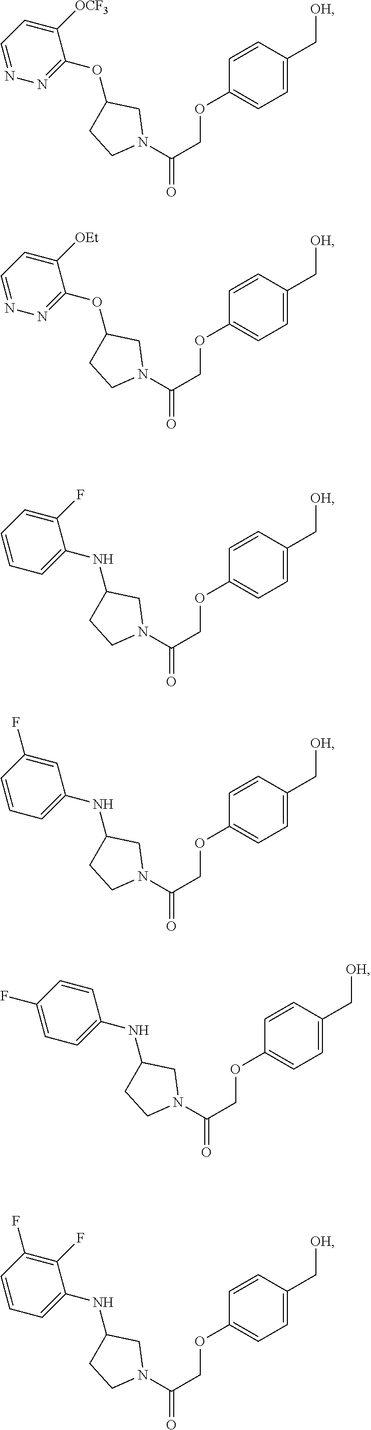 [SMILES: O=C(COc1ccc(CO)cc1)N1CCC(Oc2nnccc2OC(F)(F)F)C1 ; CCOc1ccnnc1OC1CCN(C(=O)COc2ccc(CO)cc2)C1 ; O=C(COc1ccc(CO)cc1)N1CCC(Nc2ccccc2F)C1 ; O=C(COc1ccc(CO)cc1)N1CCC(Nc2cccc(F)c2)C1 ; O=C(COc1ccc(CO)cc1)N1CCC(Nc2ccc(F)cc2)C1 ; O=C(COc1ccc(CO)cc1)N1CCC(Nc2cccc(F)c2F)C1] 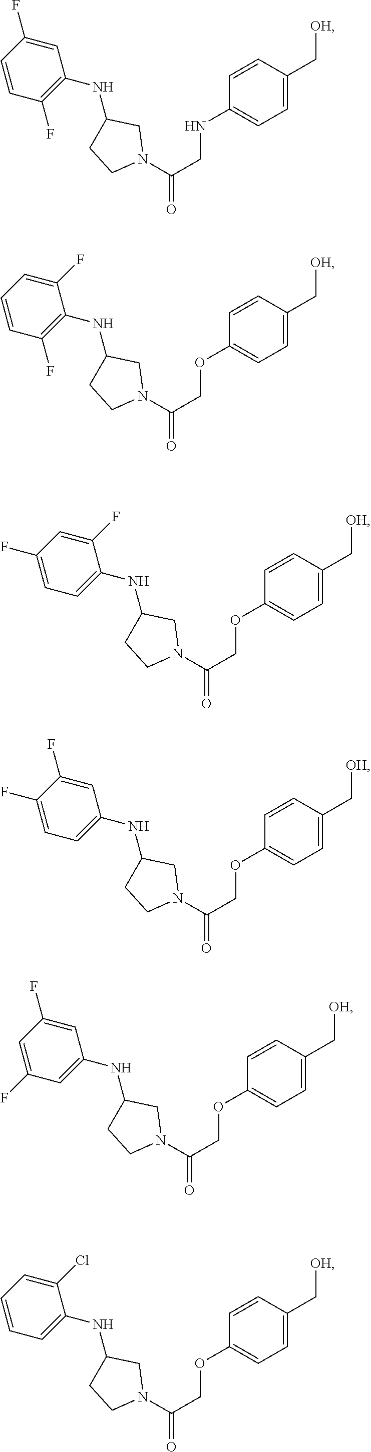 [SMILES: O=C(CNc1ccc(CO)cc1)N1CCC(Nc2cc(F)ccc2F)C1 ; O=C(COc1ccc(CO)cc1)N1CCC(Nc2c(F)cccc2F)C1 ; O=C(COc1ccc(CO)cc1)N1CCC(Nc2ccc(F)cc2F)C1 ; O=C(COc1ccc(CO)cc1)N1CCC(Nc2ccc(F)c(F)c2)C1 ; O=C(COc1ccc(CO)cc1)N1CCC(Nc2cc(F)cc(F)c2)C1 ; O=C(COc1ccc(CO)cc1)N1CCC(Nc2ccccc2Cl)C1] 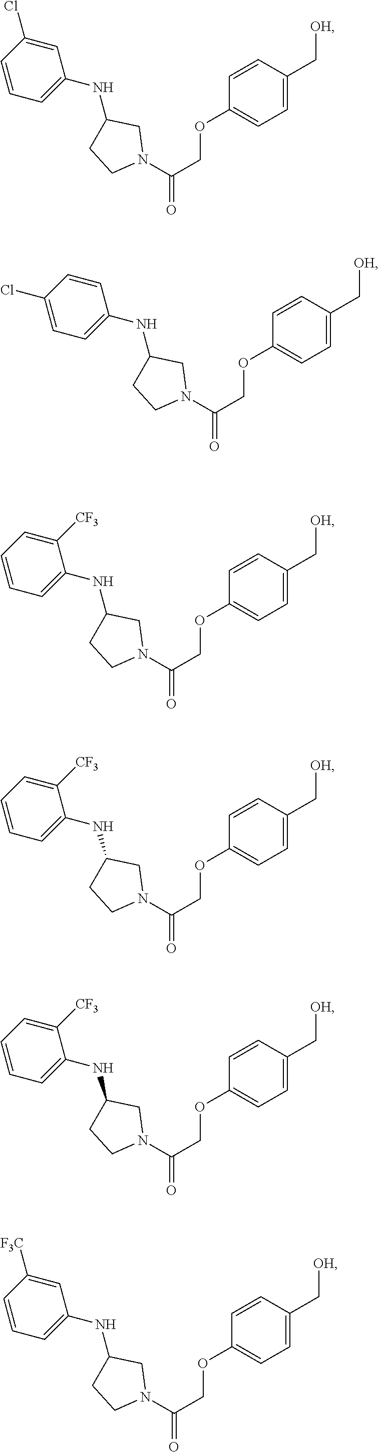 [SMILES: O=C(COc1ccc(CO)cc1)N1CCC(Nc2cccc(Cl)c2)C1 ; O=C(COc1ccc(CO)cc1)N1CCC(Nc2ccc(Cl)cc2)C1 ; O=C(COc1ccc(CO)cc1)N1CCC(Nc2ccccc2C(F)(F)F)C1 ; O=C(COc1ccc(CO)cc1)N1CC[C@H](Nc2ccccc2C(F)(F)F)C1 ; O=C(COc1ccc(CO)cc1)N1CC[C@@H](Nc2ccccc2C(F)(F)F)C1 ; O=C(COc1ccc(CO)cc1)N1CCC(Nc2cccc(C(F)(F)F)c2)C1] 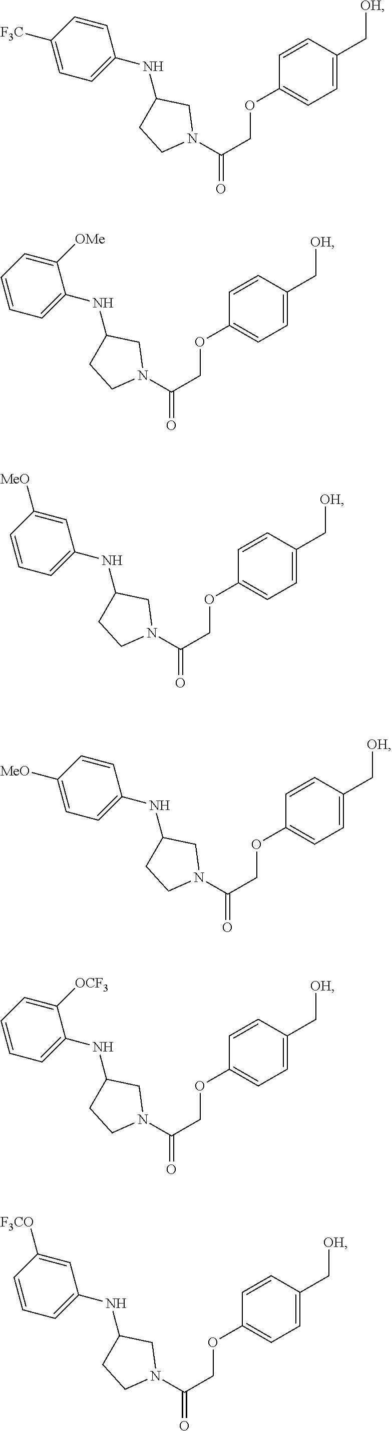 [SMILES: O=C(COc1ccc(CO)cc1)N1CCC(Nc2ccc(C(F)(F)F)cc2)C1 ; COc1ccccc1NC1CCN(C(=O)COc2ccc(CO)cc2)C1 ; COc1cccc(NC2CCN(C(=O)COc3ccc(CO)cc3)C2)c1 ; COc1ccc(NC2CCN(C(=O)COc3ccc(CO)cc3)C2)cc1 ; O=C(COc1ccc(CO)cc1)N1CCC(Nc2ccccc2OC(F)(F)F)C1 ; O=C(COc1ccc(CO)cc1)N1CCC(Nc2cccc(OC(F)(F)F)c2)C1] 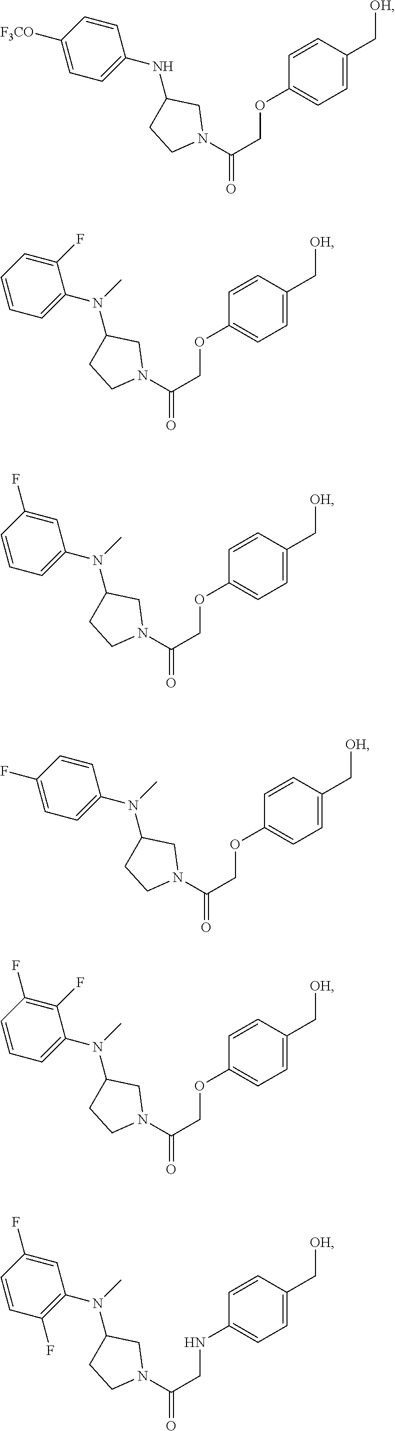 [SMILES: O=C(COc1ccc(CO)cc1)N1CCC(Nc2ccc(OC(F)(F)F)cc2)C1 ; CN(c1ccccc1F)C1CCN(C(=O)COc2ccc(CO)cc2)C1 ; CN(c1cccc(F)c1)C1CCN(C(=O)COc2ccc(CO)cc2)C1 ; CN(c1ccc(F)cc1)C1CCN(C(=O)COc2ccc(CO)cc2)C1 ; CN(c1cccc(F)c1F)C1CCN(C(=O)COc2ccc(CO)cc2)C1 ; CN(c1cc(F)ccc1F)C1CCN(C(=O)CNc2ccc(CO)cc2)C1] 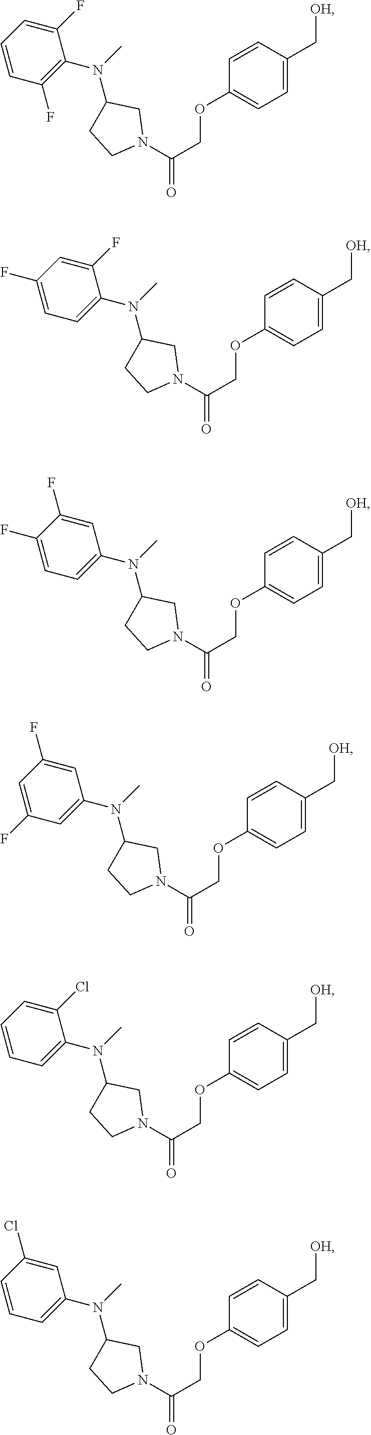 [SMILES: CN(c1c(F)cccc1F)C1CCN(C(=O)COc2ccc(CO)cc2)C1 ; CN(c1ccc(F)cc1F)C1CCN(C(=O)COc2ccc(CO)cc2)C1 ; CN(c1ccc(F)c(F)c1)C1CCN(C(=O)COc2ccc(CO)cc2)C1 ; CN(c1cc(F)cc(F)c1)C1CCN(C(=O)COc2ccc(CO)cc2)C1 ; CN(c1ccccc1Cl)C1CCN(C(=O)COc2ccc(CO)cc2)C1 ; CN(c1cccc(Cl)c1)C1CCN(C(=O)COc2ccc(CO)cc2)C1] 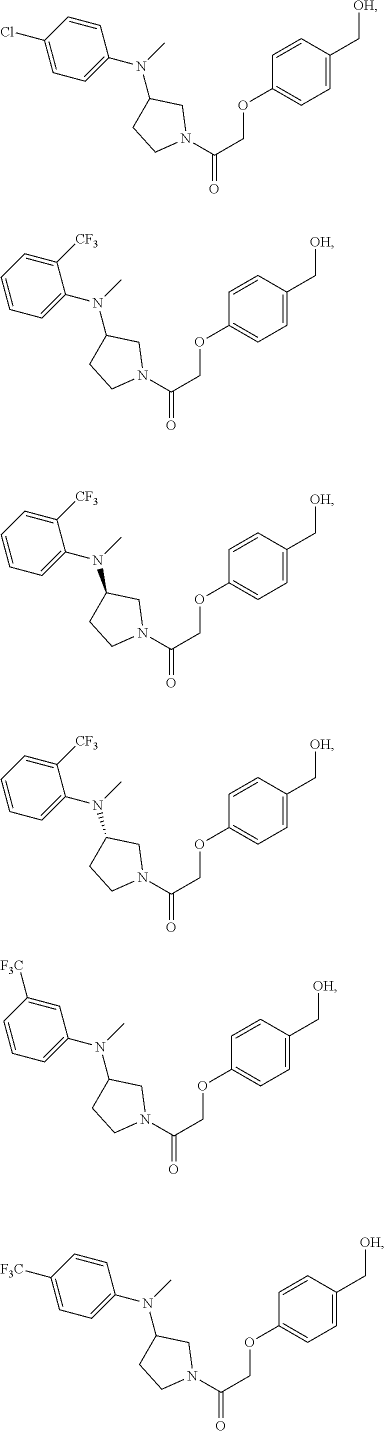 [SMILES: CN(c1ccc(Cl)cc1)C1CCN(C(=O)COc2ccc(CO)cc2)C1 ; CN(c1ccccc1C(F)(F)F)C1CCN(C(=O)COc2ccc(CO)cc2)C1 ; CN(c1ccccc1C(F)(F)F)[C@@H]1CCN(C(=O)COc2ccc(CO)cc2)C1 ; CN(c1ccccc1C(F)(F)F)[C@H]1CCN(C(=O)COc2ccc(CO)cc2)C1 ; CN(c1cccc(C(F)(F)F)c1)C1CCN(C(=O)COc2ccc(CO)cc2)C1 ; CN(c1ccc(C(F)(F)F)cc1)C1CCN(C(=O)COc2ccc(CO)cc2)C1] 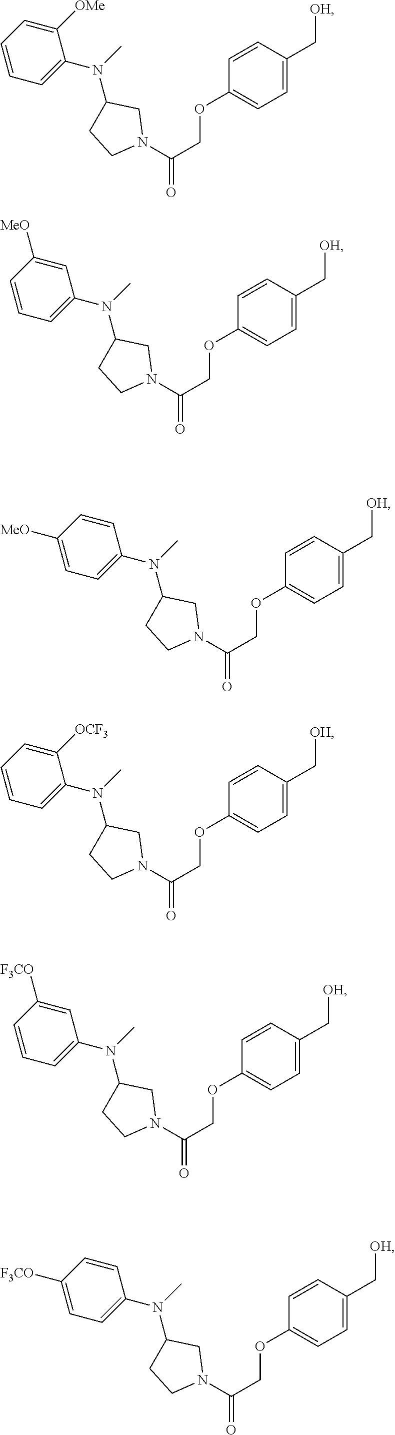 [SMILES: COc1ccccc1N(C)C1CCN(C(=O)COc2ccc(CO)cc2)C1 ; COc1cccc(N(C)C2CCN(C(=O)COc3ccc(CO)cc3)C2)c1 ; COc1ccc(N(C)C2CCN(C(=O)COc3ccc(CO)cc3)C2)cc1 ; CN(c1ccccc1OC(F)(F)F)C1CCN(C(=O)COc2ccc(CO)cc2)C1 ; CN(c1cccc(OC(F)(F)F)c1)C1CCN(C(=O)COc2ccc(CO)cc2)C1 ; CN(c1ccc(OC(F)(F)F)cc1)C1CCN(C(=O)COc2ccc(CO)cc2)C1] 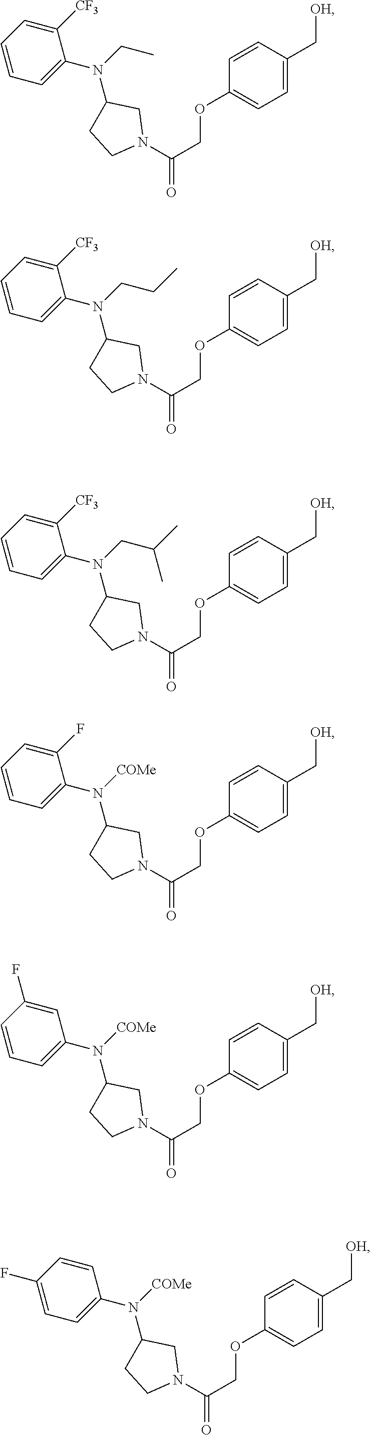 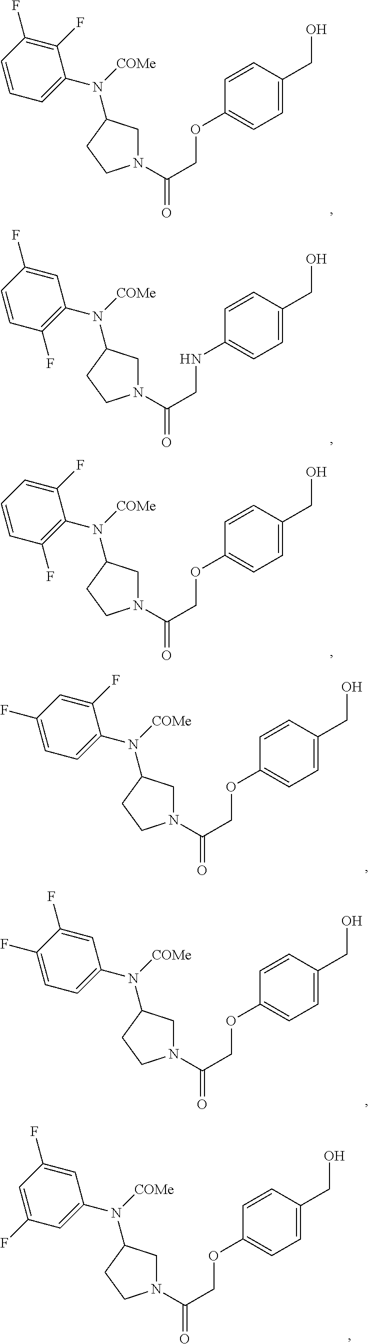 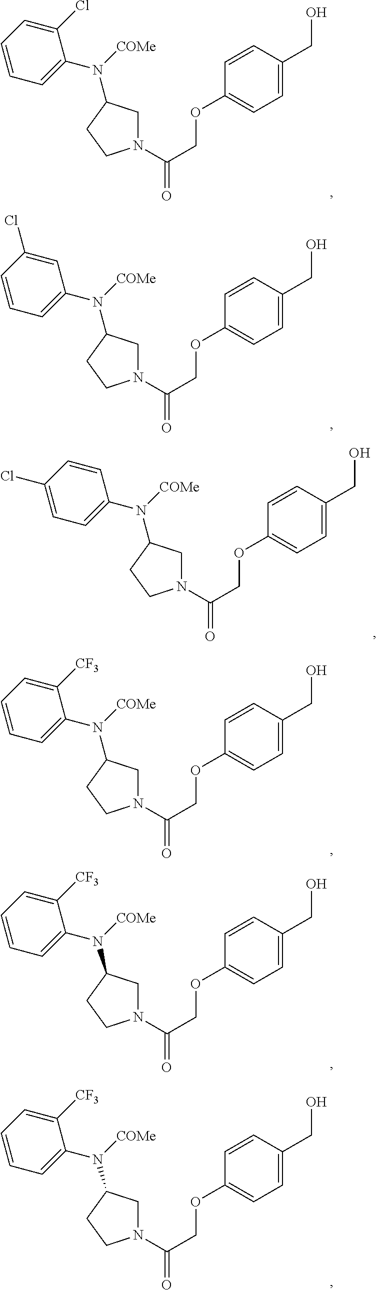 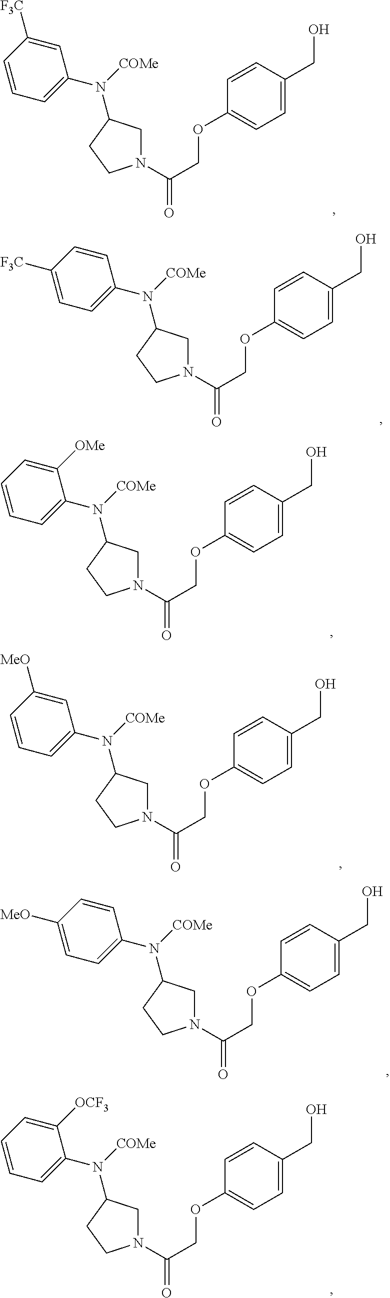 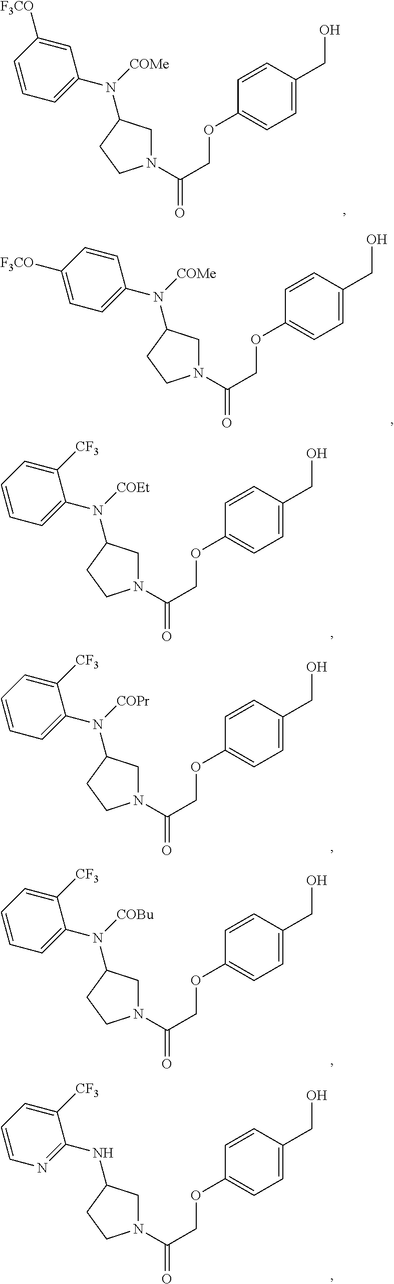 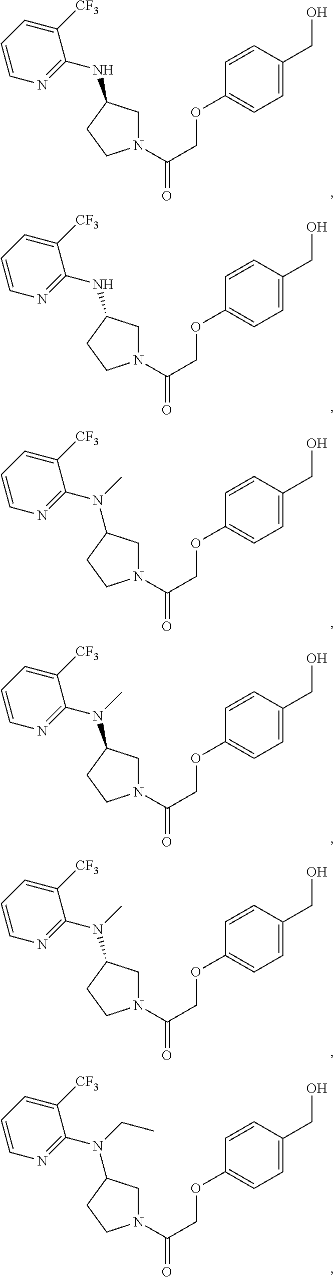 [SMILES: O=C(COc1ccc(CO)cc1)N1CC[C@@H](Nc2ncccc2C(F)(F)F)C1 ; O=C(COc1ccc(CO)cc1)N1CC[C@H](Nc2ncccc2C(F)(F)F)C1 ; CN(c1ncccc1C(F)(F)F)C1CCN(C(=O)COc2ccc(CO)cc2)C1 ; CN(c1ncccc1C(F)(F)F)[C@@H]1CCN(C(=O)COc2ccc(CO)cc2)C1 ; CN(c1ncccc1C(F)(F)F)[C@H]1CCN(C(=O)COc2ccc(CO)cc2)C1 ; CCN(c1ncccc1C(F)(F)F)C1CCN(C(=O)COc2ccc(CO)cc2)C1] 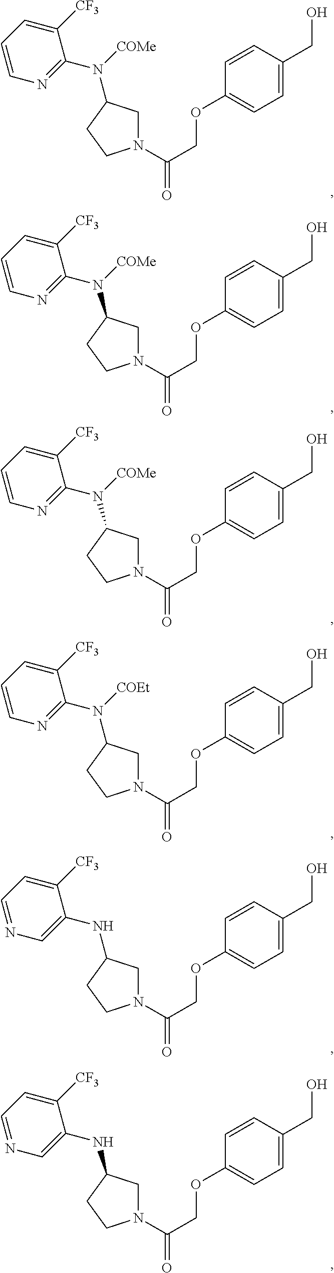 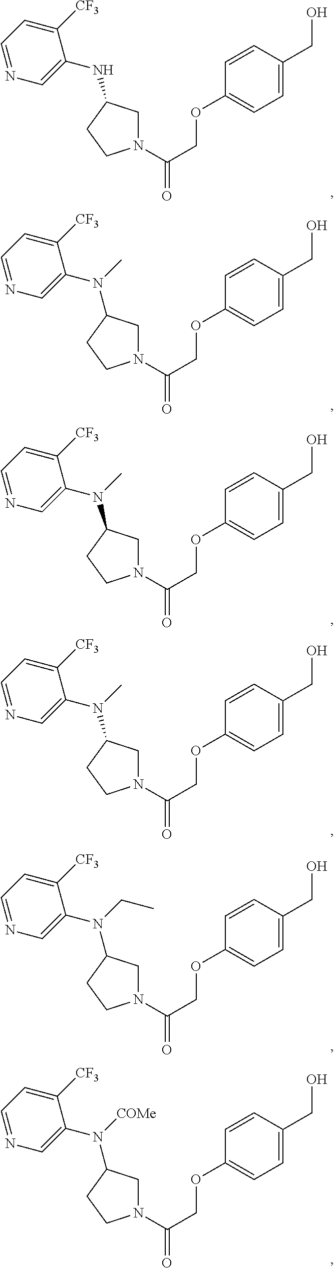 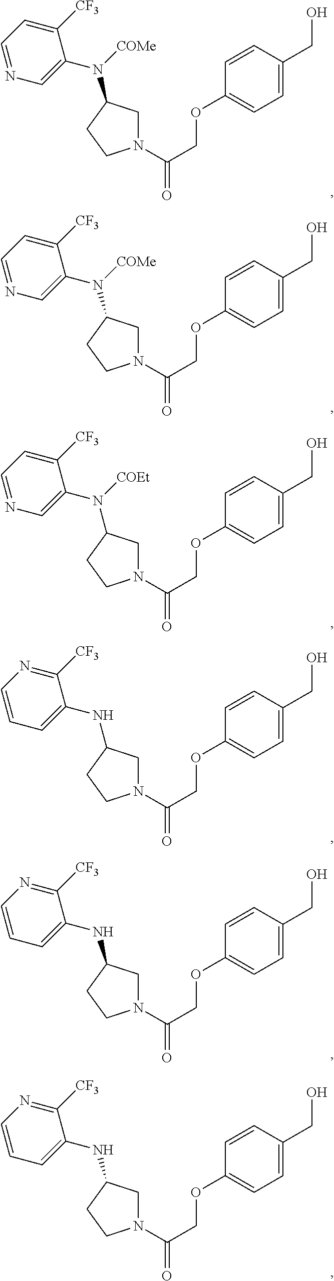 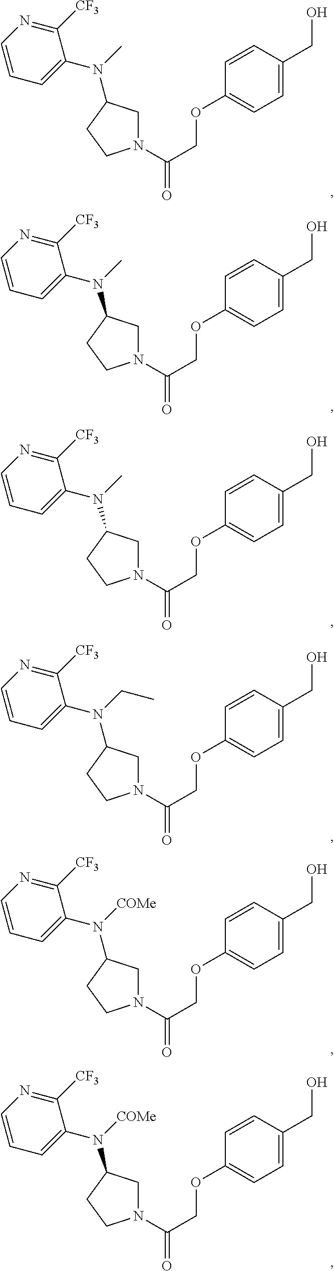 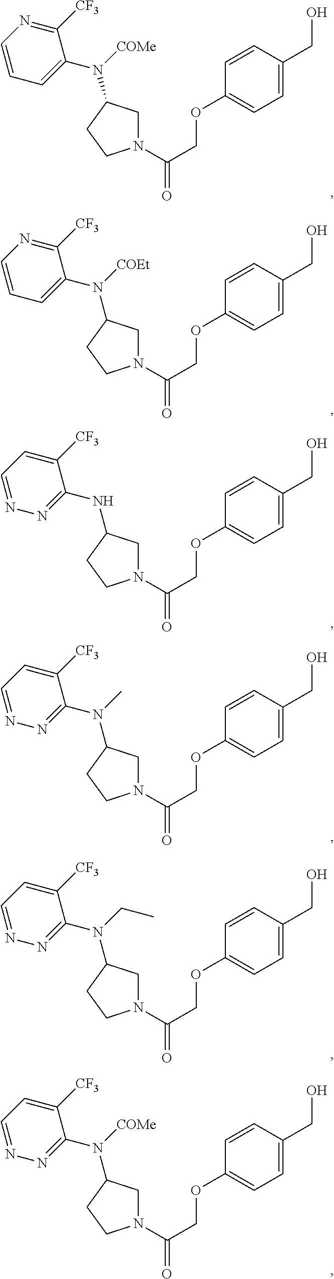 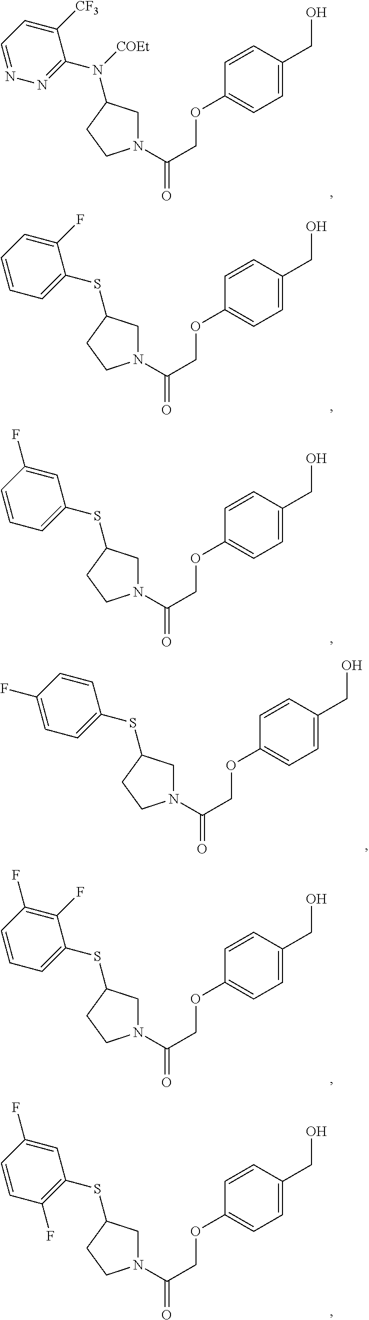 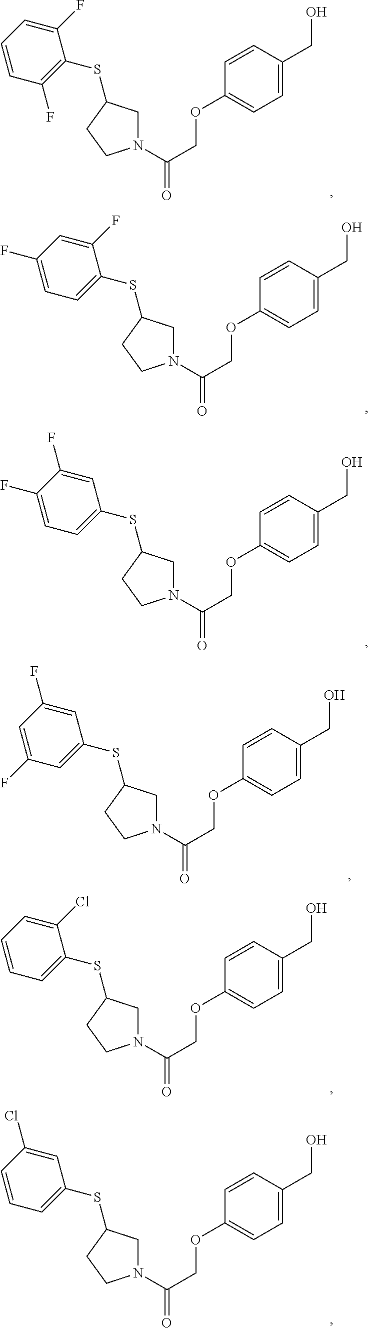 [SMILES: O=C(COc1ccc(CO)cc1)N1CCC(Sc2c(F)cccc2F)C1 ; O=C(COc1ccc(CO)cc1)N1CCC(Sc2ccc(F)cc2F)C1 ; O=C(COc1ccc(CO)cc1)N1CCC(Sc2ccc(F)c(F)c2)C1 ; O=C(COc1ccc(CO)cc1)N1CCC(Sc2cc(F)cc(F)c2)C1 ; O=C(COc1ccc(CO)cc1)N1CCC(Sc2ccccc2Cl)C1 ; O=C(COc1ccc(CO)cc1)N1CCC(Sc2cccc(Cl)c2)C1] 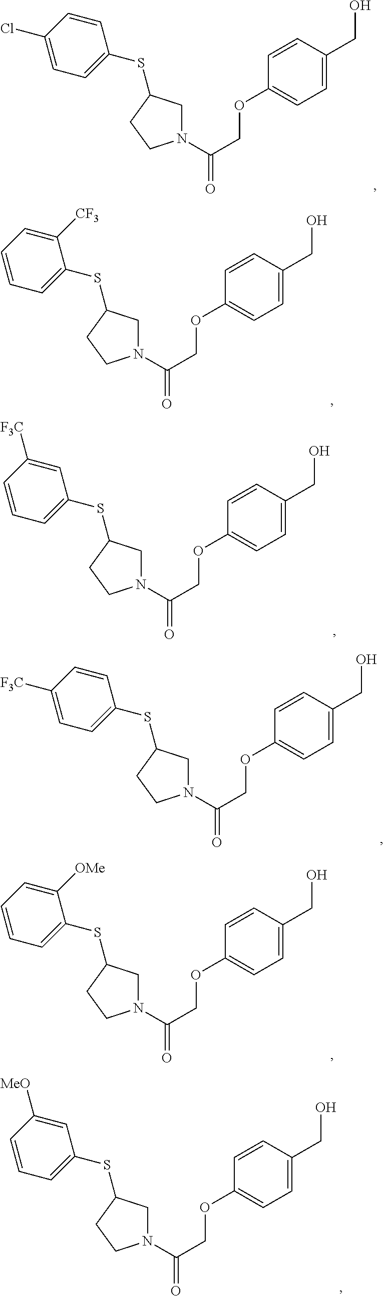 [SMILES: O=C(COc1ccc(CO)cc1)N1CCC(Sc2ccc(Cl)cc2)C1 ; O=C(COc1ccc(CO)cc1)N1CCC(Sc2ccccc2C(F)(F)F)C1 ; O=C(COc1ccc(CO)cc1)N1CCC(Sc2cccc(C(F)(F)F)c2)C1 ; O=C(COc1ccc(CO)cc1)N1CCC(Sc2ccc(C(F)(F)F)cc2)C1 ; COc1ccccc1SC1CCN(C(=O)COc2ccc(CO)cc2)C1 ; COc1cccc(SC2CCN(C(=O)COc3ccc(CO)cc3)C2)c1] 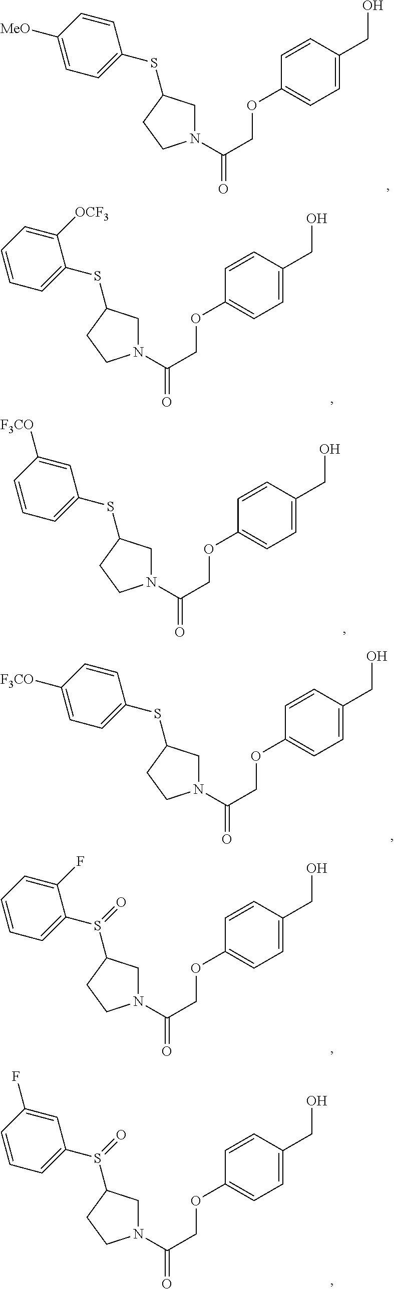 [SMILES: COc1ccc(SC2CCN(C(=O)COc3ccc(CO)cc3)C2)cc1 ; O=C(COc1ccc(CO)cc1)N1CCC(Sc2ccccc2OC(F)(F)F)C1 ; O=C(COc1ccc(CO)cc1)N1CCC(Sc2cccc(OC(F)(F)F)c2)C1 ; O=C(COc1ccc(CO)cc1)N1CCC(Sc2ccc(OC(F)(F)F)cc2)C1 ; O=C(COc1ccc(CO)cc1)N1CCC(S(=O)c2ccccc2F)C1 ; O=C(COc1ccc(CO)cc1)N1CCC(S(=O)c2cccc(F)c2)C1] 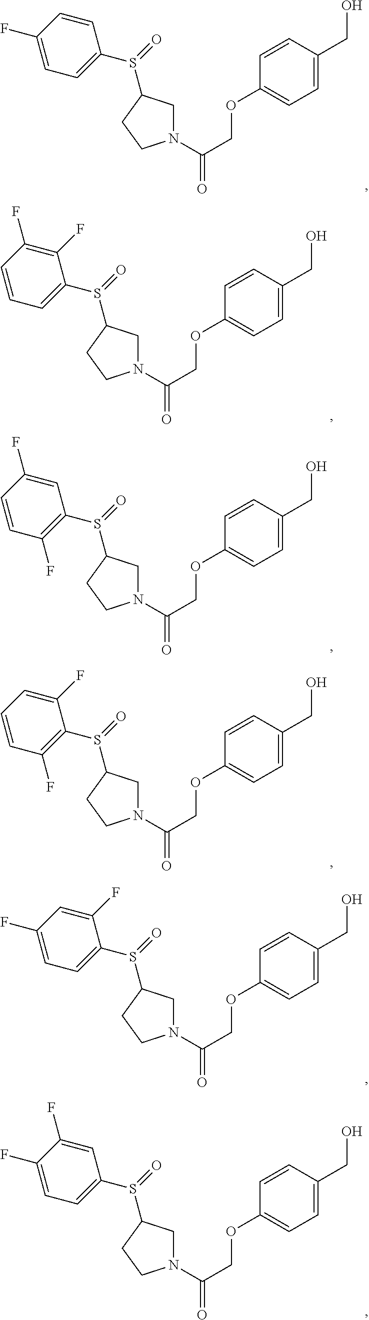 [SMILES: O=C(COc1ccc(CO)cc1)N1CCC(S(=O)c2ccc(F)cc2)C1 ; O=C(COc1ccc(CO)cc1)N1CCC(S(=O)c2cccc(F)c2F)C1 ; O=C(COc1ccc(CO)cc1)N1CCC(S(=O)c2cc(F)ccc2F)C1 ; O=C(COc1ccc(CO)cc1)N1CCC(S(=O)c2c(F)cccc2F)C1 ; O=C(COc1ccc(CO)cc1)N1CCC(S(=O)c2ccc(F)cc2F)C1 ; O=C(COc1ccc(CO)cc1)N1CCC(S(=O)c2ccc(F)c(F)c2)C1] 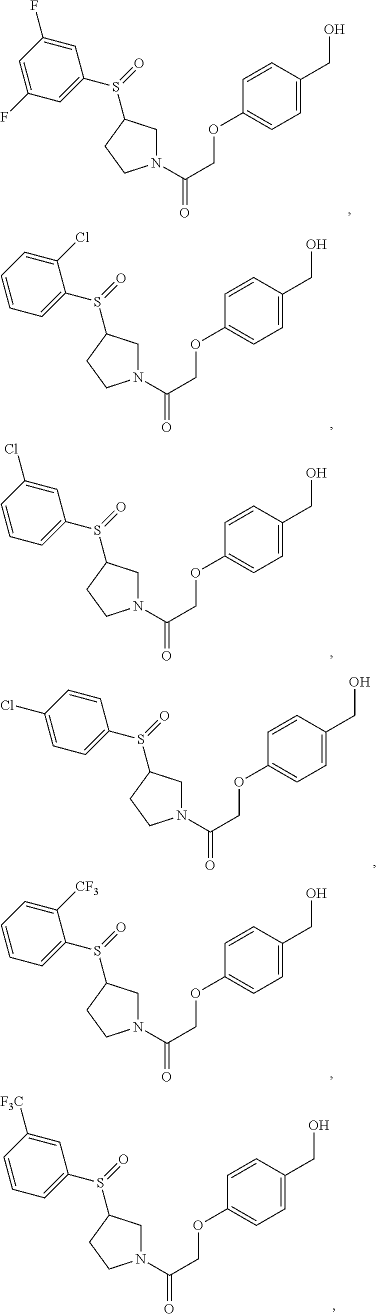 [SMILES: O=C(COc1ccc(CO)cc1)N1CCC(S(=O)c2cc(F)cc(F)c2)C1 ; O=C(COc1ccc(CO)cc1)N1CCC(S(=O)c2ccccc2Cl)C1 ; O=C(COc1ccc(CO)cc1)N1CCC(S(=O)c2cccc(Cl)c2)C1 ; O=C(COc1ccc(CO)cc1)N1CCC(S(=O)c2ccc(Cl)cc2)C1 ; O=C(COc1ccc(CO)cc1)N1CCC(S(=O)c2ccccc2C(F)(F)F)C1 ; O=C(COc1ccc(CO)cc1)N1CCC(S(=O)c2cccc(C(F)(F)F)c2)C1] 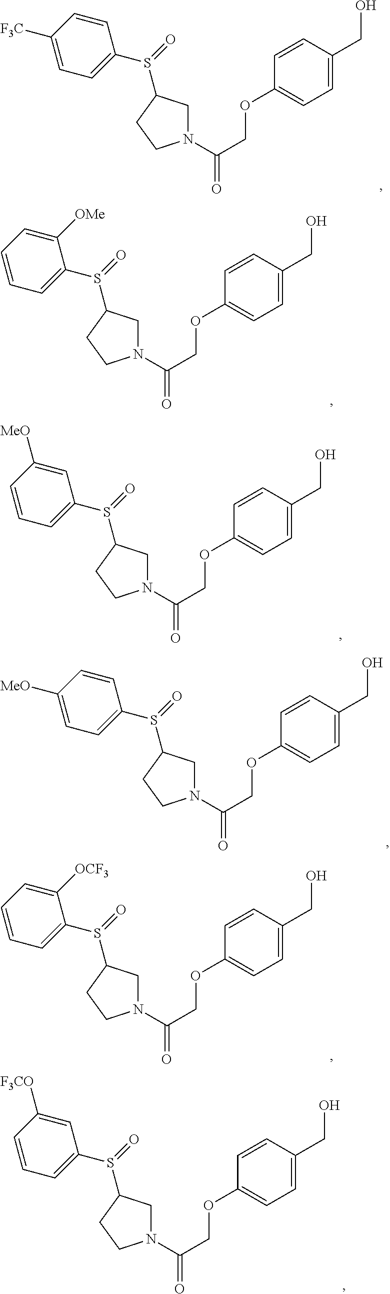 [SMILES: O=C(COc1ccc(CO)cc1)N1CCC(S(=O)c2ccc(C(F)(F)F)cc2)C1 ; COc1ccccc1S(=O)C1CCN(C(=O)COc2ccc(CO)cc2)C1 ; COc1cccc(S(=O)C2CCN(C(=O)COc3ccc(CO)cc3)C2)c1 ; COc1ccc(S(=O)C2CCN(C(=O)COc3ccc(CO)cc3)C2)cc1 ; O=C(COc1ccc(CO)cc1)N1CCC(S(=O)c2ccccc2OC(F)(F)F)C1 ; O=C(COc1ccc(CO)cc1)N1CCC(S(=O)c2cccc(OC(F)(F)F)c2)C1] 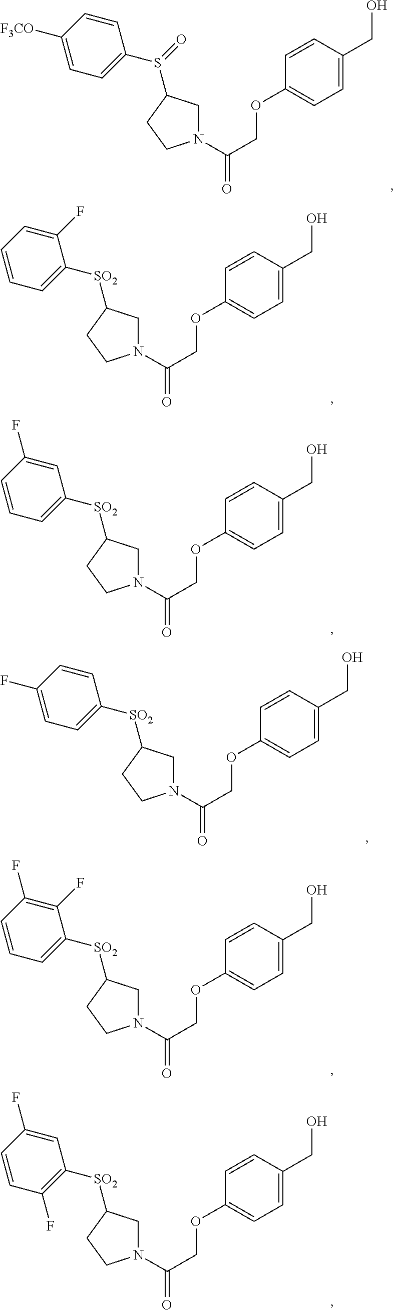 [SMILES: O=C(COc1ccc(CO)cc1)N1CCC(S(=O)c2ccc(OC(F)(F)F)cc2)C1 ; O=C(COc1ccc(CO)cc1)N1CCC(S(=O)(=O)c2ccccc2F)C1 ; O=C(COc1ccc(CO)cc1)N1CCC(S(=O)(=O)c2cccc(F)c2)C1 ; O=C(COc1ccc(CO)cc1)N1CCC(S(=O)(=O)c2ccc(F)cc2)C1 ; O=C(COc1ccc(CO)cc1)N1CCC(S(=O)(=O)c2cccc(F)c2F)C1 ; O=C(COc1ccc(CO)cc1)N1CCC(S(=O)(=O)c2cc(F)ccc2F)C1] 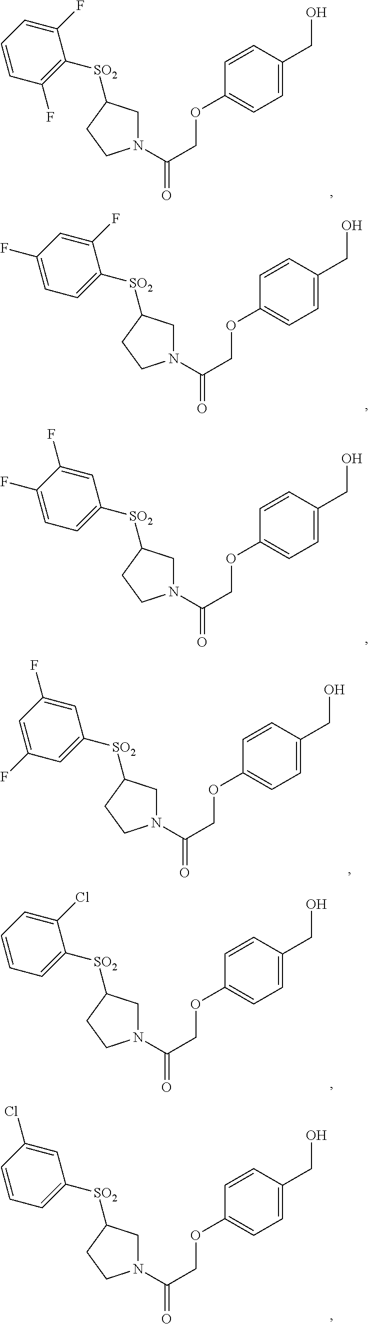 [SMILES: O=C(COc1ccc(CO)cc1)N1CCC(S(=O)(=O)c2c(F)cccc2F)C1 ; O=C(COc1ccc(CO)cc1)N1CCC(S(=O)(=O)c2ccc(F)cc2F)C1 ; O=C(COc1ccc(CO)cc1)N1CCC(S(=O)(=O)c2ccc(F)c(F)c2)C1 ; O=C(COc1ccc(CO)cc1)N1CCC(S(=O)(=O)c2cc(F)cc(F)c2)C1 ; O=C(COc1ccc(CO)cc1)N1CCC(S(=O)(=O)c2ccccc2Cl)C1 ; O=C(COc1ccc(CO)cc1)N1CCC(S(=O)(=O)c2cccc(Cl)c2)C1] 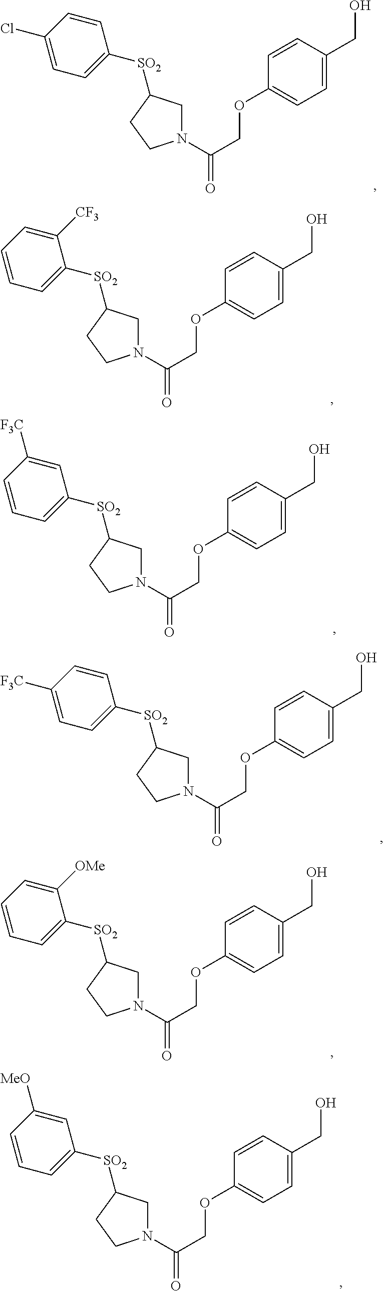 [SMILES: O=C(COc1ccc(CO)cc1)N1CCC(S(=O)(=O)c2ccc(Cl)cc2)C1 ; O=C(COc1ccc(CO)cc1)N1CCC(S(=O)(=O)c2ccccc2C(F)(F)F)C1 ; O=C(COc1ccc(CO)cc1)N1CCC(S(=O)(=O)c2cccc(C(F)(F)F)c2)C1 ; O=C(COc1ccc(CO)cc1)N1CCC(S(=O)(=O)c2ccc(C(F)(F)F)cc2)C1 ; COc1ccccc1S(=O)(=O)C1CCN(C(=O)COc2ccc(CO)cc2)C1 ; COc1cccc(S(=O)(=O)C2CCN(C(=O)COc3ccc(CO)cc3)C2)c1] 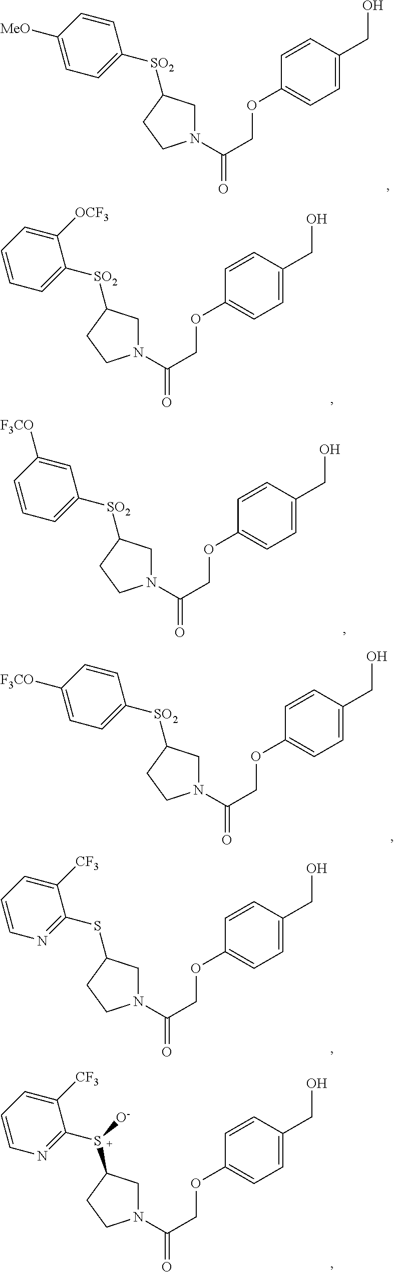 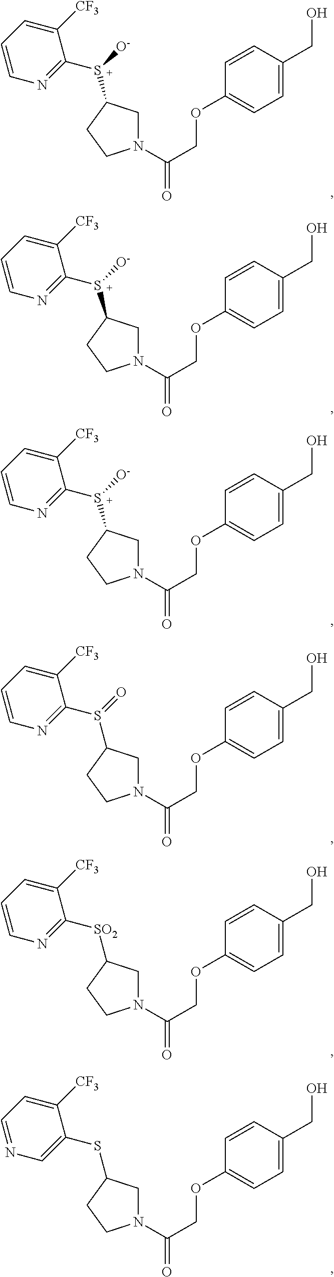 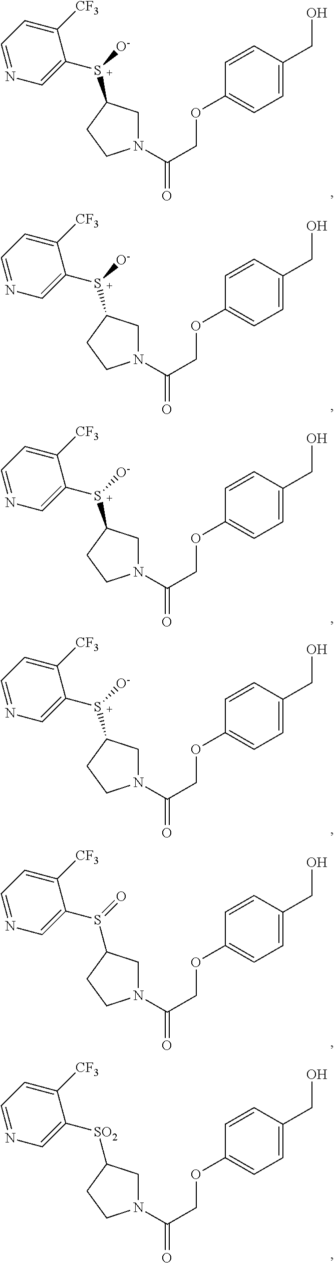 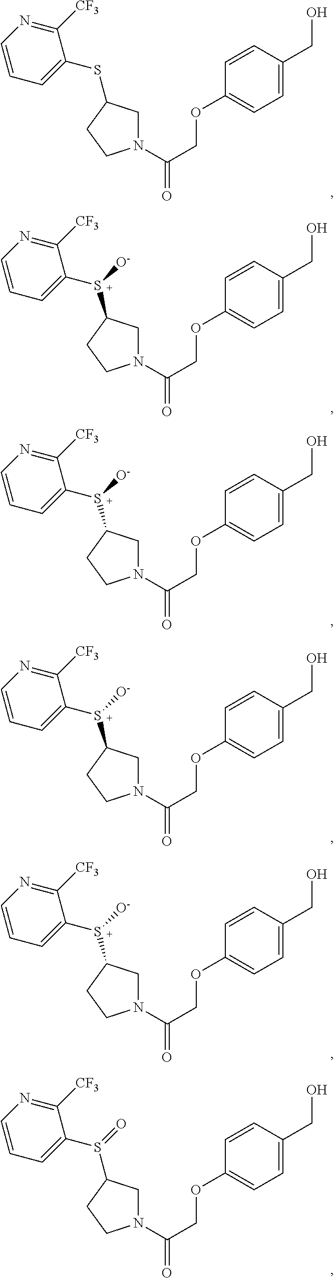 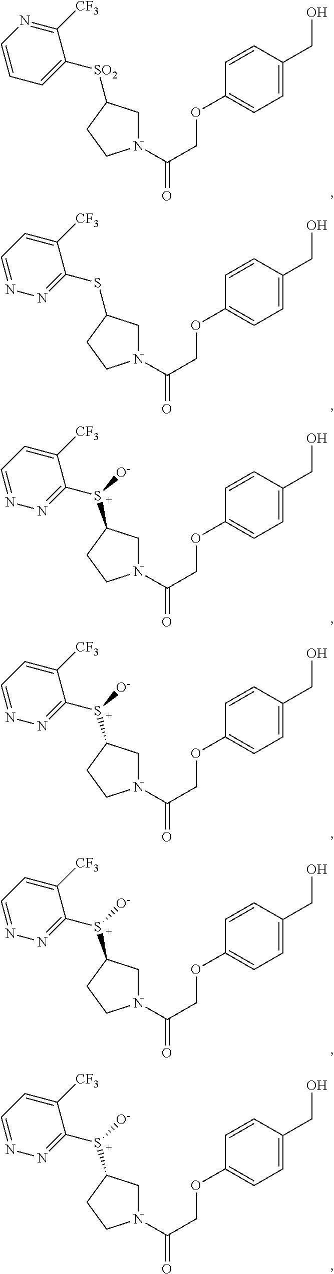 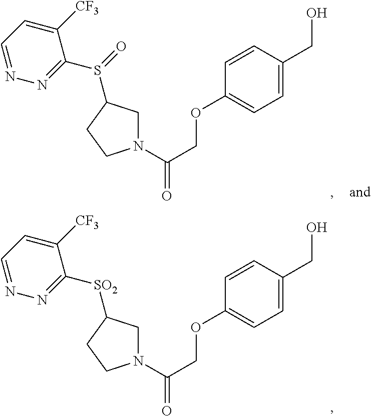 [SMILES: O=C(COc1ccc(CO)cc1)N1CCC(S(=O)c2nnccc2C(F)(F)F)C1 ; O=C(COc1ccc(CO)cc1)N1CCC(S(=O)(=O)c2nnccc2C(F)(F)F)C1]
or a pharmaceutically acceptable salt thereof.

28. The method according to any one of claims 1-27, wherein the SCD-1 inhibitor of Formula I is: 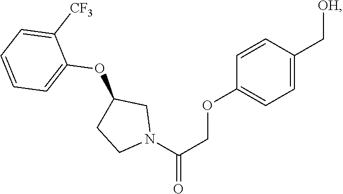 [SMILES: O=C(COc1ccc(CO)cc1)N1CC[C@@H](Oc2ccccc2C(F)(F)F)C1]
or a pharmaceutically acceptable salt thereof.
